(19) United States
(12) Patent Application Publication
(10) Pub. No.: US 2006/0211698 A1
(43) Pub. Date: Sep. 21, 2006

(54) Bicyclic heteroaryl derivatives for treating viruses

(75) Inventors: Janos Botyanszki, Fremont, CA (US); Christopher Don Roberts, Belmont, CA (US); Franz Ulrich Schmitz, Mill Valley, CA (US); Joshua Michael Gralapp, Sunnyvale, CA (US); Ronald Conrad Griffith, Escondido, CA (US); Dong-Fang Shi, Fremont, CA (US); Martin Robert Leivers, San Francisco, CA (US); Rachel Elizabeth Brewster, Menlo Park, CA (US)

Correspondence Address: FOLEY & LARDNER LLP, 1530 PAGE MILL ROAD, PALO ALTO, CA, 94304, US

(73) Assignee: Genelabs, Inc.

(21) Appl. No.: 11/331,578

(22) Filed: Jan. 13, 2006

Related U.S. Application Data
(60) Provisional application No. 60/644,343, filed on Jan. 14, 2005.

(51) Int. Cl. A61K 31/498; A61K 31/4745; A61K 31/4709; A61K 31/4184; C07D 471/02; C07D 403/02

(52) U.S. Cl. 514/249; 514/300; 514/314; 514/394; 544/333; 546/167; 546/122; 548/305.4

(57) ABSTRACT

Disclosed are compounds having formula I and related compositions and methods thereof. The compounds are useful for treating viral infections caused by the Flaviviridae family of viruses.

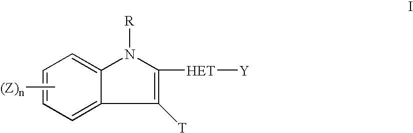

### CROSS-REFERENCE TO RELATED APPLICATION

This application claims the benefit under 35 U.S.C. 119(e) to co-pending provisional application U.S. Ser. No. 60/644,343 filed on Jan. 14, 2005, which is incorporated herein by reference in its entirety.

### BACKGROUND OF THE INVENTION

### Field of the Invention

The invention relates to the field of pharmaceutical chemistry, in particular to indole compounds, compositions, and methods for treating viral infections in mammals mediated, at least in part, by a virus in the Flaviviridae family of viruses.

### REFERENCES

The following publications are cited in this application as superscript numbers:
1. Szabo, et al., Pathol. Oncol. Res. 2003, 9:215-221.
2. Hoofnagle J H, Hepatology 1997, 26:15S-20S.
3. Thomson B J and Finch R G, Clin Microbial Infect. 2005, 11:86-94.
4. Moriishi K and Matsuura Y, Antivir. Chem. Chemother. 2003, 14:285-297.
5. Fried, et al. N. Engl. J Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, et al., Ann. Rep. Med. Chem 39, 223-237, 2004.
9. Sommadossi, et al., International Patent Application Publication No. WO01/90121, published May 23, 2001
10. Olson et al., Antimicrob Agents Chemother. 2004, 48:3944-53
11. Sarisky R. T. J Antimicrob Chemother. 2004, 54:14-6
12. Love et al., J Virol. 2003, 77:7575-81
13. Harper et al., J Med Chem. 2005, 48:4547-57
14. Hiromasa et al., U.S. Pat. No. 6,770,666 issued Aug. 3, 2004
15. Watashi, et al, Molecular Cell, 19, 111-122, 2005
16. Horsmans, et al., Hepatology, 42, 724-731, 2005

All of the above publications are herein incorporated by reference in their entirety to the same extent as if each individual publication was specifically and individually indicated to be incorporated by reference in its entirety.

### State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2—NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursuit to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4A protease/helicase and the NS5b RNA-dependent RNA polymerase are considered the most promising viral targets for new drugs.⁶⁻⁸

The NS5b RNA-dependent RNA polymerase in particular has been shown to be amenable to small-molecule inhibition. Besides several nucleoside inhibitors,^{9,10} at least three allosteric sites have been described,⁷ along with multiple inhibitor scaffolds.¹¹⁻¹⁴

Besides targeting viral genes and their transcription and translation products, antiviral activity can also be achieved by targeting host cell proteins that are necessary for viral replication. For example, Watashi et al.¹⁵ show how antiviral activity can be achieved by inhibiting host cell cyclophilins. Alternatively, a potent TLR7 agonist has been shown to reduce HCV plasma levels in humans.¹⁶

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

This invention is directed to indole compounds, compositions, and methods that are useful in the treatment of viral infections in mammals mediated at least in part by a member of the Flaviviridae family viruses such as HCV. Compounds of this invention maybe used alone or in combination with other compounds to treat viruses.

### DETAILED DESCRIPTION OF THE INVENTION

Throughout this application, the text refers to various embodiments of the present compounds, compositions, and methods. The various embodiments described are meant to provide a variety illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

Accordingly, the present invention provides a compound having formula I 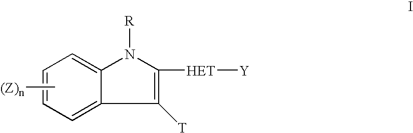
wherein:

HET is selected from arylene, substituted arylene, heteroarylene, and substituted heteroarylene;

Y is selected from substituted aryl and substituted heteroaryl;

n is an integer from 1 to 4;

Z is selected from:
(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;
(b) carboxy and carboxy ester;
(c) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(d) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted with one to three groups selected from halo, hydroxy, carboxy, carboxy ester, alkyl, alkoxy, amino, and substituted amino; or alternatively, R²¹ and R²² or R²² and R²³ together with the atoms bound thereto join together to form an optionally substituted heterocyclic group;
(e) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R² are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group;
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(f) —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(g) carboxylic acid isostere, wherein said isostere is not as defined in (a)-(f);
   R is hydrogen, alkyl, substituted alkyl, cycloalkyl, substituted cycloalkyl, heterocyclic, substituted heterocyclic, aryl, substituted aryl, heteroaryl, substituted heteroaryl;
   T is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, and —NR¹⁴R¹⁵;
      where each R¹⁴ and R¹⁵ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or alternatively, R¹⁴ and R¹⁵ may optionally be joined together with the nitrogen atom bound thereto to form a heterocyclic, substituted heterocyclic, heteroaryl or substituted heteroaryl; or
         pharmaceutically acceptable salts, partial salts or tautomers thereof.

In another embodiment, the invention provides a compound of formula Ia: 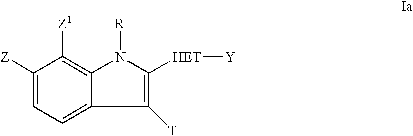
wherein:

Y is selected from the group consisting of substituted aryl and substituted heteroaryl;

HET is selected from the group consisting of a 6-membered arylene ring, a 6-membered heteroarylene ring containing 1, 2, or 3 heteroatoms selected from N, O, or S, and a bicyclic ring having the formula 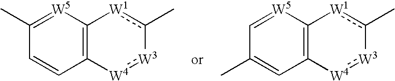
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;

R is selected from the group consisting of hydrogen, alkyl, and substituted alkyl;

T is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl;

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted with one to three groups selected from halo, hydroxy, carboxy, carboxy ester, alkyl, alkoxy, amino, and substituted amino; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere, wherein said isostere is not as defined in (a)-(e);
Z¹ is selected from the group consisting of hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino; or a
   pharmaceutically acceptable salt, partial salt, or tautomer thereof.

In another embodiment the invention provides a compound having formula Ib: 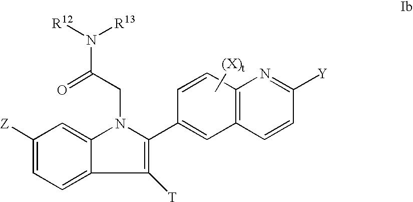
wherein:

Y is selected from the group consisting of substituted aryl and substituted heteroaryl;

X is independently selected from the group consisting of amino, nitro, alkyl, haloalkyl, and halo;

t is an integer equal to 0, 1 or 2;

T is selected from the group consisting of cyclohexyl and cyclopentyl;

R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms;
Z is selected from the group consisting of carboxy, carboxy ester, and a carboxylic acid isostere; or a
   pharmaceutically acceptable salt, partial salt, or tautomer thereof.

In other embodiments, the present invention provides compounds of formulae Ic-Il: 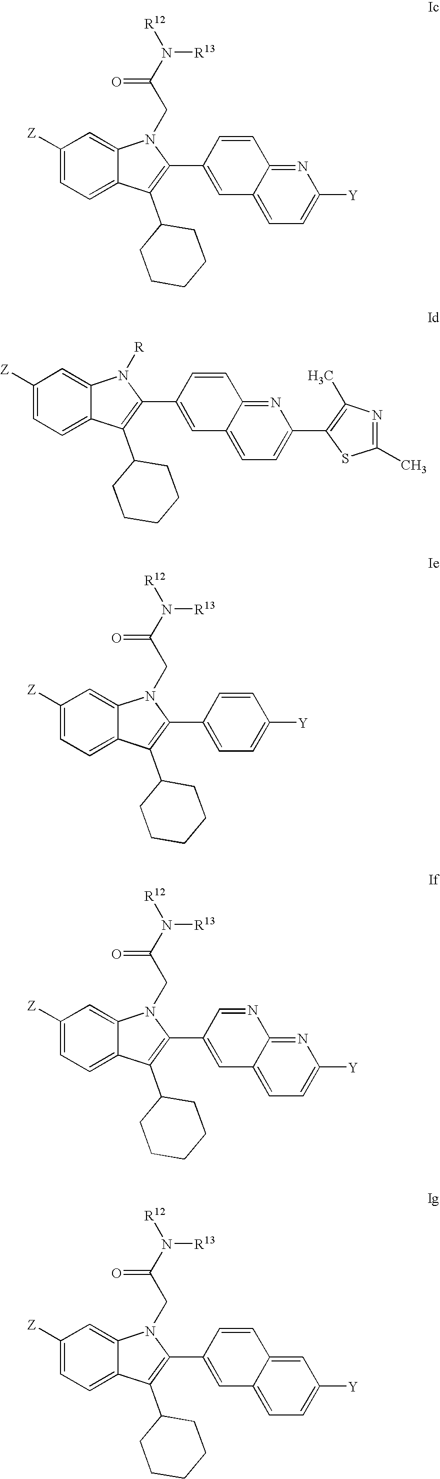 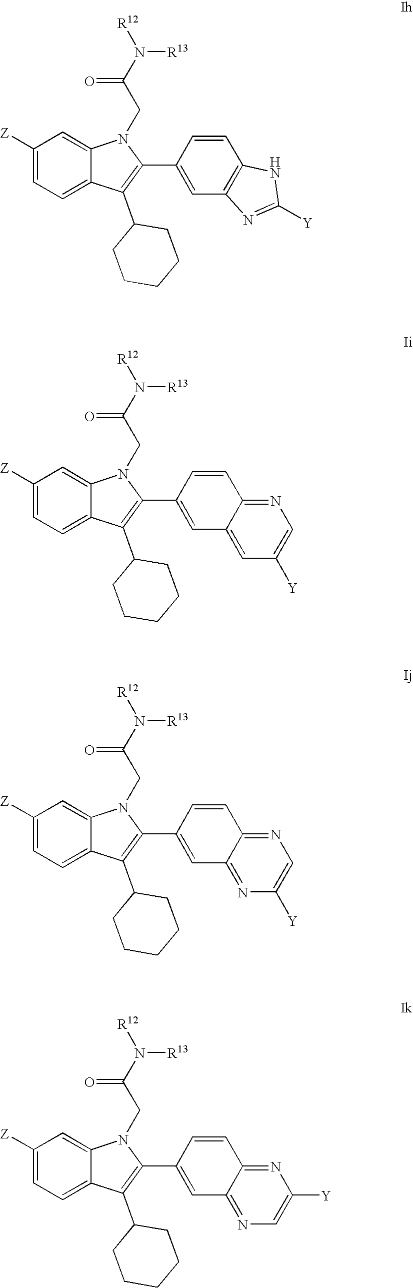
wherein Z, R, and Y are as previously defined in formula Ia and R¹² and R¹³ are as previously defined for formula Ib.

In other embodiments, the present invention provides compounds of formulae II and IIa-IIk: 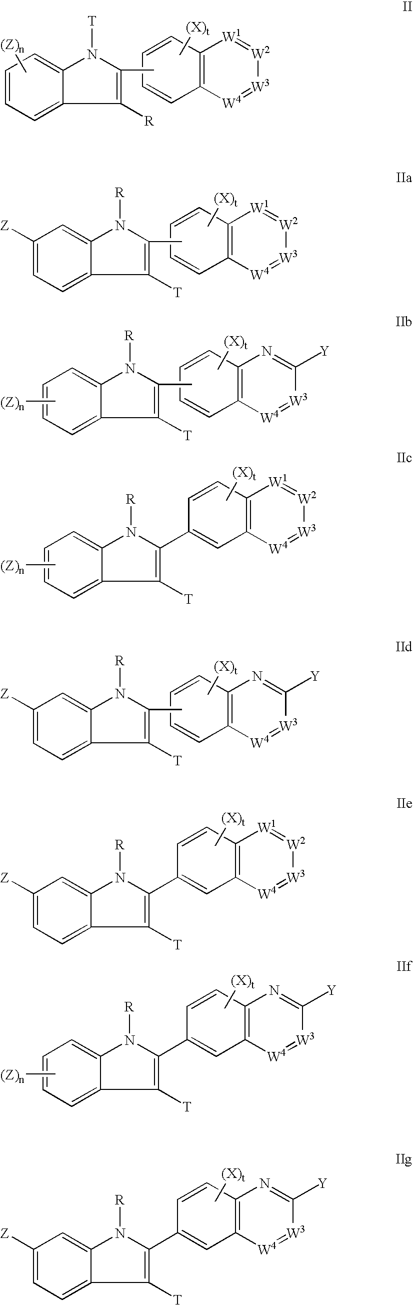 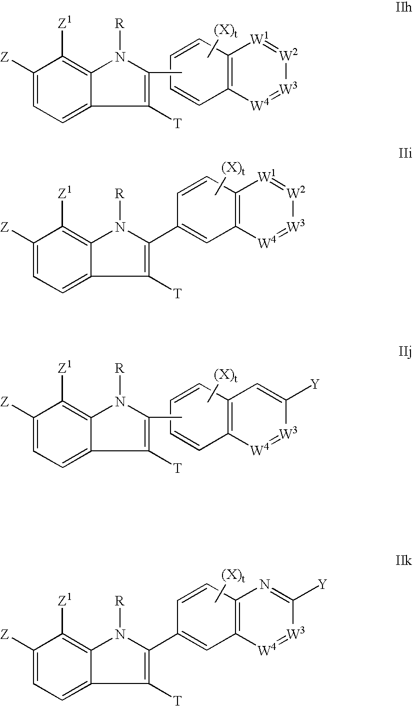
wherein Y, Z, T, R and n are as defined above for formula I; each W¹, W², W³ and W⁴ are independently selected from N, CH, and C—Y, provided that no more than 2 of W¹, W², W³ and W⁴ are N, and further wherein no more than one N in the ring system is optionally oxidized to form the N-oxide; where Z¹ is selected from halo, alkyl, substituted alkyl, alkenyl, substituted alkenyl, alkoxy, substituted alkoxy, cyano, aryl, substituted aryl, heteroaryl, substituted heteroaryl, amino and substituted amino; X is chosen from alkyl, substituted alkyl, alkoxy, substituted alkoxy, amino, substituted amino, halo, hydroxy, and nitro; t is an integer equal to 0, 1 or 2

In other embodiments, the present invention provides compounds of formulae III and IIIa: 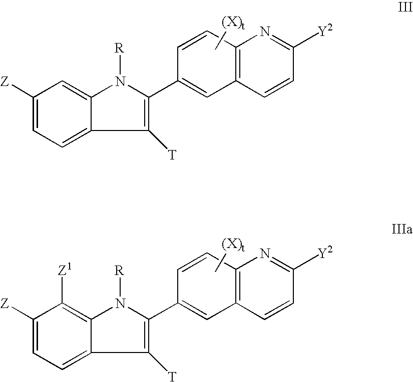
wherein:
Y² is Y, and Y is selected from optionally substituted aryl and optionally substituted heteroaryl;
Z is selected from:
(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;
(b) carboxy and carboxy ester;
(c) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(d) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted halo, hydroxy, carboxy, alkyl, alkoxy, amino, substituted amino; or alternatively, R²¹ and R²² or R²² and R²³ together with the atoms bound thereto join together to form an optionally substituted heterocyclic group;
(e) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group, or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(f) —C(X²)—N(R³)CR²⁵R²⁶R²⁷, wherein X² and R³ are defined above, and R²⁵, R²⁶ and R²⁷ are substituted aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(g) carboxylic acid isostere;
R is hydrogen, alkyl, substituted alkyl, cycloalkyl, substituted cycloalkyl, heterocyclic, substituted heterocyclic, aryl, substituted aryl, heteroaryl, substituted heteroaryl;
T is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, and —NR¹⁴R¹⁵;
   where each R¹⁴ and R¹⁵ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or alternatively, R¹⁴ and R¹⁵ may optionally be joined together with the nitrogen atom bound thereto to form a heterocyclic, substituted heterocyclic, heteroaryl or substituted heteroaryl;
Z¹ is selected from halo, alkyl, substituted alkyl, alkenyl, substituted alkenyl, alkoxy, substituted alkoxy, cyano, aryl, substituted aryl, heteroaryl, substituted heteroaryl, amino and substituted amino;
X is chosen from alkyl, substituted alkyl, alkoxy, substituted alkoxy, halo, hydroxy, and nitro;
t is an integer equal to 0, 1 or 2; or
   pharmaceutically acceptable salts, partial salts, or tautomers thereof.

In some embodiments of each of formula I-IIIa where appropriate, T is selected from the group consisting of hydrogen, alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl. In another embodiment, T is selected from the group consisting of hydrogen, alkyl, substituted alkyl, cycloalkyl, and substituted cycloalkyl. In another embodiment T is selected from the group consisting of hydrogen, ethyl, iso-propyl, sec-butyl, 3-methyl-n-butyl, cyclopropyl, cyclopentyl, cyclohexyl, cyclopropylmethyl, and 2-(N,N-dimethylamino)eth-1-yl. In another embodiment T is cycloalkyl. In another embodiment T is cyclohexyl. In yet another embodiment T is cyclopentyl.

In some embodiments of each of formula I-IIIa where appropriate, Z is carboxy or carboxy ester. In another embodiment Z is selected from —C(═O)OH, and —C(═O)OR″ where R″ is alkyl. In another embodiment Z is selected from carboxy, methyl carboxylate, and ethyl carboxylate. In yet another embodiment Z is —C(═O)OH.

In another embodiment Z is a carboxylic acid isostere. In another embodiment the carboxylic acid isostere is a carboxylic acid bioisostere. In another embodiment the carboxylic acid isostere is selected from 1H-tetrazol-5-yl and 5-oxo-4,5-dihydro-1,2,4-oxadiazol-3-yl.

In another embodiment Z is —C(═O)NR⁸R⁹ where R⁸ is hydrogen and R⁹ is selected from the group consisting of alkyl, substituted alkyl, aryl, substituted aryl, heteroaryl, substituted heteroaryl, heterocyclic and substituted heterocyclic. In another embodiment where Z is —C(═O)NR⁸R⁹ and R⁸ is hydrogen, R⁹ is substituted alkyl. In another embodiment where Z is —C(═O)NR⁸R⁹ and R⁸ is hydrogen, and R⁹ is substituted alkyl, the substituted alkyl comprises 1 to 2 substituents selected from the group consisting of sulfonic acid (SO₃H), carboxy, carboxy ester, amino, substituted amino, aryl, substituted aryl, heteroaryl and substituted heteroaryl. In another embodiment where Z is —C(═O)NR⁸R⁹ and R⁸ is hydrogen, and R⁹ is substituted alkyl, the substituted alkyl group is selected from the group consisting of 3,4-dimethoxybenzyl, 3,4-dihydroxybenzyl, 3-methoxy-4-hydroxybenzyl, 4-aminosulfonylbenzyl, 4-methylsulfonylbenzyl, (1-methyl-piperidin-3-yl)methyl, (1-methyl-pyrrolidin-3-yl)methyl, fur-2-ylmethyl, 6-methylpyridin-2-ylmethyl, 2-(1-methyl-pyrrolidin-3-yl)ethyl, 1-phenylethyl, 1-(3-methoxyphenyl)-ethyl, 1-(4-methoxyphenyl)-ethyl, N′,N′-dimethylaminoethyl, and 2-(1H-pyrazol-1-yl)ethyl.

In another embodiment Z is selected from N-methyl carboxamide, N,N-dimethylcarboxamido, N-isopropyl-carboxamido, N-allyl-carboxamido, and 5-hydroxytryptophan-carbonyl.

In another embodiment Z is —C(═O)NR⁸R⁹ wherein R⁹ is aryl or substituted aryl. In another embodiment where Z is —C(═O)NR⁸R⁹, R⁹ is substituted aryl. In another embodiment where Z is —C(═O)NR⁸R⁹, R⁹ is selected from the group consisting of 7-hydroxynaphth-1-yl, 6-hydroxynaphth-1-yl, 5-hydroxynaphth-1-yl, 6-carboxynaphth-2-yl, (4-HOOCCH₂-)phenyl, (3,4-dicarboxy)phenyl, 3-carboxyphenyl, 3-carboxy-4-hydroxyphenyl and 2-biphenyl.

In another embodiment Z is —C(═O)NR⁸R⁹ where R⁹ is heteroaryl or substituted heteroaryl. In another embodiment where Z is —C(═O)NR⁸R⁹, R⁹ is substituted heteroaryl. In another embodiment where Z is —C(═O)NR⁸R⁹ and R⁹ is substituted heteroaryl, the substituted heteroaryl is selected from the group consisting of 4-methyl-2-oxo-2H-chromen-7-yl, 1-phenyl-4-carboxy-1H-pyrazol-5-yl, 5-carboxypyrid-2-yl, 2-carboxypyrazin-3-yl, and 3-carboxythien-2-yl.

In another embodiment Z is —C(═O)NR⁸R⁹ where R⁹ is heterocyclic. In another embodiment where Z is —C(═O)NR⁸R⁹ and R⁹ is heterocyclic, the heterocyclic group is N-morpholino, tetrahydrofuranyl, and 1,1-dioxidotetrahydrothienyl.

In another embodiment Z is —C(═O)NR⁸R⁹ where R⁸ and R⁹, together with the nitrogen atom pendent thereto, form a heterocyclic or substituted heterocyclic ring. In another embodiment where Z is —C(═O)NR⁸R⁹ and R⁸ and R⁹, together with the nitrogen atom pendent thereto form a ring, the heterocyclic and substituted heterocyclic rings comprise 4 to 8 membered rings containing 1 to 3 heteroatoms. In another embodiment where Z is —C(═O)NR⁸R⁹ and R⁸ and R⁹, together with the nitrogen atom pendent thereto form an optionally substituted heterocyclic ring, the 1 to 3 heteroatoms comprises 1 to 2 nitrogen atoms. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of piperidine, substituted piperidine, piperazine, substituted piperazine, morpholino, substituted morpholino, thiomorpholino and substituted thiomorpholino wherein the sulfur atom of the thiomorpholino or substituted thiomorpholino ring is optionally oxidized to provide for sulfoxide and sulfone moieties. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of 4-hydroxypiperidin-1-yl, 1,2,3,4-tetrahydro-3-carboxy-isoquinolin-2-yl, 4-methylpiperizin-1-yl, morpholin-4-yl, thiomorpholin-4-yl, 4-methyl-piperazin-1-yl, and 2-oxo-piperazinyl.

In another embodiment, Z is —C(X)N(R³)CR²R^{2′}C(═O)R¹.

In another embodiment, Z is —C(O)NHCHR²C(═O)R¹.

In another embodiment when Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, substituted cycloalkyl, aryl, substituted aryl, heteroaryl and substituted heteroaryl. In another embodiment where Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, and substituted cycloalkyl. In another embodiment where Z is —C(X)N(R³)CR²R^{2′}C(═O)R¹ or —C(O)NHCHR²C(═O)R¹, R² is selected from the group consisting of hydrogen, methyl, 1-methylprop-1-yl, sec-butyl, hydroxymethyl, 1-hydroxyeth-1-yl, 4-amino-n-butyl, 2-carboxyeth-1-yl, carboxymethyl, benzyl, (1H-imidazol-4-yl)methyl, (4-phenyl)benzyl, (4-phenylcarbonyl)benzyl, cyclohexylmethyl, cyclohexyl, 2-methylthioeth-1-yl, iso-propyl, carbamoylmethyl, 2-carbamoyleth-1-yl, (4-hydroxy)benzyl, and 3-guanidino-n-propyl.

In another embodiment when Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino, and R² and R³, together with the carbon atom and nitrogen atom bound thereto respectively, are joined to form a heterocyclic or substituted heterocyclic group. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino and R² and R³, together with the carbon atom and nitrogen atom bound thereto respectively, are joined to form a heterocyclic or substituted heterocyclic group, the heterocyclic and substituted heterocyclic groups are selected from the group consisting of pyrrolidinyl, 2-carboxy-pyrrolidinyl, 2-carboxy-4-hydroxypyrrolidinyl, and 3-carboxy-1,2,3,4-tetrahydroisoquinolin-3-yl.

In another embodiment, Z is selected from 1-carboxamidocyclopent-1-ylaminocarbonyl, 1-carboxamido-1-methyl-eth-1-ylaminocarbonyl, 5-carboxy-1,3-dioxan-5-ylaminocarbonyl, 1-(N-methylcarboxamido)-1-(methyl)-eth-1-ylaminocarbonyl, 1-(N,N-dimethylcarboxamido)-1-(methyl)-eth-1-ylaminocarbonyl, 1-carboxy-1-methyl-eth-1-ylaminocarbonyl, 1-(N-methylcarboxamido)-cyclobutanaminocarbonyl, 1-carboxamido-cyclobutanaminocarbonyl, 1-(N,N-dimethylcarboxamido)-cyclobutanaminocarbonyl, 1-(N-methylcarboxamido)-cyclopentanaminocarbonyl, 1-(N,N-dimethylcarboxamido)-cyclopentanaminocarbonyl, 1-(carboxamido)-cyclopentanaminocarbonyl, 3-[N-(4-(2-aminothiazol-4-yl)phenyl)aminocarbonyl]-piperidin-3-ylaminocarbonyl, 3-carboxamido-pyrrolidin-3-ylaminocarbonyl, [1-(4-(acrylic acid)-phenyl)aminocarbonyl)-cyclobutan-1-yl]aminocarbonyl, and [1-methyl-1-(4-(acrylic acid)-phenyl)aminocarbonyl)-eth-1-yl]aminocarbonyl.

In another embodiment, Z is —C(O)NR²¹S(O)₂R⁴. In another embodiment where Z is —C(O)NR²¹S(O)₂R⁴, R⁴ is selected from the group consisting of alkyl, substituted alkyl, aryl, substituted aryl, heteroaryl and substituted heteroaryl. In another embodiment where Z is —C(O)NR²¹S(O)₂R⁴, R⁴ is methyl, ethyl, isopropyl, propyl, trifluoromethyl, 2,2,2-trifluoroethyl, phenyl, benzyl, phenethyl, 4-bromophenyl, 4-nitrophenyl or 4-methylphenyl, 4-methoxyphenyl, 2-aminoethyl, 2-(dimethylamino)ethyl, 2-N-benzyloxyaminoethyl, pyridinyl, thienyl, 2-chlorothien-5-yl, 2-methoxycarbonylphenyl, naphthyl, 3-chlorophenyl, 2-bromophenyl, 2-chlorophenyl, 4-trifluoromethoxyphenyl, 2,5-difluorophenyl, 4-fluorophenyl, 2-methylphenyl, 6-ethoxybenzo[d]thiazo-2-yl, 4-chlorophenyl, 3-methyl-5-fluorobenzo[b]thiophen-1-yl, 4-acetylaminophenyl, quinolin-8-yl, 4-t-butylphenyl, cyclopropyl, 2,5-dimethoxyphenyl, 2,5-dichloro-4-bromo-thien-3-yl, 2,5-dichloro-thien-3-yl, 2,6-dichlorophenyl, 1,3-dimethyl-5-chloro-1H-pyrazol-4-yl, 3,5-dimethylisoxazol-4-yl, benzo[c][1,2,5]thiadiazol-4-yl, 2,6-difluorophenyl, 6-chloro-imidazo[2,1-b]thiazol-5-yl, 2-(methylsulfonyl)phenyl, isoquinolin-8-yl, 2-methoxy-4-methylphenyl, 1,3,5-trimethyl-1H-pyrazol-4-yl, 1-phenyl-5-methyl-1H-pyrazol-4-yl, 2,4,6-trimethylphenyl, and 2-carbamoyl-eth-1-yl.

In another embodiment, Z is selected from hydrogen, halo, alkyl, alkoxy, amino, substituted amino, and cyano.

In another embodiment, Z is —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group.

In another embodiment, Z is selected from 1-(6-(3-carboxyprop-2-en-1-yl)-1H-benzo[d]imidazol-2-yl)cyclobutanaminocarbonyl, 3-(6-(3-carboxyprop-2-en-1-yl)-1H-benzo[d]imidazol-2-yl)-1-methylpyrrolidin-3-aminocarbonyl, 1-(1-methyl-6-(3-carboxyprop-2-en-1-yl)-1H-benzo[d]imidazol-2-yl)cyclobutanaminocarbonyl, 1-(benzofuran-2-yl)-5-carboxy-cyclobutanaminocarbonyl, 1-(2-methylthiazol-4-yl)-cyclobutanaminocarbonyl, 1-(2-acetylamino-thiazol-4-yl)-cyclobutanamino, 1-(2-methylamino-thiazol-4-yl)-cyclobutanaminocarbonyl, 1-(2-ethylthiazol-4-yl)-cyclobutanaminocarbonyl, and 1-(cyano)-cyclobutanaminocarbonyl.

In still other embodiments of each of formula I-IIIa where appropriate, Z is carboxy, carboxy ester, carboxylic acid isostere, —C(O)NR⁸R⁹, or —C(O)NHS(O)₂R⁴, wherein R⁸ and R⁹ are as defined above and R⁴ is alkyl or aryl. In other embodiments Z is carboxy, methyl carboxylate, ethyl carboxylate, 6-(β-D-glucuronic acid)ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl. In still other embodiments Z is carboxy. In yet other embodiments Z is —C(═O)OH.

In some embodiments of each of formula I-IIIa where appropriate, Z¹ is selected from the group consisting of hydrogen, halo, alkyl, and haloalkyl.

In some embodiments of each of formula I-IIIa where appropriate, R is CᵥH₂ᵥ—C(O)—OR²³ where v is 1, 2 or 3; and R²³ is hydrogen, alkyl or substituted alkyl. In another embodiment where R is CᵥH₂ᵥ—C(O)—OR²³, v is 1. In another embodiment where R is CᵥH₂ᵥ—C(O)—OR²³, R is carboxymethyl or methylcarboxymethyl.

In another embodiment R is hydrogen.

In another embodiment R is CᵥH₂ᵥ—C(O)—NR¹²R¹³ where v is 1, 2 or 3; R¹² and R¹³ are selected from hydrogen, alkyl, substituted alkyl, alkenyl, substituted alkenyl, alkynyl, substituted alkynyl alkoxy, substituted alkoxy and —(CH₂)₀₋₃R¹⁶; and R¹⁶ is aryl, heteroaryl, heterocyclic, —NR¹⁷R¹⁸; and R¹⁷ and R¹⁸ are independently selected from hydrogen, and alkyl, or alternatively, R¹⁷ and R¹⁸ together with the nitrogen atom to which they are attached join to form a heterocyclic ring with 4 to 7 ring atoms; or, alternatively, R¹² and R¹³ and the nitrogen atom to which they are attached form a heterocyclic or substituted heterocyclic ring; provided that both R¹² and R¹³ are not alkoxy and/or substituted alkoxy. In another embodiment v is 1. In another embodiment where R is CᵥH₂ᵥ—C(O)—NR¹²R¹³, the NR¹²R¹³ group is selected from N,N-dimethylamino-carbonylmethyl, [N-(4-hydroxy-1,1-dioxidotetrahydro-3-thienyl)amino]-carbonylmethyl, (cyclopropylmethylamino)-carbonylmethyl, (prop-2-yn-1-ylamino)-carbonylmethyl, (2-(morpholino)eth-1-ylamino)-carbonylmethyl, (phenylsulfonylamino)-carbonylmethyl, [N-benzylamino]-carbonylmethyl, (N-(4-methylsulfonyl-benzyl)amino)-carbonylmethyl, (tryptophanyl)-carbonylmethyl, (tyrosine)-carbonylmethyl, (N-(1-carboxyprop-1-ylamino)-carbonylmethyl, (N-(2-carboxyeth-1-yl)-amino)-carbonylmethyl, (N-(4-carboxybenzyl)-amino)-carbonylmethyl, N-[3-(N′-(4-(acrylic acid)-phenyl)carboxamido)pyrrolidin-3-yl]amino-carbonylmethyl, N-[4-(N′-(4-(acrylic acid)-phenyl)carboxamido)piperidin-4-yl]amino-carbonylmethyl, [2-(N,N-dimethylamino)eth-1-ylamino]-carbonylmethyl, [(1-(5-methyl-4H-1,2,4-triazol-3-yl)ethyl)amino]-carbonylmethyl, (1-methyl-1-[N-(1-methyl-2-carboxy-1H-indol-5-yl)aminocarbonyl]eth-1-ylamino-carbonylmethyl, [N-(1-methylpyrrolidin-3-yl-ethyl)amino]-carbonylmethyl, (1-methyl-1-[N-(4-(acrylic acid)phenyl)aminocarbonyl]eth-1-ylamino-carbonylmethyl, (1-methyl-1-[N-(4-(2-carboxy-furan-5-yl)phenyl)aminocarbonyl]eth-1-ylamino-carbonylmethyl, (1-methyl-1-[N-(4-(4-carboxy-thiazol-2-yl)phenyl)aminocarbonyl]eth-1-ylamino-carbonylmethyl, (2-(4-methylpiperazin-1-yl)eth-1-ylamino)-carbonylmethyl, [(1-methylpyrrolidin-3-yl)methylamino]-carbonylmethyl, [N-(1-methylpiperidin-3-yl-methyl)-amino]-carbonylmethyl, (1-piperidin-1-ylcyclopentyl)methylamino]-carbonylmethyl, (1-(acetyl)-pyrrolidin-2-ylmethyl)amino)-carbonylmethyl, [(2-(N,N-dimethylamino)-carbonyl)methylamino]-carbonylmethyl, [N-(1,1-dioxidotetrahydro-3-thienyl)methylamino]-carbonylmethyl, (N-methyl-N-cyclohexyl-amino)-carbonylmethyl, (N-methyl-N-carboxymethyl-amino)-carbonylmethyl, [N-methyl-N-benzyl-amino]-carbonylmethyl, (N-methyl-N—(N′,N′-dimethylaminoacetyl)-amino)-carbonylmethyl, [N-methyl-N-phenyl-amino]-carbonylmethyl, (N-methyl-N-isopropyl-amino)-carbonylmethyl, (N-methyl-N—(N′-methylpiperidin-4-yl)amino)-carbonylmethyl, [N-methyl-N-(1-methylpiperidin-4-yl)amino]-carbonylmethyl, [N-methyl-N-(1-methylpiperidin-4-yl-methyl)-amino]-carbonylmethyl, [N-methyl-N-(1-methylpiperidin-3-yl-methyl)-amino]-carbonylmethyl, [N-methyl-N-(1-methylpyrazin-2-yl-methyl)-amino]-carbonylmethyl, [N-methyl-N-(5-methyl-1H-imidazol-2-ylmethyl)-amino]-carbonylmethyl, (N-methyl-N-[2-(hydroxy)eth-1-yl]amino)-carbonylmethyl, (N-methyl-N-[2-(N′,N′-dimethylamino)eth-1-yl]amino)-carbonylmethy, N-methyl-N-[2-(N′,N′-diethylamino)eth-1-yl]amino)-carbonylmethyl, (N-methyl-N-[2-(pyridin-2-yl)eth-1-yl]amino)-carbonylmethyl, (N-methyl-N-[2-(pyridin-4-yl)eth-1-yl]amino)-carbonylmethyl, [N-methyl-N-(1-(1,3-thiazol-2-yl)ethyl)-amino]-carbonylmethyl, (N-methyl-N-[3-(N′,N′-dimethylamino)prop-1-yl]amino)-carbonylmethyl, (N-methyl-N-(1-carboxy-2-methylprop-1-yl)-amino)-carbonylmethyl, (N-ethyl-N-propyl-amino)-carbonylmethyl, (N-ethyl-N-[2-(methoxy)eth-1-yl]amino)-carbonylmethyl, (N-ethyl-N-[2-(N′,N′-diethylamino)eth-1-yl]amino)-carbonylmethyl, [7-methyl-2,7-diazaspiro[4.4]non-2-yl]-carbonylmethyl, (5-methyl-2,5-diazabicyclo[2.2.1]heptyl-2-yl)-carbonylmethyl, (4-methyl-1,4-diazepan-1-yl)-carbonylmethyl, (piperidinyl)-carbonylmethyl, (4-carboxy-piperidinyl)-carbonylmethyl, (3-carboxypiperidinyl)-carbonylmethyl, (4-hydroxypiperidinyl)-carbonylmethyl, (4-(2-hydroxyeth-1-yl)piperidin-1-yl)-carbonylmethyl, [4-(N,N-dimethylamino)-piperidin-1-yl]-carbonylmethyl, (3-(N,N-dimethylamino)-methylpiperidin-1-yl)-carbonylmethyl, (2-(2-(N,N-dimethylamino)-eth-1-yl)piperidin-1-yl)-carbonylmethyl, [4-(4-methyl-4H-1,2,4-triazol-3-yl)piperidin-1-yl]-carbonylmethyl, (4-pyrrolidinyl-piperidinyl)-carbonylmethyl, (3-pyrrolidinyl-piperidinyl)-carbonylmethyl, [4-(N,N-diethylamino)-piperidin-1-yl]-carbonylmethyl, (4-(azetidin-1-yl)-piperidin-1-yl)-carbonylmethyl, (4-(piperidin-1-yl)-piperidin-1-yl)-carbonylmethyl, (hexahydropyrrolo[1,2-a]pyrazin-2(1H)-yl)-carbonylmethyl, [(2-(N,N-dimethylamino)-methyl)morpholino]-carbonylmethyl, (3,5-dimethylmorpholino)-carbonylmethyl, (thiomorpholino)-carbonylmethyl, morpholino-carbonylmethyl, (pyrrolidinyl)-carbonylmethyl, (2-carboxy-pyrrolidin-1-yl)-carbonylmethyl, (2-(carboxy)-4-hydroxy-pyrrolidin-1-yl)-carbonylmethyl, (2-carboxamide-pyrrolidin-1-yl)-carbonylmethyl, (2-(N,N-dimethylaminocarbonyl)-pyrrolidin-1-yl)-carbonylmethyl, (3-(N′,N′-dimethylamino)-pyrrolidin-1-yl)-carbonylmethyl, (3-(N′,N′-diethylamino)-pyrrolidin-1-yl)-carbonylmethyl, (3-(pyridin-3-yl)-pyrrolidin-1-yl)-carbonylmethyl, (2-pyidin-4-ylpyrrolidin-1-yl)-carbonylmethyl, piperazin-1-yl-carbonylmethyl, (4-methylpiperazinyl)-carbonylmethyl, (4-(carboxymethyl)-piperazin-1-yl)-carbonylmethyl, (4-(2-hydroxyeth-1-yl)piperazin-1-yl)-carbonylmethyl, (4-(isopropyl)piperazin-1-yl)-carbonylmethyl, (4-(2-methoxyeth-1-yl)piperazin-1-yl)-carbonylmethyl, (4-(ethyl)piperazin-1-yl)-carbonylmethyl, (4-(N′,N′-dimethylaminoacetyl)-piperazin-1-yl)-carbonylmethyl, and (4-(6-methoxypyridin-2-yl)piperazin-1-yl)-carbonylmethyl.

In another embodiment, R is selected from morpholinocarbonylmethyl, N,N-dimethylaminocarbonylmethyl, (4-pyrrolidinyl-piperidin-1-yl)carbonylmethyl, piperazinylcarbonylmethyl. In some aspects, R is an oxide of morpholinocarbonylmethyl, N,N-dimethylaminocarbonylmethyl, (4-pyrrolidinyl-piperidin-1-yl)carbonylmethyl, piperazinylcarbonylmethyl.

In another embodiment, R is selected from [(N,N-dimethylamino)prop-2-en-1-yl]-carbonylmethyl, (N,N-dimethylpiperidin-4-aminium trifluoroacetate)acetyl, 2-(N,N-dimethylpiperidin-4-aminium trifluoroacetate)morpholino acetyl, (2-(diisopropyl)eth-1-yl)-carbonylmethyl, (pyridin-4-ylcarbonylhydrazino)-carbonylmethyl, (N-(4-carboxybenzyl)-amino)carbonylhydrazino)-carbonylmethyl, (acetylhydrazino)-carbonylmethyl, ((N′,N′-dimethylaminomethyl-carbonyl)hydrazino)-carbonylmethyl.

In still other embodiments of each of formula I-IIIa where appropriate, R is substituted alkyl, wherein said substituted alkyl is selected from the group consisting of aminoalkyl, substituted aminoalkyl, arylalkyl, substituted arylalkyl, heteroarylalkyl, substituted heteroarylalkyl, heterocyclylalkyl, substituted heterocyclylalkyl, —CH₂COOH, and —CH₂CONR¹²R¹³, wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

In other embodiments, R is —CH₂CONR¹²R¹³ and at least one of R¹² or R¹³ is alkyl, substituted alkyl, or heteroaryl. In some aspects at least one of R¹² or R¹³ is methyl, carboxymethyl, 2-hydroxyethyl, 2-morpholin-4-ylethyl, or tetrazoyl-5-yl.

In yet other embodiments, R is —CH₂CONR¹²R¹³ and R¹² and R¹³ and the nitrogen atom to which they are attached form a substituted or unsubstituted heterocyclic ring. In some aspects R¹² and R¹³ and the nitrogen atom to which they are attached form a substituted or unsubstituted morpholino, substituted or unsubstituted piperidinyl, or a substituted or unsubstituted pyrrolidinyl ring. In other aspects the substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

In some embodiments of formula Ib, R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-benzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido)pyrrolidin-3-yl]amino, N-[4-(N′-(4-(acrylic acid)-phenyl)carboxamido)piperidin-4-yl]amino, 2-(N,N-dimethylamino)eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, (1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N—(N′,N′-dimethylaminoacetyl)-amino, N-methyl-N-phenyl-amino, N-methyl-N-isopropyl-amino, N-methyl-N—(N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino)eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino)-piperidin-1-yl, 3-(N,N-dimethylamino)-methylpiperidin-1-yl, 2-(2-(N,N-dimethylamino)-eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl)-pyrrolidin-1-yl, 3-(N′,N′-dimethylamino)-pyrrolidin-1-yl, 3-(N′,N′-diethylamino)-pyrrolidin-1-yl, 3-(pyridin-3-yl)-pyrrolidin-1-yl, 2-pyidin-4-ylpyrrolidin-1-y, piperazin-1-yl, 4-methylpiperazinyl, 4-(carboxymethyl)-piperazin-1-yl, 4-(2-hydroxyeth-1-yl)piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, and 4-(6-methoxypyridin-2-yl)piperazin-1-yl.

In some embodiments of formula I or Ia, HET is selected from quinolinylene and substituted quinolinylene. In another embodiment HET is selected from quinolinylene, isoquinolinylene, 7-methyl-quinolinylene, 7-trifluoromethyl-quinolinylene, 8-fluoro-quinolinylene and 7-fluoro-quinolinylene. In yet another embodiment HET is 2-[substituted]-quinolin-6-yl, 2-[substituted]-7-methyl-quinolinyl, 2-[substituted]-7-fluoro-quinolinyl, 2-[substituted]-7-trifluoromethyl-quinolinyl, and 2-[substituted]-8-fluoro-quinolinyl.

In some embodiments of formula I or Ia, HET is 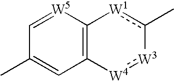
optionally substituted with (X)ₜ where X, t, W¹, W³, W⁴, and W⁵ are previously defined. In some aspects, W¹ is nitrogen. In other aspects where HET is selected from the group consisting of

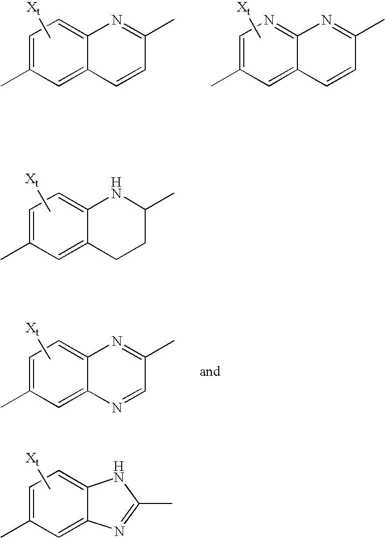

In some embodiments of formula I or Ia, HET is 1,4-phenylene optionally substituted with (X)ₜ where X and t are previously defined.

In some embodiments of each of formula I-IIIa where appropriate, t is 0.

In another embodiment, t is 1 and X is amino, nitro, methyl or halo.

In some embodiments of each of formula I-IIIa where appropriate, Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized. In some embodiments Y is substituted 5-membered heteroaryl ring optionally fused to a phenyl ring and having one, two, or three heteroatoms independently selected from the group consisting of N, O, or S wherein the heteroatoms N or S are optionally oxidized.

In another embodiment —Y is —Ar¹-(G¹)_{q} where Ar¹ is selected from arylene and heteroarylene, G¹ is selected from halo, hydroxy, nitro, cyano, alkyl, substituted alkyl, alkoxy, substituted alkoxy, acyl, acylamino, aminoacyl, amino, substituted amino, carboxy and carboxy ester; and q is an integer from 1 to 3. In another embodiment where —Y is —Ar¹-(G¹)_{q}, Ar¹ is selected from phenyl, thiazolyl, furanyl, thienyl, pyridinyl, pyrazinyl, oxazolyl, isoxazolyl, pyrrolyl, imidazolyl, and pyrrolidinyl. In another embodiment where —Y is —Ar¹-(G¹)_{q}, G¹ is selected from bromo, chloro, methyl, hydroxy, methoxy, ethoxy, acetyl, acetamido, carboxy, and amino. In another embodiment Y is selected from 2,4-dimethylthiazol-5-yl, 3-bromo-4-aminophenyl, 3-amido-4-hydroxy-phenyl, 2-hydroxy-6-methoxy-phenyl, 4-(acetylamino)-phenyl, 2,4-dihydroxyphenyl, 2,4-dimethoxy-6-hydroxyphenyl, and 7-hydroxybenzofuranyl.

In another embodiment Y is —Ar¹—Ar²— where the —Ar¹—Ar²— group is selected from the group consisting of -aryl-aryl, -aryl-substituted aryl, -substituted aryl-aryl, -substituted aryl-substituted aryl, -aryl-heteroaryl, -aryl-substituted heteroaryl, -substituted aryl-heteroaryl, -substituted aryl-substituted heteroaryl, heteroaryl-aryl, heteroaryl-substituted aryl, substituted heteroaryl-aryl, substituted heteroaryl-substituted aryl, -aryl-cycloalkyl, -aryl-substituted cycloalkyl, -substituted aryl-cycloalkyl, -substituted aryl-substituted cycloalkyl, -aryl-heterocyclic, aryl-substituted heterocyclic, substituted aryl-heterocyclic, and substituted aryl-substituted heterocyclic.

In another embodiment where Y is —Ar¹—Ar²—, the —Ar¹—Ar²— group is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphenyl, 4-methoxybiphenyl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

In another embodiment where Y is —Ar¹—Ar²—, the —Ar¹—Ar²— group is selected from the group consisting of 4-(1H-imidazol-1-yl)phenyl, 2-furan-2-yl-5-methoxyphenyl, 5-methoxy-2-thiophen-2-ylphenyl, 2-(2,4-dimethoxypyrimidin-5-yl)-4-methoxyphenyl, 2-(pyrid-4-yl)phenyl, 3-amino-5-phenylthiophen-2-yl, 5-(4-chlorophenyl)-2-methylfuran-2-yl, 3-(4-chlorophenyl)-5-methylisoxazol-4-yl, 2-(4-chlorophenyl)-4-methylthiazol-5-yl, 3-(3,4-dichloro-phenyl)isoxazol-5-yl, 3,5-dimethyl-1-phenyl-1H-pyrazol-4-yl, 5-methyl-2-phenylthiophen-3-yl, and 1-phenyl-1H-pyrazol-4-yl.

In another embodiment where Y is —Ar¹—Ar²—, the —Ar¹—Ar²— group is selected from the group consisting of 2-cyclohexyl-N,N-dimethylamino-carbonylmethyl-5-methoxyphenyl, and 4-morpholinophenyl.

In still other embodiments of each of formula I-IIIa where appropriate, Y is is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl. In some aspects, Y is substituted with one to three subsitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester. In still other aspects, Y is 2,4-dimethylthiazol-5-yl.

Preferred compounds of this invention or the pharmaceutically acceptable salts, partial salts, or tautomers thereof include those set forth in Tables I-VI below:

TABLE I
Indole Derivatives

| Z | R | Y | X | NAME |
|---|---|---|---|---|
| CO₂H | H | 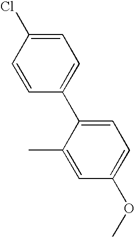 | | 2-[2-(4′-chloro-4-methoxy-biphen-2-yl)- quinolin-6-yl]-3-cyclohexyl- 1H-indole-6-carboxylic acid (Compound 200) |
| CO₂H | 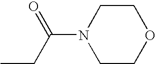 | 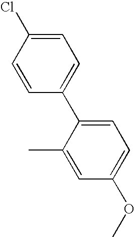 | | 1-[morpholinocarbonylmethyl]-2-[2-(4′- chloro-4-methoxy-biphen-2-yl)-quinolin-6- yl]-3-cyclohexyl-1H-indole-6-carboxylic acid (Compound 203) |
| CO₂H | 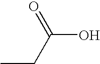 | 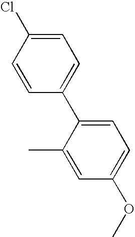 | | 1-[carboxymethyl]-2-[2-(4′-chloro- 4-methoxy-biphen-2-yl)-quinolin-6- yl]-3-cyclohexyl-1H-indole- 6-carboxylic acid (Compound 202) |
| CO₂H | 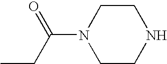 | 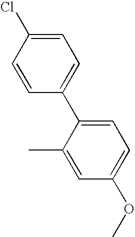 | | 1-[N-piperazinylcarbonylmethyl]-2-[2-(4′- chloro-4-methoxy-biphen-2- yl)-quinolin-6-yl]-3-cyclohexyl-1H- indole-6-carboxylic acid (Compound 204) |
| CO₂H | 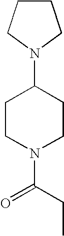 | 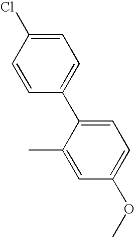 | | 1-[(4-pyrrolidin-1-yl-piperidin- 1-yl)carbonylmethyl]-2-[2-(4′- chloro-4-methoxy-biphen-2-yl)- quinolin-6-yl]-3-cyclohexyl-1H- indole-6-carboxylic acid (Compound 205) |
| CO₂H | 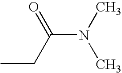 | 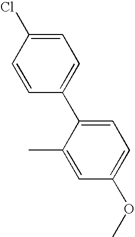 | | 1-[N,N-dimethylaminocarbonyl- methyl]-2-[2-(4′-chloro-4-methoxy- biphen-2-yl)-quinolin-6-yl]-3-cyclohexyl- 1H-indole-6-carboxylic acid (Compound 206) |
| CO₂Me | 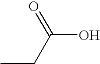 | 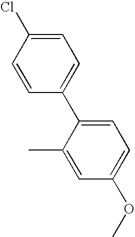 | | 1-[carboxymethyl]-2-[2-(4′- chloro-4-methoxybiphen-2-yl)- quinolin-6-yl]-3-cyclohexyl- 1H-indole-6-carboxylic acid methyl ester (Compound 201) |
| CO₂H | 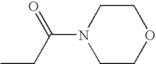 | 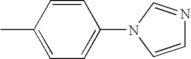 | | 1-[N-morpholinocarbonylmethyl]- 2-[2-(4-(1H-imidazolyl)phenyl)- quinolin-6-yl]-3-cyclohexyl- 1H-indole-6- carboxylic acid (Compound 209) |
| CO₂H | 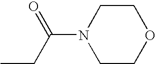 | 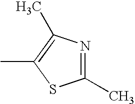 | | 1-[N-morpholinocarbonylmethyl]-2- [2-(2,4-dimethylthiazol-5-yl)-quinolin- 6-yl]-3-cyclohexyl- 1H-indole-6-carboxylic acid (Compound 210) |
| CO₂H | 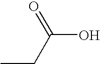 | 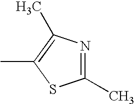 | | 1-[carboxymethyl]-2-[2-(2,4- dimethylthiazol-5-yl)- quinolin-6-yl]-3-cyclohexyl-1H- indole-6-carboxylic acid (Compound 211) |
| CO₂H | 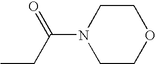 | 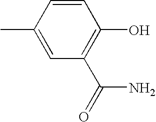 | | 1-[N-morpholinocarbonylmethyl]- 2-[2-(3-amido-4-hydroxy-phenyl)-quinolin- 6-yl]-3-cyclohexyl-1H-indole-6-carboxylic acid (Compound 207) |
| CO₂H | 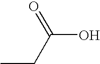 | 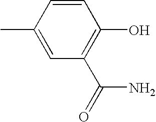 | | 1-carboxylmethyl-2-[2-(3-Carbamoyl- 4-hydroxy-phenyl)-quinolin-6-yl]- 3-cyclohexyl-1H-indole-6-carboxylic acid (Compound 208) |
| CO₂H | 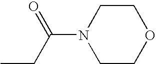 | 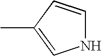 | | 1-[N-morpholinocarbonylmethyl]-2-[2- (pyrrol-3-yl)-quinolin-6-yl]-3-cyclohexyl- 1H-indole-6-carboxylic acid |
| CO₂H | 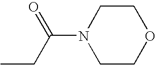 | 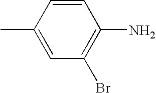 | | 1-[N-morpholinocarbonylmethyl]-2-[2- (3-bromo-4-aminophenyl)-quinolin-6-yl]- 3-cyclohexyl-1H-indole-6-carboxylic acid |
| CO₂H | 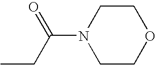 | 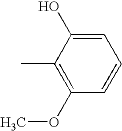 | | 1-[N-morpholinocarbonylmethyl]-2-[2- (2-hydroxy-6-methoxy-phenyl)-quinolin- 6-yl]-3-cyclohexyl-1H-indole-6- carboxylic acid |
| CO₂H | 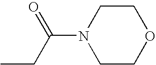 | 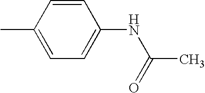 | | 1-]N-morpholinocarbonylmethyl]-2-[2- (4-acetylaminophenyl)-quinolin-6- yl]-3-cyclohexyl-1H-indole-6- carboxylic acid |
| CO₂H | 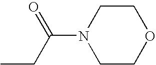 | 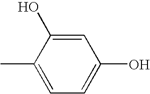 | | 1-[N-morpholinocarbonylmethyl]-2-[2- (2,4-dihydroxyphenyl)-quinolin- carboxylic acid |
| CO₂H | 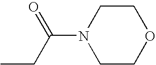 | 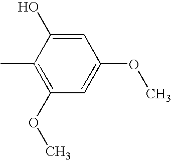 | | 1-[N-morpholinocarbonylmethyl]-2-[2- (2,4-dimethoxy-6-hydroxyphenyl)- quinolin-6-yl]-3-cyclohexyl-H-indole- 6-carboxylic acid |
| CO₂H | 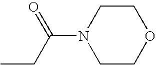 | 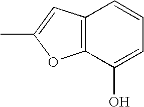 | | 1-[N-morpholinocarbonylmethyl]-2-[2- (7-hydroxybenzofuran-2-yl)- quinolin-6-yl]-3-cyclohexyl-1H-indole-6- carboxylic acid |
| 1H-tetrazol-5-yl | 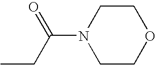 | 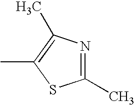 | | 1-[N-morpholinocarbonylmethyl]-2-[2- (2,4-dimethylthiazol-5-yl)- quinolin-6-yl]-3-cyclohexyl-1H- indole-6-1H-tetrazol-5-yl |
| 5-oxo-4,5-dihydro-1,2,4- oxadiazol-3-yl | 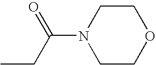 | 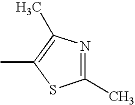 | | 1-[N-morpholinocarbonylmethyl]-2-[2- (2,4-dimethylthiazol-5-yl)-quinolin- 6-yl]-3-cyclohexyl-1H-indole- 6-(5-oxo-4,5-dihydro-1,2,4-oxadiazol-3-yl) |
| methyl- sulfonylaminocaronyl | 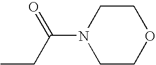 | 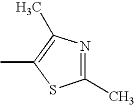 | | 1-[N-morpholinocarbonylmethyl]-2-[2- (2,4-dimethylthiazol-5-yl)-quinolin- 6-yl]-3-cyclohexyl-1H-indole-6- (methylsulfonylaminocarbonyl) |
| trifluoromethyl- sulfonylaminocaronyl | 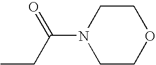 | 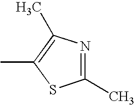 | | 1-[N-morpholinocarbonylmethyl]-2- [2-quinolin-6-yl]-3- cyclohexyl-1H-indole-6-(trifluoromethyl- sulfonylaminocarbonyl) |
| phenyl- sulfonylaminocarbonyl | 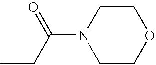 | 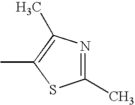 | | 1-[N-morpholinocarbonylmethyl]-2-[2- (2,4-dimethylthiazol-5-yl)-quinolin- 6-yl]-3-cyclohexyl-1H-indole- 6-(phenyl-sulfonylaminocarbonyl) |
| n = 2 6-CO₂H 7-methyl | 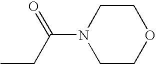 | 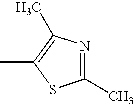 | | 1-[N-morpholinocarbonylmethyl]-2-[2- (2,4-dimethylthiazol-5-yl)-quinolin- 6-yl]-3-cyclohexyl-7-methyl-1H- indole-6-carboxylic acid |
| n = 2 6-CO₂H 7-fluoro | 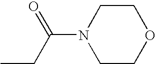 | 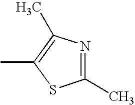 | | 1-[N-morpholinocarbonylmethyl]-2-[2- (2,4-dimethylthiazol-5-yl)-quinol- 6-yl]-3-cyclohexyl-7-fluoro- 1H-indole-6-carboxylic acid |
| CO₂H | 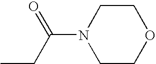 | 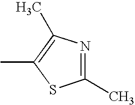 | | 1-[N-morpholinocarbonyl]-2-[2- (2,4-dimethylthiazol-5-yl)-quinolin- 6-yl]-3-cyclohexyl-1H-indole- 6-carboxylic acid |
| CO₂H | 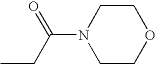 | 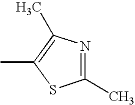 | t = 1 7-fluoro | 1-[N-morpholinocarbonylmethyl]-2-[2- (2,4-dimethylthiazol-5-yl)-7-fluoro- quinolin-6-yl]-3-cyclohexyl- 1H-indole-6-carboxylic acid |
| CO₂H | 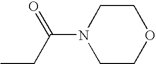 | 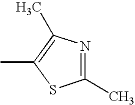 | t = 1 7-methyl | 1-[N-morpholinocarbonylmethyl]-2-[2- (2,4-dimethylthiazol-5-yl)-7-methylmethyl- quinolin-6-yl]-3-cyclohexyl- 1H-indole-6-carboxylic acid |
| CO₂H | 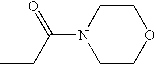 | 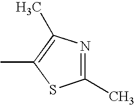 | t = 1 5-fluoro | 1-[N-morpholinocarbonylmethyl]-2-[2- (2,4-dimethylthiazol-5-yl)-5- fluoro-quinolin-6-yl]-3-cyclohexyl- 1H-indole-6-carboxylic acid |
| n = 2 6-CO₂H 7-methyl | 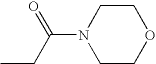 | 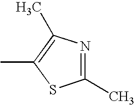 | t = 1 7-fluoro | 1-[N-morpholinocarbonylmethyl]-2-[2- (2,4-dimethylthiazol-5-yl)-7-fluoro-quinolin- 6-yl]-3-cyclohexyl-7-methyl- 1H-indole-6-carboxylic acid (Compound 238) |
| CO₂H | 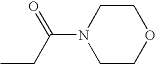 | 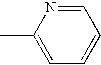 | | 1-[N-morpholinocarbonylmethyl]-2-[2- (pyrid-2-yl)-quinolin-6-yl]- 3-cyclohexyl-1H-indole-6- carboxylic acid (Compound 217) |
| CO₂H | 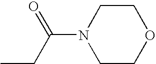 | 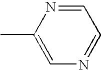 | | 1-[N-morpholinocarbonylmethyl]-2-[2- (pyrazin-2-yl)-quinolin-6-yl]-3- cyclohexyl-1H-indole-6- carboxylic acid (Compound 218) |
| CO₂H | 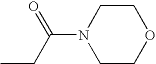 | 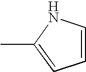 | | 1-[N-morpholinocarbonylmethyl]-2-[2- (pyrrol-2-yl)-quinolin-6-yl]-3- cyclohexyl-1H-indole-6- carboxylic acid (Compound 219) |
| CO₂H | 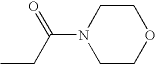 | 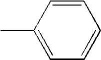 | | 1-[N-morpholinocarbonylmethyl]-2-[2- phenylquinolin-6-yl]-3- cyclohexyl-1H-indole-6- carboxylic acid (Compound 220) |
| CO₂H | 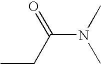 | 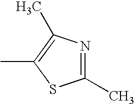 | | 1-[N,N-dimethylaminocarbonyl-methyl]- 2-[2-(2,4-dimethylthiazol- 5-yl)-quinolin-6-yl]-3-cyclohexyl- 1H-indole-6-carboxylic acid (Compound 212) |
| CO₂H | 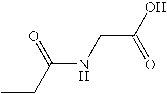 | 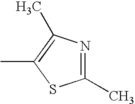 | | 1-[(N-carboxymethycarbamoyl)-methyl]- [2-(2,4-dimethyl-thiazol-5- yl)-quinolin-6-yl]-3-cyclohexyl- 1H-indole-6-carboxylic acid, (Compound 213) |
| CO₂H | 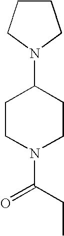 | 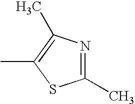 | | 1-[2-oxo-2-(4-pyrrolidin-1- yl-piperidin-1-yl)-ethyl]- 2-[2- (2,4-dimethylthiazol-5-yl)-quinolin- 6-yl]-3-cyclohexyl-1H-indole- 6-carboxylic acid (Compound 214) |
| CO₂H | 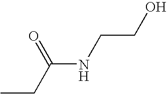 | 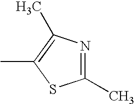 | | 1-[(2-hydroxyethylcarbamoyl)-methyl]-2- [2-(2,4-dimethyl-thiazol-5-yl)- quinolin-6-yl]-3-cyclohexyl- 1H-indole-6-carboxylic acid (Compound 215) |
| CO₂H | 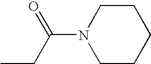 | 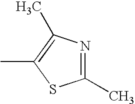 | | 1-(2-oxo-2-piperidin-1-yl-ethyl)-2- [2-(2,4-dimethylthiazol-5- yl)-3-cyclohexylquinolin-6- yl]-1H-indole-6-carboxylic acid (Compound 216) |
| CO₂H | 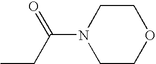 | 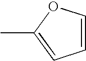 | | 1-[N-morpholinocarbonylmethyl]- 2-(2-furan-2-yl-quinolin-6-yl)- 3-cyclohexyl-1H-indole-6-carboxylic acid (Compound 222) |
| CO₂H | 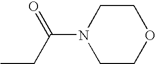 | 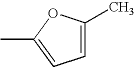 | | 1-[N-morpholinocarbonylmethyl]-2-[2- (5-methyl-furan-2-yl)-quinolin- 6-yl]-3-cyclohexyl-1H-indole-6- carboxylic acid (Compound 223) |
| CO₂H | 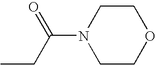 | 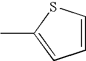 | | 1-[N-morpholinocarbonylmethyl]-2-[2- thien-2-ylquinolin-6-yl]- 3-cyclohexyl-1H-indole-6- carboxylic acid (Compound 224) |
| CO₂H | 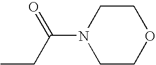 | 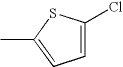 | | 1-[N-morpholinocarbonylmethyl]-2-[2- (5-chlorothien-2-yl)quinolin- 6-yl]-3-cyclohexyl-1H- indole-6-carboxylic acid (Compound 225) |
| CO₂H | 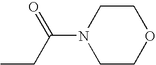 | 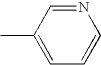 | | 1-[N-morpholinocarbonylmethyl]-2-[2- pyrid-3-ylquinolin-6-yl]-3- cyclohexyl-1H-indole-6- carboxylic acid (Compound 226) |
| CO₂H | 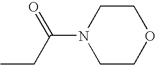 | 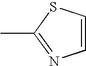 | | 1-[N-morpholinocarbonylmethyl]-2-[2- thiazol-2-ylquinolin-6-yl]- 3-cyclohexyl-1H-indole- 6-carboxylic acid (Compound 227) |
| CO₂H | 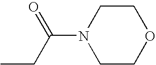 | 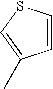 | | 1-[N-morpholinocarbonylmethyl]-2-[2- thien-3-ylquinolin-6-yl]- 3-cyclohexyl-1H-indole-6- carboxylic acid (Compound 228) |

t is 0 and n is 1 unless otherwise indicated- when n is 1, Z is at the 6 position of the indole ring

TABLE II

| Cmpd | STRUCTURE | COMPOUND NAME |
|---|---|---|
| 221 | 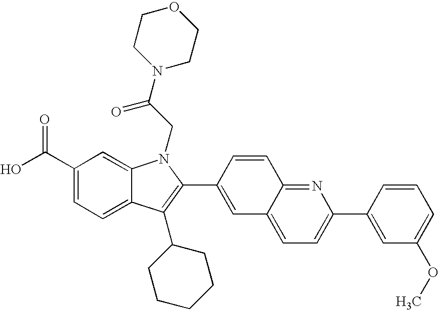 | 3-Cyclohexyl-2-[2-(3-methoxy-phenyl)- quinolin-6-yl]-1-(2-morpholin- 4-yl-2-oxo-ethyl)-1H-indole- 6-carboxylic acid |
| 229 | 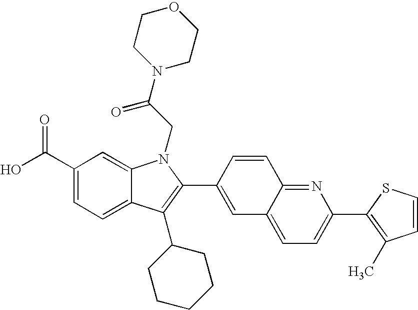 | 3-Cyclohexyl-2-[2-(3-methyl- thiophen-2-yl)-quinolin- 6-yl]-1-(2-morpholin-4-yl- 2-oxo-ethyl)-1H-indole- 6-carboxylic acid |
| 230 | 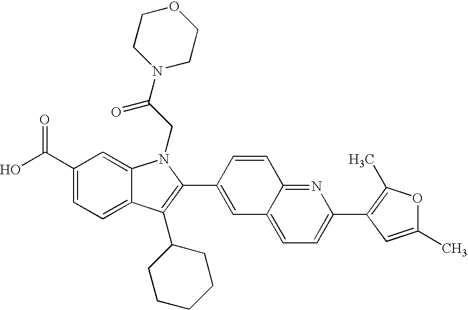 | 3-Cyclohexyl-2-[2-(2,5-dimethyl- furan-3-yl)-quinolin- 6-yl]-1-(2-morpholin- 4-yl-2-oxo-ethyl)- 1H-indole-6-carboxylic acid |
| 231 | 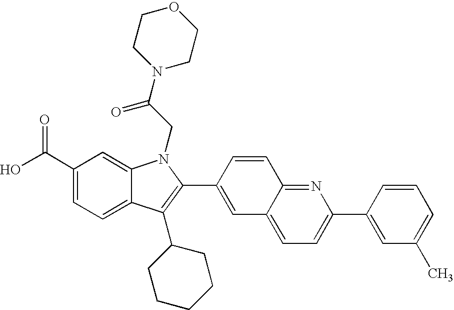 | 3-Cyclohexyl-1-(2-morpholin-4-yl- 2-oxo-ethyl)-2-(2-m-tolyl- quinolin-6-yl)-1H-indole- 6-carboxylic acid |
| 232 | 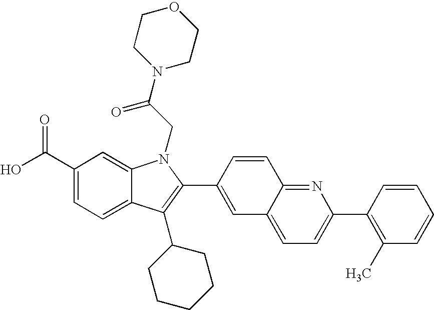 | 3-Cyclohexyl-1-(2-morpholin-4-yl- 2-oxo-ethyl)-2-(2-o-tolyl- quinolin-6-yl)-1H-indole- 6-carboxylic acid |
| 233 | 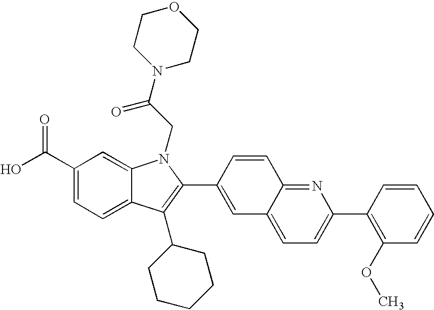 | 3-Cyclohexyl-2-[2-(2-methoxy- phenyl)-quinolin-6-yl]-1- (2-morpholin-4-yl-2-oxo-ethyl)- 1H-indole-6-carboxylic acid |
| 234 | 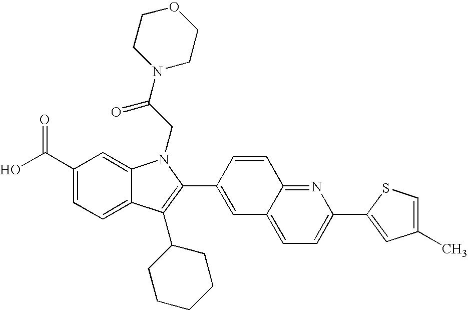 | 3-Cyclohexyl-2-[2-(4-methyl-thiophen- 2-yl)-quinolin-6-yl]-1-(2- morpholin-4-yl-2-oxo-ethyl)- 1H-indole-6-carboxylic acid |
| 235 | 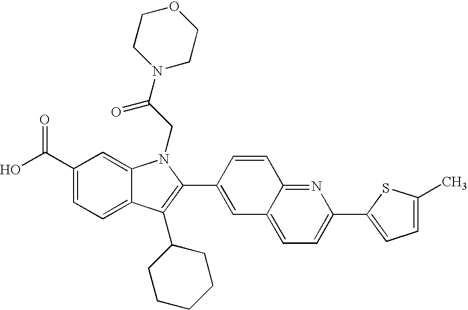 | 3-Cyclohexyl-2-[2-(5-methyl-thiophen- 2-yl)-quinolin-6-yl]-1-(2- morpholin-4-yl-2-oxo-ethyl)- 1H-indole-6-carboxylic acid |
| 236 | 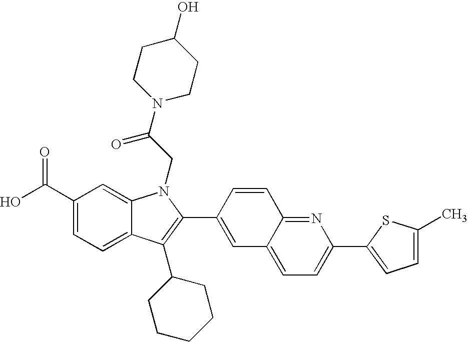 | 3-Cyclohexyl-2-[2-(2,4-dimethyl- thiazol-5-yl)-quinolin- 6-yl]-1-[2-(4-hydroxy-piperidin-1- yl)-2-oxo-ethyl]-1H- indole-6-carboxylic acid |
| 237 | 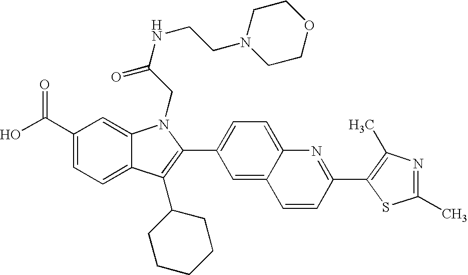 | 3-Cyclohexyl-2-[2-(2,4-dimethyl- thiazol-5-yl)-quinolin- 6-yl]-1-[(2-morpholin-4- ylethylcarbamoyl)-methyl]- 1H-indole-6-carboxylic acid |
| 238 | 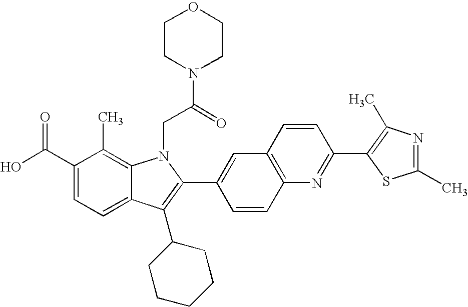 | 3-Cyclohexyl-2-[2-(2,4-dimethyl- thiazol-5-yl)-quinolin- 6-yl]-7-methyl-1-(2-morpholin- 4-yl-2-oxo-ethyl)-1H- indole-6-carboxylic acid |
| 239 | 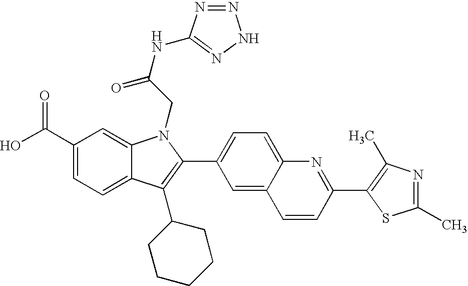 | 3-Cyclohexyl-2-[2-(2,4-dimethyl- thiazol-5-yl)-quinolin- 6-yl]-1-[(2H-tetrazol-5-ylcarbamoyl)- methyl]-1H-indole-6-carboxylic acid |
| 240 | 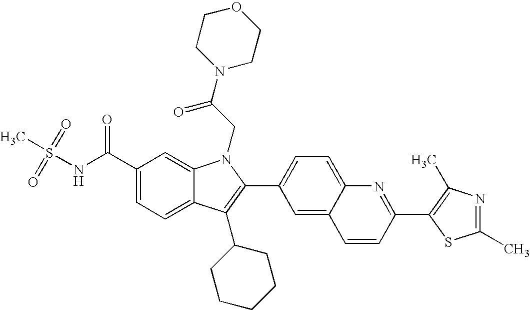 | N-[3-Cyclohexyl-2-[2-(2,4- dimethyl-thiazol-5-yl)-quinolin- 6-yl]-1-(2-morpholin-4-yl-2-oxo- ethyl)-1H-indole-6-carbonyl]- methanesulfonamide |
| 241 | 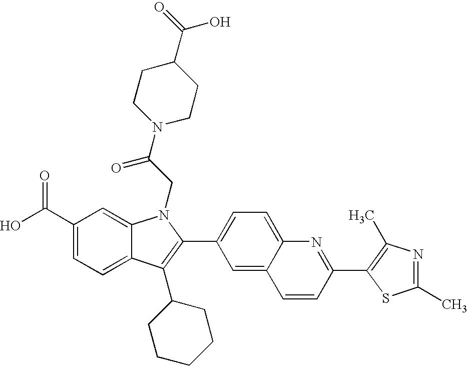 | 1-[2-(4-Carboxy-piperidin-1-yl)- 2-oxo-ethyl]-3-cyclohexyl-2- [2-(2,4-dimethyl-thiazol-5-yl)- quinolin-6-yl]-1H-indole- 6-carboxylic acid |
| 242 | 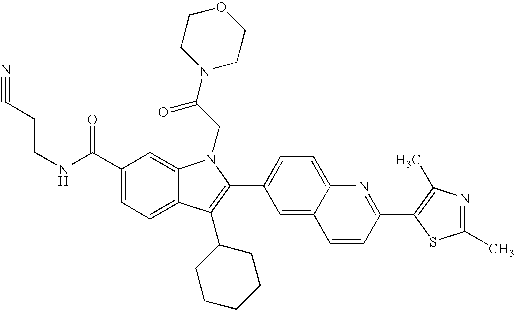 | 3-Cyclohexyl-2-[2-(2,4-dimethyl- thiazol-5-yl)-quinolin-6-yl]- 1-(2-morpholin-4-yl-2-oxo-ethyl)- 1H-indole-6-carboxylic acid (2-cyano- ethyl)-amide |
| 243 | 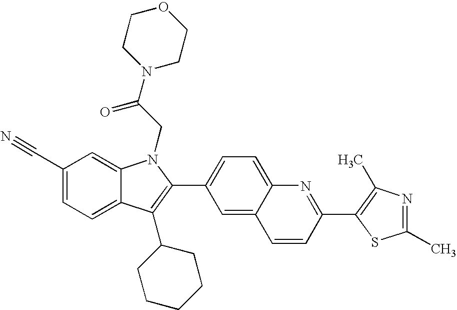 | 3-Cyclohexyl-2-[2-(2,4-dimethyl- thiazol-5-yl)-quinolin-6-yl]- 1-(2-indole-6-carbonitrile |
| 244 | 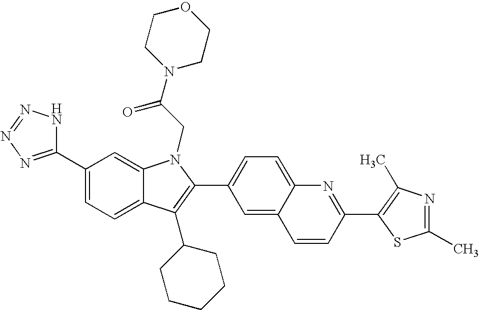 | 2-[3-Cyclohexyl-2-[2-(2,4- dimethyl-thiazol-5-yl)-quinolin-6- yl]-6-(1H-tetrazol-5-yl)- indol-1-yl]-1-morpholin-4-yl- ethanone |
| 245 | 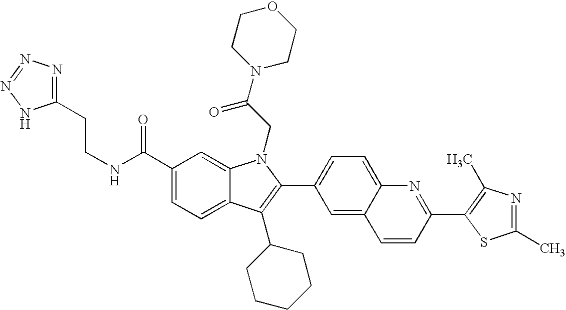 | 3-Cyclohexyl-2-[2-(2,4-dimethyl- thiazol-5-yl)-quinolin-6- yl]-1-(2-morpholin-4-yl-2-oxo- ethyl)-1H-indole-6-carboxylic acid [2- (1H-tetrazol-5-yl)-ethyl]- amide |
| 246 | 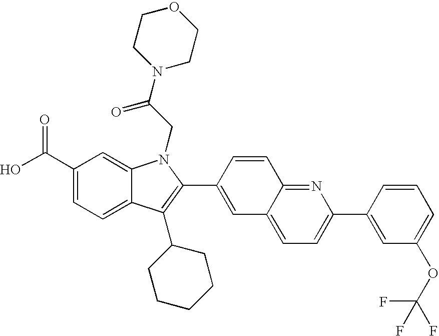 | 3-Cyclohexyl-1-(2-morpholin-4-yl-2- oxo-ethyl)-2-[2-(3-trifluoromethoxy- phenyl)-quinolin-6-yl]-1H- indole-6-carboxylic acid |
| 247 | 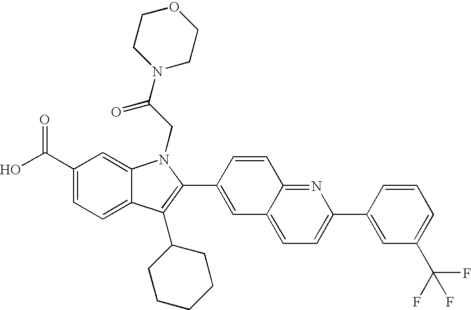 | 3-Cyclohexyl-1-(2-morpholin-4-yl- 2-oxo-ethyl)-2-[2-(3-trifluoromethyl- phenyl)-quinolin-6-yl]- 1H-indole-6-carboxylic acid |
| 248 | 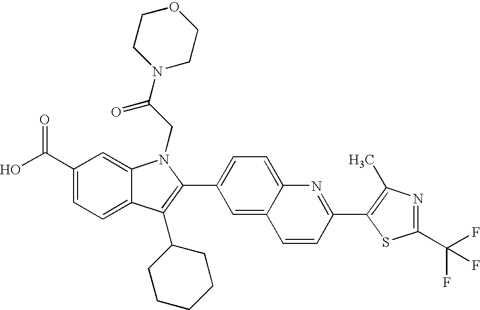 | 3-Cyclohexyl-2-[2-(4-methyl-2- trifluoromethyl-thiazol-5-yl)-quinolin-6- yl]-1-(2-morpholin-4-yl- 2-oxo-ethyl)-1H-indole- 6-carboxylic acid |
| 249 | 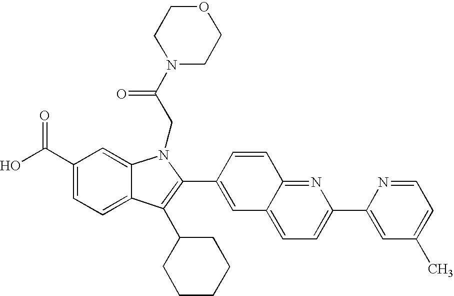 | 3-Cyclohexyl-2-[2-(4-methyl-pyridin- 2-yl)-quinolin-6-yl]-1-(2-morpholin- 4-yl-2-oxo-ethyl)-1H- indole-6-carboxylic acid |
| 250 | 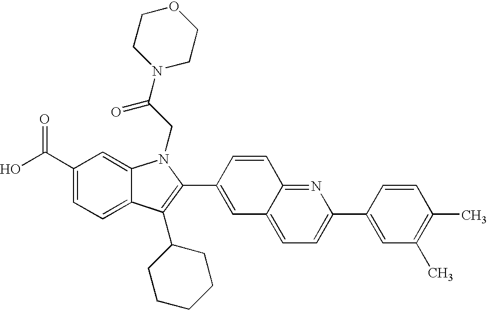 | 3-Cyclohexyl-2-[2-(3,4-dimethyl- phenyl)-quinolin-6-yl]-1-(2-morpholin-4- yl-2-oxo-ethyl)-1H-indole- 6-carboxylic acid |
| 251 | 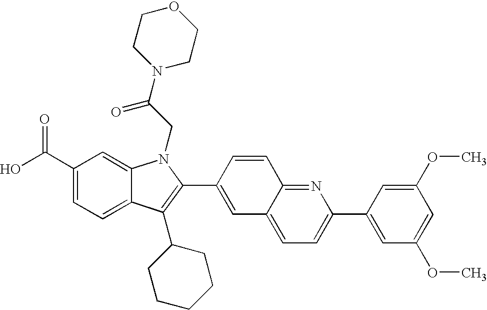 | 3-Cyclohexyl-2-[2-(3,5-dimethoxy- phenyl)-quinolin-6-yl]-1-(2-morpholin-4- yl-2-oxo-ethyl)-1H-indole-6- carboxylic |
| 252 | 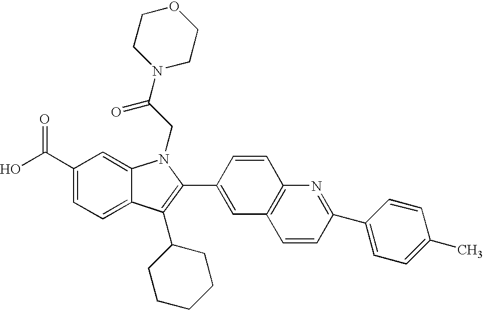 | 3-Cyclohexyl-1-(2-morpholin-4-yl-2- oxo-ethyl)-2-(2-p-tolyl-quinolin-6-yl)- 1H-indole-6-carboxylic acid |
| 253 | 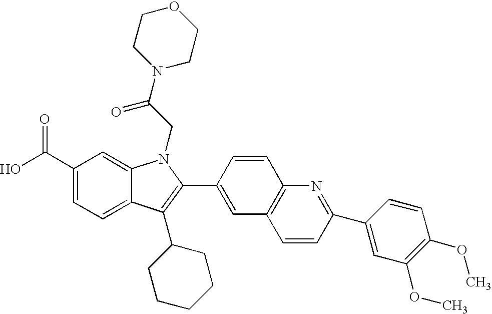 | 3-Cyclohexyl-2-(2-(3,4-dimethoxy- phenyl)-quinolin-6-yl]-1-(2-morpholin- 4-yl-2-oxo-ethyl)-1H-indole- 6-carboxylic acid |
| 254 | 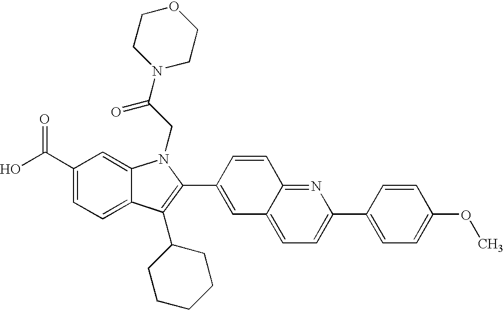 | 3-Cyclohexyl-2-[2-(4-methoxy- phenyl)-quinolin-6-yl]-1-(2- morpholin-4-yl-2-oxo-ethyl)- 1H-indole-6-carboxylic acid |
| 255 | 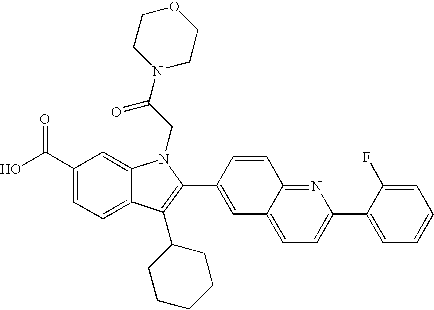 | 3-Cyclohexyl-2-[2-(2-fluoro- phenyl)-quinolin-8-yl]-1-(2- morpholin-4-yl-2-oxo-ethyl)-1H- indole-6-carboxylic acid |
| 256 | 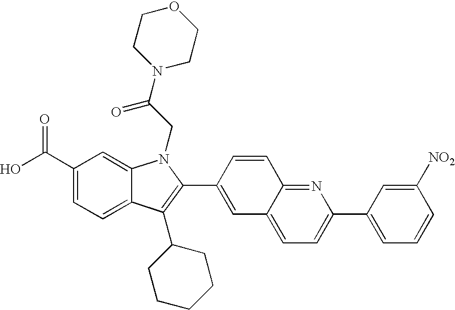 | 3-Cyclohexyl-2-[2-(3-nitro- phenyl)-quinolin-6-yl]-1-(2-morpholin- 4-yl-2-oxo-ethyl)-1H- indole-6- carboxylic acid |
| 257 | 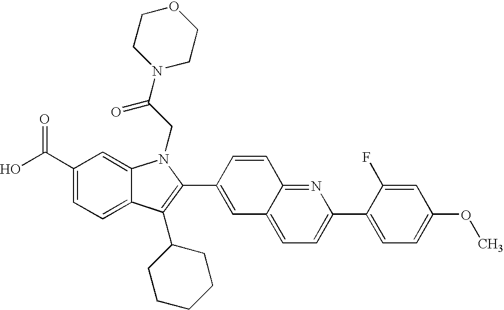 | 3-Cyclohexyl-2-[2-(2-fluoro-4-methoxy- phenyl)-quinolin-6-yl]-1- (2-morpholin-4-yl-2-oxo- ethyl)-1H-indole-6- carboxylic acid |
| 258 | 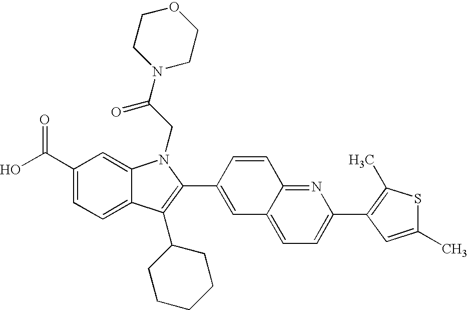 | 3-Cyclohexyl-2-[2-(2,5-dimethyl- thiophen-3-yl)-quinolin-6-yl]-1- (2-morpholin-4-yl-2-oxo- ethyl)-1H-indole-6- carboxylic acid |
| 259 | 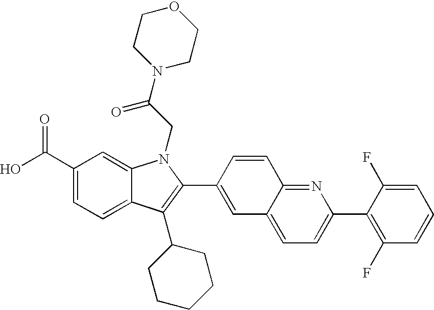 | 3-Cyclohexyl-2-[2-(2,6-difluoro- phenyl)-quinolin-6-yl]-1-(2-morpholin- 4-yl-2-oxo-ethyl)- 1H-indole-6-carboxylic acid |
| 260 | 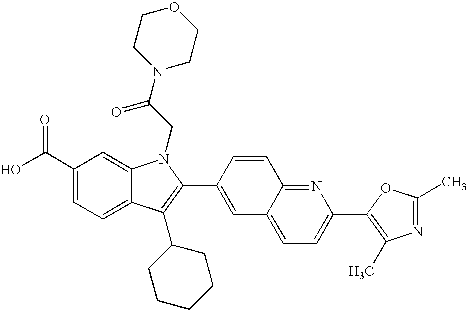 | 3-Cyclohexyl-2-[2-(2,4-dimethyl- oxazol-5-yl)-quinolin-6-yl]- 1-(2-morpholin-4-yl-2- oxo-ethyl)-1H-indole- 6-carboxylic acid |
| 261 | 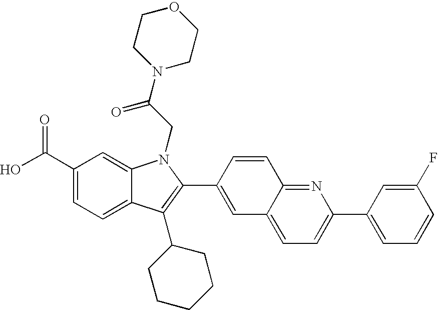 | 3-Cyclohexyl-2-[2-(3-fluoro- phenyl)-quinolin-6-yl]-1-(2- morpholin-4-yl-2-oxo-ethyl)-1H- indole-6-carboxylic acid |
| 262 | 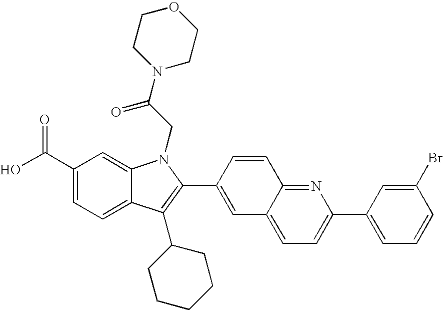 | 2-[2-(3-Bromo-phenyl)-quinolin- 6-yl]-3-cyclohexyl-1-(2- morpholin-4-yl-2-oxo-ethyl)-1H- indole-6-carboxylic acid |
| 263 | 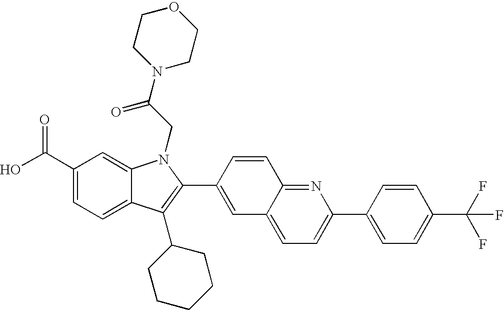 | 3-Cyclohexyl-1-(2-morpholin-4-yl- 2-oxo-ethyl)-2-[2-(4-trifluoromethyl- phenyl)-quinolin-6-yl]-1H-indole-6- carboxylic acid |
| 264 | 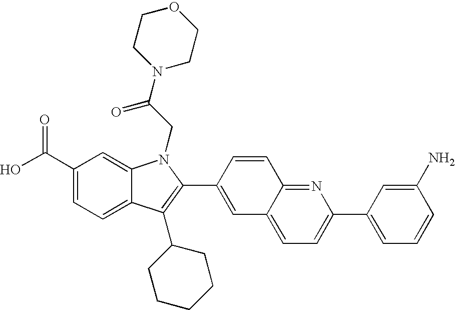 | 2-[2-(3-Amino-phenyl)-quinolin-6- yl]-3-cyclohexyl-1-(2-morpholin-4- yl-2-oxo-ethyl)-1H-indole-6- carboxylic acid |
| 265 | 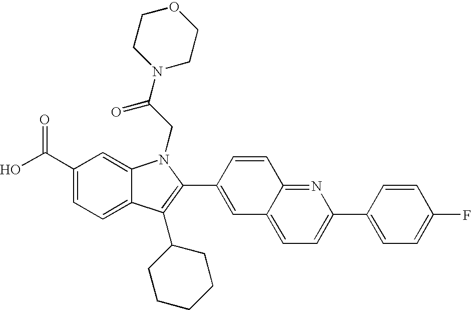 | 3-Cyclohexyl-2-(2-(4-fluoro-phenyl)- quinolin-6-yl]-1-(2-morpholin-4- yl-2-oxo-ethyl)-1H-indole- 6-carboxylic acid |
| 266 | 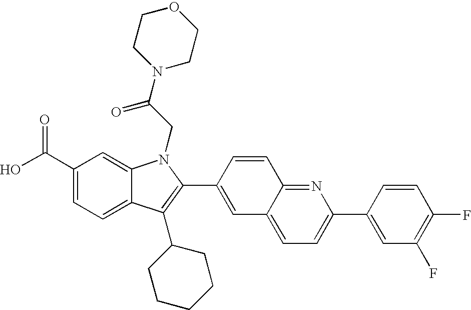 | 3-Cyclohexyl-2-[2-(3,4-difluoro- phenyl)-quinolin-6-yl]-1-(2-morpholin- 4-yl-2-oxo-ethyl)-1H-indole- 6-carboxylic acid |
| 267 | 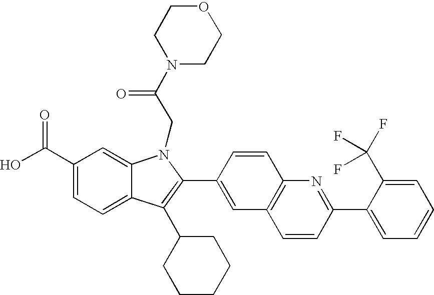 | 3-Cyclohexyl-1-(2-morpholin-4-yl- 2-oxo-ethyl)-2-[2-(2-trifluoromethyl- phenyl)-quinolin-6-yl]-1H-indole- 6-carboxytic acid |
| 268 | 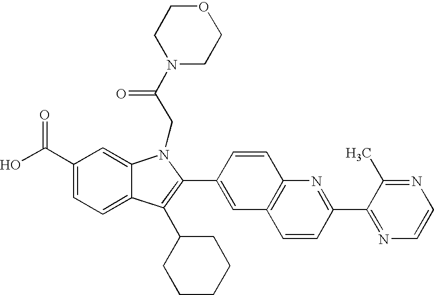 | 3-Cyclohexyl-2-[2-(3-methyl- pyrazin-2-yl)-quinolin-6-yl]-1- (2-morpholin-4-yl-2-oxo-ethyl)- 1H-indole-6-carboxylic acid |
| 269 | 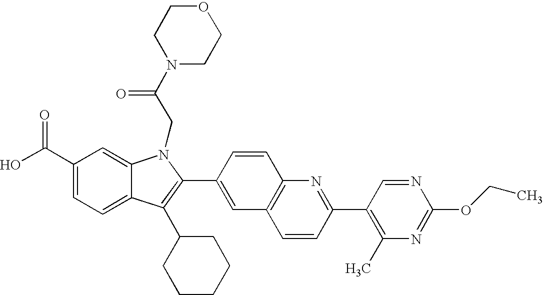 | 3-Cyclohexyl-2-[2-(2-ethoxy- 4-methyl-pyrimidin-5-yl)-quinolin-6- yl]-1-(2-morpholin-4-yl-2- oxo-ethyl)-1H-indole-6- carboxylic acid |
| 270 | 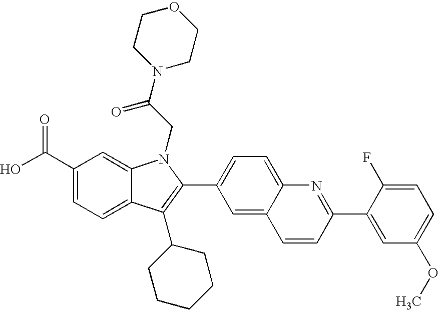 | 3-Cyclohexyl-2-[2-fluoro-5-methoxy- pheny)-quinolin-6-yl]-1-(2-morpholin- 4-yl-2-oxo-ethyl)-1H- indole-6-carboxylic acid |
| 271 | 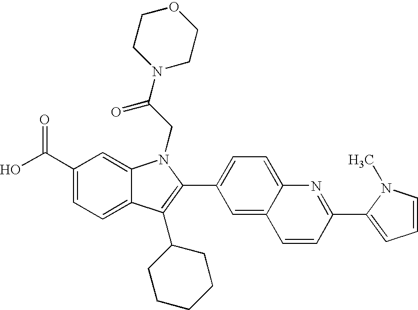 | 3-Cyclohexyl-2-[2-(1-methyl-1H- pyrrol-2-yl)-quinolin-6-yl]-1- (2-morpholin-4-yl-2-oxo-ethyl)- 1H-indole-6-carboxylic acid |
| 272 | 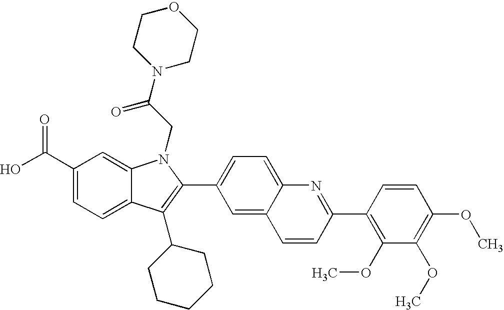 | 3-Cyclohexyl-1-(2-morpholin-4-yl- 2-oxo-ethyl)-2-[2-(2,3,4- trimethoxy-phenyl)-quinolin- 6-yl]-1H-indole- 6-carboxylic acid |
| 273 | 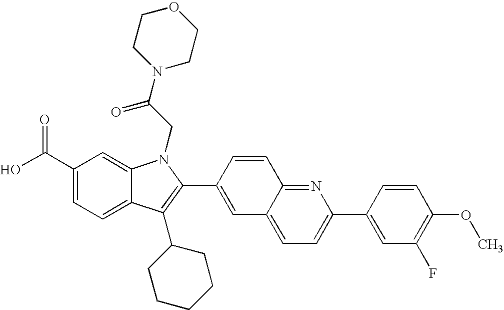 | 3-Cyclohexyl-2-[2-(3-fluoro-4- methoxy-phenyl)-quinolin-6-yl]-1-(2- morpholin-4-yl-2-oxo-ethyl)-1H- indole-6-carboxylic acid |
| 274 | 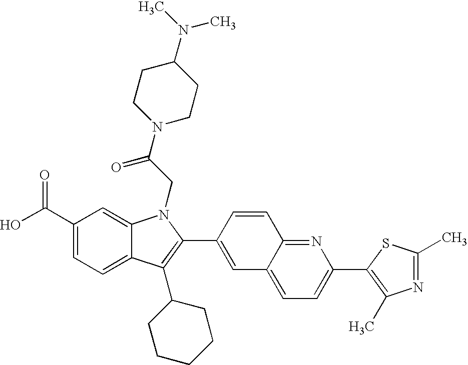 | 3-Cyclohexyl-1-[2-(4- dimethylamino-piperidin- 1-yl)-2-oxo-ethyl]-2-[2- (2,4-dimethyl-thiazol-5-yl)-quinolin-6- yl]-1H-indole-6-carboxylic acid |
| 275 | 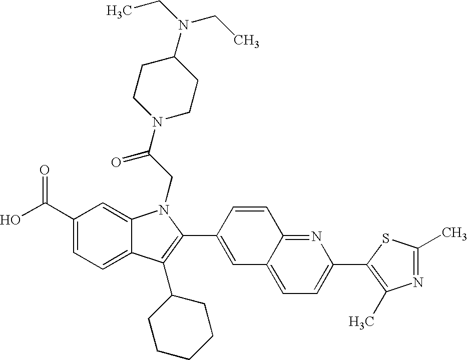 | 3-Cyclohexyl-1-[2-( 4-diethylamino-piperidin- 1-yl)-2-oxo-ethyl]-2-[2- (2,4-dimethyl-thiazol-5-yl)-quinolin-6- yl]-1H-indole- 6-carboxylic acid |
| 276 | 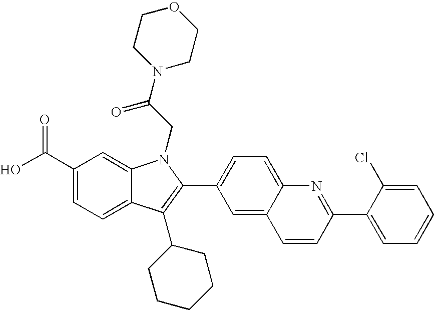 | 2-[2-(2-Chloro- phenyl)-quinolin-6- yl]-3-cyclohexyl-1-(2-morpholin-4- yl-2-oxo-ethyl)-1H-indole- 6-carboxylic acid |
| 277 | 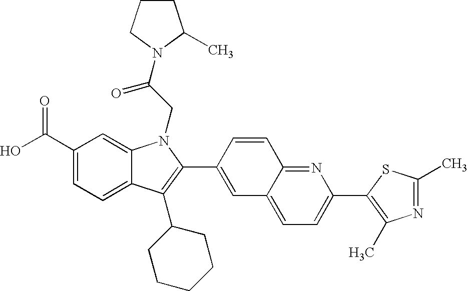 | 3-Cyclohexyl-2-[2- (2,4-dimethyl-thiazol- 5-yl)-quinolin-6-yl]-1-[2- (2-methyl-pyrrolidin-1-yl)-2-oxo- ethyl]-1H-indole-6-carboxylic acid |
| 278 | 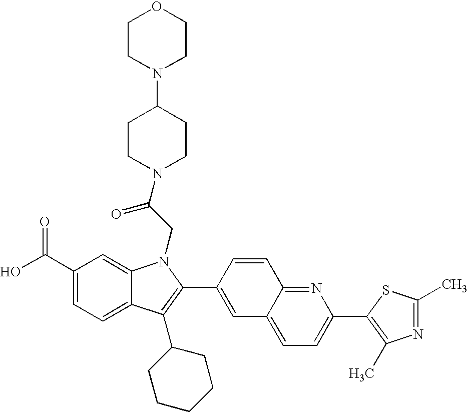 | 3-Cyclohexyl-2-[2-(2,4-dimethyl- thiazol-5-yl)-quinolin-6-yl]-1- (2-(4-morpholin-4-yl-piperidin-1- yl)-2-oxo-ethyl]-1H-indole- 6-carboxylic acid |
| 279 | 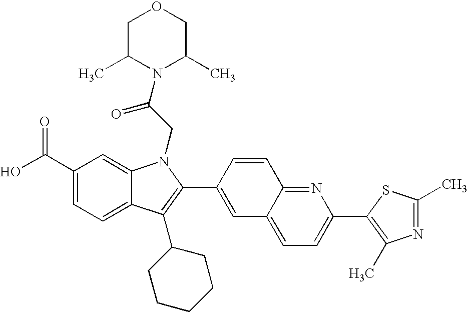 | 3-Cyclohexyl-1 -[2-(3,5-dimethyl- morpholin- 4-yl)-2-oxo-ethyl]-2- [2- (2,4-dimethyl-thiazol-5-yl)- carboxylic acid |
| 280 | 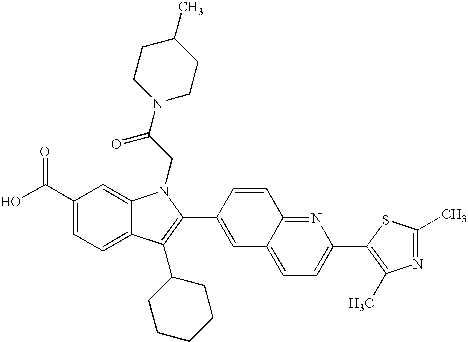 | 3-Cyclohexyl-2- [2-(2,4-dimethyl-thiazol-5- yl)-quinolin-6-yl]-1-[2-(4- methyl-piperidin-1-yl)-2-oxo- ethyl]-1H-indole-6-carboxylic acid |
| 281 | 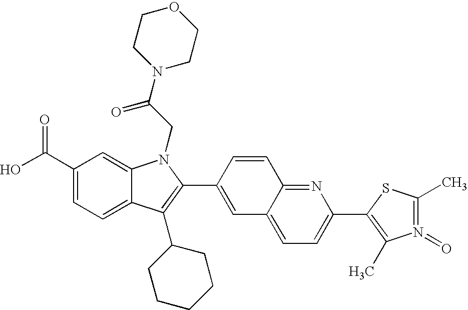 | 3-Cyclohexyl-2-[2-(2,4-dimethyl-3-oxy- thiazol-5-yl)-quinolin-6-yl]-1-(2- morpholin-4-yl-2-oxo- ethyl)-1H-indole-6-carboxylic acid |
| 282 | 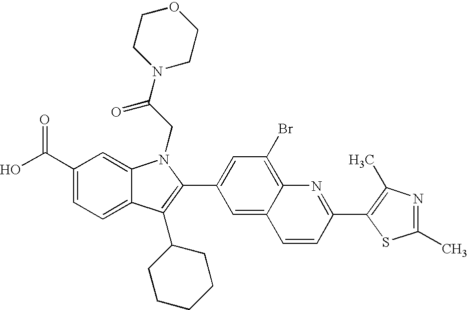 | 2-[8-Bromo-2-(2,4-dimethyl-thiazol-5-yl)- quinolin-6-yl]-3-cyclohexyl-1- (2-morpholin-4-yl-2-oxo-ethyl)- 1H-indole-6-carboxylic acid |
| 283 | 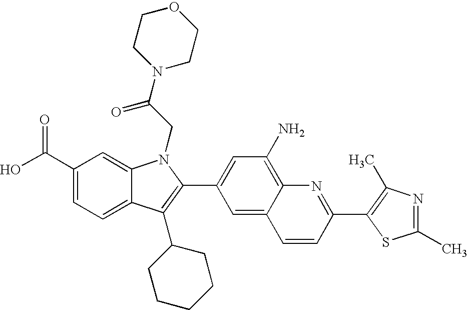 | 2-[8-amino-2-(2,4-dimethyl-thiazol- 5-yl)-quinolin-6-yl]-3-cyclohexyl- 1-(2-morpholin-4-yl-2-oxo-ethyl)- 1H-indole-6-carboxylic acid |
| 284 | 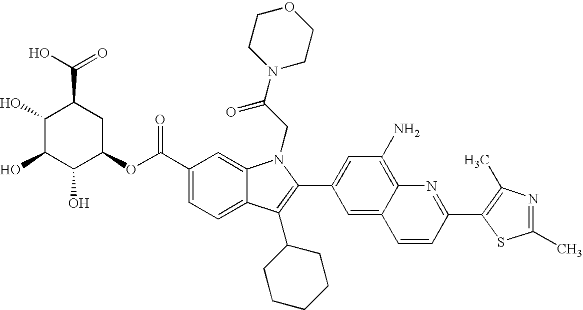 | 3-Cyclohexyl-2-[2-(2,4-dimethyl-thiazol- 5-yl)-quinolin-6-yl]-1-(2-morpholin- 4-yl-2-oxo-ethyl)-1H-indole-6-(β- D-glucuronic acid) ester |
| 285 | 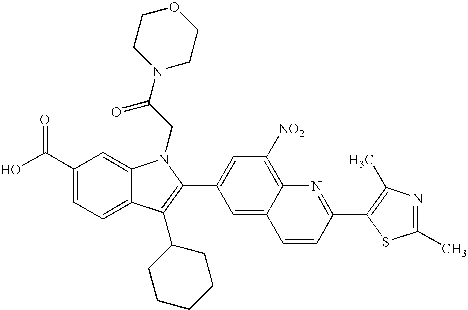 | 2-[8-nitro-2-(2,4-dimethyl-thiazol-5-yl)- quinolin-6-yl]-3-cyclohexyl-1- (2-morpholin-4-yl-2-oxo-ethyl)- 1H-indole-6-carboxylic acid |

TABLE III

| | | |
|---|---|---|
| 286 | 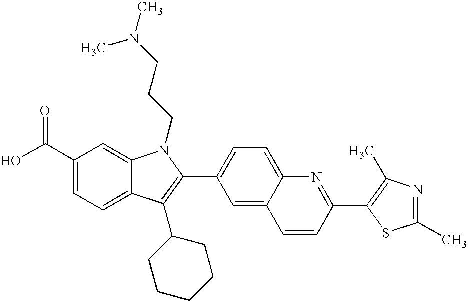 | 3-Cyclohexyl-1-(3-dimethylamino- propyl)-2-[2-(2,4-dimethyl-thiazol- 5-yl)-quinolin-6-yl]-1H-indole-6-carboxylic acid |
| 287 | 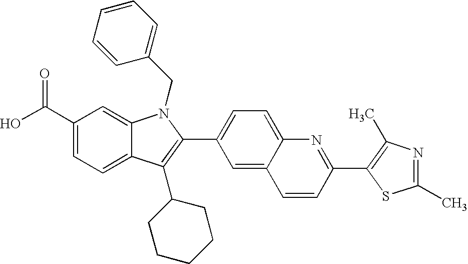 | 1-Benzyl-3-cyclohexyl-2-[2-(2,4- dimethyl-thiazol-5-yl)-quinolin- 6-yl]-1H-indole-6-carboxylic acid |
| 289 | 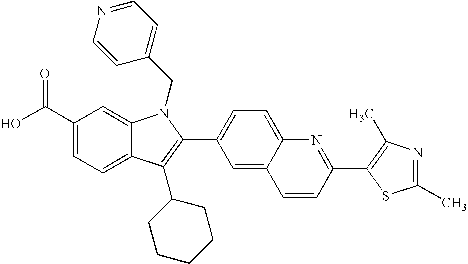 | 3-Cyclohexyl-2-[2-(2,4-dimethyl- thiazol-5-yl)-quinolin-6-yl]- 1-pyridin-4-ylmethyl-1H-indole-6-carboxylic acid |
| 290 | 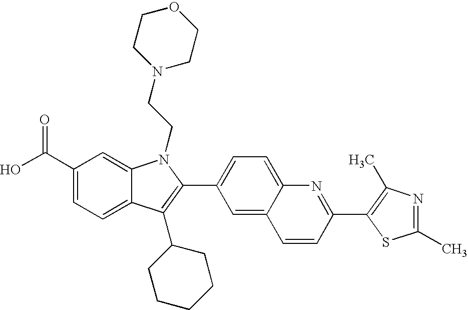 | 3-Cyclohexyl-2-[2-(2,4-dimethyl- thiazol-5-yl)-quinolin-6-yl]- 1-(2-morpholin-4-yl-ethyl)-1H-indole-6-carboxylic acid |
| 291 | 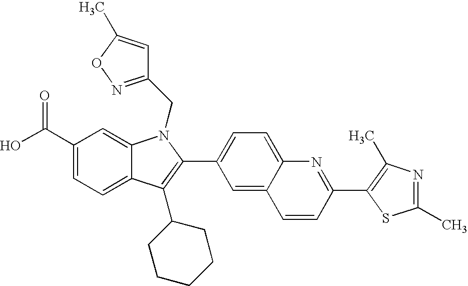 | 3-Cyclohexyl-2-[2-(2,4-dimethyl- thiazol-5-yl)-quinolin-6-yl]- 1-(5-methyl-isoxazol-3-ylmethyl)-1H-indole-6-carboxylic acid |
| 292 | 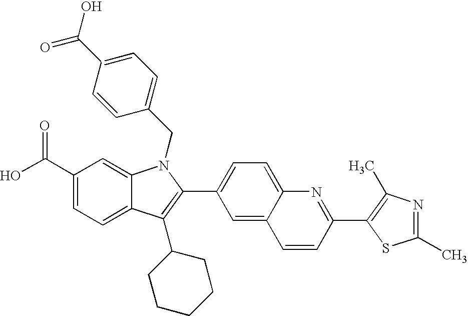 | 1-(4-Carboxy-benzyl)-3-cyclohexyl- 2-[2-(2,4-dimethyl-thiazol-5- yl)-quinolin-6-yl]-1H-indole-6-carboxylic acid |
| 293 | 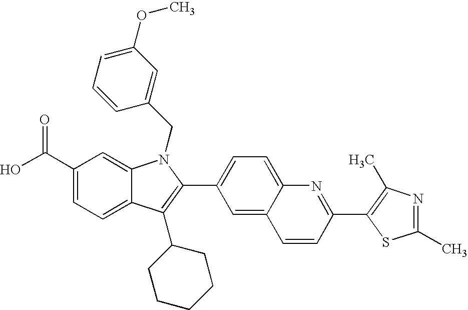 | 3-Cyclohexyl-2-[2-[2,4-dimethyl- thiazol-5-yl)-quinolin-6- yl]-1-(3-methoxy-benzyl)-1H-indole-6-carboxylic acid |

TABLE IV

| | | |
|---|---|---|
| 298 | 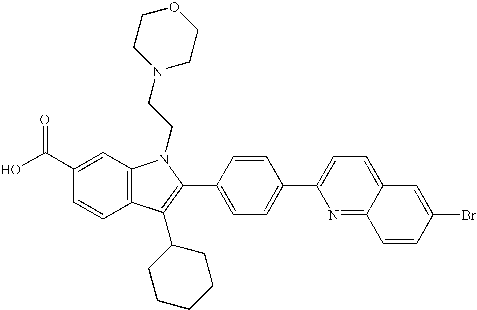 | 2-[4-(6-Bromo-quinolin-2-yl)-phenyl]- 3-cyclohexyl-1-(2-morpholin-4-yl-2-oxo-ethyl)-1H-indole- 6-carboxylic acid |
| 299 | 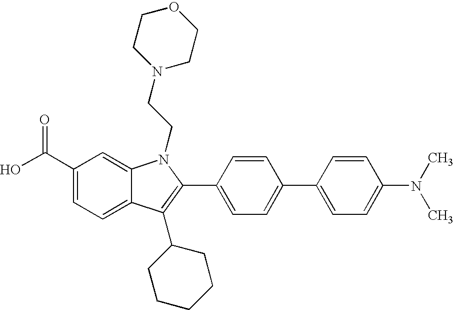 | 3-Cyclohexyl-2-(4′-dimethylamino- biphenyl-4-yl)-1-(2-morpholin-4-yl-2-oxo-ethyl)-1H-indole-6- carboxylic acid |
| 300 | 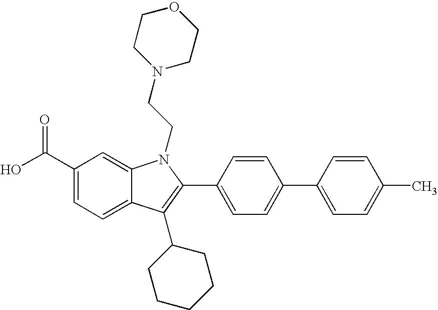 | 3-Cyclohexyl-2-(4′-methyl-biphenyl-4- yl)-1-(2-morpholin-4-yl-2-oxo-ethyl)-1H-indole- 6-carboxylic acid |
| 301 | 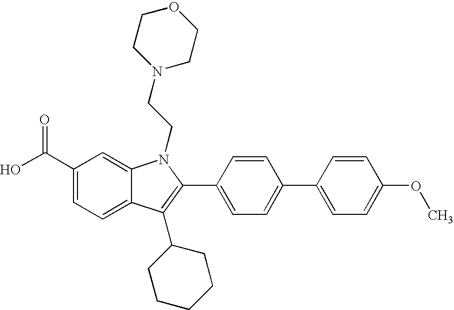 | 3-Cyclohexyl-2-(4′-methoxy-biphenyl-4- yl)-1-(2-morpholin-4-yl-2-oxo-ethyl)-1H-indole- 6-carboxylic acid |
| 302 | 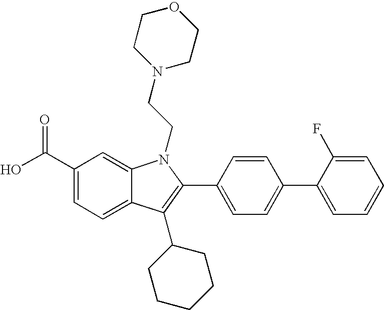 | 3-Cyclohexyl-2-(2′-fluoro-biphenyl-4- yl)-1-(2-morpholin-4-yl-2-oxo-ethyl)-1H-indole- 6-carboxylic acid |
| 303 | 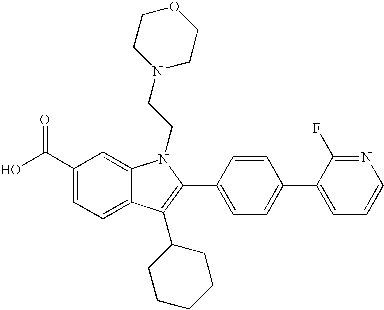 | 3-Cyclohexyl-2-[4-(2-fluoro-pyridin-3- yl)-phenyl]-1-(2-morpholin-4-yl-2-oxo-ethyl)-1H-indole- 6-carboxylic acid |
| 304 | 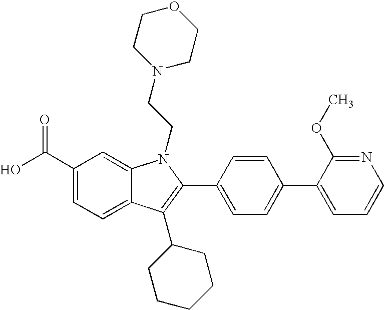 | 3-Cyclohexyl-2-[4-(2-methoxy-pyridin-3- yl)-phenyl]-1-(2-morpholin-4-yl-2-oxo-ethyl)-1H-indole- 6-carboxylic acid |
| 305 | 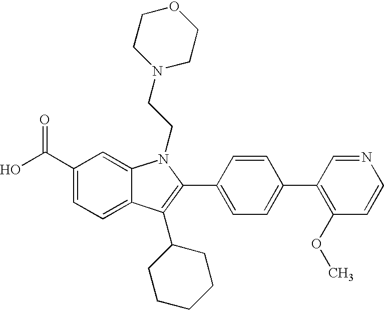 | 3-Cyclohexyl-2-[4-(4-methoxy-pyridin-3- yl)-phenyl]-1-(2-morpholin-4-yl-2-oxo-ethyl)-1H-indole- 6-carboxylic acid |
| 306 | 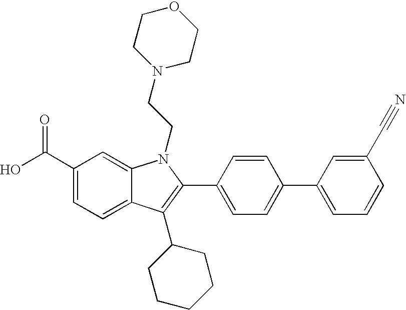 | 2-(3′-Cyano-biphenyl-4-yl)-3-cyclohexyl- 1-(2-morpholin-4-yl-2-oxo-ethyl)-1H-indole- 6-carboxylic acid |
| 307 | 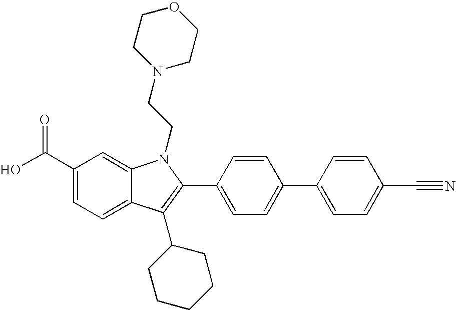 | 2-(4′-Cyano-biphenyl-4-yl)-3-cyclohexyl- 1-(2-morpholin-4-yl-2-oxo-ethyl)-1H-indole- 6-carboxylic acid |
| 308 | 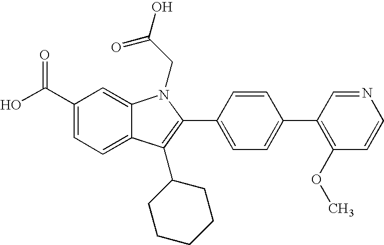 | 1-Carboxymethyl-3-cyclohexyl-2-[4-(4- methoxy-pyridin-3-yl)-phenyl]-1H-indole-6- carboxylic acid |
| 309 | 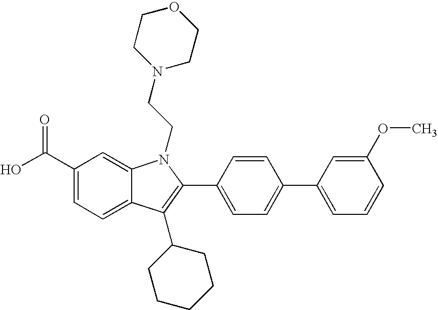 | 3-Cyclohexyl-2-(3′-methoxy-biphenyl-4-yl)- 1-(2-morpholin-4-yl-2-oxo-ethyl)-1H-indole- 6-carboxylic acid |
| 310 | 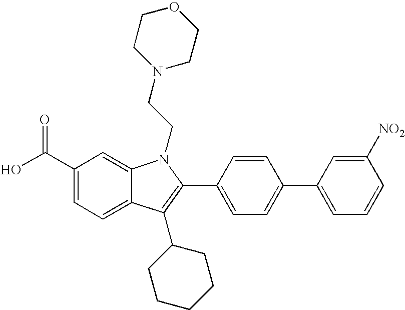 | 3-Cyclohexyl-2-(3′-nitro-biphenyl-4-yl)- 1-(2-morpholin-4-yl-2-oxo-ethyl)-1H-indole- 6-carboxylic acid |
| 311 | 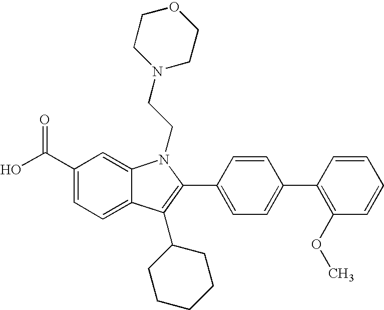 | 3-Cyclohexyl-2-(2′-methoxy-biphenyl-4-yl)- 1-(2-morpholin-4-yl-2-oxo-ethyl)-1H-indole- 6-carboxylic acid |
| 312 | 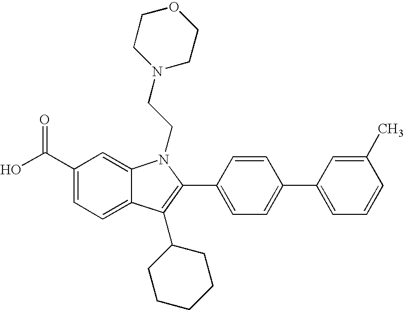 | 3-Cyclohexyl-2-(3′-methyl-biphenyl-4-yl)- 1-(2-morpholin-4-yl-2-oxo-ethyl)-1H-indole- 6-carboxylic acid |
| 313 | 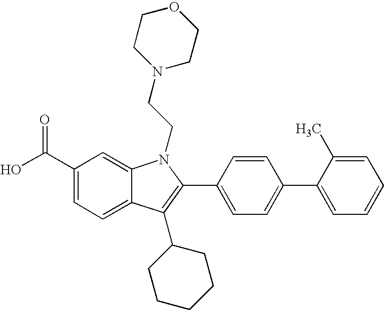 | 3-Cyclohexyl-2-(2′-methyl-biphenyl-4-yl)- 1-(2-morpholin-4-yl-2-oxo-ethyl)-1H-indole- 6-carboxylic acid |
| 314 | 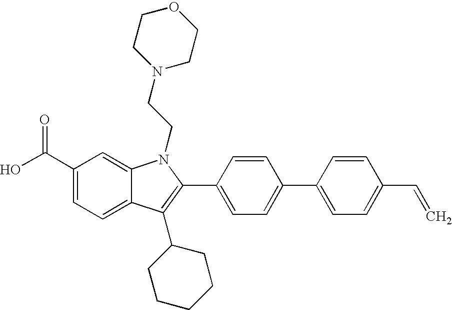 | 3-Cyclohexyl-1-(2-morpholin-4-yl-2-oxo-ethyl)- 2-(4′-vinyl-biphenyl-4-yl)-1H-indole- 6-carboxylic acid |
| 315 | 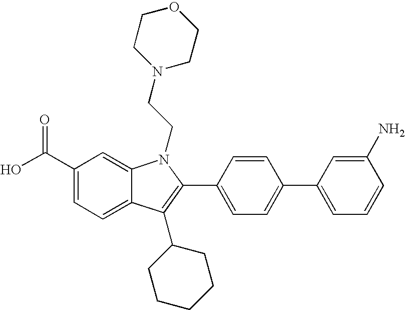 | 2-(3′-Amino-biphenyl-4-yl)-3-cyclohexyl-1- (2-morpholin-4-yl-2-oxo-ethyl)-1H-indole- 6-carboxylic acid |
| 316 | 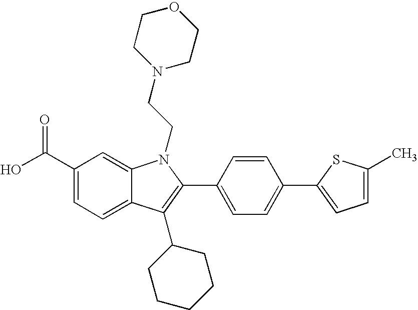 | 3-Cyclohexyl-2-[4-(5-methyl-thiophen-2-yl)- phenyl]-1-(2-morpholin-4-yl-2-oxo-ethyl)-1H-indole- 6-carboxylic acid |
| 317 | 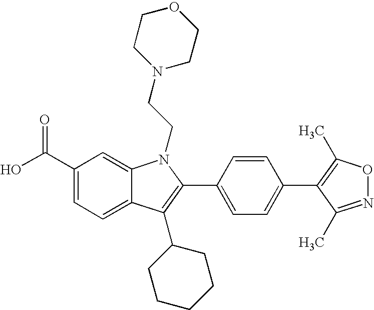 | 3-Cyclohexyl-2-[4-(3,5-dimethyl-isoxazol-4- yl)-phenyl]-1-(2-morpholin-4-yl-2-oxo-ethyl)-1H-indole- 6-carboxylic acid |
| 318 | 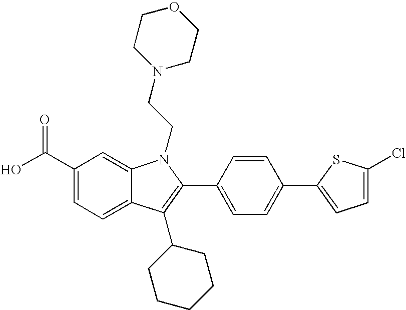 | 2-[4-(5-Chloro-thiophen-2-yl)-phenyl]-3- cyclohexyl-1-(2-morpholin-4-yl-2-oxo-ethyl)-1H-indole- 6-carboxylic acid |

TABLE V

| | | |
|---|---|---|
| 319 | 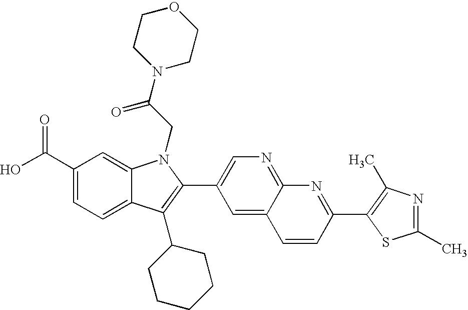 | 3-Cyclohexyl-2-[7-(2,4-dimethyl- thiazol-5-yl)-[1,8]naphthyridin-3-yl]-1-(2- morpholin-4-yl-2-oxo-ethyl)- 1H-indole-6-carboxylic acid |
| 320 | 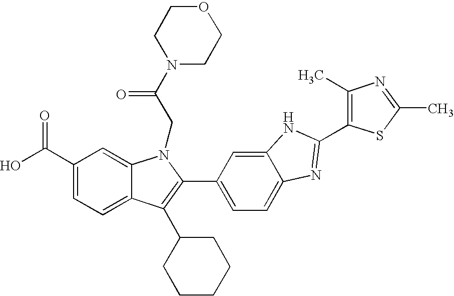 | 3-Cyclohexyl-2-[2-(2,4-dimethyl- thiazol-5-yl)-3H-benzoimidazol-5-yl]-1-(2-morpholin- 4-yl-2-oxo-ethyl)-1H- indole-6-carboxylic acid |
| 321 | 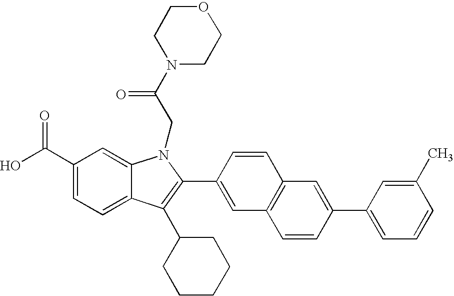 | 3-Cyclohexyl-1-(2-morpholin-4-yl- 2-oxo-ethyl)-2-(6-m-tolyl-naphthalen-2- yl)-1H-indole-6-carboxylic acid |
| 322 | 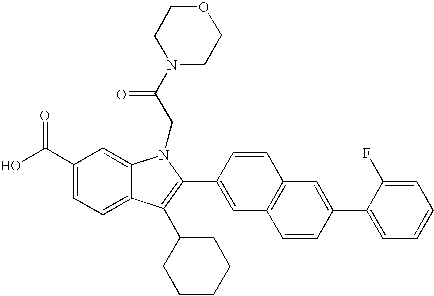 | 3-Cyclohexyl-2-[6-(2-fluoro-phenyl)- naphthalen-2-yl]-1-(2-morpholin-4- yl-2-oxo-ethyl)-1H-indole-6-carboxylic acid |
| 323 | 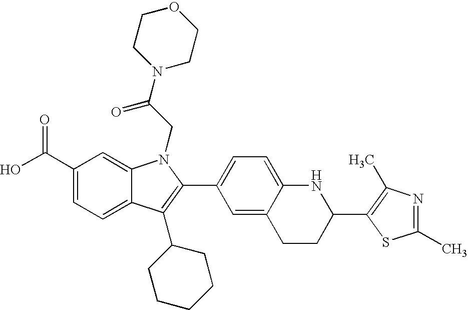 | 3-Cyclohexyl-2-[2-(2,4-dimethyl-thiazol-5- yl)-1,2,3,4-tetrahydro-quinolin-6- yl]-1-(2-morpholin-4-yl- 2-oxo-ethyl)-1H-indole-6-carboxylic acid |
| 324b 324a | 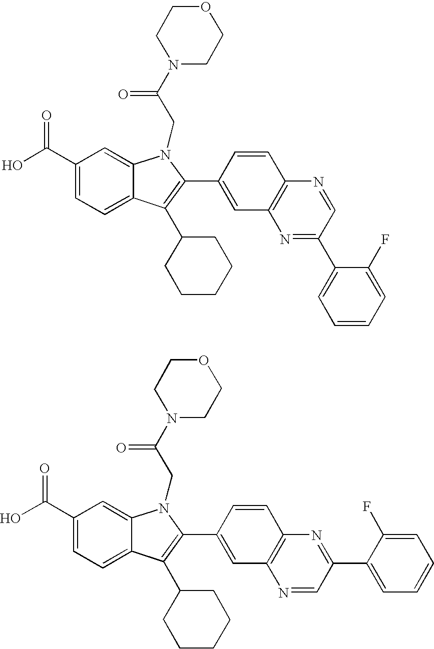 | 3-cyclohexyl-2-13-(2-fluoro-phenyl)-quinoxalin- 6-yl]-1-(2-morpholin-4-yl-2- oxo-ethyl)-1H-indole-6-carboxylic acid (324b); and 3-Cyclohexyl-2-[2-(2-fluoro-phenyl)-quinoxalin-6-yl]- 1-(2-morpholin-4-yl-2-oxo-ethyl)-1H- indole-6-carboxylic acid (324a) |

TABLE VI

| | | |
|---|---|---|
| 325 | 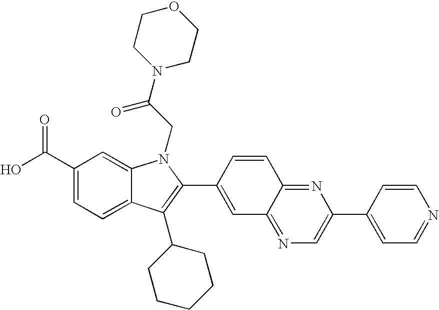 | 3-Cyclohexyl-1-(2-morpholin-4-yl-2-oxo- ethyl)-2-(2-pyridin-4-yl-quinolin-6-yl)- 1H-indole-6-carboxylic acid |
| 326 | 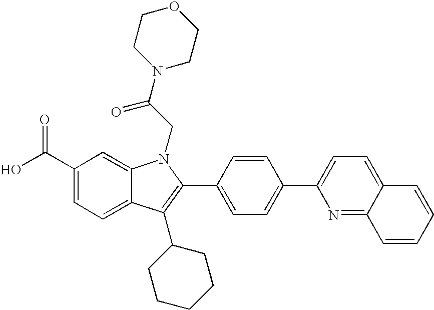 | 3-Cyclohexyl-1-(2-morpholin-4-yl-2-oxo- ethyl)-2-(4-quinolin-2-yl-phenyl)-1H-indole- 6-carboxylic acid |
| 327 | 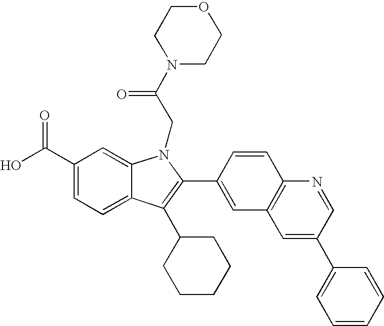 | 3-Cyclohexyl-1-(2-morpholin-4-yl-2-oxo- ethyl)-2-(3-phenyl-quinolin-6-yl)-1H-indole- 6-carboxylic acid |
| 328 | 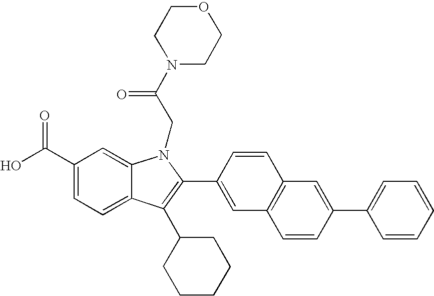 | 3-Cyclohexyl-1-(2-morpholin-4-yl-2-oxo- ethyl)-2-(6-phenyl-naphthalen-2-yl)-1H-indole- 6-carboxylic acid |
| 329 | 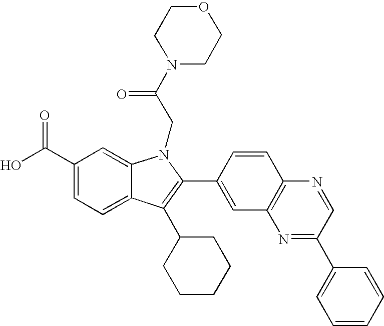 | 3-Cyclohexyl-1-(2-morpholin-4-yl-2-oxo- ethyl)-2-(3-phenyl-quinoxalin-6-yl)-1H-indole- 6-carboxylic acid |
| 330 | 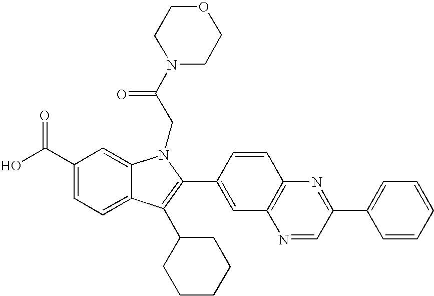 | 3-Cyclohexyl-1-(2-morpholin-4-yl-2-oxo- ethyl)-2-(2-phenyl-quinoxalin-6-yl)-1H-indole- 6-carboxylic acid |
| 331 | 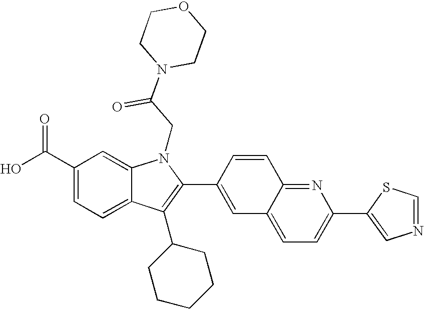 | 3-Cyclohexyl-1-(2-morpholin-4-yl-2-oxo- ethyl)-2-(2-thiazol-5-yl-quinolin-6-yl)-1H-indole- 6-carboxylic acid |
| 332 | 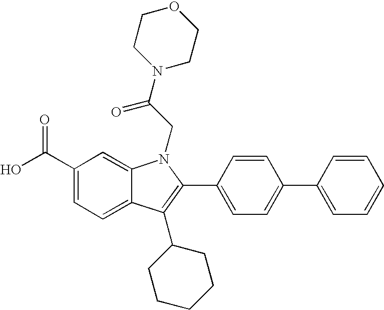 | 2-Biphenyl-4-yl-3-cyclohexyl-1-(2-morpholin- 4-yl-2-oxo-ethyl)-1H-indole-6-carboxylic acid |
| 333 | 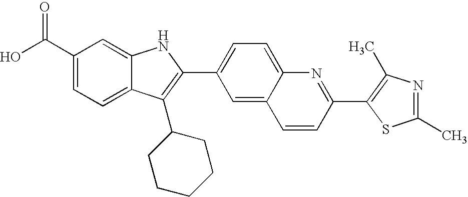 | 3-Cyclohexyl-2-[2-(2,4-dimethyl-thiazol-5- yl)-quinolin-6-yl]-1H- indole-6-carboxylic acid |
| 334 | 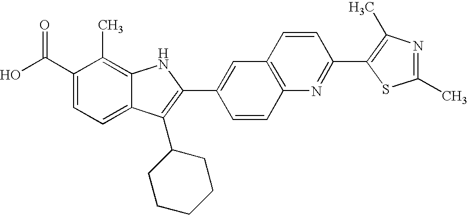 | 3-Cyclohexyl-2-[2-(2,4-dimethyl-thiazol-5- yl)-quinolin-6-yl]-7-methyl- 1H-indole-6-carboxylic acid |
| 335 | 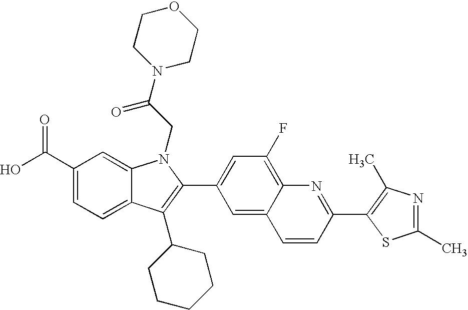 | 2-[8-fluoro-2-(2,4-dimethyl-thiazol-5-yl)- quinolin-6-yl]-3-cyclohexyl-1-(2-morpholin-4-yl-2-oxo- ethyl)-1H-indole-6-carboxylic acid |
| 336 | 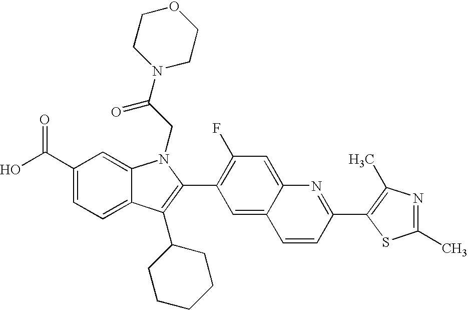 | 2-[7-fluoro-2-(2,4-dimethyl-thiazol-5-yl)- quinolin-6-yl]-3-cyclohexyl-1-(2-morpholin-4-yl-2-oxo- ethyl)-1H-indole-6-carboxylic acid |

This invention is also directed to pharmaceutical compositions comprising a pharmaceutically acceptable diluent and a therapeutically effective amount of one of the compounds described herein or mixtures of one or more of such compounds.

This invention is further directed to methods for treating a viral infection mediated at least in part by a virus in the Flaviviridae family of viruses, such as HCV, in mammals which methods comprise administering to a mammal, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds. In another aspect, present invention provides for use of the compounds of the invention for the preparation of a medicament for treating or preventing said infections.

In yet another embodiment of the invention, methods of treating or preventing viral infections in mammals are provided where in the compounds of this invention are administered in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. Preferably, the additional agent active against HCV is interferon-alpha or pegylated interferon-alpha alone or in combination with ribavirin or viramidine.

Definitions

It is to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “pharmaceutically acceptable diluent” in a composition includes two or more pharmaceutically acceptable diluents, and so forth.

In this specification and in the claims that follow, reference will be made to a number of terms that shall be defined to have the following meanings:

As used herein, “alkyl” refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, cycloalkenyl, substituted cycloalkenyl, heteroaryl, substituted heteroaryl, heterocyclic, and substituted heterocyclic.

“Alkoxy” refers to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O-”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)-cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Acylamino” refers to the group —C(O)NR^{f}R^{g} where R^{f} and R^{g} is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{f} and R^{g} are joined to form together with the nitrogen atom a heterocyclic or substituted heterocyclic ring.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

“Alkenyl” refers to alkenyl group having from 2 to 10 carbon atoms, preferably having from 2 to 6 carbon atoms, and more preferably 2 to 4 carbon atoms and having at least 1 and preferably from 1-2 sites of alkenyl unsaturation.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to a vinyl carbon atom.

“Alkynyl” refers to alkynyl group having from 2 to 10 carbon atoms, preferably having from 2 to 6 carbon atoms, and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to an acetylenic carbon atom.

“Amino” refers to the group —NH₂.

“Substituted amino” refers to the group —NR^{h}Rⁱ where R^{h} and Rⁱ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{h} and Rⁱ are joined, together with the nitrogen bound thereto to form a heterocyclic or substituted heterocyclic group provided that R^{h} and Rⁱ are both not hydrogen. When R^{h} is hydrogen and Rⁱ is alkyl, the substituted amino group is sometimes referred to herein as alkylamino. When R^{h} and Rⁱ are alkyl, the substituted amino group is sometimes referred to herein as dialkylamino.

“Aminoacyl” refers to the groups —NR^{j}C(O)alkyl, —NR^{j}C(O)substituted alkyl, —NR^{j}C(O)-cycloalkyl, —NR^{j}C(O)substituted cycloalkyl, —NR^{j}C(O)alkenyl, —NR^{j}C(O)substituted alkenyl, —NR^{j}C(O)alkynyl, —NR^{j}C(O)substituted alkynyl, —NR^{j}C(O)aryl, —NR^{j}C(O)substituted aryl, —NR^{j}C(O)heteroaryl, —NR^{j}C(O)substituted heteroaryl, —NR^{j}C(O)heterocyclic, and —NR^{j}C(O)substituted heterocyclic where R^{j} is hydrogen or alkyl.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl.

“Aralkyl” or “arylalkyl” refers to the group aryl-alkyl- and includes, for example, benzyl.

“Substituted aryl” refers to aryl groups which are substituted with from 1 to 3 substituents, and preferably 1 to 2 substituents, selected from the group consisting of hydroxy, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy esters, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Arylene” and “substituted arylene” refer to divalent aryl and substituted aryl groups as defined above. “Phenylene” is a 6-membered optionally substituted arylene group and includes, for example, 1,2-phenylene, 1,3-phenylene, and 1,4-phenylene.

“Aryloxy” refers to the group aryl-O— that includes, by way of example, phenoxy, naphthoxy, and the like.

“Substituted aryloxy” refers to substituted aryl-O— groups.

“Carboxy” refers to —C(═O)OH or salts thereof.

“Carboxy esters” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic. Preferred carboxy esters are —C(O)O-alkyl, —C(O)O-substituted alkyl, —C(O)O-aryl, and —C(O)O-substituted aryl.

“Cycloalkyl” refers to cyclic alkyl groups of from 3 to 10 carbon atoms having single or multiple cyclic rings optionally comprising 1 to 3 exo carbonyl or thiocarbonyl groups. Suitable cycloalkyl groups include, by way of example, adamantyl, cyclopropyl, cyclobutyl, cyclopentyl, cyclohexyl, cyclooctyl, 3-oxocyclohexyl, and the like. In multiple condensed rings, one or more of the rings may be other than cycloalkyl (e.g., aryl, heteroaryl or heterocyclic) provided that the point of attachment is to a carbon ring atom of the cycloalkyl group. In one embodiment, the cycloalkyl group does not comprise 1 to 3 exo carbonyl or thiocarbonyl groups. In another embodiment, the cycloalkyl group does comprise 1 to 3 exo carbonyl or thiocarbonyl groups. It is understood, that the term “exo” refers to the attachment of a carbonyl or thiocarbonyl to a carbon ring atom of the cycloalkyl group.

“Substituted cycloalkyl” refers to a cycloalkyl group, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic.

“Cycloalkenyl” refers to cyclic alkenyl but not aromatic groups of from 5 to 10 carbon atoms having single or multiple cyclic rings optionally comprising 1 to 3 exo carbonyl or thiocarbonyl groups. Suitable cycloalkenyl groups include, by way of example, cyclopentyl, cyclohexenyl, cyclooctenyl, 3-oxocyclohexenyl, and the like. In multiple condensed rings, one or more of the rings may be other than cycloalkenyl (e.g., aryl, heteroaryl or heterocyclic) provided that the point of attachment is to a carbon ring atom of the cycloalkyl group. In one embodiment, the cycloalkenyl group does not comprise 1 to 3 exo carbonyl or thiocarbonyl groups. In another embodiment, the cycloalkenyl group does comprise 1 to 3 exo carbonyl or thiocarbonyl groups. It is understood, that the term “exo” refers to the attachment of a carbonyl or thiocarbonyl to a carbon ring atom of the cycloalkenyl group.

Preferred substituted cycloalkenyl include cycloalkenyl groups, having from 1 to 5 substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic provided that for hydroxyl substituents the point of attachment is not to a vinyl carbon atom.

“Cycloalkoxy” refers to —O-cycloalkyl groups.

“Substituted cycloalkoxy” refers to —O-substituted cycloalkyl groups.

The term “guanidino” refers to the group —NHC(═NH)NH₂ and the term “substituted guanidino” refers to —NR^{p}C(═NR^{p})N(R^{p})₂ where each R^{p} is independently hydrogen or alkyl.

“Halo” or “halogen” refers to fluoro, chloro, bromo and iodo and preferably is fluoro or chloro.

“Haloalkyl” refers to an alkyl group substituted with 1 to 5 halogen groups. An example of haloalkyl is CF₃.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur, within the ring. Preferably, such heteroaryl groups are aromatic groups of from 1 to 15 carbon atoms, preferably from 1 to 10 carbon atoms, and 1 to 4 heteroatoms selected from the group consisting of oxygen, nitrogen, and sulfur within the ring. Such heteroaryl groups can have a single ring (e.g., pyridyl or furyl) or multiple condensed rings (e.g., indolizinyl or benzothienyl). The sulfur atom(s) in the heteroaryl group may optionally be oxidized to sulfoxide and sulfone moieties.

“Substituted heteroaryl” refers to heteroaryl groups that are substituted with from 1 to 3 substituents selected from the same group of substituents defined for substituted aryl.

When a specific heteroaryl is defined as “substituted”, e.g., substituted qunioline, it is understood that such a heteroaryl contains the 1 to 3 substituents as recited above.

“Heteroarylene” and “substituted heteroarylene” refer to divalent heteroaryl and substituted heteroaryl groups as defined above.

“Heteroaryloxy” refers to the group —O-heteroaryl and “substituted heteroaryloxy” refers to the group —O-substituted heteroaryl.

“Heterocycle” or “heterocyclic” or “heterocyclyl” refers to a saturated or unsaturated group having a single ring or multiple condensed rings, from 1 to 10 carbon atoms and from 1 to 4 hetero atoms selected from the group consisting of nitrogen, sulfur or oxygen within the ring which ring may optionally comprise 1 to 3 exo carbonyl or thiocarbonyl groups. Preferably, such heterocyclic groups are saturated or unsaturated group having a single ring or multiple condensed rings, from 1 to 10 carbon atoms and from 1 to 4 hetero atoms selected from the group consisting of nitrogen, sulfur, or oxygen within the ring. The sulfur atom(s) in the heteroaryl group may optionally be oxidized to sulfoxide and sulfone moieties.

In multiple condensed rings, one or more of the rings may be other than heterocyclic (e.g., aryl, heteroaryl or cycloalkyl) provided that the point of attachment is to a heterocyclic ring atom. In one embodiment, the heterocyclic group does not comprise 1 to 3 exo carbonyl or thiocarbonyl groups. In a preferred embodiment, the heterocyclic group does comprise 1 to 3 exo carbonyl or thiocarbonyl groups. It is understood, that the term “exo” refers to the attachment of a carbonyl or thiocarbonyl to a carbon ring atom of the heterocyclic group.

“Substituted heterocyclic” refers to heterocycle groups that are substituted with from 1 to 3 of the same substituents as defined for substituted cycloalkyl. Preferred substituents for substituted heterocyclic groups include heterocyclic groups having from 1 to 5 having substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic.

When a specific heterocyclic is defined as “substituted”, e.g., substituted morpholino, it is understood that such a heterocycle contains the 1 to 3 substituents as recited above.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

The term “thiol” refers to the group —SH.

The term “amino acid” refers to β-amino acids or to α-amino acids of the formula HR^{b}N[CH(R^{a})]_{c}COOH where R^{a} is as defined above, R^{b} is hydrogen, alkyl, substituted alkyl or aryl and c is one or two. Preferably, c is one, an α-amino acid, and the α-amino acid is one of the twenty naturally occurring L amino acids.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR^{k}, —PO₂(R^{k})₂, —CN, —PO₃(R^{k})₂, —OR^{k}, —SR^{k}, —NHCOR^{k}, —N(R^{k})₂, —CON(R^{k})₂, —CONH(O)R^{k}, —CONHNHSO₂R^{k}, —COHNSO₂R^{k}, and —CONR^{k}CN, where R^{k} is selected from hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, thioalkyl, alkylthio, sulfonyl, alkyl, alkenyl or alkynyl, aryl, aralkyl, cycloalkyl, heteroaryl, heterocycle, and CO₂R^{m} where R^{m} is hydrogen alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred isosteres contemplated by this invention: 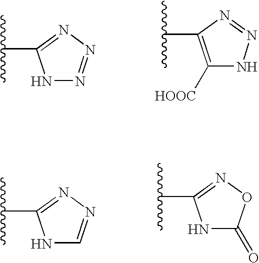
where the atoms of said ring structure may be optionally substituted at one or more positions with R^{k}. The present invention contemplates that when chemical substituents are added to a carboxylic isostere then the inventive compound retains the properties of a carboxylic isostere. The present invention contemplates that when a carboxylic isostere is optionally substituted with one or more moieties selected from R^{k}, then the substitution cannot eliminate the carboxylic acid isosteric properties of the inventive compound. The present invention contemplates that the placement of one or more R^{k} substituents upon the carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

“Carboxylic acid bioisosteres” are compounds that behave as isosteres of carboxylic acids under biological conditions.

Other carboxylic acid isosteres not specifically exemplified or described in this specification are also contemplated by the present invention

“Thiocarbonyl” refers to the group C(═S).

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—; the term “alkylaryloxy” refers to the group alkyl-aryl-O—; the term “arylalkyloxy” refers to the group aryl-alkyl-O—, “thioalkyl” refers to SH-alkyl-; “alkylthio” refers to alkyl-S— etc. Various substituents may also have alternate but equivalent names. For example, the term 2-oxo-ethyl and the term carbonylmethyl both refer to the —C(O)CH₂— group.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups or a hydroxy group alpha to ethenylic or acetylenic unsaturation). Such impermissible substitution patterns are well known to the skilled artisan.

General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

In one preferred embodiment, the compounds of this invention are prepared by convergent synthetic procedures employing a core indolyl group and a core HET-Y group. Specifically, the core indolyl group is represented by the formula: 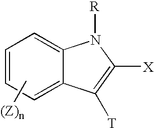
where R, T, Z and n are as defined herein and X is —B(OH)₂. The above compounds are prepared from the corresponding 2-bromoindole derivatives which are known in the art and disclosed, for example, in International Patent Application Publication No. WO 03/010141 which is incorporated herein by reference in its entirety.

Schemes 1 and 2 illustrate the conversion of 2-bromoindole derivatives to the corresponding indol-2-yl boronic acid.

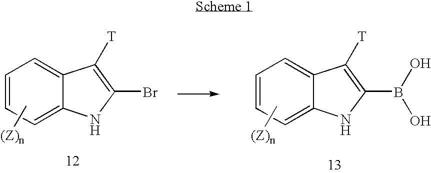

Scheme 1 illustrates the conversion of optionally further substituted [with (Z)ₙ and T] 2-bromo-1H indole, compound 12, to the corresponding indol-2-yl boronic acid, compound 13.

Specifically, compound 12 is converted to the 2-boronic acid derivative, compound 13, by contact with an excess of bis(neopentylglycolato)diboron in the presence of a catalytic amount of triphenylphosphine palladium(II) dichloride. The reaction is conducted in a suitable solvent, such as DMSO, in the presence of a suitable base such as potassium acetate under an inert atmosphere. Preferably, the reaction is conducted at a temperature of from about 60° C. to about 120° C. The reaction is continued until it is substantially complete which typically occurs within about 0.5 to 15 hours. After reaction completion, the resulting product (indol-2-yl boronic acid, compound 13) can be isolated by conventional techniques such as evaporation, extraction, precipitation, filtration, chromatography, and the like; or, alternatively, used in the next step without purification and/or isolation.

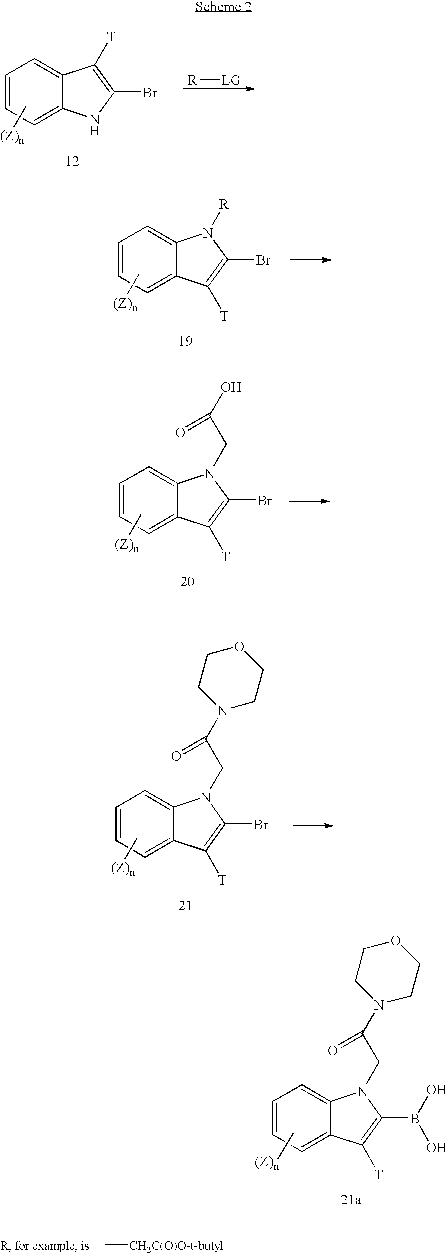

Scheme 2 parallels that of Scheme 1 with the exception that R at the indolyl nitrogen of compound 12 is initially hydrogen and is converted to a non-hydrogen group. Specifically, compound 12 is reacted under conventional conditions with a compound such as R-LG where LG is a suitable leaving group such as halo, tosyl, mesyl, and the like. This reaction provides for suitable R substitution at the indolyl nitrogen atom. In those embodiments where R contains or can be modified to contain derivatizable functionality, the R group can be modified to provide further compounds of this invention.

For the purposes of illustration only, R is Scheme 2 is depicted as a —CH₂C(O)O-t-butyl group. In this example, compound 12 is first alkylated with a suitable reagent such as commercially available t-butyl bromoacetate to provide for (1-t-butoxy-carbonylmethyl]-2-bromo-1H-indole, compound 19, where LG is bromo. The reaction proceeds by combining compound 12 with at least a stoichiometeric amount and preferably a slight excess of t-butyl bromoacetate in a suitable inert solvent in the presence of a base. Suitable solvents include, for example, DMF, THF, DMSO, and the like, and suitable bases include sodium hydride, lithium diisopropylamide, and the like. Preferably, the reaction is conducted at a temperature of from about −60° C. to about 10° C. The reaction is continued until it is substantially complete which typically occurs within about 0.1 to 1 hours. After reaction completion, the resulting product 1-t-butoxycarbonylmethyl-2-bromo-1H-indole (compound 19) can be isolated by conventional techniques such as evaporation, extraction, precipitation, filtration, chromatography, and the like; or, alternatively, used in the next step without purification and/or isolation.

Conventional removal of the t-butyl group by trifluoroacetic acid provides for 1-carboxylmethyl-2-bromo-1H-indole (compound 20).

Amidation of the carboxyl group by a suitable amine (for illustrative purposes only depicted as a morpholino group in Scheme 2) provides for compound 21. This reaction proceeds via conventional conditions using well-known coupling reagents such as carbodiimides, BOP reagent (benzotriazol-1-yloxy-tris(dimethylamino)phosphonium hexafluorophosphonate) and the like. Suitable carbodiimides include, by way of example, dicyclohexylcarbodiimide (DCC), 1-(3dimethylamino-propyl)-3-ethylcarbodiimide (EDC) and the like. If desired, polymer supported forms of carbodiimide coupling reagents may also be used including, for example, those described in Tetrahedron Letters, 34(48), 7685 (1993). Additionally, well-known coupling promoters, such as N-hydroxysuccinimide, 1-hydroxybenzotriazole and the like, may be used to facilitate the coupling reaction.

This coupling reaction is typically conducted by contacting compound 20 with about 1 to about 2 equivalents of the coupling reagent and at least one equivalent, preferably about 1 to about 1.2 equivalents, of the amino compound to be coupled to the carboxyl group (e.g., morpholine) in an inert diluent, such as dichloromethane, chloroform, acetonitrile, tetrahydrofuran, N,N-dimethylformamide and the like. Generally, this reaction is conducted at a temperature ranging from about 0° C. to about 37° C. for about 12 to about 24 hours. Upon completion of the reaction, compound 21 [(1-morpholinocarbonylmethy)-2-bromo-1H-indole] is recovered by conventional methods including neutralization, extraction, precipitation, chromatography, filtration, and the like.

Alternatively, the carboxyl group of compound 20 can be converted into an acid halide and the acid halide coupled with the amino compound to be coupled to provide for compound 21. The acid halide can be prepared by contacting compound 20 with an inorganic acid halide, such as thionyl chloride, phosphorous trichloride, phosphorous tribromide or phosphorous pentachloride, or preferably, with oxalyl chloride under conventional conditions. Generally, this reaction is conducted using about 1 to 5 molar equivalents of the inorganic acid halide or oxalyl chloride, either neat or in an inert solvent, such as dichloromethane or carbon tetrachloride, at temperature in the range of about 0° C. to about 80° C. for about 1 to about 48 hours. A catalyst, such as DMF, may also be used in this reaction.

The acid halide of compound 20 is then contacted with at least one equivalent, preferably about 1.1 to about 1.5 equivalents, of the amino compound in an inert diluent, such as dichloromethane, at a temperature ranging from about −70° C. to about 40° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like. Upon completion of the reaction, compound 21 is recovered by conventional methods including neutralization, extraction, precipitation, chromatography, filtration, and the like.

The bromo group of compound 21 can be converted to the corresponding boronic acid derivative as per above to provide for compound 21a.

It is understood that other reagents defined by R-LG can be employed in Scheme 2 above to affect alkylation, cycloalkylation, arylation, heteroarylation, and heterocyclization of the indole nitrogen atom of compound 12 using conditions described above and modified as necessary which modifications are well within the skill of the art.

It is further understood that the R substituents in Scheme 2 are included within the definition of R for the compounds of formula I.

The HET-Y group used in the convergent synthesis strategy described herein is preferably prepared by conventional procedures well known in the art. In the convergent synthetic methods, the HET-Y group contains a reactive functionality on the HET moiety to effect coupling to the indole molecule. Scheme 3 below illustrates one generic method for preparing suitable HET-Y groups for use in such convergent synthesis.

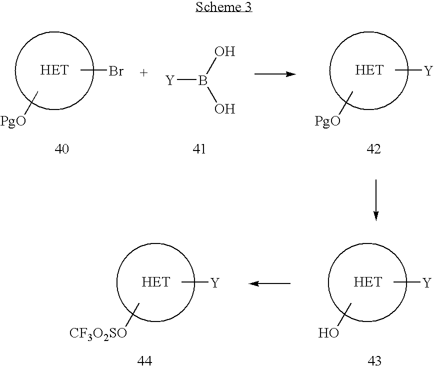

Scheme 3 employs a bromo and hydroxyl substituted aryl or heteroaryl compound, compound 40, which is optionally further substituted with one or more X groups (not shown). If necessary, the hydroxyl group can be protected by conventional protecting groups, Pg, which are well known in the art. Compound 40 is reacted under conventional Suzuki conditions with the boronic acid derivative of Y, compound 41, which can be prepared in the manner described in Scheme 1 above from the corresponding Y—Br compound, to provide for compound 42. When Pg is not hydrogen, the protecting group is removed by conventional procedures to provide for hydroxyl substituted compound 43. The hydroxyl group of compound 43 is converted under conventional conditions to the triflate of compound 44 which can be used in a Suzuki reaction with, for example, compound 13 or 21a to provide for the compounds of formula I.

As illustrated below, the preferred coupling procedure for compound 44 with, for example, compound 13, is via a conventional Suzuki reaction. Since the Y group of compound 41 is attached to compound 40 via a conventional Suzuki reaction, orthogonal substituents must be employed on compound 40 to effect the two separate Suzuki coupling chemistries employed to effect coupling of Y to Het and then to effect coupling of the indolyl moiety to Het-Y. This is accomplished in Scheme 3 by use of a hydroxyl substituent which is inert to the first Suzuki reaction effecting coupling of Y to the Het moiety. Subsequently, the hydroxyl substituent is converted into the triflate group which can participate in the second Suzuki reaction with the boronic acid moiety of compound 13. In this embodiment, the hydroxyl substituent acts as a precursor substituent for use in the Suzuki reaction.

Suitable hydroxyl and bromo substituted aryl and heteroaryl compounds are either commercially available or the synthesis of which are well known in the art. Examples of such compounds include, bromophenol, 2-bromo-3-hydroxyl-pyridine, 5-hydroxy-3-bromoindole, and the like.

Likewise, bromo-substituted, aryl and heteroaryl Y compounds, optionally further substituted, are either commercially available or can be prepared by art recognized procedures.

Alternatively, HET-Y can be prepared from core starting materials to provide for compounds suitable for convergent synthesis with the 2-bromoindoles described above. Because such methods employ selected reaction schemes, the use of orthogonal Suzuki substituents can be avoided thereby providing synthetic flexibility. The synthesis of optionally substituted aromatic and heteroaromatic compounds suitable for subsequent Suzuki reactions is well known in the art. Scheme 4 below illustrates such a synthetic scheme for the preparation of quinolinyl HET-Y group having a bromo group suitable for Suzuki coupling to the indole compound. It is understood that this quinolinyl group is depicted for illustrative purpose only.

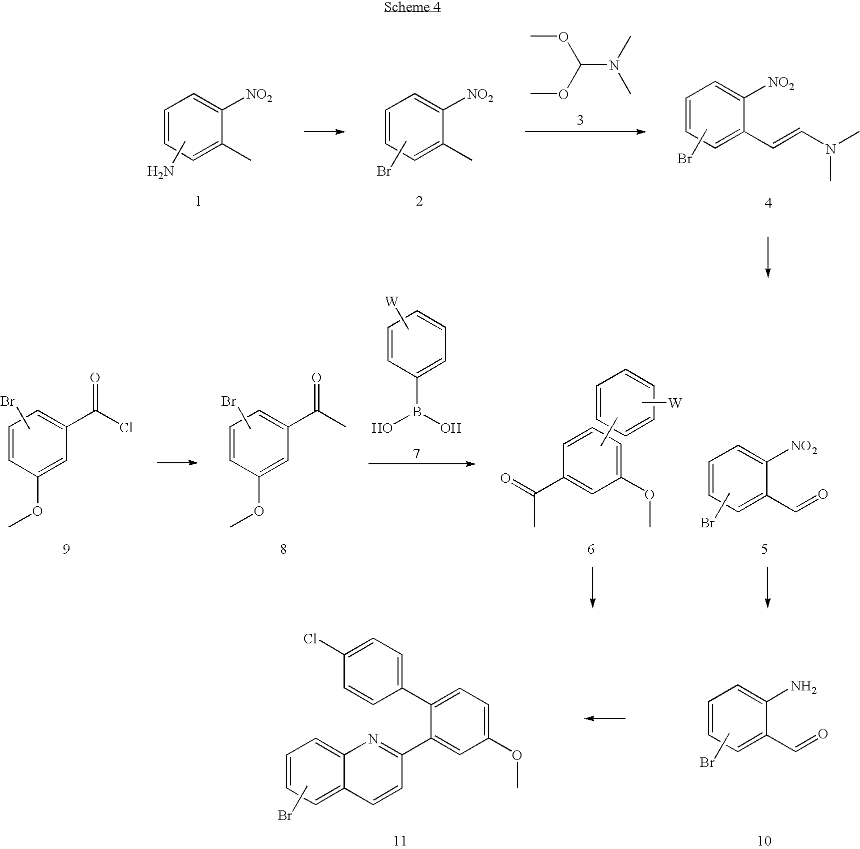

In Scheme 4, commercially available amino 2-methyl-4-nitrobenzene, compound 1, is converted to the corresponding bromo-2-methyl-nitrobenzene, compound 2, under conventional conditions using an equimolar amounts of sodium nitrite, an excess of HBr and a catalytic amount of cupric bromide. The reaction is preferably conducted by combining compound 1 with an excess of aqueous hydrogen bromide (e.g., 48% HBr) in an inert solvent at a temperature of from about −10 to 10° C. An equimolar amount of sodium nitrite dissolved in water is slowly added to the reaction mixture while maintaining the reaction temperature. A catalytic amount of solid cuprous bromide is then added to the reaction mixture and the reaction mixture is allowed to warm to slightly less than room temperature. The reaction is monitored until nitrogen evolution ceases indicating reaction completion. Afterwards, the resulting product, bromo-2-methyl-nitrobenzene, compound 2, can be isolated by conventional techniques such as evaporation, extraction, precipitation, filtration, chromatography, and the like; or, alternatively, used in the next step without purification and/or isolation.

Suitable examples of compound 1 include commercially available variants such as 2-nitro-3-methylaniline, 4-methyl-3-nitroaniline (both commercially available from Aldrich Chemical Company, Milwaukee, Wis., USA) as well as 3-methyl-4-nitroaniline (commercially available from Lancaster Synthesis Inc.).

Compound 2 is next converted to (E)-2-(bromo-2-nitrophenyl)vinyl dimethylamine, compound 4, by reaction with an excess of N,N-dimethylformamide dimethylacetal, compound 3. The reaction is typically conducted in a suitable solvent such as DMF under an inert atmosphere. Preferably, the reaction is conducted at an elevated temperature of from about 100° C. to about 160° C. The reaction is continued until it is substantially complete which typically occurs within about 1 to 6 hours. After reaction completion, the resulting product can be isolated by conventional techniques such as evaporation, extraction, precipitation, filtration, chromatography, and the like; or, alternatively, used in the next step without purification and/or isolation.

Oxidation of (E)-2-(bromo-2-nitrophenyl)vinyl dimethylamine, compound 4, proceeds via contact with a large excess of sodium periodate to provide for bromo-2-nitrobenzaldehyde. This reaction is typically conducted in an inert diluent such as an aqueous mixture of tetrahydrofuran, dioxane, and the like. Preferably, the reaction is conducted at an ambient conditions and is continued until it is substantially complete which typically occurs within about 0.5 to 6 hours. After reaction completion, the resulting product, bromo 2-nitrobenzaldehyde, compound 5, can be isolated by conventional techniques such as evaporation, extraction, precipitation, filtration, chromatography, and the like; or, alternatively, used in the next step without purification and/or isolation.

Conventional reduction of compound 5 provides for the corresponding bromo 2-aminobenzaldehyde, compound 10.

Separately, bromo-5-methoxybenzoyl chloride, compound 9 (available from Maybridge), is converted to the corresponding bromo-3-acetyl-methoxybenzene, compound 8, by reaction with dimethyl zinc. The reaction is typically conducted in a suitable inert diluent such as benzene, toluene, xylene and the like. Preferably, the dimethyl zinc is present in the solvent prior to addition of compound 9 as dimethyl zinc is pyroforic. Preferably, the reaction is initially conducted at a temperature of from about −10 to about 10° C. and then allowed to slowly proceed to room temperature. The reaction is continued until it is substantially complete which typically occurs within about 0.2 to 2 hours. After reaction completion, the resulting product, bromo-3-acetyl-methoxybenzene (compound 8) can be isolated by conventional techniques such as evaporation, extraction, precipitation, filtration, chromatography, and the like; or, alternatively, used in the next step without purification and/or isolation.

Alternatively, bromo-5-methoxybenzoyl chloride, compound 9, can be prepared from the corresponding commercially available bromo-5-methoxybenzoic acid such as 2-bromo-5-methoxybenzoic acid (available from Aldrich Chemical Company, Milwaukee, Wis., USA) by conversion into an acid halide. The acid halide can be prepared by contacting the carboxylic acid with an inorganic acid halide, such as thionyl chloride, phosphorous trichloride, phosphorous tribromide or phosphorous pentachloride, or preferably, with oxalyl chloride under conventional conditions. Generally, this reaction is conducted using about 1 to 5 molar equivalents of the inorganic acid halide or oxalyl chloride, either neat or in an inert solvent, such as dichloromethane or carbon tetrachloride, at temperature in the range of about 0° C. to about 80° C. for about 1 to about 48 hours. A catalyst, such as DMF, may also be used in this reaction.

Conversion of the aryl moiety of compound 8 to a biaryl or heteroaryl-aryl moiety, e.g., compound 6, proceeds via conventional Suzuki reaction conditions which are illustrated in Scheme 4. The to-be-coupled aryl or heteroaryl moiety employed may optionally be substituted and, in Scheme 4, optional substitution is depicted by W which is hydrogen, chloro, or other suitable substituent which is compatible with the reaction conditions employed. Post-reaction modification of W (other than hydrogen) is possible and is contemplated in the compounds of this invention.

In Scheme 4, commercially available chlorophenyl boronic acid, compound 7, is coupled with compound 8 via conventional Suzuki conditions to provide for chlorophenyl substituted 3-acetyl methoxybenzene, compound 6. 2-, 3- And 4-chlorophenyl boronic acids are commercially available from Aldrich Chemical Company, supra.

Compound 6 is then coupled with compound 10, described above, under condensation conditions to provide for 2-biaryl-6-bromoquinoline, compound 11. This reaction is preferably conducted by combining approximately stoichiometric amounts of both compounds 6 and 10 in a suitable inert diluent such as ethanol, isopropanol and the like in the presence of a suitable base such as potassium hydroxide under an inert atmosphere. Preferably, the reaction is conducted at a temperature of from about 70° C. to about 100° C. and proceeds until it is substantially complete which typically occurs within about 2 to 16 hours. After reaction completion, the resulting product, compound 11, can be isolated by conventional techniques such as evaporation, extraction, precipitation, filtration, chromatography, and the like; or, alternatively, used in the next step without purification and/or isolation.

As illustrated in Scheme 5 below, the convergent synthetic protocol proceeds via a conventional Suzuki reaction employing a suitable indole, e.g., compound 13 or 21a, together with a suitably substituted Het-Y compound to provide for the compounds of formula I.

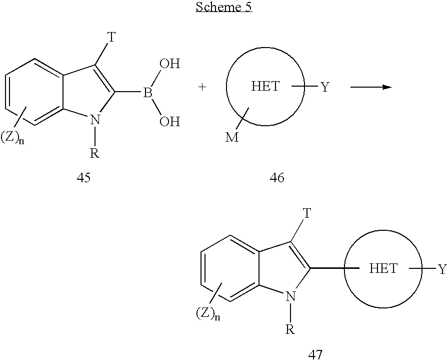

In Scheme 5, indolyl boronic acid, compound 45 (described above), is combined with Het-Y compound, 46 (described above), having a Suzuki compatible substituent, M, bound thereto. Suitable M substituents include, by way of example, bromo, iodo, triflate, and the like. The reaction proceeds via conventional Suzuki conditions to provide for the compound of formula I, compound 47. A specific illustration of this coupling reaction is provided in Scheme 6 below: 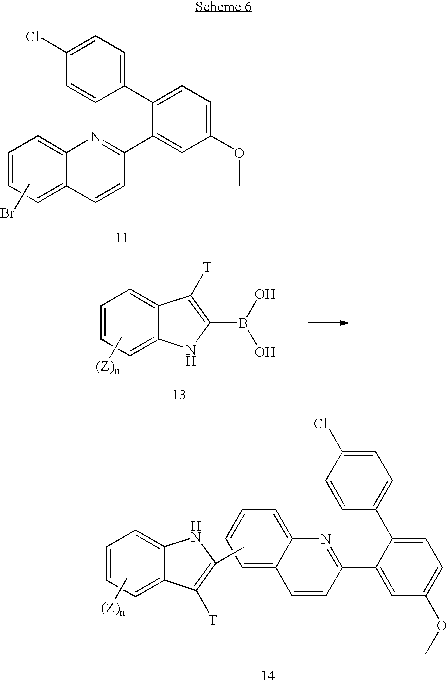
where T, Z and n are as defined above.

In Scheme 6, the Suzuki reaction proceeds via compatible boronic acid functionality on compound 13 and the bromo functionality on compound 11 to provide for compound 14, a compound of this invention. Specifically, an excess (preferably 1.1 to 3-fold excess) of compound 11 is combined with compound 13 in a suitable inert solvent such as toluene, a mixture of toluene/methanol (e.g., 4:1 mixture), and the like in the presence of both a catalytic amount of tetrakis(triphenylphosphino)palladium and a base such as sodium bicarbonate under an inert atmosphere. The reaction is preferably conducted at an elevated temperature of from about 60 to 100° C. for a period of time to effect substantial completion of the reaction which typically occurs within 0.1 to 0.5 hours. After reaction completion, the resulting product, compound 14, can be isolated by conventional techniques such as evaporation, extraction, filtration, chromatography, and the like.

In another embodiment, the preparation of substituted indole-quinoline compounds of formula I can proceed via a linear synthetic pathway as illustrated in Schemes 7 and 8 below wherein starting material for Scheme 8 is prepared in Scheme 7.

Scheme 7 illustrates the synthesis of bromo 2-dimethoxymethyl-1-nitrobenzene (compound 17), which is used in synthetic Scheme 9:

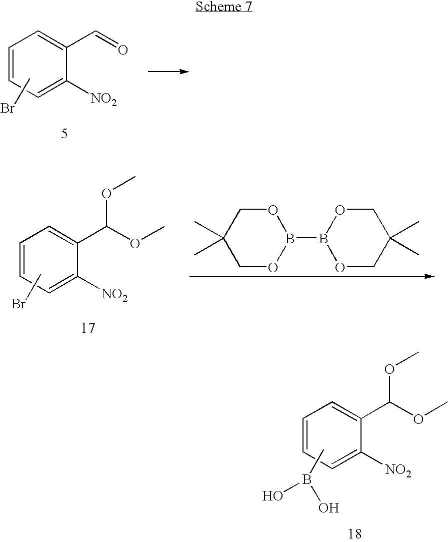

In Scheme 7, the bromo-2-nitrobenzaldehyde, compound 5, is provided as described above. Alternatively, it is contemplated that compound 5 can also be prepared from the commercially available bromo-2-nitrobenzoic acid (not shown—available from Aldrich Chemical Co., Milwaukee, Wis., USA) by conventional reduction of the carboxyl group to the aldehyde.

The aldehyde group of compound 5 is converted to the corresponding dimethoxymethyl group of compound 17 by conventional contact with methanol/HCl. The reaction is preferably conducted at an elevated temperature of from about 60 to 100° C. for a period of time to effect substantial completion of the reaction which typically occurs within 0.1 to 0.5 hours. After reaction completion, the resulting product, bromo 2-dimethoxymethyl-1-nitrobenzene (compound 17), can be isolated by conventional techniques such as evaporation, extraction, filtration, chromatography, and the like; or, alternatively, used in the next step without purification and/or isolation.

Bromo 2-dimethoxymethyl-1-nitrobenzene, compound 17, is subsequently converted to the boronic acid derivative, compound 18, by contact with a approximately a stoichiometric amount of bis(neopentylglycolato)diboron in the presence of a catalytic amount of triphenylphosphine palladium(II) dichloride. The reaction is conducted in a suitable solvent, such as DMSO, under an inert atmosphere. Preferably, the reaction is conducted at a temperature of from room temperature to 60° C. The reaction is continued until it is substantially complete which typically occurs within about 0.5 to 8 hours. After reaction completion, the resulting product, 3-dimethoxymethyl-4-nitrophenylboronic acid (compound 18) can be isolated by conventional techniques such as evaporation, extraction, filtration, chromatography, and the like; or, alternatively, used in the next step without purification and/or isolation.

Scheme 8 below illustrates the step wise synthesis of compounds of Formula I of this invention. This scheme employs for illustrative purposes the following: n=one, Z=methoxycarbonyl, T=cyclohexyl and R=morpholinocarbonylmethyl.

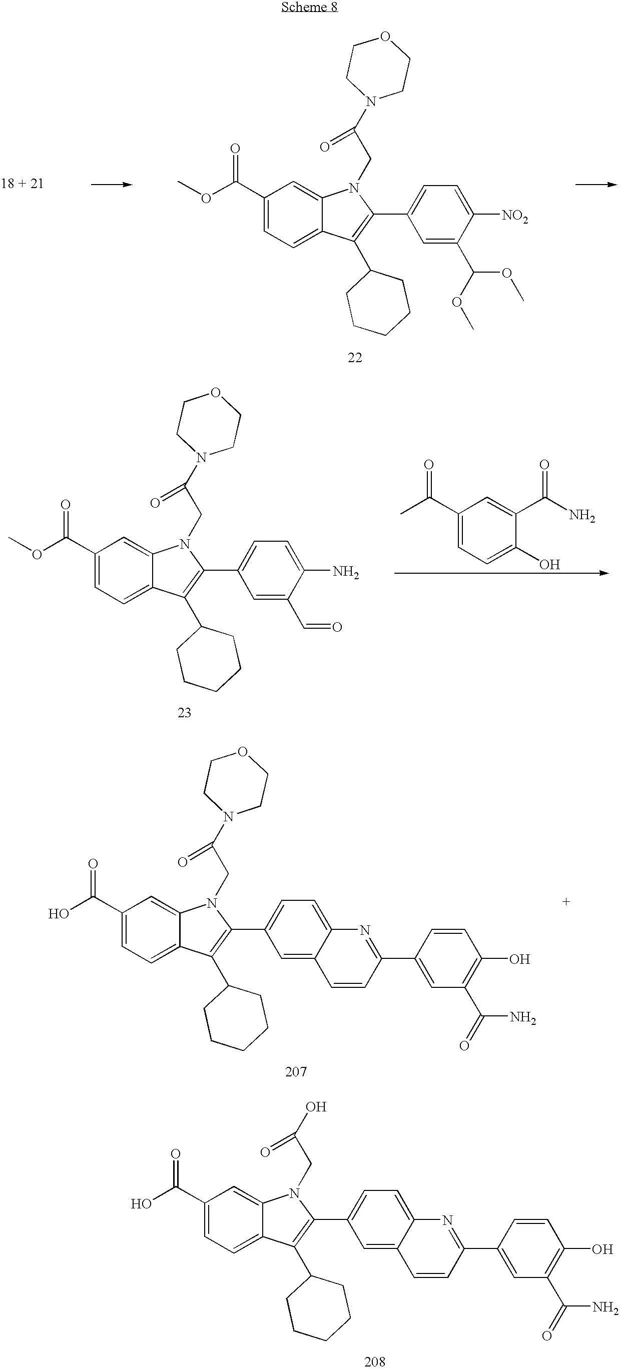

In Scheme 8, compounds 18 and 21, described above, are coupled via conventional Suzuki reaction conditions also described above to provide for compound 22. Conventional reduction of the nitro group of compound 22 via hydrogen and Pd/C catalyst at elevated pressures in anhydrous methanol, followed by a treatment with aqueous acid, provides for both the 4-amino and the 3-formyl substituents of compound 23. In turn, compound 23 is employed in a condensation procedure using an excess of 3-carboxamido-4-acetylphenol in a suitable solvent such as an ethanolic solution comprising 10% KOH provides for a mixture of both compounds 207 and 208. The reaction typically proceeds at elevated temperatures and preferably at reflux for a period of from 2 to 12 hours. The decomposition of the morpholino amide by the basic solution is responsible for generation of the N-carboxylmethyl group on the indole nitrogen atom of compound 208.

The free carboxyl group of compound 207 provides a basis for further modification of this compound as illustrated in Scheme 9 below:

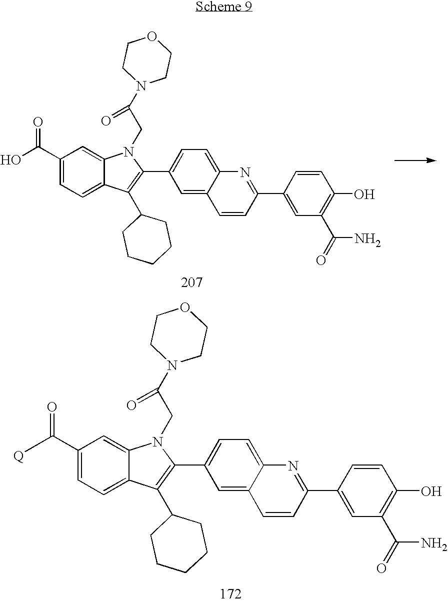

Compound 207 is optionally further derivatized with a suitable moiety, Q. Preferred Q groups include those which give rise to Z groups as recited for the compounds of Formula I when Z is a), b), c), d), e), f), and g). Preferably, compound 207 is coupled with Q wherein Q is a heteroatom containing group, preferably an amino or substituted amino group including, for example, substituted amino acids such as L-5-hydroxytryptophane. Suitable amino groups are well known in the art and include a variety of commercially available primary or secondary amines, and preferably, an amino acid or substituted amino acid derived from an L isomer of an amino acid. Compound 207 is activated by conventional means, such as treatment with HBTU and DIEA at room temperature for a time sufficient to promote activation, typically from 5 to 20 minutes. The activated compound is then treated with Q, for example, a nitrogen containing group, in an inert diluent such as N,N-dimethylformamide at room temperature for a period of time to effect substantial completion of the reaction which typically occurs within 30 minutes to 1 hour. After reaction completion, the resulting product, compound 172, can be isolated by conventional techniques such as extraction, filtration, chromatography, and the like. The purified product may also be converted to the acid salt by treatment of 172 with an appropriate acid salt, such as HCl, for a time sufficient for substantial reaction completion.

In another embodiment, the preparation of compounds of formula I-IIIa is accomplished according to Scheme 14.

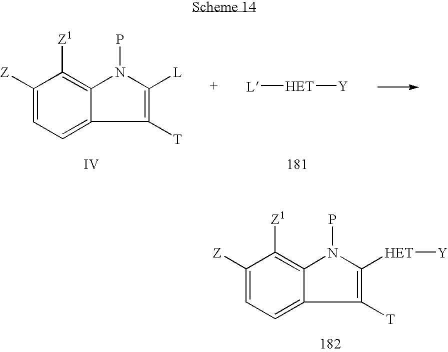

The reaction is carried out in the presence of a transition metal catalyst such as Pd(0). P is a H or a nitrogen protecting group. One of L and L′ is halo and the other of L and L′ is B(R³⁰)₂ or Sn(R³¹)₃ where R³⁰ is independently hydroxy, alkoxy, halo, or a suitable boron ligand and R³¹ is independently alkyl or aryl. Suitable borinates include —B(OH)₂, cyclic boronic esters, cyclic organoboranes, and BF₃⁻K⁺ (see, for example, G. A. Molander, C. R. Bernardi, J. Org. Chem., 2002, 67, 8424-8429; E. Vedejs, R. W. Chapman, S. C. Fields, S. Lin, M. R. Schrimpf. J. Org. Chem. 60, 3020, 1995, and D. S. Matteson Pure Appl. Chem. 75, 1249, 2003). When P is a H, compound 182 can optionally be reacted with L″-R where L″ is halo or —OSO₂R³² and where R³² is alkyl, substituted alkyl, aryl, or substituted aryl. When P is a nitrogen protective group, the nitrogen protective group is removed first and then reacted with L″-R. This synthetic approach is further illustrated in Scheme 6 above and Schemes 15 and 16 below where HET is exemplified as 2,6-quinoline, R is 2-dimethylamino-2-oxo-ethyl, and the nitrogen protecting group is t-butyloxycarbonyl. The reactions can also be carried out for other R groups defined herein such as 2-morpholin-4-yl-2-oxo-ethyl, 2-(4-hydroxy-piperdin-1-yl)-2-oxo-ethyl, and 2-(2-methyl-pyrrolidin-1-yl)-2-oxo-ethyl.

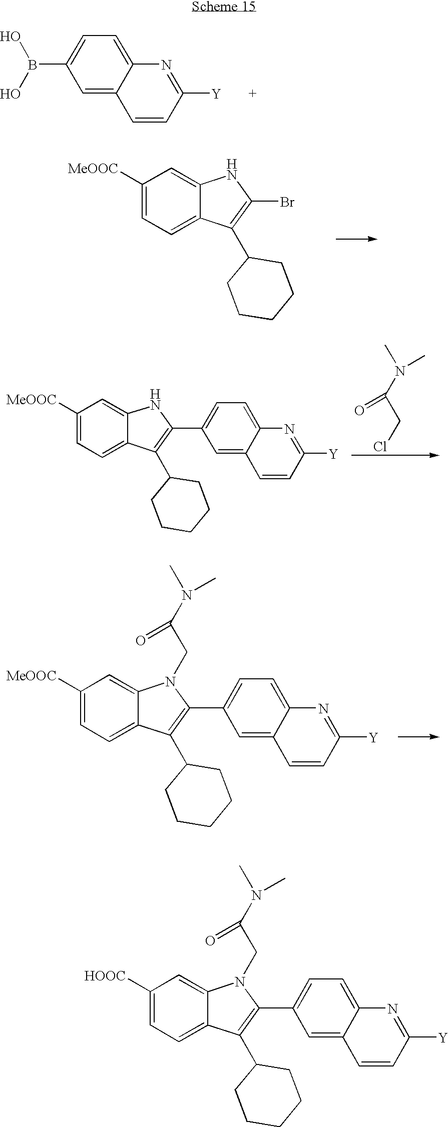

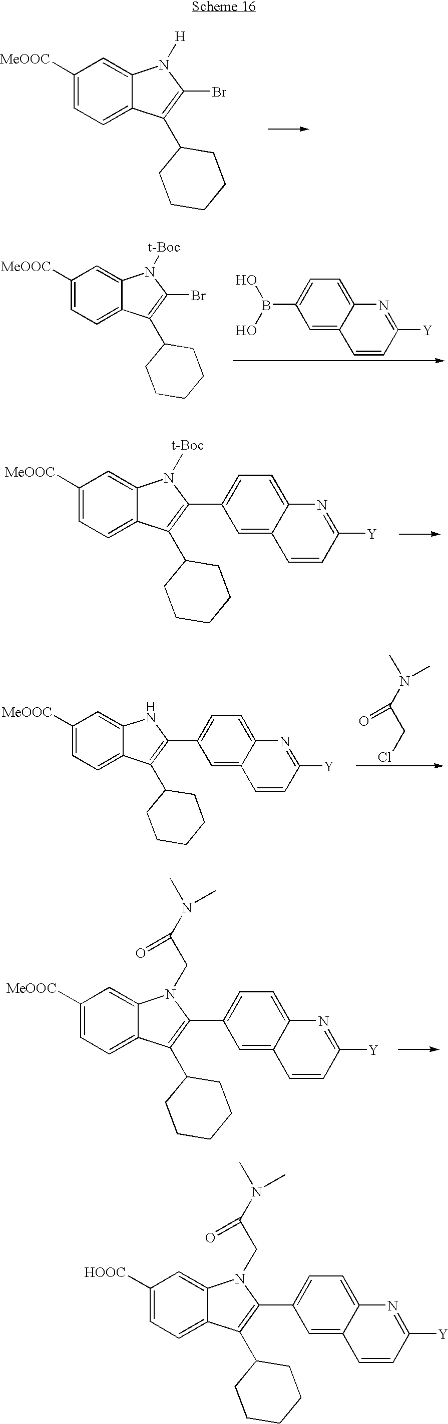

The present invention further provides an intermediate compound having the formula VI or VII

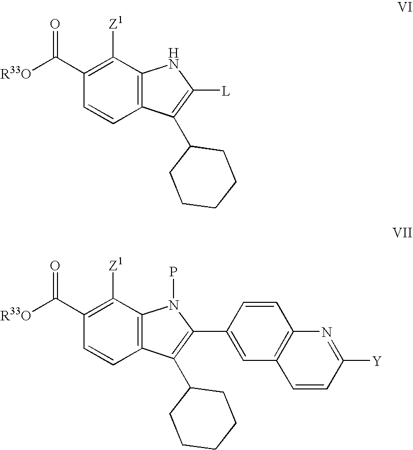

wherein R³³ is alkyl or arylalkyl;

Z¹ is selected from the group consisting of hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;

L is halo;

P is H or a nitrogen protecting group; and

Y is substituted aryl or substituted heteroaryl.

In some embodiments, Y is a group described herein. In other embodiments R³³ is methyl. In still other embodiments the nitrogen protecting tert-butylcarbonyloxy.

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-70 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

### EXAMPLES

In the examples below and the synthetic schemes above, the following abbreviations have the following meanings. If an abbreviation is not defined, it has its generally accepted meaning.
μL=microliters
μM=micromolar
μg=micrograms
NMR=nuclear magnetic resonance
boc=t-butoxycarbonyl
br=broad
d=doublet
δ=chemical shift
dd=doublet of doublets
DIEA=diisopropylethylamine
DMAP=4-N,N-dimethylaminopyridine
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
ESI=electrospray ionization
g=gram
h or hr=hours
HATU=O-(7-Azabenzotriazol-1-yl)-N,N, N′,N′-tetramethyluronium hexafluorophosphate
HBTU=O-Benzotriazol-1-yl-N,N, N′,N′-tetramethyluronium hexafluorophosphate
HCV=hepatitus C virus
HPLC=high performance liquid chromatography
Hz=hertz
IPTG=isopropyl-β-D-thiogalactopyranoside
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
mg=milligram
mL=milliliter
mM=millimolar
mmol=millimole
MS=mass spectrum
nm=nanometer
nM=nanomolar
ng=nanogram
NTA=nitrilotriacetic acid
NTP=nucleoside triphosphate
PCR=Polymerase chain reaction
ppm=parts per million
psi=pounds per square inch
Rp-HPLC=reversed phase high performance liquid chromatography
s=singlet
t=triplet
TC₅₀=Toxic concentration at 50% cell toxicity
tetrakis or tetrakis=tetrakis(triphenylphosphine)palladium(0) palladium
TFA=trifluoroacetic acid
THF=tetrahydrofuran
Tris=Tris(hydroxymenthyl)aminomethane
UTP=uridine triphosphate

Set forth in the examples below are compounds and intermediates useful for making compounds of the present invention. An overview of the synthetic protocols employed to prepare these compounds is set forth above.

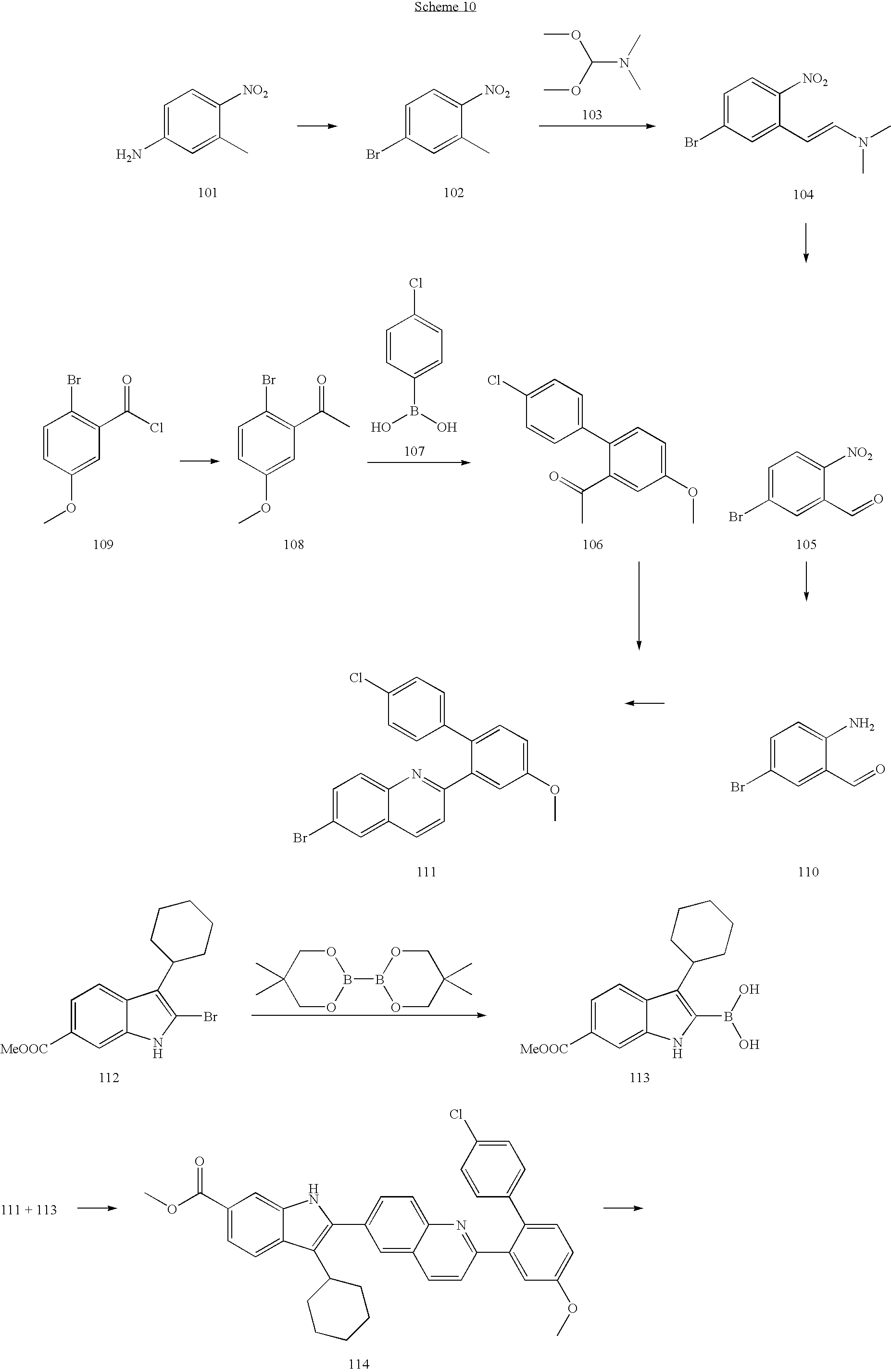

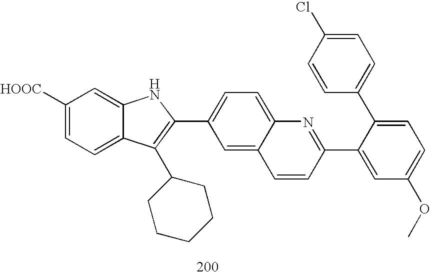

### Example 1

### 2-[2-(4′-chloro-4-methoxy-biphenyl-2-yl)-quinolin-6-yl]-3-cyclohexyl-1H-indole-6-carboxylic acid (Compound 200)

### Preparation of 4-bromo-2-methyl-1-nitro-benzene (102)

To an ice cold solution of 10.0 g (65.7 mmol) 3-methyl-4-nitro-phenylamine in 200 mL acetone, was added 21 mL (197.2 mmol) 48% HBr. 4.54 g (65.7 mmol) NaNO₂ was dissolved in 20 mL water and was added dropwise to the amine solution at a rate to keep the temperature under 5° C. The mixture was stirred at this temperature for an additional 10 minutes then 1.5 g (10 mmol) solid CuBr was added portion-wise at a rate to keep the temperature under 15° C. The reaction was complete when no more nitrogen evaluated (about 15 minutes). The reaction mixture was evaporated to dryness; the residue was dissolved in a mixture of 500 mL water and 750 mL ethyl acetate. The organic phase was separated, washed with water (2×), saturated NaCl (2×) and was dried (Na₂SO₄). It was then evaporated to dryness to give the crude product as a yellow solid which was purified by filtering through 400 mL silica gel pad using toluene elution;

Yield: 10.45 g (73%);

¹H-NMR (CDCl₃): δ (ppm) 7.87 (d, 1H, J=8.7 Hz), 7.51-7.46 (m, 2H), 2.61 (s, 3H).

### Preparation of [(E)-2-(5-bromo-2-nitro-phenyl)-vinyl]-dimethyl-amine (104)

A mixture of 9.26 g (42.9 mmol) of compound 102, 14.3 mL (107.2 mmol) N,N-dimethylformamide dimethylacetal and 11 mL DMF was heated under a slow argon flow at 145° C. (bath) for two hours. The reaction mixture was then evaporated to dryness. The dark pink product crystallized upon standing; MS: 271.01 & 273.01 (M+H⁺); ¹H-NMR (DMSO-d₆): δ (ppm) 7.88 (d, 1H), 7.68 (dd, 1H), 7.58 (d, 1H), 7.05 (d, 1H), 5.59 (d, 1H), 2.90 (s, 6H).

### Preparation of 5-bromo-2-nitro-benzaldehyde (105)

Compound 104 (11.63 g (42.9 mmol)) was dissolved in 500 mL 1:1 mixture of THF and water. To this solution 34.3 g (160 mmol) NaIO₄ was added and the mixture was stirred at room temperature for 1 hr while the dark solution became pale yellow with a heavy precipitate. The solid material was filtered off, washed twice with 100 mL ethyl acetate and the organic phases were pooled and evaporated to dryness. The residue was filtered through a 400 mL silicagel pad using toluene for elution to get 7.08 g (71%) of the title compound; H¹-NMR (DMSO-d₆): δ (ppm) 10.10 (s, 1H), 8.09-7.99 (m, 3H).

### Preparation of 2-amino-5-bromo-benzaldehyde (110)

Compound 110 was synthesized from 5.45 g (23.7 mmol) of compound 105 using the procedure of L. I. Smith and J. W. Opie (Org. Synth. Coll. Vol. 3, 56) in 55% yield (2.6 g); MS: 199.97 & 201.97 (M+H⁺); H¹-NMR (CDCl₃): δ (ppm) 9.75 (s, 1H), 7.71 (s, 1H), 7.39 (d, 1H, J=9.3 Hz), 7.22 (s, 2H), 6.72 (d, 1H, J=9.3 Hz).

### Preparation of 1-(2-bromo-5-methoxy-phenyl)-ethanone (108)

To an ice cold solution of 8.75 g (35 mmol) 2-bromo-5-methoxy-benzoyl chloride in 40 mL toluene, 9.63 mL (19.25 mmol) of a 2M toluene solution of dimethylzinc was added under argon atmosphere (dimethylzinc is pyrophoric—contact with air should be avoided!). The ice bath was removed and the mixture slowly warmed up to room temperature. Once the reaction starts it proceeds rapidly resulting in a turbid solution. The reaction was complete in 30 minutes. It was then cooled back to 0° C. and was quenched by adding 10 mL ethanol. The mixture was evaporated to dryness, the residue was dissolved in a mixture of 50 mL 1M HCl and 100 mL ethyl acetate. The organic phase was separated and washed with 50 mL water (2×), brine (2×) and was dried (Na₂SO₄). The final solution was evaporated and the oil dried overnight in high vacuum to give 7.96 g (99%) of the title compound as a colorless liquid; ¹H-NMR (CDCl₃): δ (ppm) 7.46 (d, 1H), 6.96 (d, 1H), 6.83 (dd, 1H), 3.80 (s, 3H), 2.63 (s, 3H).

### Preparation of 1-(4′-chloro-4-methoxy-biphenyl-2-yl)-ethanone (106)

A mixture of compound 108 (6.0 g, 26.19 mmol), 4-chlorobenzeneboronic acid (4.51 g, 28.81 mmol) and Pd(PPh₃)₄ (0.303 g, 0.262 mmol) in toluene (250 mL), MeOH (60 mL) and 2 M NaHCO₃ (25 mL) was stirred under argon at 80° C. for 16 h. After removal of the solvent, the dry residue was dissolved in CHCl₃ (150 mL) and filtered. The solvent was evaporated and the residue was purified by chromatography using CHCl₃-MeOH (70:1) as eluent to give the title compound (6.33 g, 93%); ¹H NMR (CDCl₃): 7.36 (d, 2H, J=8.4 Hz), 7.27-7.21 (m, 4H), 7.02 (d, 1H, J=2.7 Hz), 3.86 (s, 3H), 2.05 (s, 3H). MS (ESI) 261.07 (M+H).

### Preparation of 6-bromo-2-(4′-chloro-4-methoxy-biphenyl-2-yl)-quinoline (111)

Compound 110 (100 mg (0.5 mmol)) and compound 106 (130 mg (0.5 mmol)) were dissolved in 5 mL ethanol, 800 μL 10% KOH (1.5 mmol) was added and the mixture was kept in a 90° C. bath under argon overnight. The solvent was evaporated and the residue triturated with water. The semi solid compound 111 was purified on a 400 mL silica gel pad using toluene for elution to give 2.03 g (44%) yellow gummy material; MS: 424.03 & 426.03 (M+H⁺); ¹H-NMR (DMSO-d₆): δ (ppm) 8.20 (d, 1H, J=2.1 Hz), 8.10 (d, 1H, J=9.0 Hz), 7.93-7.83 (m, 2H), 7.40 (d, 1H, J=8.4 Hz), 7.26-7.23 (m, 3H0, 7.16-7.03 (m, 4H), 3.85 (s, 3H).

### Preparation of 2-boronic acid derivative of 3-Cyclohexyl-1H-indole-6-carboxylic acid methyl ester (113)

Compound 112 (1 g (3 mmol) synthesized as described in International Patent Application Publication Number WO 03/010141), 890 mg (9 mmol) potassium acetate, 105 mg (0.15 mmol) [P(Ph₃)]₂Pd(II)Cl₂ and 6.7 g (30 mmol) bis(neopentyl glycolato)diboron were dissolved in 20 mL DMSO and the mixture was heated overnight at 95 C.°. The crude product was precipitated by addition of 30 mL water and was purified on a silica gel pad using toluene-ethylacetate solvent gradient elution to yield 391 mg (43%) of the title compound; ¹H-NMR (DMSO-d₆): δ (ppm) 11.06 (s, 1H), 8.01 (d, 1H, J=1.5 Hz), 7.78 (d, 1H, J=8.4 Hz), 7.47 (dd, 1H, J=8.4 and 1.8 Hz), 3.81 (s, 3H), 1.98-1.33 (m, 11H).

### Preparation of 2-[2-(4′-chloro-4-methoxy-biphenyl-2-yl)-quinolin-6-yl]-3-cyclohexyl-1H-indole-6-carboxylic acid (200)

A mixture of 106 g (0.25 mmol) compound 111, 180 mg (0.6 mmol) compound 113, 58 mg (0.05 mmol) tetrakis-(triphenylphosphino)palladium, 6 mL toluene, 1.5 mL methanol and 600 μL saturated sodium bicarbonate was heated under argon overnight at 80° C. The solution was then evaporated to dryness to provide for compound 114 which was used without isolation. Compound 114 was dissolved in 5 mL ethanol, 3 mL 1M NaOH was added and was heated at 85° C. for 30 minutes. It was evaporated to dryness. The pure product was isolated using RP-HPLC followed by converting to HCL salt as follows: Purified compound 200 was dissolved in acetonitrile, 1 mL 4M HCl/1,4-dioxane was added and the mixture was evaporated to dryness. The residue was suspended in water and lyophilized overnight to yield 27.5 mg (19%) yellow solid; MS: 587.23 (M+H⁺); ¹H-NMR (DMSO-d₆): δ (ppm) 11.66 (s, 1H), 8.39 (d, 1H, J=8.4 Hz), 8.20 (d, 1H, J=8.7 Hz), 8.12 (d, 1H, J=1.5 Hz), 8.00-7.95 (m, 2H), 7.86 (d, 1H, J=8.4 Hz), 7.59 (dd, 1H, J=8.7 and 1.5 Hz), 4.47 (d, 1H, J=8.7 Hz), 7.34-7.28 (m, 3H), 7.22-7.18 (m, 2H), 7.14-7.11 (m, 2H), 3.88 (s, 3H), 2.96 (m, 1H), 2.05-1.22 (m, 10H).

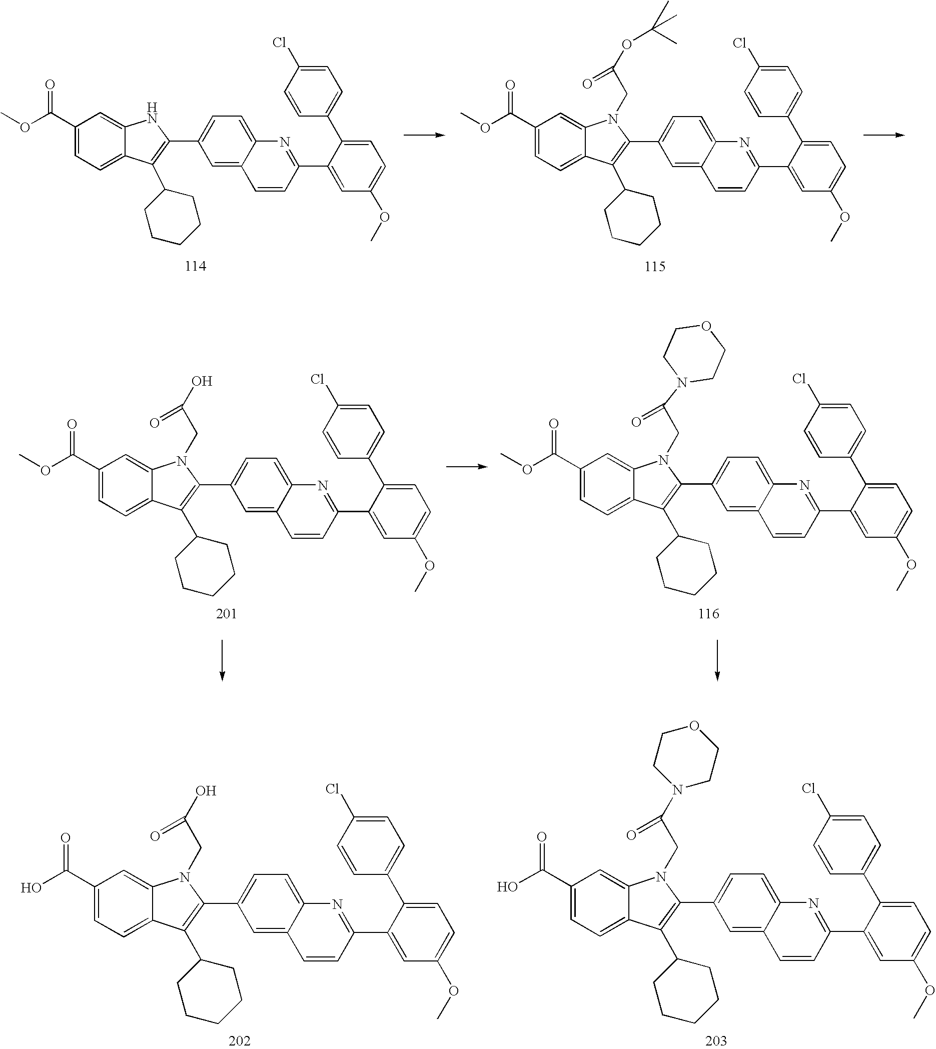

### Example 2

### 1-carboxymethyl-2-[2-(4′-chloro-4-methoxy-biphenyl-2-yl)-quinolin-6-yl]-3-cyclohexyl-1H-indole-6-carboxylic acid methyl ester (Compound 201)

### Preparation of 1-tert-butoxycarbonylmethyl-2-[2-(4′-chloro-4-methoxy-biphenyl-2-yl)-quinolin-6-yl]-3-cyclohexyl-1H-indole-6-carboxylic acid methyl ester (115)

To an ice cold solution of 590 mg (0.985 mmol) of compound 114 in 18 mL DMF, 47.5 mg (1.97 mmol) NaH was added. The mixture was stirred under vacuum at this temperature for 30 minutes, then at room temperature for 15 minutes. 366 μL (2.5 mmol) bromoacetic acid tert-butyl ester was added and stirred at room temperature for 15 minutes when the reaction was complete. The solvent was evaporated and the residue triturated with water to give 648 mg (90%) of the title compound after drying. The compound was judged pure enough by HPLC to use without further purification; MS: 715.29 (M+H⁺).

### Preparation of 1-carboxymethyl-2-[2-(4′-chloro-4-methoxy-biphenyl-2-yl)-quinolin-6-yl]-3-cyclohexyl-1H-indole-6-carboxylic acid methyl ester (201)

Compound 115 (648 mg (0.9 mmol)) was dissolved in a mixture of 20 mL TFA and 2 mL anisole. The mixture was allowed to stand at room temperature for 1 h. After the volatiles were evaporated, the residue was co-evaporated with DMF and was dried. The crude product was purified using RP-HPLC. The product was converted to the HCl salt as described for compound 200 to give 501 mg (84%); MS: 659.26 (M+H⁺); ¹H-NMR (DMSO-d₆): δ (ppm) 8.23-8.15 (m, 2H), 8.08 (s, 1H), 7.92-7.67 (m, 2H), 7.70-7.67 (m, 2H), 7.46-7.42 (m, 2H), 7.33-7.30 (m, 2H), 7.20-7.08 (m, 4H), 4.83 (s, 2H), 3.87 (s, 6H), 2.59 (m, 1H), 1.90-1.19 (m, 10H).

### Example 3

### 1-[N-morpholinocarbonylmethyl]-2-[2-(4′-chloro-4-methoxy-biphen-2-yl)-quinolin-6-yl]-3-cyclohexyl-1H-indole-6-carboxylic acid (Compound 203)

### Preparation of 2-[2-(4′-chloro-4-methoxy-biphenyl-2-yl)-quinolin-6-yl]-3-cyclohexyl-1-(2-morpholin-4-yl-2-oxo-ethyl)-1H-indole-6-carboxylic acid methyl ester (116)

A mixture of compound 201 (128 mg (0.194 mmol)), 92.24 mg (0.243 mmol) HATU and 84.6 μL (0.485 mmol) DIEA in 2 mL DMF was stirred at room temperature for 15 minutes. 25.5 μL (0.291 mmol) morpholine was added and the mixture was stirred for 10 more minutes. The solvent was evaporated, the residue was triturated with water. The solid product was pure enough (by HPLC) to be used without further purification; MS: 728.28 (M+H⁺).

### Preparation of 1-[N-morpholinocarbonylmethyl]-2-[2-(4′-chloro-4-methoxy-biphen-2-yl)-quinolin-6-yl]-3-cyclohexyl-1H-indole-6-carboxylic acid (203)

Compound 116 (141 mg (0.194 mmol)) was dissolved in 15 mL of a 1:1 methanol-ethanol mixture. Then 3 mL (3 mmol) of a 1M NaOH solution was added and the mixture was stirred at 50° C. for 1.5 h. The volatiles were evaporated under vacuum and the residue was purified with RP-HPLC. The product was then converted to HCl salt as described for compound 200 to give 14 mg (10%) of the title compound; MS: 714.28 (M+H⁺); ¹H-NMR (DMSO-d₆): δ (ppm) 8.20 (d, 1H, J=8.7 Hz), 8.13 (d, 1H, J=8.7 Hz), 8.01 (s, 1H), 7.86-7.83 (m, 2H), 7.67-7.64 (m, 2H), 7.44 (d, 1H, J=8.4 Hz), 7.30-7.27 (m, 3H), 7.19-7.09 (m, 4H), 4.98 (s, 2H), 3.87 (s, 3H), 3.55-3.29 (m, 8H), 2.62 (m, 1H), 1.92-1.17 (m, 10H).

### Example 4

### 1-carboxymethyl-2-[2-(4′-chloro-4-methoxy-biphenyl-2-yl)-quinolin-6-yl]-3-cyclohexyl-1H-indole-6-carboxylic acid (Compound 202)

The title compound was synthesized from compound 201 as described for compound 203; MS: 644.21 (M+H⁺); ¹H-NMR (DMSO-d₆): δ (ppm) 8.25-8.16 (m, 2H), 8.03 (s, 1H), 7.93 (d, 1H), 7.87 (d, 1H, J=8.1 Hz), 7.12-7.66 (m, 2H), 7.45 (d, 1H, J=8.4 Hz), 7.33-7.30 (m, 3H), 7.20-7.08 (m, 4H), 4.81 (s, 2H), 3.87 (s, 3H), 2.59 (m, 1H), 1.90-1.19 (m, 10H).

### Example 5

### 1-[N-piperazinylcarbonylmethyl]-2-[2-(4′-chloro-4-methoxy-biphen-2-yl)-quinolin-6-yl]-3-cyclohexyl-1H-indole-6-carboxylic acid (Compound 204)

The title compound was synthesized from compound 201 in two steps as described for compound 116 and compound 203 replacing morpholine with piperazine in the first step; MS: 713.30 (M+H⁺); H¹-NMR (DMSO-d₆): δ (ppm) 9.02 (br, m, 2H), 8.23 (d, 1H, J=8.7 Hz), 8.15 (d, 1H, J=8.7 Hz), 8.01 (s, 1H), 7.87-7.84 (m, 2H), 7.68-7.62 (m, 2H), 7.44 (d, 1H, J=8.4 Hz), 7.32-7.29 (m, 3H), 7.2-7.09 (m. 4H), 5.05 (s, 2H), 3.87 (s, 3H), 3.01-2.93 (m, 4H), 2.61 (m, 1H), 1.93-1.20 (m, 12H).

### Example 6

### 1-[(4-pyrrolidin-1-yl-piperidin-1-yl)carbonylmethyl]-2-[2-(4′-chloro-4-methoxy-biphen-2-yl)-quinolin-6-yl]-3-cyclohexyl-1H-indole-6-carboxylic acid (Compound 205)

The title compound was synthesized from compound 201 in two steps as described for compound 116 and compound 203 replacing morpholine with 4-[1-pyrrolidino]-piperidine in the first step; MS: 781.37 (M+H⁺); ¹H-NMR (DMSO-d₆): δ (ppm) 10.49 (m, 1H), 8.26 (d, 1H, J=8.4 hz), 8.18-8.15 (m, 1H), 7.96 (s, 1H), 7.87-7.84 (m, 2H), 7.67-7.644 (m, 2H), 7.44 (d, 1H, J=8.4 Hz), 7.32-7.29 (m, 3H), 7.20-7.10 (m, 4H), 4.97 (m, 1H), 4.38-4.33 (m, 1H), 4.03-3.87 (m, 5H), 3.37 (m, 2H), 2.95 (m, 3H), 2.56 (m, 1H), 2.10-1.22 (m, 18H).

### Example 7

### 1-[N,N-dimethylaminocarbonyl-methyl]-2-[2-(4′-chloro-4-methoxy-biphen-2-yl)-quinolin-6-yl]-3-cyclohexyl-1H-indole-6carboxylic acid (Compound 206)

The title compound was synthesized from compound 201 in two steps as described for compound 116 and compound 203 replacing morpholine with dimethylamine in the first step; MS: 672.27 (M+H⁺); H¹-NMR (DMSO-d₆): δ (ppm) 8.21 (d, 1H, J=8.7 Hz), 8.11 (d, 1H, J=9.0 Hz), 7.96 (m, 1H), 7.87-7.83 (m, 2H), 7.66-7.63 (m, 2H), 7.44 (d, 1H, J=8.4 Hz), 7.31-7.28 (m, 3H), 7.19-7.09 (m, 4H), 4.94 (s, 2H), 2.87 (s, 3H), 2.86 (s, 3H), 2.76 (s, 3H), 2.59 (m, 1H), 1.92-1.16 (m, 10H).

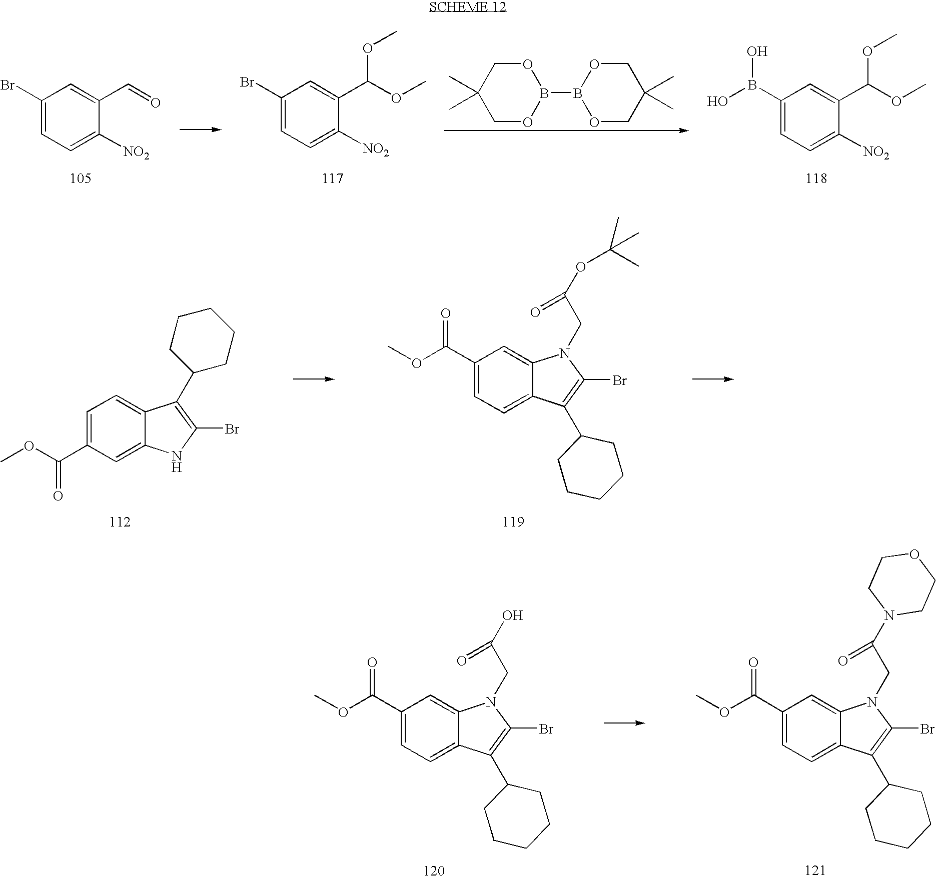

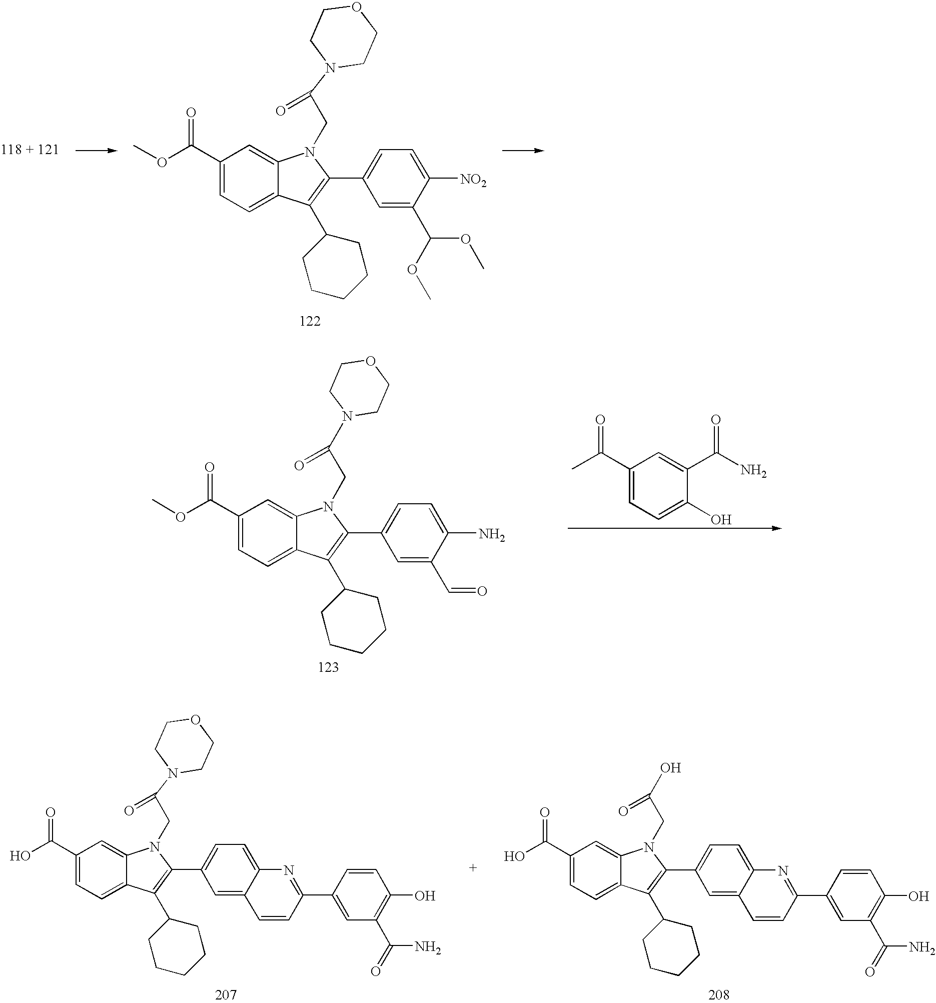

### Example 8

### 1-[N-morpholinocarbonylmethyl]-2-[2-(3-amido-4-hydroxy-phenyl)-quinolin-6-yl]-3-cyclohexyl-1H-indole-6-carboxylic acid (Compound 207) and 1-carboxylmethyl-2-[2-(3-Carbamoyl-4-hydroxy-phenyl)-quinolin-6-yl]-3-cyclohexyl-1H-indole-6-carboxylic acid (Compound 208)

### Preparation of 4-bromo-2-dimethoxymethyl-1-nitro-benzene (117)

To a solution of 5 g (21.73 mmol) of compound 105 in 100 mL methanol, 2.5 mL 4M HCl/dioxane was added and the mixture was kept in a 90° C. bath for 10 minutes. The solvents were evaporated and the residue was co-evaporated with methanol. The brown oil was dried in high vacuum overnight to give the title compound in quantitative yield; ¹H-NMR (DMSO-d₆): δ (ppm) 7.85 (m. 2H), 7.78 (m, 1H), 5.78 (s, 1H), 3.30 (s, 6H).

### Preparation of 3-dimethoxymethyl-4-nitro-phenylboronic acid (118)

A mixture of 6.0 g (21.73 mmol) of compound 117, 6.42 g (65.5 mmol) potassium acetate, 750 mg (1.07 mmol) P(Ph)₃Pd(II)Cl₂ catalyst and 14.7 g (65 mmol) bis(neopentylglycolato)diboron in 120 mL DMSO was heated at 50° C. under argon for 4 h. After 150 mL water and 150 mL ethyl acetate was added, the organic phase was separated. The aqueous phase was extracted one more time with 50 mL ethyl acetate. The organic phases were pooled and washed with water (2×), brine (2×) and dried (sodium sulfate). The solvent was evaporated and the residue was purified by filtering through a 400 mL silica pad using toluene-ethyl acetate gradient to get 4.4 g (84%) of the title compound; MS: 240.07 (M+H⁺).

### Preparation of 2-bromo-1-tert-butoxycarbonylmethyl-3-cyclohexyl-1H-indole-6-carboxylic acid methyl ester (119)

To an ice cold solution of 2.5 g (7.44 mmol) of compound 112 dissolved in DMF, 223 mg (9.3 mmol) NaH was added and the mixture was stirred at this temperature for 30 minutes under vacuum, then 1.16 mL (7.81 mmol) bromoacetic acid tert-butylester was added. The reaction was complete in 5 minutes. The solvent was evaporated immediately. The residue was treated with ice and water, the solid was filtered off and washed with water (3×) then dried overnight under high vacuum to give 3.18 g (95%) of the title compound as a yellow solid; ¹H-NMR (DMSO-d₆): δ (ppm) 8.12 (s, 1H), 7.80 (d, 1H, J=8.7 Hz), 7.63 (d, 1H, J=8.7 Hz)), 5.08 (s, 2H), 3.85 (s, 3H), 2.83 (m, 1H), 1.93-1.35(m, 19H).

### Preparation of 2-bromo-1-carboxymethyl-3-cyclohexyl-1H-indole-6-carboxylic acid methyl ester (120)

Compound 119 (3.18 g (7.06 mmol)) was dissolved in a mixture of 25 mL TFA and 5 mL anisole. The mixture was allowed to stand at room temperature for 1 h. The volatiles were evaporated and the residue was co-evaporated with toluene (1×), DMF (1×) and was then dried to give the title compound in quantitative yield (2.78 g); MS: 394.06 (M+H⁺); ¹H-NMR (DMSO-d₆): δ (ppm) 8.104 (s, 1H), 7.80 (d, 1H, J=8.7 Hz), 7.63 (dd, 1H, J=8.4 Hz), 5.10 (s, 2H), 3.84 (s, 3H), 2.83 (m, 1H), 1.92-1.24 (m, 10H).

### Preparation of 2-bromo-3-cyclohexyl-1-(2-morpholin-4-yl-2-oxo-ethyl)-1H-indole-6-carboxylic acid methyl ester (121)

A mixture of 2.78 g (7.05 mmol) of compound 120, 3.35 g (8.82 mmol) HBTU and 3.07 mL (17.6 mmol) DIEA in 50 mL DMF was stirred at room temperature for 15 minutes. Then 1.23 mL (14.1 mmol) morpholine was added and was stirred for 10 more minutes. The solvent was evaporated; the residue was filtered through a silica gel pad (400 mL) using toluene-ethyl acetate gradient to give 2.88 g (88%) of the title compound as a white solid; MS: 463.12 (M+H⁺); H¹-NMR (DMSO-d₆): δ (ppm) 8.06 (d, 1H, J=0.9 Hz), 7.78 (d, 1H, J=8.4 Hz), 7.61 (dd, 1H, J=8.4 Hz, 1.5 Hz), 5.29 (s, 2H), 3.84 (s, 3H), 3.68-3.42 (m, 8H), 2.83 (m, 1H), 1.93-1.35 (m, 10H).

### Preparation of 3-cyclohexyl-2-(3-dimethoxymethyl-4-nitro-phenyl)-1-(2-morpholin-4-yl-2-oxo-ethyl)-1H-indole-6-carboxylic acid methyl ester (122)

The mixture of 337 mg (0.73 mmol) of compound 121, 308.5 mg (1.28 mmol) of compound 118, 46 mg (0.04 mmol) tetrakis(triphenylphosphino)palladium 2 mL saturated NaHCO₃ in 16 mL methanol was heated under argon at 80° C. for four hours. The solvents were evaporated and the residue was filtered through a silica pad (200 mL) using toluene-ethyl acetate gradient to give 400 mg (94.5%) of the title compound as a yellow solid; ¹H-NMR (DMSO-d₆): δ (ppm) 8.08 (d, 1H, J=8.1 Hz), 8.01 (d, 1H, J=1.2 Hz, 7.89 (d, 1H, J=8.7 Hz), 7.66 (dd, 1H, J=8.1 Hz, 1.2 Hz), 7.60 (d, 1H, J=1.8 Hz)), 7.56 (dd, 1H, J=8.1 Hz, 1.8 Hz), 5.85 (s, 1H), 5.00 (br, s, 2H), 3.86 (s, 3H), 3.51-3.30 (m, 14H), 2.63 (m, 1H), 1.90-1.16 (m, 10H).

### Preparation of 2-(4-amino-3-formyl-phenyl)-3-cyclohexyl-1-(2-morpholin-4-yl-2-oxo-ethyl)-1H-indole-6-carboxylic acid methyl ester (123)

A mixture of 20 mL methanol, 500 mg MgSO₄ and 100 mg 10% Pd—C catalyst were hydrogenated at 30 psi for 15 minutes. Then 1 mL triethylamine was added followed by 400 mg (0.69 mmol) of compound 122 dissolved in 20 mL methanol. The hydrogenation was continued for 1 h until the reduction was complete. The catalyst was filtered off and the solution was evaporated to dryness resulting in a light brown oil which was dissolved in 40 mL solvent mixture of 2:2:1 ethanol:acetic acid:water. The solvent was evaporated and the residue was dried overnight in high vacuum to yield 359 mg (quantitative) of the title compound; MS: 504.24 (M+H⁺); H¹-NMR (DMSO-d₆): δ (ppm) 7.96 (s, 1H), 7.79 (d, 1H, J=8.4 Hz), 7.62 (dd, 1H, J=8.1 Hz), 7.43 (d, 1H, 2.1 Hz), 7.37 (s, 2H), 7.18 (dd, 1H, J=8.4 Hz, 1.5 Hz), 6.86 (d, 1H, J=8.7 Hz), 4.94 (s, 2H), 3.84 (s, 3H), 3.48-3.40 (m, 8H), 2.59 (m, 1H), 1.88-1.25 (m, 10H).

### Preparation of 1-[N-morpholinocarbonylmethyl]-2-[2-(3-amido-4-hydroxy-phenyl)-quinolin-6-yl]-3-cyclohexyl-1H-indole-6-carboxylic acid (Compound 207) and 1-carboxylmethyl-2-[2-(3-carbamoyl-4-hydroxy-phenyl)-quinolin-6-yl]-3-cyclohexyl-1H-indole-6-carboxylic acid (Compound 208)

A mixture of 100 mg (0.2 mmol) compound 123, 64.4 mg (0.4 mmol) 5-acetyl salicilamide, and 650 μL (1.44 mmol) 10% KOH was refluxed overnight under argon. The solvent was evaporated and the residue was purified by RP-HPLC using 10 mM ammonium acetate/water-10 mM ammonium acetate/acetonitrile eluent system. The reaction resulted in two products, 8.6 mg of compound 207 and 5.1 mg of compound 208. They were converted to HCl salt as described for compound 200. Compound 207: MS: 633.27 (M+H⁺); ¹H-NMR (DMSO-d₆): δ (ppm) 8.76 (m, 2H), 8.56 (d, 1H, J=9.0 Hz), 8.47 (dd, 1H, J=8.4 Hz), 8.28 (d, 1H, J=8.7 Hz), 8.16 (d, 1H, J=8.4 Hz), 8.08 (m, 1H), 8.01 (s, 1H), 7.92 (d, 1H), 7.86 (d, 1H, J=8.4 Hz), 7.66 (m, 2H), 7.09 (d, 1H, J=8.7 Hz), 4.99 (s, 2H), 3.47-3.33 (m, 8H), 2.65 (m, 1H), 1.93-1.23 (m, 10H); Compound 208: MS: 564.20 (M+H⁺); ¹H-NMR (DMSO-d₆): δ (ppm) 8.76-8.72 (m, 2H), 8.57 (d, 1H, J=9.0 Hz), 8.47 (dd, 1H, J=8.4 Hz), 8.30 (d, 1H, J=8.7 Hz), 8.18 (d, 1H, J=8.7 Hz), 8.09 (m, 1H), 8.04 (s, 1H), 7.98 (d, 1H), 7.87 (d, 1H, J=8.1 Hz), 7.68 (m, 2H), 7.10 (d, 1H, J=8.7 Hz), 4.83 (s, 2H), 2.63 (m, 1H), 1.91-1.23 (m, 10H).

### Example 9

### 1-[N-morpholinocarbonylmethyl]-2-[2-(4-(1H-imidazolyl)phenyl)-quinolin-6-yl]-3-cyclohexyl-1H-indole-6-carboxylic acid (Compound 209)

The title compound was synthesized from compound 123 as described for compound 207 replacing 5-acetyl salicilamide with 4′-(imidazol-1-yl)acetophenone; MS: 640.25 (M+H⁺); H¹-NMR (DMSO-d₆): δ (ppm) 9.91 (s, 1H), 8.64 (d, 1H, J=8.7 Hz), 8.59-8.55 (m, 2H), 8.44 (m, 1H), 8.37 (d, 1H, J=8.7 Hz), 8.25 (d, 1H, J=8.7 Hz), 8.07-7.98 (m, 5H), 7.86 (d, 1H, J=8.1 Hz), 7.72-7.65 (m, 2H), 5.01 (s, 2H), 3.46-3.33 (m, 8H), 2.64 (m, 1H), 1.91-1.16 (m, 10H).

### Example 10

### 1-[N-morpholinocarbonylmethyl]-2-[2-(2,4-dimethylthiazol-5-yl)-quinolin-6-yl]-3-cyclohexyl-1H-indole-6-carboxylic acid (Compound 210) and 1-[carboxymethyl]-2-[2-(2,4-dimethylthiazol-5-yl)-quinolin-6-yl]-3-cyclohexyl-1H-indole-6-carboxylic acid (Compound 211)

The title compounds were synthesized from compound 123 as described for compound 207 and compound 208, respectively, replacing 5-acetyl salicilamide with 5-acetyl-2,4-dimethylthiazole; Compound 210: MS: 609.24 (M+H⁺); H¹-NMR (DMSO-d₆): δ (ppm) 8.52 (d, 1H, J=8.7 Hz), 8.07 (d, 1H, J=8.7 Hz), 8.00 (d, 1H, J=0.6 Hz), 7.93-7.90 (m, 2H), 7.85 (d, 1H, J=8.7 Hz), 7.67-7.62 (m, 2H), 4.99 (s, 2H), 3.36-3.33 (m, 8H), 2.72 (s, 3H), 2.67 (s, 3H), 2.62 (m, 1H), 1.91-1.15 (m, 10H); Compound 211: MS: 540.18 (M+H⁺); H¹-NMR (DMSO-d₆): δ (ppm) 8.52 (d, 1H, J=8.4 Hz), 8.07 (d, 1H, J=8.7 Hz), 8.03 (s, 1H,), 7.9 (d, 1H, J=8.7 Hz), 7.86 (d, 1H, J=8.1 Hz), 7.69-7.65 (m, 2H), 4.81 (s, 2H), 2.72 (s, 3H), 2.67 (s, 3H), 2.60 (m, 1H), 1.90-1.19 (m, 10H).

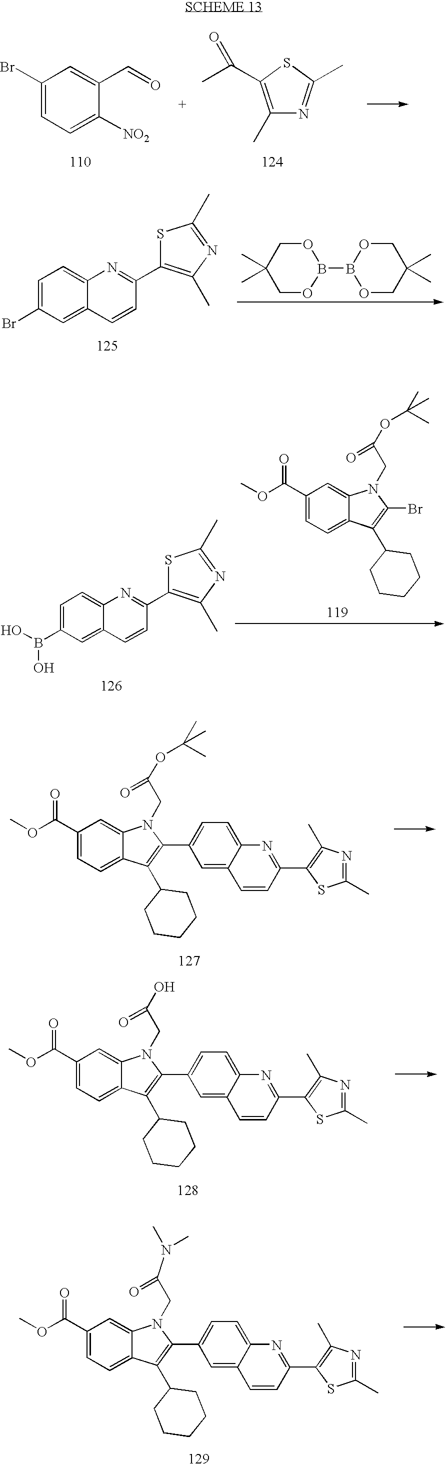

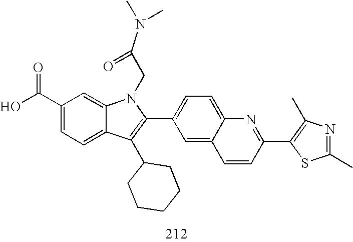

### Example 11

### 1-[N,N-dimethylaminocarbonyl-methyl]-2-[2-(2,4-dimethylthiazol-5-yl)-quinolin-6-yl]-3-cyclohexyl-1H-indole-6-carboxylic acid (Compound 212)

### Preparation of 6-bromo-2-(2,4-dimethyl-thiazol-5-yl)-quinoline (125)

A mixture of 1.071 g (5.354 mmol) compound 110, 723 μL (5.354 mmol) 5-acetyl-2,4-dimethylthiazole and 9.0 mL 10% KOH/ethanol (16.062 mmol KOH) in 60 mL ethanol was refluxed overnight under argon. It was then evaporated and the residue triturated with water. The solid crude product was filtered through a 250 mL silica pad using a 10% to 60% toluene-ethylacetate gradient to give 1.164 g (68%) compound 125; ¹H-NMR (DMSO-d₆): δ (ppm) 8.39 (d, 1H, J=8.7 Hz), 8.27 (m, 1H), 7.88-7.86 (m, 3H), 2.68 (s, 3H), 2.64 (s, 3H).

### Preparation of 2-(2,4-dimethyl-thiazol-5-yl)-quinoline-6-boronic acid (126)

Compound 126 was synthesized from compound 125 as described for compound 118 MS: 285.08 (M+H⁺); ¹H-NMR (DMSO-d₆): δ (ppm) 8.47 (d, 1H, J=8.7 Hz), 8.33 (s, 1H), 7.97 (m, 1H), 7.88-7.79 (m, 2H), 2.69 (s, 3H), 2.64 (s, 3H).

### Preparation of 1-tert-butoxycarbonylmethyl-3-cyclohexyl-2-[2-(2,4-dimethyl-thiazol-5-yl)-quinolin-6-yl]-1H-indole-6-carboxylic acid methyl ester (127)

Compound 127 was synthesized from compound 126 and compound 119 as described for compound 122; MS: 610.27 (M+H⁺).

### Preparation of 1-carboxymethyl-3-cyclohexyl-2-[2-(2,4-dimethyl-thiazol-5-yl)-quinolin-6-yl]-1H-indole-6-carboxylic acid methyl ester (128)

Compound 128 was synthesized from compound 127 as described for compound 120; MS: 554.20 (M+H⁺).

### Preparation of 3-cyclohexyl-1-dimethylcarbamoylmethyl-2-[2-(2,4-dimethyl-thiazol-5-yl)-quinolin-6-yl]-1H-indole-6-carboxylic acid methyl ester (129)

Compound 129 was synthesized from compound 128 as described for compound 121 replacing morpholine with dimethylamine; MS: 581.26 (M+H⁺).

### Preparation of 1-[N,N-dimethylaminocarbonyl-methyl]-2-[2-(2,4-dimethylthiazol-5-yl)-quinolin-6-yl]-3-cyclohexyl-1H-indole-6-carboxylic acid (compound 212)

Compound 129 was saponified as described for compound 203. The crude product was purified using RP-HPLC; MS: 567.24 (M+H⁺); ¹H-NMR (DMSO-d₆): δ (ppm) 8.50 (d, 1H, J=8.7 Hz), 8.04 (d, 1H, J=8.4 Hz), 7.97-7.82 (m, 4H), 7.66 (m, 1H), 4.94 (s, 2H), 2.85 (s, 3H), 2.77 (s, 1H), 2.72 (s, 1H), 2.67 (s, 1H), 2.60 (m, 1H), 1.95-1.10 (m, 10H).

### Example 12

### 1-[(N-carboxymethycarbamoyl)-methyl]-[2-(2,4-dimethyl-thiazol-5-yl)-quinolin-6-yl]-3-cyclohexyl-1H-indole-6-carboxylic acid, (Compound 213)

The title compound was synthesized from compound 128 in two steps as described for compound 129 and compound 210 replacing dimethylamine with glycinamide in the first step; MS: 597.24 (M+H⁺); ¹H-NMR (DMSO-d₆): (ppm) 8.54 (d, 1H, J=8.4 Hz), 8.42 (t, 1H, J=6 Hz), 8.07-7.98 (m, 3H), 7.91-7.84 (m, 2H), 7.75 (d, 1H, J=8.7 Hz), 7.67 (d, 1H, J=8.7 Hz), 4.68 (s, 2H), 3.77 (d, 2H, J=4.8 Hz), 2.72 (s, 3H), 2.68 (s, 3H), 2.64 (m, 1H), 1.93-1.20 (m, 10H).

### Example 13

### 1-[2-oxo-2-(4-pyrrolidin-1-yl-piperidin-1-yl)-ethyl]-2-[2-(2,4-dimethylthiazol-5-yl)-quinolin-6-yl]-3-cyclohexyl-1H-indole-6-carboxylic acid (Compound 214)

The title compound was synthesized from compound 128 in two steps as described for compound 129 and compound 210 replacing dimethylamine with 4-(pyrrolidin-1-yl)-piperidine in the first step; MS: 676.35(M+H⁺); ¹H-NMR (DMSO-d₆): δ (ppm) 10.81 (s, br, 1H), 8.54 (d, 1H, J=8.7 Hz), 8.09 (d, 1H, J=8.1 Hz), 7.98-7.91 (m, 3H), 7.85 (d, 1H, J=8.7 Hz), 7.64 (m, 2H), 5.02 (m, 2H), 4.35 (m, 1H), 3.90 (m, 1H), 3.33 (m, 4H), 2.88 (m, 3H), 2.72 (s, 3H), 2.67 (s, 3H), 2.55 (m, 1H), 2.1-1.06 (m, 20H).

### Example 14

### 1-[(2-hydroxyethylcarbamoyl)-methyl]-2-[2-(2,4-dimethyl-thiazol-5-yl)-quinolin-6-yl]-3-cyclohexyl-1H-indole-6-carboxylic acid (Compound 215)

The title compound was synthesized from compound 128 in two steps as described for compound 129 and compound 210 replacing dimethylamine with ethanolamine in the first step; MS: 583.26 (M+H⁺); ¹H-NMR (DMSO-d₆): δ (ppm) 8.50 (d, 1H, J=8.7 Hz), 8.10-8.04 (m, 2H), 7.98 (m, 2H), 7.91-7.83 (m, 2H), 7.73-7.65 (m, 2H), 4.62 (s, 2H), 3.33 (m, 2H), 3.10 (m, 2H), 2.72 (s, 3H), 2.67 (s, 3H), 2.60 (m, 1H), 1.89-1.08 (m,10H).

### Example 15

### 1-(2-oxo-2-piperidin-1-yl-ethyl)-2-[2-(2,4-dimethylthiazol-5-yl)-3-cyclohexylquinolin-6-yl]-1H-indole-6-carboxylic acid (Compound 216)

The title compound was synthesized from compound 128 in two steps as described for compound 129 and compound 210 replacing dimethylamine with piperidine in the first step; MS: 607.30 (M+H⁺); ¹H-NMR (DMSO-d₆): δ (ppm) 8.50 (d, 1H, J=8.7 Hz), 8.05 (d, 1H, J=8.7 Hz), 7.96-7.83 (m. 4H), 7.65 (m, 2H), 4.94 (s, 2H), 3.35 (m, 2H), 3.26 (m, 2H), 2.72 (s, 3H), 2.66 (s, 3H), 2.60 (m, 1H), 1.90-1.08 (m, 16H).

### Example 16

### 1-[N-morpholinocarbonylmethyl]-2-[2-(pyrid-2-yl)-quinolin-6-yl]-3-cyclohexyl-1H-indole-6-carboxylic acid (Compound 217)

Following the full procedure and workup for compound 207, compound 123 (100 mg, 0.2 mmol) was reacted with 1-pyridin-2-yl-ethanone (24 mg, 0.2 mmol) to produce the title compound (18 mg, 12% yield); MS: 575.27 (M+H⁺); ¹H-NMR (DMSO d₆): δ 8.80 (m, 1H), 8.69 (d, 1H, J=7.8), 8.65 (s, 2H), 8.25 (d, 1H, J=9), 8.15 (m, 1H), 8.01 (s, 1H), 7.85 (d, 1H, J=8.4), 7.68 (m, 3H), 5.01 (s, 2H), 3.38 (m, 8H), 2.65 (m, 1H), 1.76 (m, 7H), 1.22 (m, 3H).

### Example 17

### 1-[N-morpholinocarbonylmethyl]-2-[2-(pyrazin-2-yl)-quinolin-6-yl]-3-cyclohexyl-1H-indole-6-carboxylic acid (Compound 218)

Following the full procedure and workup for compound 207, compound 123 (100 mg, 0.2 mmol) was reacted with 1-pyrazin-2-yl-ethanone (24 mg, 0.2 mmol) to produce the title compound (9 mg, 7% yield); MS: 576.27 (M+H⁺); ¹H-NMR (DMSO d₆): δ 9.76 (s, 1H), 8.82 (m, 2H), 8.65 (d, 1H, J=8.4), 8.55 (d, 1H, J=8.4), 8.26 (d, 1H, J=8.7), 8.01 (s, 2H), 4.86 (d, 1H, J=8.4), 7.68 (m, 2H), 5.01 (s, 1H), 3.46 (m, 8H), 2.65 (m, 1H), 1.80 (m, 7H), 1.22 (m, 3H).

### Example 18

### 1-[N-morpholinocarbonylmethyl]-2-[2-(pyrrol-2-yl)-quinolin-6-yl]-3-cyclohexyl-1H-indole-6-carboxylic acid (Compound 219)

Following the full procedure and workup for compound 207, compound 123 (100 mg, 0.2 mmol) was reacted with 1-(1H-pyrrol-2-yl)-ethanone (22 mg, 0.2 mmol) to produce the title compound (5.2 mg, 4% yield); MS: 563.27 (M+H⁺); H¹-NMR (DMSO d₆): δ 8.59 (m, 1H), 8.17 (m, 2H), 8.01 (s, 1H), 7.93 (s, 1H), 7.85 (d, 1H, J=8.7), 7.69 (m, 2H), 7.31 (m, 2H), 6.36 (s, 1H), 5.00 (s, 2H), 3.40 (m, 8H), 2.62 (m, 1H), 1.76 (m, 7H), 1.23 (m, 3H).

### Example 19

### 1-[N-morpholinocarbonylmethyl]-2-[2-phenylquinolin-6-yl]-3-cyclohexyl-1H-indole-6-carboxylic acid (Compound 220)

Following the full procedure and workup for compound 207, compound 123 (100 mg, 0.2 mmol) was reacted with 1-phenyl-ethanone (24 mg, 0.2 mmol) to produce the title compound (22 mg, 20% yield); MS: 574.28 (M+H⁺); ¹H-NMR (DMSO d₆): δ 8.62 (d, 1H, J=9), 8.28 (m, 4H), 8.01 (m, 2H), 7.85 (d, 1H, J=8.4), 7.70 (m, 2H), 7.60 (m, 3H), 5.01 (s, 2H), 3.42 (m, 8H), 2.64 (m, 1H), 1.79 (m, 7H), 1.22 (m, 3H).

### Example 20

### 1-[N-morpholinocarbonylmethyl]-2-(2-furan-2-yl-quinolin-6-yl)-3-cyclohexyl-1H-indole-6-carboxylic acid (Compound 222)

Following the full procedure and workup for compound 207, compound 123 (100 mg, 0.2 mmol) was reacted with 1-furan-2-yl-ethanone (22 mg, 0.2 mmol) to produce the title compound (9 mg, 8% yield); MS: 564.28 (M+H⁺); ¹H-NMR (DMSO d₆): 8.50 (d, 1H, J=8.4), 8.11 (d, 1H, J=8.4), 8.01 (m, 3H), 7.91 (s, 1H), 7.86 (d, 1H, J=8.7), 7.65 (m, 2H), 7.44 (d, 1H, J=3.3), 6.75 (m, 1H), 4.99 (s, 2H), 3.40 (m, 8H), 2.64 (m, 1H), 1.76 (m, 7H), 1.23 (m, 3H).

### Example 21

### 1-(N-morpholinocarbonylmethyl-2-[2-(5-methyl-furan-2-yl)-quinolin-6-yl]-3-cyclohexyl-1H-indole-6-carboxylic acid (Compound 223)

Following the full procedure and workup for compound 207, compound 123 (100 mg, 0.2 mmol) was reacted with 1-(5-methyl-furan-2-yl)-ethanone (25 mg, 0.2 mmol) to produce the title compound (8 mg, 7% yield); MS: 578.29 (M+H⁺); ¹H-NMR (DMSO d₆): δ 8.49 (d, 1H, J=8.7), 8.12 (d, 1H, J=9), 7.99 (m, 2H), 7.87 (m, 2H), 7.65 (m, 2H), 7.39 (s, 1H), 6.40 (s, 1H), 4.99 (s, 2H), 3.39 (m, 8H), 2.64 (m, 1H), 2.49 (s, 3H), 1.76 (m, 7H), 1.23 (m, 3H).

### Example 22

### 1-[N-morpholinocarbonylmethyl]-2-[2-thien-2-ylquinolin-6-yl]-3-cyclohexyl-1H-indole-6-carboxylic acid (Compound 224)

Following the full procedure and workup for compound 207, compound 123 (100 mg, 0.2 mmol) was reacted with 1-thiophen-2-yl-ethanone (25 mg, 0.2 mmol) to produce the title compound (8 mg, 7% yield); MS: 580.26 (M+H⁺); ¹H-NMR (DMSO d₆): δ 8.47 (d, 1H, J=9), 8.19 (m, 1H), 8.05 (m, 3H), 7.87 (m, 2H), 7.76 (d, 1H, J=5.4), 7.64 (m, 2H), 7.23 (m, 1H), 4.99 (s, 2H), 3.42 (m, 8H), 2.64 (m, 1H), 1.76 (m, 7H), 1.22 (m, 3H).

### Example 23

### 1-[N-morpholinocarbonylmethyl]-2-[2-(5-chlorothien-2-yl)quinolin-6-yl]-3-cyclohexyl-1H-indole-6-carboxylic acid (Compound 225)

Following the full procedure and workup for compound 207, compound 123 (100 mg, 0.2 mmol) was reacted with 1-(5-chloro-thiophen-2-yl)-ethanone (32 mg, 0.2 mmol) to produce the title compound (5 mg, 4% yield); MS: 614.22 (M+H⁺); ¹H-NMR (DMSO d₆): δ 8.50 (d, 1H, J=8.1), 8.18 (m, 1H), 8.03 (m, 2H), 7.90 (m, 3H), 7.64 (m, 2H), 7.27 (m, 1H), 4.98 (s, 2H), 3.41 (m, 8H), 2.63 (m, 1H), 1.77 (m, 7H), 1.21 (m, 3H).

### Example 24

### 1-[N-morpholinocarbonylmethyl]-2-[2-pyrid-3-ylquinolin-6-yl]-3-cyclohexyl-1H-indole-6-carboxylic acid (Compound 226)

Following the full procedure and workup for compound 207, compound 123 (100 mg, 0.2 mmol) was reacted with 1-pyridin-3-yl-ethanone (24 mg, 0.2 mmol) to produce the title compound (12 mg, 11% yield); MS: 575.29 (M+H⁺); ¹H-NMR (DMSO d₆): δ9.53 (s, 1H), 8.86 (m, 1H), 8.79 (d, 1H, J=5.1), 8.64 (d, 1H, J=8.4), 8.35 (d, 1H, J=8.7), 8.23 (d, 1H, J=8.7), 8.00 (m, 2H), 7.86 (d, 1H, J=8.4), 7.77 (m, 1H), 7.69 (m, 2H), 5.01 (s, 2H), 3.41 (m, 8H), 2.64 (m, 1H), 1.77 (m, 7H), 1.21 (m, 3H).

### Example 25

### 1-[N-morpholinocarbonylmethyl]-2-[2-thiazol-2-ylquinolin-6-yl]-3-cyclohexyl-1H-indole-6-carboxylic acid (Compound 227)

Following the full procedure and workup for compound 207, compound 123 (100 mg, 0.2 mmol) was reacted with 1-thiazol-2-yl-ethanone (25 mg, 0.2 mmol) to produce the title compound (8 mg, 7% yield); MS: 581.25 (M+H⁺); ¹H-NMR (DMSO d₆): δ 8.61 (d, 1H, J=9), 8.36 (d, 1H, J=8.4), 8.18 (d, 1H, J=8.7), 8.09 (m, 1H), 7.99 (m, 3H), 7.86 (d, 1H, J=8.7), 7.68 (m, 2H), 5.01 (s, 2H), 3.40 (m, 8H), 2.64 (m, 1H), 1.76 (m, 7H), 1.21 (m, 3H).

### Example 26

### 1-[N-morpholinocarbonylmethyl]-2-[2-thien-3-ylquinolin-6-yl]-3-cyclohexyl-1H-indole-6-carboxylic acid (Compound 228)

Following the full procedure and workup for compound 207, compound 123 (100 mg, 0.2 mmol) was reacted with 1-thiophen-3-yl-ethanone (25 mg, 0.2 mmol) to produce the title compound (12 mg, 11% yield); MS: 580.25 (M+H⁺); ¹H-NMR (DMSO d₆): δ 8.49 (m, 2H), 8.15 (m, 2H), 7.99 (m, 2H), 7.91 (s, 1H), 7.85 (d, 1H, J=8.4), 7.72 (m, 1H), 7.65 (m, 2H), 4.99 (s, 2H), 3.43 (m, 8H), 2.64 (m, 1H), 1.77 (m, 7H), 1.25 (m, 3H).

### Example 28

### 3-Cyclohexyl-2-[2-(3-methoxy-phenyl)-quinolin-6-yl]-1-(2-morpholin-4-yl-2-oxo-ethyl)-1H-indole-6-carboxylic acid (221)

Following the full procedure and workup for compound 207, 123 (100 mg, 0.2 mmol) was reacted with 1-(3-Methoxy-phenyl)-ethanone (31, 0.2 mmol) to produce compound 221 (5 g, 4% yield). MS: 604.29 (M+H⁺); H¹-NMR (DMSO d₆): 8.54 (d, 1H, J=8.7), 8.21 (m, 2H), 8.00 (s, 1H), 7.96 (s, 1H), 7.87 (m, 3H), 7.66 (m, 2H), 7.47 (m, 1H), 7.10 (dd, 1H, J=8.1, J=2.7), 5.00 (s, 2H), 3.89 (s, 3H), 3.41 (m, 8H), 2.65 (m, 1H), 1.80 (m, 7H), 1.23 (m, 3H).

### Example 29

### 3-Cyclohexyl-2-[2-(3-methyl-thiophen-2-yl)-quinolin-6-yl]-1-(2-morpholin-4-yl-2-oxo-ethyl)-1H-indole-6-carboxylic acid (229)

Following the full procedure and workup for compound 207, 123 (200 mg, 0.4 mmol) was reacted with 1-(3-Methyl-thiophen-2-yl)-ethanone (56 mg, 0.4 mmol) to produce compound 229 (5 mg, 4% yield). MS: 594.26 (M+H⁺); H¹-NMR (DMSO d₆): 8.49 (d, 1H, J=8.7), 8.08 (d, 1H, J=8.7), 8.00 (d, 1H, J=1.2), 7.92 (m, 2H), 7.86 (d, 1H, J=8.4), 7.65 (m, 3H), 7.08 (d, H, J=4.8), 5.00 (s, 2H), 3.43 (m, 8H), 2.63 (s, 3H), 2.51 (m, 1H), 1.76 (m, 7H), 1.23 (m, 3H).

### Example 30

### 3-Cyclohexyl-2-[2-(2,5-dimethyl-furan-3-yl)-quinolin-6-yl]-1-(2-morpholin-4-yl-2-oxo-ethyl)-1H-indole-6-carboxylic acid (230)

Following the full procedure and workup for compound 207, 123 (200 mg, 0.4 mmol) was reacted with 1-(2,5-Dimethyl-furan-3-yl)-ethanone (55 mg, 0.4 mmol) to produce compound 230 (12 mg, 8% yield). MS: 592.29 (M+H⁺); H¹-NMR (DMSO d₆): 8.44 (d, 1H, J=8.7), 8.06 (d, 1H, J=8.4), 7.99 (s, 1H), 7.85 (m, 3H), 7.64 (m, 2H), 6.72 (s, 1H), 4.98 (s, 2H), 3.40 (m, 8H), 2.75 (s, 3H), 2.63 (m, 1H), 2.31 (s, 3H), 1.75 (m, 7H), 1.21 (m, 3H).

### Example 31

### 3-Cyclohexyl-1-(2-morpholin-4-yl-2-oxo-ethyl)-2-(2-m-tolyl-quinolin-6-yl)-1H-indole-6-carboxylic acid (231)

Following the full procedure and workup for compound 207, 123 (200 mg, 0.4 mmol) was reacted with 1-m-Tolyl-ethanone (54 mg, 0.4 mmol) to produce compound 231 (14 mg, 7% yield). MS: 588.29 (M+H⁺); H¹-NMR (DMSO d₆): 8.54 (d, 1H, J=8.4), 8.20 (m, 2H), 8.10 (m, 2H), 8.00 (s, 1H), 7.95 (s, 1H), 7.85 (d, 1H, J=8.7), 7.65 (d, 2H, J=8.7), 7.45 (m, 1H), 7.33 (m, 1H), 5.00 (s, 2H), 3.42 (m, 8H), 2.64 (m, 1H), 2.45 (s, 1H), 1.76 (m, 7H), 1.24 (m, 3H).

### Example 32

### 3-Cyclohexyl-1-(2-morpholin-4-yl-2-oxo-ethyl)-2-(2-o-tolyl-quinolin-6-yl)-1H-indole-6-carboxylic acid (232)

Following the full procedure and workup for compound 207, 123 (200 mg, 0.4 mmol) was reacted with 1-o-Tolyl-ethanone (54 mg, 0.4 mmol) to produce compound 232 (8 mg, 5% yield). MS: 588.30 (M+H⁺); H¹-NMR (DMSO d₆): 8.55 (d, 1H, J=8.4), 8.15 (d, 1H, J=8.4), 8.00 (s, 2H), 7.86 (d, 1H, J=8.7), 7.80 (d, 1H, J=8.7), 7.67 (m, 2H), 7.52 (m, 1H), 7.36 (m, 3H), 5.01 (s, 2H), 3.40 (m, 8H), 2.65 (m, 1H), 2.42 (s, 3H), 1.77 (m, 7H), 1.23 (m, 3H).

### Example 33

### 3-Cyclohexyl-2-[2-(2-methoxy-phenyl)-quinolin-6-yl]-1-(2-morpholin-4-yl-2-oxo-ethyl)-1H-indole-6-carboxylic acid (233)

Following the full procedure and workup for compound 207, 123 (200 mg, 0.4 mmol) was reacted with 1-(2-Methoxy-phenyl)-ethanone (60 mg, 0.4 mmol) to produce compound 233 (10 mg, 4% yield). MS: 604.29 (M+H⁺); H¹-NMR (DMSO d₆): 8.51 (d, 1H, J=9), 8.17 (d, 1H, J=8.4), 8.00 (m, 3H), 7.82 (m, 2H), 7.67 (m, 2H), 7.51 (m, 1H), 7.23 (d, 1H, J=8.1), 7.14 (m, 1H), 5.00 (s, 2H), 3.87 (s, 3H), 3.43 (m, 8H), 2.64 (m, 1H), 1.78 (m, 7H), 1.23 (m, 3H).

### Example 34

### 3-Cyclohexyl-2-[2-(4-methyl-thiophen-2-yl)-quinolin-6-yl]-1-(2-morpholin-4-yl-2-oxo-ethyl)-1H-indole-6-carboxylic acid (234)

Following the full procedure and workup for compound 207, 123 (200 mg, 0.4 mmol) was reacted with 1-(4-Methyl-thiophen-2-yl)-ethanone (56 mg, 0.4 mmol) to produce compound 234 (10 mg, 5% yield). MS: 594.25 (M+H⁺); H¹-NMR (DMSO d₆): 8.44 (d, 1H, J=8.1), 8.12 (d, 1H, J=8.7), 8.03 (m, 2H), 7.85 (m, 3H), 7.62 (m, 2H), 7.34 (s, 1H), 4.98 (s, 2H), 3.40 (m, 8H), 2.63 (m, 1H), 2.29 (s, 3H), 1.76 (m, 7H), 1.24 (m, 3H).

### Example 35

### 3-Cyclohexyl-2-[2-(5-methyl-thiophen-2-yl)-quinolin-6-yl]-1-(2-morpholin-4-yl-2-oxo-ethyl)-1H-indole-6-carboxylic acid (235)

Following the full procedure and workup for compound 207, 123 (200 mg, 0.4 mmol) was reacted with 1-(5-Methyl-thiophen-2-yl)-ethanone (56 mg, 0.4 mmol) to produce compound 235 (13 mg, 7% yield). MS: 594.25 (M+H⁺); H¹-NMR (DMSO d₆): 8.42 (d, 1H, J=8.4), 8.11 (d, 1H, J=9), 8.01 (m, 2H), 7.84 (m, 3H), 7.62 (m, 2H), 6.93 (m, 1H), 4.98 (s, 2H), 3.41 (m, 8H), 2.63 (m, 1H), 2.53 (s, 3H), 1.75 (m, 7H), 1.24 (m, 3H).

### Example 36

### 3-Cyclohexyl-2-[2-(2,4-dimethyl-thiazol-5-yl)-quinolin-6-yl]-1-[2-(4-hydroxy-piperidin-1-yl)-2-oxo-ethyl]-1H-indole-6-carboxylic acid (236)

Compound 236 was synthesized from compound 128 as described for compound 121 replacing morpholine with piperidin-4-ol, followed by saponification as in compound 203. Yield 13.8 mg, 11%. MS: 623.3 (M+H⁺); H¹-NMR (DMSO-d₆): δ (ppm) 8.51 (d, 1H, J=9.0), 8.06-7.83 (m, 5H), 7.65 (m, 2H), 4.96 (s, 2H), 3.75 (m, 4H), 3.05 (m, 4H), 2.72 (s, 3H), 2.66 (s, 3H), 2.63 (m, 1H), 2.40 (m, 1H), 1.84-1.07 (m, 11H).

### Example 37

### 3-Cyclohexyl-2-[2-(2,4-dimethyl-thiazol-5-yl)-quinolin-6-yl]-1-[(2-morpholin-4-yl-ethylcarbamoyl)-methyl]-1H-indole-6-carboxylic acid (237)

Compound 237 was synthesized from compound 128 as described for compound 121 replacing morpholine with 2-morpholin-4-yl-ethylamine, followed by saponification as in compound 203. Yield 47.1 mg, 36%. MS: 652.3 (M+H⁺); H¹-NMR (DMSO-d₆): δ (ppm) 8.45 (d, 1H, J=8.7), 8.25 (t, 1H, J=5.7), 8.01 (d, 1H, J=8.7) 8.03-7.61 (m, 6H), 4.62 (s, 2H), 3.85 (d, 2H, J=12), 3.53 (t, 1H, J=12.3), 3.34 (m, 4H), 3.03 (m, 4H), 2.62 (s, 3H), 2.61 (s, 3H), 2.52 (m, 1H) 1.84-1.07 (m, 10H).

### Example 38

### 3-Cyclohexyl-2-[2-(2,4-dimethyl-thiazol-5-yl)-quinolin-6-yl]-7-methyl-1-(2-oxo-2-morpholino-4-yl-ethyl)-1H-indole-6-carboxylic acid (238)

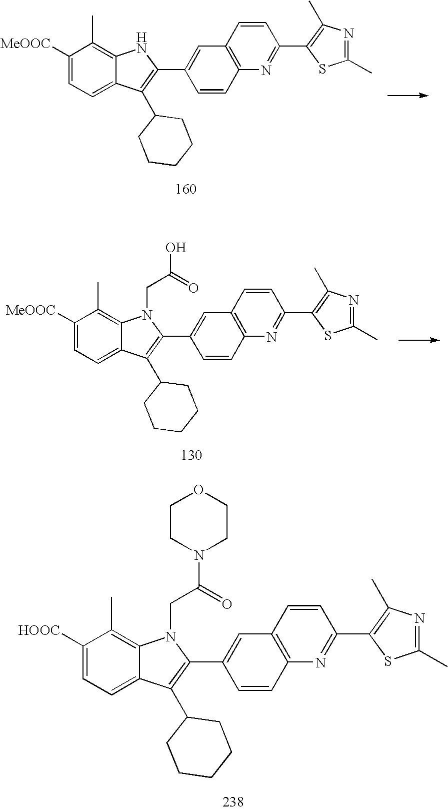

### Step 1. 1-Carboxymethyl-3-cyclohexyl-2-[2-(2,4-dimethyl-thiazol-5-yl)-quinolin-6-yl]-7-methyl-1H-indole-6-carboxylic acid methyl ester (130)

To a suspension of compound 160 (see Example 134 for synthesis, 0.465 g, 0.912 mmol) in anhydrous DMF (9 mL) was added NaH (44 mg, 1.824 mmol) under Ar at 0° C. The reaction mixture was stirred at room temperature with a vacuum for 30 min and cooled in an ice-bath. tert-Butyl bromoacetate (0.34 mL, 2.28 mmol) was added in one portion. The reaction mixture was then stirred at room temperature under Ar for 2.5 h. After evaparation of solvent, the residue was dissolved in CH₂Cl₂ (100 mL), washed with brine (30 mL), and dried over Na₂SO₄. Solvent was evaporated. To the residue was added a mixture of TFA (5 mL) and anisole (0.5 mL). The mixture was stirred at room temperature for 1 h. After evaporation of solvent, compound 130 was obtained (0.50 g, 97%). MS: 568.41 (M+H⁺).

### Step 2. 3-Cyclohexyl-2-[2-(2,4-dimethyl-thiazol-5-yl)-quinolin-6-yl-7-methyl-1-(2-oxo-2-morpholino-4-yl-ethyl)-1]-indole-6-carboxylic acid (238)

Compound 130 (0.3 g, 0.528 mmol) was dissolved in anhydrous DMF (3 mL). HATU (0.26 g, 0.686 mmol) and DIEA (0.23 mL, 1.32 mmol) were added. The reaction mixture was stirred at room temperature for 1 h. Morpholine (0.092 mL, 1.056 mmol) was added. The mixture was stirred at room temperature for 1 h. The solvent was evaporated to dryness. The residue was dissolved in THF (8 mL) and MeOH (4 mL), and 4 N NaOH (2.5 mL) was added. The mixture was stirred at 55° C. for 16 h and cooled down to room temperature. The mixture was neutralized to pH 7 with 5 N HCl. After evaporation of solvent, the residue was purified by reverse phase HPLC to give compound 238 (0.232 g, 71%). MS: 623.29 (M+H⁺). ¹H-NMR (DMSO-d₆): δ (ppm) 8.53 (d, 1H, J=8.7 Hz), 8.06 (d, 1H, J=9.0 Hz), 7.93-7.90 (m, 2H), 7.66-7.61 (m, 2H), 7.43 (d, 1H, J=8.7 Hz), 5.01 (s, 2H), 3.48-3.43 (m, 4H), 3.25 (br s, 4H), 2.73 (br s, 6H), 2.68 (s, 3H), 1.77-1.59 (m, 7H), 1.31-1.28 (m, 3H).

### Example 39

### 3-Cyclohexyl-2-[2-(2,4-dimethyl-thiazol-5-yl)-quinolin-6-yl]-1-[(2H-tetrazol-5-ylcarbamoyl)-methyl]-1H-indole-6-carboxylic acid (239)

Compound 239 was synthesized from compound 128 as described for compound 121 replacing morpholine with 2H-tetrazol-5-ylamine, followed by saponification as in compound 203. Yield 12.3 mg, 16%. MS: 607.2 (M+H⁺); H¹-NMR (DMSO-d₆): δ (ppm) 12.19 (s, 1H), 8.47 (d, 1H, J=8.7), 8.13-7.85 (m, 5H), 7.5 (m, 3H), 5.05 (s, 2H), 2.71 (s, 3H), 2.66 (s, 3H), 2.57 (m, 1H) 1.84-1.07 (m, 10H).

### Example 40

### N-[3-Cyclohexyl-2-[2-(2,4-dimethyl-thiazol-5-yl)-quinolin-6-yl]-1-(2-morpholin-4-yl-2-oxo-ethyl)-1H-indole-6-carbonyl]-methanesulfonamide (240)

To a solution of 126 mg (0.21 mmole) compound 210 in 2 mL of DMF 51 mg (0.32 mmole) CDI was added. The solution was kept at 55 C.° for 1 h when 39.1 mg (0.42 mmole) methanesulfonamide and 48 μL (0.32 mmole) DBU were introduced. The mixture was agitated for 1 h at the same temperature when it was evaporated to dryness. The residue was purified be RP-HPLC to yield 61 mg compound 240. MS: 686.24 (M+H⁺); H¹-NMR (DMSO-d₆): δ (ppm) 11.88 (s, 1H), 8.10 (m, 2H0, 7.96-7.87 (m, 3H), 7.68-7.65 (m, 2H), 4.98 (s, 2H), 3.45-3.32 (m, 11H), 2.74 (s, 3H), 2.69 (s, 3H), 2.65 (m, 1H), 2.0-1.1 (m, 10H).

### Example 41

### 1-[2-(4-Carboxy-piperidin-1-yl)-2-oxo-ethyl]-3-cyclohexyl-2-[2-(2,4-dimethyl-thiazol-5-yl)-quinolin-6-yl]-1H-indole-6-carboxylic acid (241)

Compound 241 was synthesized from compound 128 as described for compound 121 replacing morpholine with piperidine-4-carboxylic acid, followed by saponification as in compound 203. Yield 18.2 mg, 15%. MS: 651.2 (M+H⁺); H¹-NMR (DMSO-d₆): δ (ppm) 8.51 (d, 1H, J=8.7), 8.06-7.83 (m, 5H), 7.65 (d, 2H, J=8.4), 4.98 (m, 2H), 4.08 (d, 1H, J=12.0), 3.71 (d, 1H, J=13.8), 2.95 (t, 1H, J=13.8), 2.72 (s, 3H), 2.71 (s, 3H), 2.63 (m, 1H), 2.40 (m, 1H), 1.84-1.07 (m, 10H).

### Example 42

### 3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid (2-cyano-ethyl)-amide (242)

Compound 242 was synthesized from compound 210 as described for compound 121 replacing morpholine with 3-amino-.propionitrile. MS: 661.2 (M+H⁺); H¹-NMR (DMSO-d₆): δ (ppm) 8.76 (t, 1H), 8.52 (d, 1H, J=8.7 Hz), 8.06 (d, 1H, J=9.0 Hz), 7.93-7.90 (m, 3H), 7.83 (d, 1H, J=8.4 Hz), 7.6 (dd, 1H, J=8.4 and 1.5 Hz), 7.60 (d, 1H, J=8.7 Hz), 4.93 (s, 2H), 3.53 (m, 2H), 3.44-3.34 (m, 8H), 2.81 (t, 2H, J=6.3 Hz), 2.73 (s, 3H), 2.68 (s, 3H), 2.65 (m, 1H), 1.91-1.20 (m, 10H).

### Example 43

### Step 1. 3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid amide (131)

Compound 131 was synthesized from compound 210 as described for compound 121 replacing morpholine with ammonia/methanol solution. MS: 608.2 (M+H⁺);

### Step 2. 3-Cyclohexyl-2-[2-(2,4-dimethyl-thiazol-5-yl)-quinolin-6-yl]-1-(2-morpholin-4-yl-2-oxo-ethyl)-1H-indole-6-carbonitrile (243)

To a cooled solution (0 C.°) of 125 mg (0.2 mmole) 131 in 1.5 mL DMF 129 μL (0.76 mmole) Tf₂O was added. The mixture was stirred for 30 minutes then excess water was added. The precipitate was spun down, washed with sat. NaHCO₃, water then was purified on RP-HPLC to yield 10.5 mg compound 243. MS: 590.2 (M+H⁺); H¹-NMR (DMSO-d₆): δ (ppm) 8.53 (d, 1H, J=8.7 Hz), 8.08-8.03 (m, 2H), 7.96-7.90 (m, 3H), 7.62 (dd, 1H, J=8.4 Hz), 7.37 (dd, 1H, J=8.4 Hz), 4.98 (s, 2H), 3.45-3.32 (m, 8H), 2.72 (s, 3H), 2.66 (s, 3H), 2.65 (m, 1H), 1.95-1.10 (m, 10H).

### Example 44

### 2-[3-Cyclohexyl-2-[2-(2,4-dimethyl-thiazol-5-yl)-quinolin-6-yl]-6-(1H-tetrazol-5-yl)-indol-1-yl]-1-morpholin-4-yl-ethanone (244)

A solution of 75 mg (0.128 mmole) compound 243 and 78 mg (0.383 mmole) trimethyltin azide in 1.5 mL NMP was heated under argon at 120 C.° for 2 days. The NMP was evaporated and the residue purified with RP-HPLC to give 40.2 mg compound 244. MS: 633.2 (M+H⁺); H¹-NMR (DMSO-d₆): δ (ppm) 8.52 (d, 1H, J=8.7 Hz), 8.12 (d, 1H, J=1.2 Hz), 8.08 (d, 1H, J=8.7 Hz), 7.99 (d, 1H, J=8.4 Hz), 7.95 (D, 1 h, J=1.8 Hz), 7.92 (d, 1H, J=8.4 Hz), 7.72 (dd, 1H, J=8.7 and 1.5 Hz), 7.66 (dd, 1H, J=8.7 and 2.1 Hz), 4.98 (s,2H), 3.47-3.36 (m, 8H), 2.73 (s, 3H), 2.68 (s, 3H), 2.65 (m, 1H), 1.95-1.10 (m, 10H).

### Example 45

### 3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid [2-(1H-tetrazol-5-yl)-ethyl]-amide (245)

Compound 245 was synthesized from 242 as described for compound 244. MS: 704.3 (M+H⁺); H¹-NMR (DMSO-d₆): δ (ppm) 8.56 (m, 1H), 8.51 (d, 1H, J=8.4 Hz), 8.06 (d, 1H, J=9.0 Hz), 7.92-7.88 (m, 3H), 7.80 (d, 1H, J=8.7 Hz), 7.64 (dd, 1H, J=8.7 and 1.5 Hz), 7.53 (dd, 1H, J=8.4 and 1.2 Hz), 4.92 (s, 2H), 3.65 (m, 2H), 3.44-3.34 (m, 8H), 3.18 (t, 2H, J=7.2 Hz, 2.73 (s, 3H), 2.68 (s, 3H), 2.65 (m, 1H), 1.95-1.10 (m, 10H).

### Example 46

### 3-Cyclohexyl-1-(2-morpholin-4-yl-2-oxo-ethyl)-2-[2-(3-trifluoromethoxy-phenyl)-quinolin-6-yl]-1H-indole-6-carboxylic acid (246)

Following the full procedure and workup for compound 207, 123 (100 mg, 0.2 mmol) was reacted with 1-(5-Methyl-thiophen-2-yl)-ethanone (41 mg, 0.2 mmol) to produce compound 246 (7.3 mg, 5% yield). MS: 658.23 (M+H⁺); H¹-NMR (DMSO d₆): 8.59 (d, 1H, J=9), 8.32 (m, 3H), 8.20 (d, 1H, J=8.7), 8.00 (m, 2H), 7.85 (d, 1H, J=8.4), 7.67 (m, 3H), 7.52 (m, 1H), 5.00 (s, 2H), 3.42 (m, 8H), 2.64 (m, 1H), 1.77 (m, 7H), 1.22 (m, 3H).

### Example 47

### 3-Cyclohexyl-1-(2-morpholin-4-yl-2-oxo-ethyl)-2-[2-(3-trifluoromethyl-phenyl)-quinolin-6-yl]-1H-indole-6-carboxylic acid (247)

Following the full procedure and workup for compound 207, 123 (100 mg, 0.2 mmol) was reacted with 1-(3-Trifluoromethyl-phenyl)-ethanone (37 mg, 0.2 mmol) to produce compound 247 (7.2 mg, 6% yield). MS: 642.22 (M+H⁺); H¹-NMR (DMSO d₆): 8.60 (m, 3H), 8.35 (d, 1H, J=8.7), 8.24 (d, 1H, J=8.7), 8.00 (m, 2H), 7.84 (m, 3H), 7.67 (m, 2H), 5.01 (s, 2H), 2.63 (m, 1H), 1.76 (m, 7H), 1.23 (m, 3H).

### Example 48

### 3-Cyclohexyl-2-[2-(4-methyl-2-trifluoromethyl-thiazol-5-yl)-quinolin-6-yl]-1-(2-morpholin-4-yl-2-oxo-ethyl)-1H-indole-6-carboxylic acid (248)

Following the full procedure and workup for compound 207, 123 (100 mg, 0.2 mmol) was reacted with 1-(4-Methyl-2-trifluoromethyl-thiazol-5-yl)-ethanone (41.8 mg, 0.2 mmol) to produce compound 248 (34 mg, 25% yield). MS: 661.1 (M−H⁺); H¹-NMR (DMSO d₆): 8.66 (d,1H, J=9.0), 8.12 (t, 1H, J=8.1), 8.80 (s, 1H), 7.86 (d, 1H, J=7.8), 7.62 (m, 2H), 5.01 (s, 2H), 3.71 (m, 8H), 2.55 (s, 3H), 2.65 (m, 1H), 1.80 (m, 7H), 1.23 (m, 3H).

### Example 49

### 3-Cyclohexyl-2-[2-(4-methyl-pyridin-2-yl)-quinolin-6-yl]-1-(2-morpholin-4-yl-2-oxo-ethyl)-1H-indole-6-carboxylic acid (249)

Compound 249 was synthesized from compound 123 as described for compound 207 replacing 5-acetyl salicylaldehyde with 2-acetyl-4-methylpyridine. MS: 589.2 (M+H⁺); H¹-NMR (DMSO-d₆): δ (ppm) 8.67 (d, 1H, J=5.1 Hz), 8.63 (s, 2H), 8.57 (s, 1H), 8.26 (d, 1H, J=8.4 Hz), 8.00 (br s, 2H), 7.86 (d, 1H, J=8.4 Hz), 7.71 (dd, 1H, J=1.8,8.7), 7.66 (dd, 1H, J=1.2,6.0), 7.50 (d, 1H, J=5.1 Hz), 5.01 (s, 2H), 3.43-3.33 (m, 8H), 2.64 (m, 1H), 2.54 (s, 3H), 1.91-1.62 (m, 7H), 1.30-1.16 (m, 3H).

### Example 50

### 3-Cyclohexyl-2-[2-(3,4-dimethyl-phenyl)-quinolin-6-yl]-1-(2-morpholin-4-yl-2-oxo-ethyl)-1H-indole-6-carboxylic acid (250)

Following the full procedure and workup for compound 207, 123 (100 mg, 0.2 mmol) was reacted with 1-(3,4-Dimethyl-phenyl)-ethanone (30 mg, 0.2 mmol) to produce compound 250 (35 mg, 30% yield). MS: 602.30 (M+H⁺); H¹-NMR (DMSO d₆): 8.50 (d, 1H, J=8.7), 8.15 (m, 2H), 8.05 (s, 1H), 7.90 (m, 3H), 7.80 (d, 1H, J=8.7), 7.61 (d, 2H, J=8.1), 7.29 (d, 1H, J=7.8), 4.94 (s, 2H), 3.37 (m, 8H), 2.31 (s, 3H), 2.26 (s, 3H), 1.71 (m, 7H), 1.17 (m, 3H).

### Example 51

### 3-Cyclohexyl-2-[2-(3,5-dimethoxy-phenyl)-quinolin-6-yl]-1-(2-morpholin-4-yl-2-oxo-ethyl)-1H-indole-6-carboxylic acid (251)

Following the full procedure and workup for compound 207, 123 (100 mg, 0.2 mmol) was reacted with 1-(3,5-Dimethoxy-phenyl)-ethanone (30 mg, 0.2 mmol) to produce compound 251 (15 mg, 12% yield). MS: 634.30 (M+H⁺); H¹-NMR (DMSO d₆): 8.49 (d, 1H, J=9), 8.17 (m, 2H), 7.96 (s, 1H), 7.90 (s, 1H), 7.81 (d, 1H, J=8.4), 7.61 (d, 2H, J=9), 7.40 (d, 2H, J=2.1), 6.62 (m, 1H), 4.96 (s, 2H), 3.82 (s, 6H), 3.37 (m, 8H), 2.51 (m, 1H) 1.70 (m, 7H), 1.18 (m, 3H).

### Example 52

### 3-Cyclohexyl-1-(2-morpholin-4-yl-2-oxo-ethyl)-2-(2-p-tolyl-quinolin-6-yl)-1H-indole-6-carboxylic acid (252)

Compound 252 was synthesized from compound 123 as described for compound 207 replacing 5-acetyl salicylaldehyde with 4′-methylacetophenone. MS: 588.2 (M+H⁺); H¹-NMR (DMSO-d₆): δ (ppm) 8.57 (d, 1H, J=9.0 Hz), 8.24-8.18 (m, 4H), 8.00 (s, 1H), 7.95 (s, 1H), 7.85 (d, 1H, J=8.4), 7.69-7.64 (m, 2H), 7.39 (m, 2H), 5.00 (s, 2H), 3.43-3.33 (m, 8H), 2.64 (m, 1H), 2.41 (s, 3H), 1.91-1.62 (m, 7H), 1.30-1.16 (m, 3H).

### Example 53

### 3-Cyclohexyl-2-[2-(3,4-dimethoxy-phenyl)-quinolin-6-yl]-1-(2-morpholin-4-yl-2-oxo-ethyl)-1H-indole-6-carboxylic acid (253)

Compound 253 was synthesized from compound 123 as described for compound 207 replacing 5-acetyl salicylaldehyde with 3′,4′-dimethoxyacetophenone. MS: 634.2 (M+H⁺); H¹-NMR (DMSO-d₆): δ (ppm) 8.51 (d, 1H, J=9.0 Hz), 8.23 (d, 1H, J=8.7 Hz), 8.16 (d, 1H, J=8.7 Hz), 8.00 (s, 1H), 7.92-7.84 (m, 4H), 7.66 (m, 2H), 7.14 (d, 1H, J=8.1 Hz), 5.00 (s, 2H), 3.91 (s, 3H), 3.85 (s, 3H), 3.43-3.33 (m, 8H), 2.64 (m, 1H), 1.91-1.62 (m, 7H), 1.30-1.16 (m, 3H).

### Example 54

### 3-Cyclohexyl-2-[2-(4-methoxy-phenyl)-quinolin-6-yl]-1-(2-morpholin-4-yl-2-oxo-ethyl)-1H-indole-6-carboxylic acid (254)

Following the full procedure and workup for compound 207, 123 (100 mg, 0.2 mmol) was reacted with 1-(4-Methoxy-phenyl)-ethanone (32 mg, 0.2 mmol) to produce compound 254 (45 mg, 36% yield). MS: 604.2 (M+H⁺); H¹-NMR (DMSO d₆): 8.52 (d, 1H, J=8.7), 8.27 (m, 2H), 8.17 (m, 2H), 8.01 (s, 1H), 7.93 (s, 1H), 7.85 (d, 1H, J=9), 7.65 (m, 2H), 7.12 (m, 2H), 5.00 (s, 2H), 3.86 (s, 3H), 3.42 (m, 8H), 2.64 (m, 1H), 1.76 (m, 7H), 1.24 (m, 3H).

### Example 55

### 3-Cyclohexyl-2-[2-(2-fluoro-phenyl)-quinolin-6-yl]-1-(2-morpholin-4-yl-2-oxo-ethyl)-1H-indole-6-carboxylic acid (255)

Following the full procedure and workup for compound 207, 123 (100 mg, 0.2 mmol) was reacted with 1-(2-Fluoro-phenyl)-ethanone (27.8 mg, 0.2 mmol) to produce compound 255 (22.4 mg, 19% yield). MS: 592.7 (M+H⁺); H¹-NMR (DMSO d₆): 8.57 (d,1H, J=8.7), 8.19 (d,1H, J=8.4), 8.00 (m, 3H), 7.80 (d, 1H, 8.1), 7.69-7.51 (m, 3H), 7.41 (m, 2H), 5.01 (s, 2H), 3.71 (m, 8H), 2.65 (m, 1H), 1.80 (m, 7H), 1.23 (m, 3H). F¹⁹-NMR (DMSO d₆): -117.4.

### Example 56

### 3-Cyclohexyl-1-(2-morpholin-4-yl-2-oxo-ethyl)-2-[2-(3-nitro-phenyl)-quinolin-6-yl]-1H-indole-6-carboxylic acid methyl ester (256)

Compound 256 was synthesized from compound 123 as described for compound 207 replacing 5-acetyl salicylaldehyde with 3′-nitroacetophenone. MS: 619.2 (M+H⁺); H¹-NMR (DMSO-d₆): δ (ppm) 9.11 (s, 1H), 8.75 (d, 1H, J=7.8 Hz), 8.64 (d, 1H, J=8.4 Hz), 8.41-8.35 (m, 2H), 8.26 (d, 1H, J=8.4 Hz), 8.01 (m, 2H), 7.90-7.85 (m, 2H), 7.72-7.65 (m, 2H), 5.01 (s, 2H), 3.43-3.33 (m, 8H), 2.65 (m, 1H), 1.91-1.62 (m, 7H), 1.30-1.16 (m, 3H).

### Example 57

### 3-Cyclohexyl-2-[2-(2-fluoro-4-methoxy-phenyl)-quinolin-6-yl]-1-(2-morpholin-4-yl-2-oxo-ethyl)-1H-indole-6-carboxylic acid (257)

Compound 257 was synthesized from compound 123 as described for compound 207 replacing 5-acetyl salicilaldehyde with 1-(2-Fluoro-4-methoxy-phenyl)-ethanone. MS: 622.2 (M+H⁺); H¹-NMR (DMSO-d₆): δ (ppm) 8.54 (d, 1H), 8.18 (d, 1H), 8.09-7.95 (m, 4H), 7.86 (d, 1H), 7.66 (m, 2H), 7.06-6.99 (m, 2H), 4.99 (s, 2H), 3.87 (s, 3H), 3.87-3.50 (m, 8H), 2.63 (m, 1H), 1.92-1.20 (m, 10H); F¹⁹-NMR (DMSO-d₆): δ (ppm) −75.36.

### Example 58

### 3-Cyclohexyl-2-[2-(2,5-dimethyl-thiophen-3-yl)-quinolin-6-yl]-1-(2-morpholin-4-yl-2-oxo-ethyl)-1H-indole-6-carboxylic acid (258)

Following the full procedure and workup for compound 207, 123 (100 mg, 0.2 mmol) was reacted with 1-(2,5-Dimethyl-thiophen-3-yl)-ethanone (30 mg, 0.2 mmol) to produce compound 258 (34 mg, 29% yield). MS: 608.2 (M+H⁺); H¹-NMR (DMSO d₆): 8.50 (d, 1H, J=8.7), 8.11 (d, 1H, J=9), 8.00 (s, 1H), 7.87 (m, 3H), 7.65 (m, 2H), 7.28 (s, 1H), 5.00 (s, 2H), 2.74 (s, 3H), 2.63 (m, 1H), 2.46 (s, 3H), 1.75 (m, 7H), 1.23 (m, 3H).

### Example 59

### 3-Cyclohexyl-2-[2-(2,6-difluoro-phenyl)-quinolin-6-yl]-1-(2-morpholin-4-yl-2-oxo-ethyl)-1H-indole-6-carboxylic acid (259)

Following the full procedure and workup for compound 207, 123 (100 mg, 0.2 mmol) was reacted with 1-(2,6-Difluoro-phenyl)-ethanone (31.2 mg, 0.2 mmol) to produce compound 259 (16.6 mg, 12% yield). MS: 610.7 (M+H⁺); H¹-NMR (DMSO d₆): 8.59 (d,1H, J=8.7), 8.17 (d,1H, J=8.4), 8.00 (s, 2H), 7.87-7.58 (m, 5H), 7.30 (m, 2H), 5.01 (s, 2H), 3.71 (m, 8H), 2.65 (m, 1H), 1.80 (m, 7H), 1.23 (m, 3H). F¹⁹-NMR (DMSO d₆): −115.0

### Example 60

### 3-Cyclohexyl-2-[2-(2,4-dimethyl-oxazol-5-yl)-quinolin-6-yl]-1-(2-morpholin-4-yl-2-oxo-ethyl)-1H-indole-6-carboxylic acid (260)

Following the full procedure and workup for compound 207, 123 (100 mg, 0.2 mmol) was reacted with 1-(2,4-Dimethyl-oxazol-5-yl)-ethanone (28.0 mg, 0.2 mmol) to produce compound 260 (31.3 mg, 22% yield). MS: 593 (M+H⁺); H¹-NMR (DMSO d₆): 8.52 (d,1H, J=8.7), 8.08 (d, 1H, J=8.7), 8.00 (s, 1H), 7.91-7.84 (m, 3H), 7.67 (m, 2H), 5.00 (s, 2H), 3.71 (m, 8H), 2.65 (s, 4H), 2.52 (s, 3H), 1.80 (m, 7H), 1.23 (m, 3H).

### Example 61

### 3-Cyclohexyl-2-[2-(3-fluoro-phenyl)-quinolin-6-yl]-1-(2-morpholin-4-yl-2-oxo-ethyl)-1H-indole-6-carboxylic acid (261)

Compound 261 was synthesized from compound 123 as described for compound 207 replacing 5-acetyl salicylaldehyde with 3′-fluoroacetophenone. MS: 592.2 (M+H⁺); H¹-NMR (DMSO-d₆): δ (ppm) 8.58 (d, 1H, J=7.8 Hz), 8.28 (d, 1H, J=8.1 Hz), 8.22-8.10 (m, 3H), 8.01-7.96 (m, 2H), 7.86 (d, 1H, J=9.0 Hz), 7.70-7.60 (m, 3H), 7.40-7.30 (m, 1H), 5.01 (s, 2H), 3.43-3.33 (m, 8H), 2.65 (m, 1H), 1.91-1.62 (m, 7H), 1.30-1.16 (m, 3H).

### Example 62

### 2-[2-(3-Bromo-phenyl)-quinolin-6-yl]-3-cyclohexyl-1-(2-morpholin-4-yl-2-oxo-ethyl)-1H-indole-6-carboxylic acid (262)

Compound 262 was synthesized from compound 123 as described for compound 207 replacing 5-acetyl salicilaldehyde with 1-(3-Bromo-phenyl)-ethanone. MS: 652.17 (M+H⁺); H¹-NMR (DMSO-d₆): δ (ppm) 8.58 (d, 1H), 8.50 (m, 1H), 8.32-8.16 (m, 2H), 8.21 (d, 1H), 8.01 (d, 1H), 7.97 (d, 1H), 7.86 (d, 1H), 7.73-7.64 (m, 3H), 7.56-7.51 (m, 1H), 5.00 (s, 2H), 3.43 (m, 8H), 2.64 (m, 1H), 2.01-1.20 (m, 10H).

### Example 63

### 3-Cyclohexyl-1-(2-morpholin-4-yl-2-oxo-ethyl)-2-[2-(4-trifluoromethyl-phenyl)-quinolin-6-yl]-1H-indole-6-carboxylic acid (263)

Compound 263 was synthesized from compound 123 as described for compound 207 replacing 5-acetyl salicilaldehyde with 1-(4-trifluoromethyl-phenyl)-ethanone. MS: 542.2 (M+H⁺); H¹-NMR (DMSO-d₆): δ (ppm) 8.62 (d, 1H), 8.52 (d, 1H), 8.31 (d, 1H), 8.22 (d, 1H), 7.99 (m, 2H), 7.93 (m, 2H), 7.86 (d, 1H), 7.71-7.67 (m, 2H), 5.01 (s, 2H), 3.44-3.33 (m, 8H), 2.64 (m, 1H), 1.92-1.16 (m, 10H); F¹⁹-NMR (DMSO-d₆): δ (ppm) −61.61.

### Example 64

### 2-[2-(3-Amino-phenyl)-quinolin-6-yl]-3-cyclohexyl-1-(2-morpholin-4-yl-2-oxo-ethyl)-1H-indole-6-carboxylic acid (264)

Following the full procedure and workup for compound 207, 123 (100 mg, 0.2 mmol) was reacted with 1-(2,5-Dimethyl-thiophen-3-yl)-ethanone (28 mg, 0.2 mmol) to produce compound 264 (46 mg, 36% yield). MS: 589.2 (M+H⁺); H¹-NMR (DMSO d₆): 8.55 (m, 2H), 8.16 (d, 2H, J=8.1), 7.99 (m, 3H), 7.86 (d, 2H, J=8.4), 7.66 (m, 2H), 5.00 (s, 2H), 2.64 (m, 1H), 1.77 (m, 7H), 1.20 (m, 3H).

### Example 65

### 3-Cyclohexyl-2-[2-(4-fluoro-phenyl)-quinolin-6-yl]-1-(2-morpholin-4-yl-2-oxo-ethyl)-1H-indole-6-carboxylic acid (265)

Following the full procedure and workup for compound 207, 123 (100 mg, 0.2 mmol) was reacted with 1-(4-Fluoro-phenyl)-ethanone (27.8 mg, 0.2 mmol) to produce compound 265 (17.5 mg, 12% yield). MS: 592.7 (M+H⁺); H¹-NMR (DMSO d₆): 8.55 (d,1H, J=8.4), 8.35 (m,2H), 8.24 (d,1H, J=8.4), 8.15 (d,1H, J=8.4), 8.00 (s, 1H), 7.92 (s, 1H), 7.85 (d, 1H, 8.4), 7.63 (m, 2H), 7.40 (m, 2H), 5.01 (s, 2H), 3.77 (m, 8H), 2.65 (m, 1H), 1.80 (m, 7H), 1.23 (m, 3H). F¹⁹-NMR (DMSO d₆): -112.1.

### Example 66

### 3-Cyclohexyl-2-[2-(3,4-difluoro-phenyl)-quinolin-6-yl]-1-(2-morpholin-4-yl-2-oxo-ethyl)-1H-indole-6-carboxylic acid (266)

Compound 266 was synthesized from compound 123 as described for compound 207 replacing 5-acetyl salicylaldehyde with 3′,4′-difluoroacetophenone. MS: 610.2 (M+H⁺); H¹-NMR (DMSO-d₆): δ (ppm) 8.58 (d, 1H, J=9.0), 8.41-8.34 (m, 1H), 8.28 (d, 1H, J=9.0 Hz), 8.22-8.18 (m, 2H), 8.01 (s, 1H), 7.96 (s, 1H), 7.86 (d, 1H, J=8.4 Hz), 7.69-7.62 (m, 3H), 5.00 (s, 2H), 3.43-3.33 (m, 8H), 2.65 (m, 1H), 1.91-1.62 (m, 7H), 1.30-1.16 (m, 3H).

### Example 67

### 3-Cyclohexyl-1-(2-morpholin-4-yl-2-oxo-ethyl)-2-[2-(2-trifluoromethyl-phenyl)-quinolin-6-yl]-1H-indole-6-carboxylic acid (267)

Following the full procedure and workup for compound 207, 123 (100 mg, 0.2 mmol) was reacted with 1-(2-Trifluoromethyl-phenyl)-ethanone (33 mg, 0.2 mmol) to produce compound 267 (37 mg, 29% yield). MS: 642.27 (M+H⁺); H¹-NMR (DMSO d₆): 8.56 (d, 1H, J=8.1), 8.13 (m, 1H), 8.01 (s, 2H), 7.86 (m, 4H), 7.68 (m, 5H), 5.01 (s, 2H), 2.66 (m, 1H), 1.81 (m, 7H), 1.24 (m, 3H).

### Example 68

### 3-Cyclohexyl-2-[2-(3-methyl-pyrazin-2-yl)-quinolin-6-yl]-1-(2-morpholin-4-yl-2-oxo-ethyl)-1H-indole-6-carboxylic acid (268)

Following the full procedure and workup for compound 207, 123 (100 mg, 0.2 mmol) was reacted with 1-(3-Methyl-pyrazin-2-yl)-ethanone (27.2 mg, 0.2 mmol) to produce compound 268 (42.8 mg, 35% yield). MS: 590.27 (M+H⁺); H¹-NMR (DMSO d₆): 8.67 (m, 3H), 8.20 (m, 2H), 8.04 (d,1H, J=1.2), 7.88 (d,1H, J=8.7), 7.67 (m, 1H), 5.01 (s, 2H), 3.77 (m, 8H), 2.90 (s, 3H), 2.65 (m, 1H) 1.80 (m, 7H), 1.23 (m, 3H).

### Example 69

### 3-Cyclohexyl-2-[2-(2-ethoxy-4-methyl-pyrimidin-5-yl)-quinolin-6-yl]-1-(2-morpholin-4-yl-2-oxo-ethyl)-1H-indole-6-carboxylic acid (269)

Compound 269 was synthesized from compound 123 as described for compound 207 replacing 5-acetyl salicylaldehyde with 1-(4-methyl-2-methylsulfanyl-pyrimidin-5-yl)-ethanone. MS: 634.3 (M+H⁺); H¹-NMR (DMSO-d₆): δ (ppm) 8.77 (s, 1H), 8.58 (d, 1H, J=9.3 Hz), 8.17 (d, 1H, J=8.1 Hz), 8.00-7.98 (m, 2H), 7.92-7.85 (m, 2H), 7.70-7.65 (m, 2H), 5.00 (s, 2H), 4.43 (q, 2H, J=8.1 Hz), 3.43-3.33 (m, 8H), 2.63 (m, 1H), 1.91-1.62 (m, 7H), 1.38 (t, 3H, J=6.5 Hz), 1.30-1.16 (m, 3H).

### Example 70

### 3-Cyclohexyl-2-[2-(2-fluoro-5-methoxy-phenyl)-quinolin-6-yl]-1-(2-morpholin-4-yl-2-oxo-ethyl)-1H-indole-6-carboxylic acid (270)

Following the full procedure and workup for compound 207, 123 (100 mg, 0.2 mmol) was reacted with 1-(2-Fluoro-5-methoxy-phenyl)-ethanone (33.6 mg, 0.2 mmol) to produce compound 270 (21 mg, 16% yield). MS: 622 (M−H⁺); H¹-NMR (DMSO d₆): 8.66 (d,1H, J=9.0), 8.29 (d, 1H, J=8.7), 8.02 (m, 3H), 7.94 (d, 1H, J=8.7), 7.74 (m, 2H), 7.61 (m, 1H), 7.41 (m, 1H), 7.14 (m, 1H), 5.08 (s, 2H), 3.71 (m, 8H), 3.62, (s, 3H), 2.65 (m, 1H), 1.80 (m, 6H), 1.23 (m, 3H).

### Example 71

### 3-Cyclohexyl-2-[2-(1-methyl-1H-pyrrol-2-yl)-quinolin-6-yl]-1-(2-morpholin-4-yl-2-oxo-ethyl)-1H-indole-6-carboxylic acid (271)

Following the full procedure and workup for compound 207, 123 (100 mg, 0.2 mmol) was reacted with 1-(1-Methyl-1H-pyrrol-2-yl)-ethanone (25 mg, 0.2 mmol) to produce compound 271 (6 mg, 4% yield). MS: 577.2 (M+H⁺); H¹-NMR (DMSO d₆): 8.35 (d, 1H, J=8.4), 7.99 (m, 3H), 7.84 (m, 2H), 7.62 (m, 2H), 7.05 (m, 1H), 6.98 (m, 1H), 6.17 (m, 1H), 4.98 (s, 2H), 4.17 (s, 3H), 3.40 (m, 8H), 2.65 (m, 1H), 1.74 (m, 7H), 1.23 (m, 3H).

### Example 72

### 3-Cyclohexyl-1-(2-morpholin-4-yl-2-oxo-ethyl)-2-[2-(2,3,4-trimethoxy-phenyl)-quinolin-6-yl]-1H-indole-6-carboxylic acid (272)

Following the full procedure and workup for compound 207, 123 (100 mg, 0.2 mmol) was reacted with 1-(2,3,4-Trimethoxy-phenyl)-ethanone (42 mg, 0.2 mmol) to produce compound 272 (39 mg, 30% yield). MS: 664.3 (M+H⁺); H¹-NMR (DMSO d₆): 8.56 (d, 1H, J=9), 8.18 (d, 1H, J=8.7), 7.99 (m, 3H), 7.86 (d, 1H, J=8.4), 7.67 (m, 2H), 7.03 (d, 1H, J=9.3), 5.00 (s, 2H), 3.89 (s, 3H), 3.84 (s, 3H), 3.77 (s, 3H), 3.45 (m, 8H), 2.63 (m, 1H), 1.79 (m, 7H), 1.23 (m, 3H).

### Example 73

### 3-Cyclohexyl-2-[2-(3-fluoro-4-methoxy-phenyl)-quinolin-6-yl]-1-(2-morpholin-4-yl-2-oxo-ethyl)-1H-indole-6-carboxylic acid (273)

Following the full procedure and workup for compound 207, 123 (100 mg, 0.2 mmol) was reacted with 1-(3-Fluoro-4-methoxy-phenyl)-ethanone (37 mg, 0.2 mmol) to produce compound 273 (15 mg, 12% yield). MS: 622.2 (M+H⁺); H¹-NMR (DMSO d₆): 8.52 (d, 1H, J=8.7), 8.20 (m, 4H), 8.00 (d, 1H, J=1.2), 7.92 (d, 1H, J=1.5), 7.85 (d, 1H, J=8.4), 7.66 (m, 2H), 7.35 (t, 1H), 4.99 (s, 2H), 3.94 (s, 3H), 3.41 (m, 8H), 2.64 (m, 1H), 1.75 (m, 7H), 1.22 (m, 3H).

### Example 74

### 3-Cyclohexyl-1-[2-(4-dimethylamino-piperidin-1-yl)-2-oxo-ethyl]-2-[2-(2,4-dimethyl-thiazol-5-yl)-quinolin-6-yl]-1H-indole-6-carboxylic acid (274)

Following the full procedure and workup for compound 212, 129 (70 mg, 0.11 mmol) was reacted with Dimethyl-piperidin-4-yl-amine (56 mg, 0.44 mmol). The product was then saponified and purified via HPLC to produce compound 274 (32 mg, 44% yield). MS: 650.2 (M+H⁺); H¹-NMR (DMSO d₆): 8.53 (d, 1H, J=8.7), 8.06 (d, 1H, J=8.7), 7.98 (s, 1H), 7.87 (m, 3H), 7.65 (m, 2H), 5.01 (s, 2H), 4.40 (m, 1H), 3.31 (m, 2H), 2.89 (m, 1H), 2.72 (s, 3H), 2.67 (s, 3H), 2.56 (m, 6H), 1.80 (m, 10H), 1.23 (m, 4H).

### Example 75

### 3-Cyclohexyl-1-[2-(4-diethylamino-piperidin-1-yl)-2-oxo-ethyl]-2-[2-(2,4-dimethyl-thiazol-5-yl)-quinolin-6-yl]-1H-indole-6-carboxylic acid (275)

Following the full procedure and workup for compound 212, 129 (70 mg, 0.11 mmol) was reacted with Dimethyl-piperidin-4-yl-amine (68 mg, 0.44 mmol). The product was then saponified and purified via HPLC to produce compound 275 (26 mg, 35% yield). MS: 678.3 (M+H⁺); H¹-NMR (DMSO d₆): 8.56 (d, 1H, J=9), 8.09 (d, 1H, J=8.7), 7.98 (m, 3H), 7.84 (d, 1H, J=8.4), 7.67 (m, 2H), 5.00 (m, 2H), 4.36 (m, 1H), 3.90 (m, 1H), 3.48 (m, 1H), 2.90 (M, 4H), 2.73 (m, 6H), 2.60 (m, 2H), 1.80 (m, 9H), 1.19 (m, 12H).

### Example 76

### 2-[2-(2-Chloro-phenyl)-quinolin-6-yl]-3-cyclohexyl-1-(2-morpholin-4-yl-2-oxo-ethyl)-1H-indole-6-carboxylic acid (276)

Following the full procedure and workup for compound 207, 123 (100 mg, 0.2 mmol) was reacted with 1-(2-Chloro-phenyl)-ethanone (26.6 mg, 0.2 mmol) to produce compound 276 (5 mg, 4% yield). MS: 641 (M−H⁺); H¹-NMR (DMSO d₆): 8.55 (d,1H, J=8.4), 8.19 (d, 1H, J=9.3), 8.02 (s, 2H), 7.86 (m, 2H), 7.74-7.50 (m, 6H), 5.00 (s, 2H), 3.71 (m, 8H), 2.65 (m, 1H), 1.80 (m, 6H), 1.23 (m, 3H).

### Example 77

### 3-Cyclohexyl-2-[2-(2,4-dimethyl-thiazol-5-yl)-quinolin-6-yl]-1-[2-(2-methyl-pyrrolidin-1-yl)-2-oxo-ethyl]-1H-indole-6-carboxylic acid (277)

Following the full procedure and workup for compound 212, 129 (210 mg, 0.38 mmol) was reacted with 2-Methyl-pyrrolidine (44 mg, 0.52 mmol) to produce compound 277 (19 mg, 9% yield). MS: 607.2 (M+H⁺); H¹-NMR (DMSO d₆): 8.50 (d, 1H, J=9 Hz), 8.03 (m, 2H), 7.88 (m, 3H), 7.64 (m, 2H), 4.84 (m, 4H), 3.8 (s, 1H), 3.24 (m, 2H), 2.72 (s, 3H), 2.66 (s, 3H), 2.61 (m, 1H), 1.86-0.77 (m, 13H).

### Example 78

### 3-Cyclohexyl-2-[2-(2,4-dimethyl-thiazol-5-yl)-quinolin-6-yl]-1-[2-(4-morpholin-4-yl-piperidin-1-yl)-2-oxo-ethyl]-1H-indole-6-carboxylic acid (278)

Following the full procedure and workup for compound 212, 129 (210 mg, 0.38 mmol) was reacted with 4-Piperidin-4-yl-morpholine (89 mg, 0.52 mmol) to produce compound 278 (38 mg, 16% yield). MS: 692.3 (M+H); H¹-NMR (DMSO d₆): 8.52 (d, 1H, J=8.7 Hz), 8.07 (d, 1H, J=8.4 Hz), 7.95 (m, 3H), 7.83 (d, 1H, J=8.1 Hz), 7.64 (d, 2H, J=8.4 Hz), 5.02 (s, 2H), 4.36 (m, 2H), 3.357 (m, 2H), 3.00 (m, 7H), 2.71 (s, 3H), 2.65 (s, 3H), 2.55 (m, 3H), 1.86 (m, 10H), 1.23 (m, 6H).

### Example 79

### 3-Cyclohexyl-1-[2-(3,5-dimethyl-morpholin-4-yl)-2-oxo-ethyl]-2-[2-(2,4-dimethyl-thiazol-5-yl)-quinolin-6-yl]-1H-indole-6-carboxylic acid (279)

Following the full procedure and workup for compound 212, 129 (210 mg, 0.38 mmol) was reacted with 2,6-Dimethyl-piperidine (63 mg, 0.52 mmol) to produce compound 279 (13 mg, 6% yield). MS: 637.2 (M+H⁺); H¹-NMR (DMSO d₆): 8.50 (d, 1H, J=8.4 Hz), 8.45 (d, 1H, J=8.7 Hz), 7.99 (s, 1H), 7.91 (m, 2H), 7.84 (d, 1H, J=8.1 Hz), 7.64 (m, 2H), 4.98 (m, 2H), 3.67 (m, 1H), 2.72 (s, 3H), 2.67 (s, 3H), 2.52 (m, 1H), 2.21 (m, 1H), 1.16 (m, 7H), 1.10 (m, 9H).

### Example 80

### 3-Cyclohexyl-2-[2-(2,4-dimethyl-thiazol-5-yl)-quinolin-6-yl]-1-[2-(4-methyl-piperidin-1-yl)-2-oxo-ethyl]-1H-indole-6-carboxylic acid (280)

Following the full procedure and workup for compound 212, 129 (210 mg, 0.38 mmol) was reacted with 4-Methyl-piperidine (52 mg, 0.52 mmol) to produce compound 280 (16 mg, 7% yield). MS: 621.2 (M+H⁺); H¹-NMR (DMSO d₆): 8.25 (d, 1H, J=8.7 Hz), 7.78 (m, 3H), 7.61 (m, 3H), 7.40 (d, 1H, J=8.4 Hz), 4.60 (m, 2H), 3.91 (m, 1H), 3.40 (m, 1H), 2.45 (s, 3H), 2.41 (s, 3H), 1.53 (m, 7H), 1.08 (m, 7H), 0.32 (s, 3H).

### Example 81

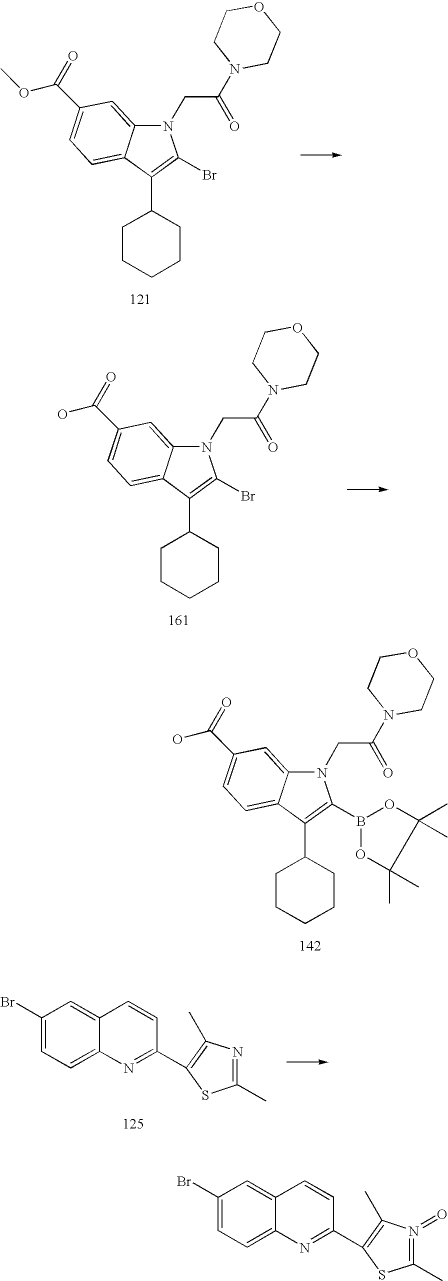

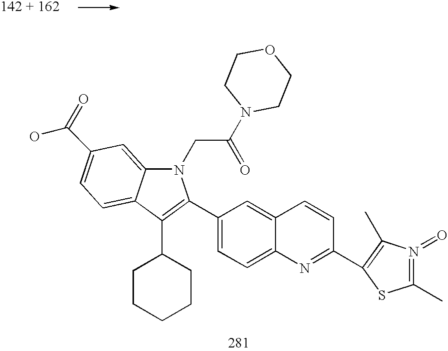

### Step 1. 2-Bromo-3-cyclohexyl-1-(2-morpholin-4-yl-2-oxo-ethyl)-1H-indole-6-carboxylic acid (161)

A solution of 2-Bromo-3-cyclohexyl-1-(2-morpholin-4-yl-2-oxo-ethyl)-1H-indole-6-carboxylic acid methyl ester (121, 1.1 g, 2.2 mmol) in THF (20 mL) was treated with LiOH (4 mL of 2 M solution) and methanol (10 mL). The solution was heated to 60° C. overnight. Once the reaction was complete, the solvents were removed, taken up in DMF (10 mL), acidified and purified by RP-HPLC to give the product 161 760 mg, 72%. MS: 449.2 (M+H⁺).

### Step 2. 3-Cyclohexyl-1-(2-morpholin-4-yl-2-oxo-ethyl)-2-(4,4,5,5-tetramethyl-[1,3,2]dioxaborolan-2-yl)-1H-indole-6-carboxylic acid (142)

A solution of 2-Bromo-3-cyclohexyl-1-(2-morpholin-4-yl-2-oxo-ethyl)-1H-indole-6-carboxylic acid (161, 704 mg, 0.11 mol) dipinacolato diborane (4.5 eq., 1.6 g), potassium acetate (2 eq. 305 mg) and Pd[P(Ph)₃]₂Cl₂ (10 mol %, 110 mg) in DMSO (15 mL) was degassed with Argon and heated to 80° C. overnight. The solution was cooled, precipitated with water (35 mL) and the solids taken up in DMF and purified by silica gel chromatography (EtOAc-Hexanes 50-100%) to yield compound 142 153 mg, 20%. MS: 497.7 (M+H⁺);

### Step 3. 6-Bromo-2-(2,4-dimethyl-thiazol-5-yl n-oxide)-quinoline (162)

To a solution of 6-Bromo-2-(2,4-dimethyl-thiazol-5-yl)-quinoline (125, 1.21 g, 3.8 mmol) in THF (25 mL) was added mCPBA (3 eq. 2.8 g) in MeCN (20 mL). After 5 hrs another 2 g of mCPBA was added and the reaction was monitored by HPLC. After overnight stirring, the mixture was purified by RP-HPLC to give the thiazole n-oxide 162. MS: 335.2 (M+H⁺);

### Step 4. 3-Cyclohexyl-2-[2-(2,4-dimethyl-3-oxy-thiazol-5-yl)-quinolin-6-yl]-(2-morpholin-4-yl-2-oxo-ethyl)-1H-indole-6-carboxylic acid (281)

A mixture of compound 142 (49.6 mg, 0.1 mmol), 6-Bromo-2-(2,4-dimethyl-thiazol-5-yl n-oxide)-quinoline (162, 1 eq., 33.5 mg), Pd[P(Ph)₃]₄ (5 mol %, 6 mg), NaHCO₃ (250 μL of a saturated aqueous solution.) and MeOH (3 mL) was degassed with Argon and heated to 70° C. overnight. The crude mixture was purified by RP-HPLC to give the product (281, 7.2 mg). MS: 625.3 (M+H⁺); H¹-NMR (DMSO-d₆): δ (ppm) 12.0 (s, 1H), 8.63 (d, 1H, J=9.0), 8.13 (d, 1H, 8.4 Hz), 8.02-7.99 (m, 3H), 7.80 (d, 1H, 8.7 Hz), 7.62 (m, 2H), 5.05 (s, 2H), 2.87 (s, 3H), 2.56 (s, 3H), 2.57 (m, 1H) 1.84-1.07 (m, 10H).

### Example 82

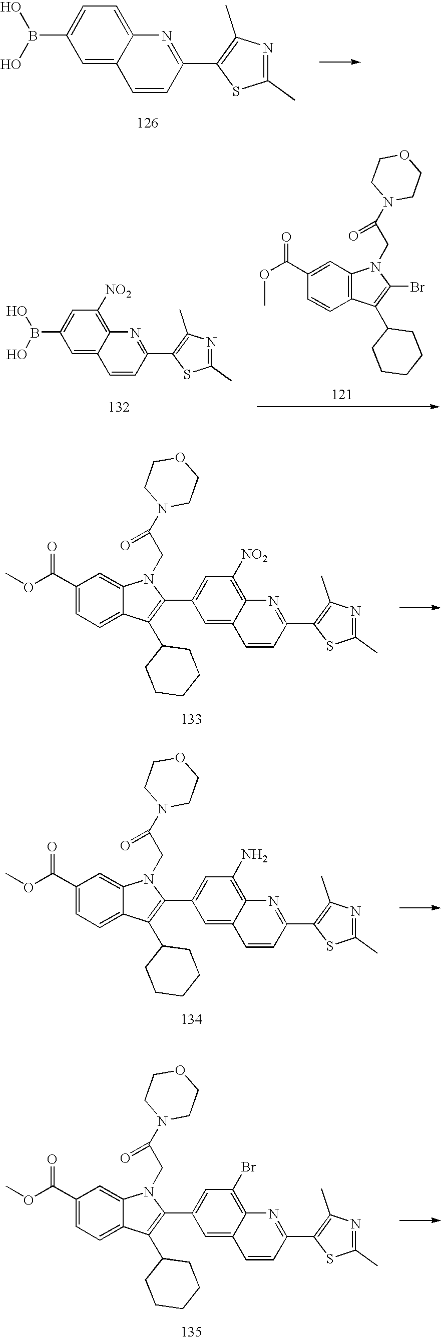

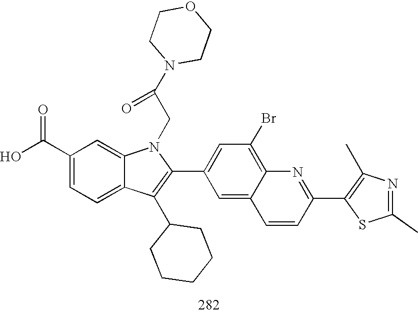

### Step 1. 2-(2,4-Dimethyl-thiazol-5-yl)-8-nitro-quinoline-6-boronic acid (132)

3.0 g (10.56 mmole) compound 126 was dissolved in 30 mL c. sulfuric acid then was cooled to 0 C.°. 1 mL 90% HNO₃ was added dropwise than the cooling bath was removed. The reaction was complete in 15 minutes. The mixture was poured on crushed ice. A gelatinous precipitate formed which was spun down, washed with water by re-suspension until all the acids were removed then it was dried. Yield 1.6 g (46%) compound 132. MS: 330.0 (M+H⁺); H¹-NMR (DMSO-d₆): δ (ppm) 8.65-8.62 (m, 2H), 8.46 (d, 1H, J=1.2 Hz), 7.97 (d, 1H, J=8.4 Hz), 2.75 (s, 3H), 2.72 (s, 3H).

### Step 2. 3-Cyclohexyl-2-[2-(2,4-dimethyl-thiazol-5-yl)-8-nitro-quinolin-6-yl]-1-(2-morpholin-4-yl-2-oxo-ethyl)-1H-indole-6-carboxylic acid methyl ester (133)

Compound 133 was synthesized from compounds 132 and 121 using the conditions described for compound 139. MS: 668.2 (M+H⁺).

### Step 3. 2-[8-Amino-2-(2,4-dimethyl-thiazol-5-yl)-quinolin-6-yl]-3-cyclohexyl-1-(2-morpholin-4-yl-2-oxo-ethyl)-1H-indole-6-carboxylic acid methyl ester (134)

900 mg (1.36 mmole) 133 was dissolved in 50 mL methanol-DMF 1:1 mixture and was hydrogenated in the presence of 100 mg 10% Pd/C catalyst at 30 psi overnight. The catalyst was filtered off, washed with DMF, the solution was evaporated to dryness to give 200 mg compound 134 as semisolid which was used without further purification. MS: 638.2 (M+H⁺).

### Step 4. 2-[8-Bromo-2-(2,4-dimethyl-thiazol-5-yl)-quinolin-6-yl]-3-cyclohexyl-1-(2-morpholin-4-yl-2-oxo-ethyl)-1H-indole-6-carboxylic acid methyl ester (135)

To the solution of 200 mg (0.314 mmole) 134 in 5 mL acetone 150 μL (1.412 mmole) 48% aqueous HBr was added. The solution was cooled to 0 C.° and 22 mg NaNO₂, dissolved in 1 mL water, was added slowly. It was stirred at the same temperature for 10 more minutes when 45 mg CuBr was added as solid and the stirring was continued for 30 min. The solvent was evaporated. The residue was purified with RP-HPLC to give 100 mg (45%) compound 135. MS: 701.1, 703.1 (M+H⁺).

### Step 5. 2-[8-Bromo-2-(2,4-dimethyl-thiazol-5-yl)-quinolin-6-yl]-3-cyclohexyl-1-(2-morpholin-4-yl-2-oxo-ethyl)-1H-indole-6-carboxylic acid (282)

Compound 282 was synthesized from compound 135 as described for compound 299. MS: 687.1 (M+H⁺); H¹-NMR (DMSO-d₆): δ (ppm) 8.55 (d, 1H, J=8.7 Hz), 8.00-7.88 (m, 4H), 7.82 (d, 1H, J=8.1 Hz), 7.62 (dd, 1H, J=8.4, 1.5 Hz), 5.95 (s, 2H), 3.48-3.31 (m, 8H), 2.79 (s, 3H), 2.64 (s, 3H), 2.58 (m, 1H), 1.88-1.12 (m, 10H).

### Example 84

### 3-Cyclohexyl-2-[2-(2,4-dimethyl-thiazol-5-yl)-quinolin-6-yl]-1-(2-morpholin-4-yl-2-oxo-ethyl)-1H-indole-6-(β-D-glucuronic acid)ester (284)

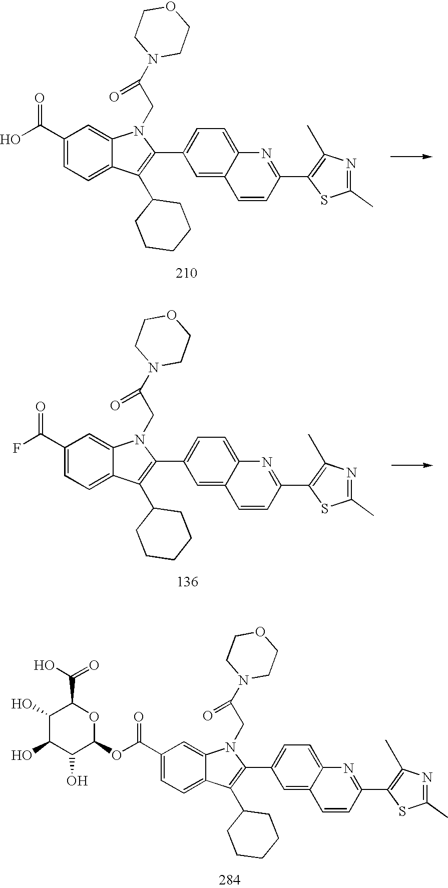

### Step 1. 3-Cyclohexyl-2-[2-(2,4-dimethyl-thiazol-5-yl)-quinolin-6-yl]-1-(2-morpholin-4-yl-2-oxo-ethyl)-1H-indole-6-carbonyl fluoride (136)

A solution of 100 mg (0.164 mmole) compound 210 in 3 mL THF was treated with 46 μL (0.344 mmole) DAST at −78 C.° under argon. The reaction was complete in 20 minutes. The solvent was evaporated and was purified on a silica pad using ethyl acetate for elution. Yield: 30 mg (30%) compound 136. MS: 611.2 (M+H⁺);

### Step 2. 3-Cyclohexyl-2-[2-(2,4-dimethyl-thiazol-5-yl)-quinolin-6-yl]-1-(2-morpholin-4-yl-2-oxo-ethyl)-1H-indole-6-(β-D-glucuronic acid)ester (284)

30 mg (0.049 mmole) compound 136, 69 mg (0.294 mmole) glucuronic acid sodium salt monohydrate was dissolved in a mixture of 2 mL acetone and 1 mL water. 50 mg solid NaHCO₃ was added and the mixture was stirred at room temperature for 1.5 h. The solvent was evaporated and the residue purified with RP-HPLC to give 11 mg compound 284 as pure β-anomer. MS: 785.2 (M+H⁺); H¹-NMR (DMSO-d₆): δ (ppm) 8.50 (d, 1H, J=9.0), 8.08-8.05 (m, 2H), 7.96-7.88 (m, 3H), 7.74-7.64 (m, 2H), 5.62 (d, 2H, J=7.5 Hz), 5.35 (d, 1H, J=4.5 Hz), 5.1-4.9 (m, 4H), 3.6-3.24 (m, 11H), 2.72 (s, 3H), 2.66 (s, 3H), 2.64 (m, 1H), 1.9-1.16 (m, 10H).

### Example 86

### 3-Cyclohexyl-1-(3-dimethylamino-propyl)-2-[2-(2,4-dimethyl-thiazol-5-yl)-quinolin-6-yl]-1H-indole-6-carboxylic acid (286)

Following the full procedure and workup for compound 289, 137 (65 mg, 0.13 mmol) in DMF (2 mL) was added NaH (27 mg, 5 eq). After 5 minutes stirring at room temperature, it was reacted with (3-Chloro-propyl)-dimethyl-amine hydrochloride (52 mg, 2.5 eq.) to produce compound 286 after saponification. Yield 5 mg, 8%. MS: 567.2 (M+H⁺); H¹-NMR (DMSO-d₆): δ (ppm) 9.8 (br s, 1H), 8.58 (d, 2H, J=8.4), 8.11-7.64 (m,7H), 4.1 (m, 2H), 2.73 (m, 2H), 2.45 (s, 6), 1.95-1.10 (m, 12H).

### Example 87

### 1-Benzyl-3-cyclohexyl-2-[2-(2,4-dimethyl-thiazol-5-yl)-quinolin-6-yl]-1H-indole-6-carboxylic acid (287)

Following the full procedure and workup for compound 289, 137 (65 mg, 0.11 mmol) in DMF (2 mL) was added NaH (27 mg, 5 eq). After 5 minutes stirring at room temperature, it was reacted with benzyl chloride (62 uL, 4 eq.) to produce compound 287 after saponification. Yield 20 mg, 23%. MS: 572.2 (M+H⁺); H¹-NMR (DMSO-d₆): 1 (ppm) 8.47 (d, 2H, J=8.1), 8.01-7.64 (m,7H), 7.2 (m, 3H), 6.8 (d, 2H, J=6.9), 5.3 (s, 2H), 2.70 (s, 3H), 2.66 (s, 3H), 2.62 (m, 1H), 1.95-1.10 (m, 10H).

### Example 88

### Step 1. 6-Bromo-2-(2,4-dimethyl-thiazol-5-yl)-quinoline (125)

To a solution of KOH (10.32 (85%) g, 156.27 mmol) in anhydrous EtOH (700 mL) was added 2-amino-5-bromobenzaldehyde (10.42 g, 52.09 mmol) and 5-acetyl-2,4-dimethylthiazole (8.16 mL, 60.42 mmol). The mixture was stirred under Ar at 78° C. for 16 h and then cooled down in an ice-bath. It was neutralized to pH 7 with 5 N HCl and then evaporated to about 60 mL. Water (500 mL) was added. The precipitate formed were collected by filtration, washed thoroughly with water, and dried to give 125 (15.62 g, 94%).

### Step 2. 2-(2,4-Dimethyl-thiazol-5-yl)-quinoline-6-boronic acid (126)

A mixture of 6-bromo-2-(2,4-dimethyl-thiazol-5-yl)-quinoline (125, 15 g, 46.99 mmol), bis(neopentylglucolato)diboron (31.83 g, 141 mmol), bis(triphenylphosphine)-palladium (II) chloride (1.65 g, 2.35 mmol), and potassium acetate (13.81 g, 141 mmol) in anhydrous DMSO (260 mL) was stirred under Ar at 90° C. for 2 h then was cooled down to room temperate. The mixture was poured into water (1.2 L) and the precipitate were collected by filtration, washed with water, and dried. To the dried solid was added EtOAc (600 mL) and the insoluble solid was filtered off. The filtrate was evaporated and the product was adsorbed onto silica gel and purified by a short silica pad eluting with EtOAc-hexane (5:2) to give compound 126 (16.4 g, still containing about 30% bis(neopentyl glucolato)diboron indicated by NMR-94% yield), which was directly used in step 7 without further purification.

### Step 3. 3-Cyclohex-1-enyl-1H-indole-6-carboxylic acid (163)

In a 3 L round-bottomed flask a solution made of indole 6-carboxylic acid (50.5 g), cyclohexanone (96.4 mL, 3 eq.) sodium methoxide (25 wt %, 433 mL 6.0 eq) and MeOH (1 L) was refluxed under argon atmosphere for 17 hrs. Some precipitation was noted after 3-4 hrs and became pronounced at the end of the reaction. The solution was worked up by adding 300 mL H₂O, removing the majority of MeOH via vacuum distillation, adding conc. HCl (160 mL) to pH 1, filtering, washing the precipitate with water and drying to get compound 163 in quantitative yield (75.5 g). It was pure enough to be used without further purification in Step 4.

### Step 4. 3-Cyclohexyl-1H-indole-6-carboxylic acid (164)

Compound 163 from Step 3 (75.5 g) was split into two batches. Each batches were suspended in 600 mL solvent (1:1 MeOH:THF). Pd catalyst (10% on carbon, 1 g/batch) was added as a slurry in CH₂Cl₂ (5 mL) and the mixture was hydrogenated for 15 hr at 50-60 psi. The catalyst was filtered off by means of Celite and the solvents were removed via vacuum distillation to a give compound 164 as a yellow solid. Yield 63 g (83%).

### Step 5. 3-Cyclohexyl-1H-indole-6-carboxylic acid methyl ester (165)

63 g (0.259 mmole) 164 was dissolved in MeOH (1 L) and HCl (100 mL, 4M in dioxane) was added slowly. The mixture was refluxed for 3 hrs. The purple solution was then cooled and the solvents removed under vacuum. The residue was dissolved in EtOAc (500 mL), was washed with NaHCO₃ (sat. 2×150 mL). The purple color was replaced with a light yellow one. The organic layer was further washed with saturated NaCl solution, dried (Na₂SO₄) and the solvents removed. The crude solid was recrystallized from a mixture of MeOH (2 L) and water (500 mL). The crystals were recovered on a filter, washed with water and dried to yield compound 165 as a light yellow solid (63.8 g, 95%).

### Step 6. 2-Bromo-3-cyclohexyl-1H-indole-6-carboxylic acid methyl ester (112)

Reaction vessel: 1 L 3-neck round bottom flask equipped with argon inlet/outlet and thermometer to monitor the inside temperature; Cooling bath: dry ice/ethanol

500 mL 1:1 THF-chloroform mixture was degassed before charging in the reaction vessel. The reaction was kept all the time under argon atmosphere.

30.00 g (116.7 mmole) 165 was dissolved in the 500 mL solvent mixture. The solution was cooled to −10 C.° (inner temperature) then 56.1 g (157.5 mmole) pyridinium tribromide was added as a solid in one portion. The mixture was agitated for 3 h while keeping the temperature between −7 C.° and −14 C.°. 450 mL 10% NaHSO₃ solution was added and was stirred vigorously for 5 minutes. The two phases were separated, the organic phase was successively washed with 100 mL portions of water (2×), saturated NaHCO₃ (3×), brine (2×) then was dried (Na₂SO₄) and evaporated to dryness. The dark brown crude product was dissolved in 50 mL methanol by gentle heating. The product crystallized overnight at 4 C.°. The crystals were filtered off, washed with small amount of cold methanol (2×) and dried to yield 22 g (56%) light brown crystals. The mother liquid was evaporated and was purified on a 1 L silica gel pad using hexane-ethyl-acetate solvent system and a stepwise (0.5 L/step) 6% to 30% ethyl-acetate gradient. The product elutes at around 20% ethyl-acetate content. Total yield: 31.41 g (80%).

MS, NMR are consistent with the structure.

### Step 7. 3-Cyclohexyl-2-[2-(2,4-dimethyl-thiazol-5-yl)-quinolin-6-yl]-1H-indole-6-carboxylic acid methyl ester (137)

A mixture of compound 126 (see Example 11 for synthesis, 70% pure; 58.6 g, 144 mmol), compound 112 (1 eq, 48.5 g), NaHCO₃ (sat. aq., 210 mL), methanol (1.5 L) and Pd[P(Ph)₃]₄ (5 mol %, 8.3 g) degassed by sparging with argon for 20 minutes, was refluxed under argon for 16 hrs. The yellow mixture never went all into solution. The reaction was then cooled to 0° C., filtered and the yellow cake washed with cold methanol. The precipitate was >96% by QC-RP-HPLC and the mother liquor was <10% product and was discarded. The solid was dried to give the product which was combined with another batch to give 92.95 g (95% yield). NMR d6-DMSO δ (ppm): 11.7 (1H, s), 8.55 (1H, d, J=8.7), 8.11-7.88 (5H, m) 7.59 (1H, d, J=8.7), 3.86 (3H, s), 2.96 (1H, m), 2.72 (3H, s), 2.66 (3H, s), 2.01-1.7 (10H, m). MS-ESI (496, M+H, 100%).

### Step 8. 3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid methyl ester (166)

In a 1 L round bottom flask, 3-cyclohexyl-2-[2-(2,4-dimethyl-thiazol-5-yl)-quinolin-6-yl]-1H-indole-6-carboxylic acid methyl ester (10.1 g, 20.38 mmol) and KI (300 mg, 1.81 mmol) were dissolved in DMF (200 mL). The flask was then placed in an ice water bath, and stirred under argon until the reaction mixture reached 0° C. NaH (978 mg, 40.76 mmol) was then added in one portion. A vacuum was applied to the flask until the bubbling had stopped. Finally, 2-Chloro-1-morpholin-4-yl-ethanone (4 g, 24.46 mmol) was added, and the reaction was stirred at 0° C. until no starting material remained (monitored by RP-HPLC). The mixture was then poured into 1 L ice water, neutralized with aq HCl, filtered, and dried, resulting in 3-cyclohexyl-2-[2-(2,4-dimethyl-thiazol-5-yl)-quinolin-6-yl]-1-(2 morpholin-4-yl-2-oxo-ethyl)-1H-indole-6-carboxylic acid methyl ester (12 g, 94% yield).

### Step 9. 3-Cyclohexyl-2-[2-(2,4-dimethyl-thiazol-5-yl)-quinolin-6-yl]—(2-morpholin-4-yl-2-oxo-ethyl)-1H-indole-6-carboxylic acid (210)

To a suspension of 104 g (0.167 mole) compound 166 in 1.5 L dioxane 28 g (0.7 mole) a solution of NaOH (in 700 mL water) was added. The mixture was heated at 55 C.° for 5 h by the time it became a clear solution. It was evaporated to dryness. To the residue 500 mL water and 500 mL EtOAc was added and the pH was adjusted to 2 by means of 6M HCl. The material partially got dissolved, partially crystallized. The crystals were filtered off, washed thoroughly with water, dried then were suspended in 300 mL acetinitrile, boiled for 5 min, cooled down, filtered off and dried again to give 69.4 g (68%) 210 as a 99+% pure yellow solid.

The EtOAc phase was washed with water, brine, dried (sodium sulfate) and evaporated. The residue was treated with 150 mL acetonitrile similarly to the crystals to give an additional 16.9 g (17%) compound 210 as a 90% pure yellow solid. Total yield was 85%.

MS, NMR are consistent with the structure.

### Example 89

### 3-Cyclohexyl-2-[2-(2,4-dimethyl-thiazol-5-yl)-quinolin-6-yl]-1-pyridin-4-ylmethyl-1H-indole-6-carboxylic acid (289)

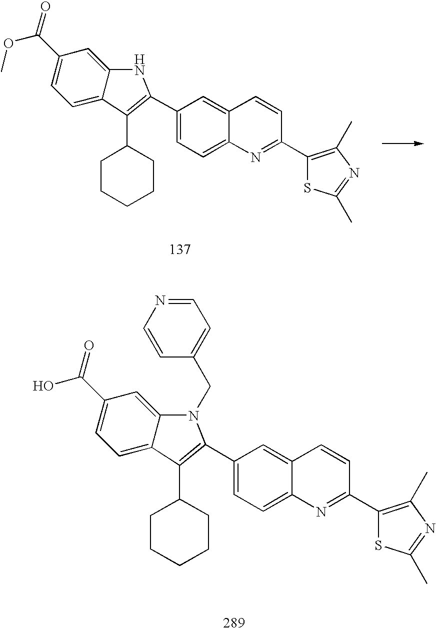

To a solution of the indole compound 137 (94 mg, 0.19 mmol) in DMF (2 mL) was added NaH (27 mg, 5 eq). After 5 minutes stirring at room temperature 4-bromomethylpyridine hydrobromide was added and the mixture stirred for 4 hours. The reaction was then quenched with water (1 mL) which precipitated the product. It was then spun down to a pellet and redissolved in 5 mL of methanol:water (5% LiOH) and heated to 50 C for 8 hrs. The product was then purified by RP-HPLC. Yield 35.2 mg, 30%. MS: 573.2 (M+H⁺); H¹-NMR (DMSO-d₆): δ (ppm) 8.61 (d, 2H, J=5.1), 8.44 (d, 1H, J=8.1), 8.01-7.62 (m,7H), 7.2 (d, 1H, J=5.1), 5.6 (s, 2H), 2.69 (s, 3H), 2.65 (s, 3H), 2.62 (m, 1H) 1.95-1.10 (m, 10H).

### Example 90

### 3-Cyclohexyl-2-[2-(2,4-dimethyl-thiazol-5-yl)-quinolin-6-yl]-1-(2-morpholin-4-yl-ethyl)-1H-indole-6-carboxylic acid (290)

Following the full procedure and workup for 289, 137 (90 mg, 0.18 mmol) in DMF (2 mL) was added NaH (27 mg, 5 eq). After 5 minutes stirring at room temperature, it was reacted with 4-(2-Chloro-ethyl)-morpholine hydrochloride (67 mg, 2 eq.) to produce compound 290 after saponification. Yield 26 mg, 29%. MS: 595.2 (M+H⁺); H¹-NMR (DMSO-d₆): δ (ppm) 8.47 (d, 1H, J=9.0), 8.21-7.70 (m, 7H), 4.46 (m, 2H), 3.55-3.29 (m, 8H), 3.00 (m, 2H), 2.73 (s, 3H), 2.68 (s, 3H), 2.56 (m, 1H), 1.95-1.10 (m, 10H).

### Example 91

### 3-Cyclohexyl-2-[2-(2,4-dimethyl-thiazol-5-yl)-quinolin-6-yl]-1-(5-methyl-isoxazol-3-ylmethyl)-1H-indole-6-carboxylic acid (291)

Following the full procedure and workup for compound 289, 137 (80 mg, 0.16 mmol) in DMF (2 mL) was added NaH (27 mg, 5 eq). After 5 minutes stirring at room temperature, it was reacted with 3-Chloromethyl-5-methyl-isoxazole (85 mg, 3 eq.) to produce compound 291 after saponification. Yield 21 mg, 22%. MS: 577.2 (M+H⁺); H¹-NMR (DMSO-d₆): δ (ppm) 8.47 (d, 1H, J=9.0), 8.01-7.64 (m,8H), 5.71 (s, 1H), 5.23 (s, 1H), 2.68 (s, 3H), 2.62 (s, 3H), 2.56 (m, 1H), 2.23 (s, 3H), 1.95-1.10 (m, 10H).

### Example 92

### 1-(4-Carboxy-benzyl)-3-cyclohexyl-2-[2-(2,4-dimethyl-thiazol-5-yl)-quinolin-6-yl]-1H-indole-6-carboxylic acid (292)

Following the full procedure and workup for compound 289, to a mixture of compound 137 (72 mg, 0.14 mmol) in DMF (2 mL) was added NaH (27 mg, 5 eq). After 5 minutes stirring at room temperature, it was reacted with 4-Bromomethyl-benzoic acid (103 mg, 2 eq) to produce compound 292 after saponification. Yield 17 mg, 22%. MS: 616.2 (M+H⁺); H¹-NMR (DMSO-d₆): δ (ppm) 8.77 (s, 1H), 8.45 (d, 1H, J=9.0), 8.01-7.63 (m, 9H), 6.9 (d, 2H, J=8.1), 5.42 (s, 2H) 2.70 (s, 3H), 2.67 (s, 3H), 2.62 (m, 1H), 1.95-1.10 (m, 9H).

### Example 93

### 3-Cyclohexyl-2-[2-(2,4-dimethyl-thiazol-5-yl)-quinolin-6-yl]-1-(3-methoxy-benzyl)-1H-indole-6-carboxylic acid (293)

Following the full procedure and workup for compound 289, 137 (80 mg, 0.16 mmol) in DMF (2 mL) was added NaH (27 mg, 5 eq). After 5 minutes stirring at room temperature, it was reacted with 1-Bromomethyl-3-methoxy-benzene (67 uL, 3 eq.) to produce compound 293 after saponification. Yield 16 mg, 16%. MS: 602.2 (M+H⁺); H¹-NMR (DMSO-d₆): δ (ppm) 8.47 (d, 1H, J=8.4), 8.01-7.63 (m, 8H), 7.08 (m, 1H), 6.70 (m, 1H), 6.33 (m, 2H), 5.31 (s, 2H), 3.55 (s, 3H), 2.70 (s, 3H), 2.65 (s, 3H), 2.56 (m, 1H), 1.95-1.10 (m, 10H).

### Example 98

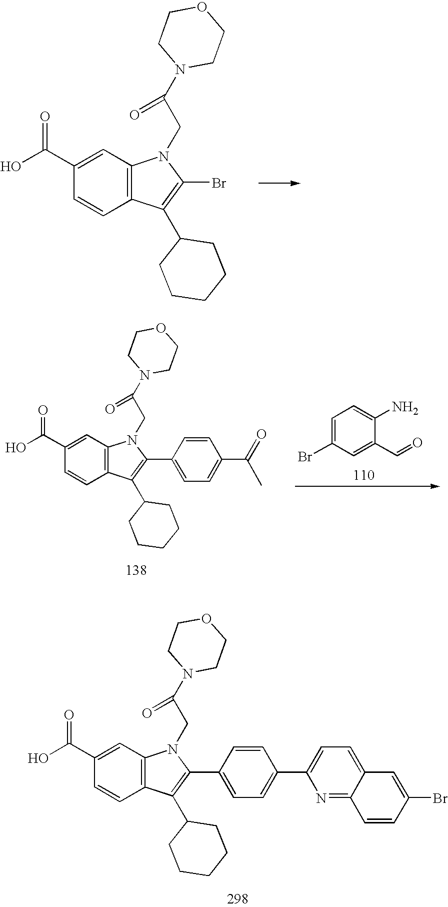

### Step 1. 2-(4-Acetyl-phenyl)-3-cyclohexyl-1-(2-morpholin-4-yl-2-oxo-ethyl)-1H-indole-6-carboxylic acid (138)

Compound 138 was synthesized as described for compound 139 in Example 99 replacing compound 121 with 2-Bromo-3-cyclohexyl-1-(2-morpholin-4-yl-2-oxo-ethyl)-1H-indole-6-carboxylic acid (Beaulieu, P. et al., PCT application WO 030141) and 4-hydroxy-phenylboronic acid with 4-acetyl-phenylboronic acid. MS: 489.1 (M+H⁺);

### Step 2. 2-[4-(6-Bromo-quinolin-2-yl)-phenyl]-3-cyclohexyl-1-(2-morpholin-4-yl-2-oxo-ethyl)-1H-indole-6-carboxylic acid (298)

Compound 298 was synthesized as described for compound 111 replacing compound 106 with compound 138. MS: 652.1 (M+H⁺); H¹-NMR (DMSO-d₆): δ (ppm) 8.50 (d, 1H, J=8.7 Hz), 8.43-8.40 (m, 2H), 8.33-8.29 (m, 2H), 8.03 (d, 1H, J=8.7 Hz), 7.97 (s, 2H), 7.92 (dd, 2H, J=8.7, 1.8 Hz), 7.85 (d, 1H, J=8.7 Hz), 7.65 (d, 1H, J=8.1 Hz), 7.50-7.48 (m, 2H), 4.99 (s, 2H), 3.60-3.33 (m, 8H), 2.65 (m, 1H0, 1.93-1.15 (m, 10H).

### Example 99

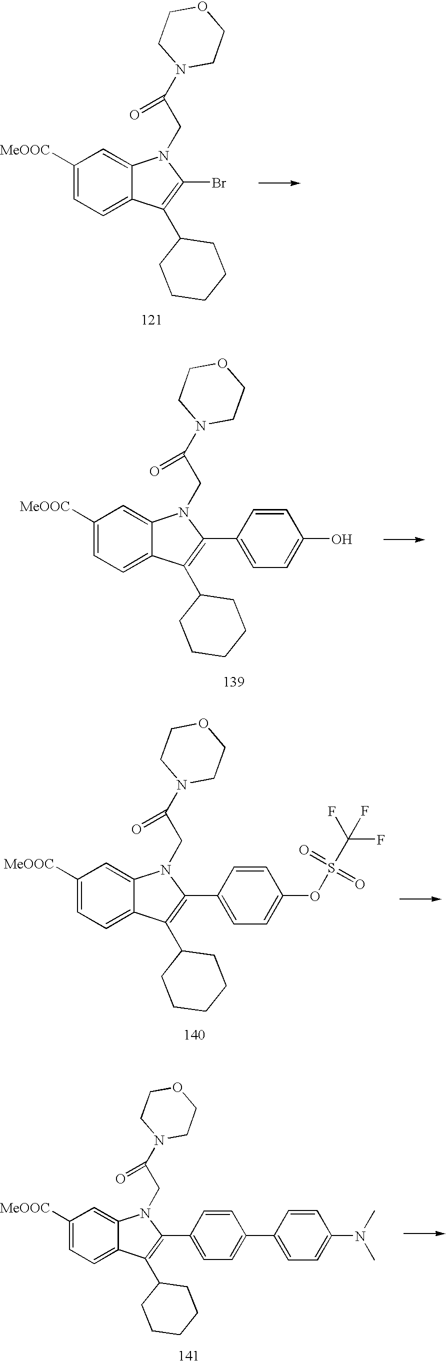

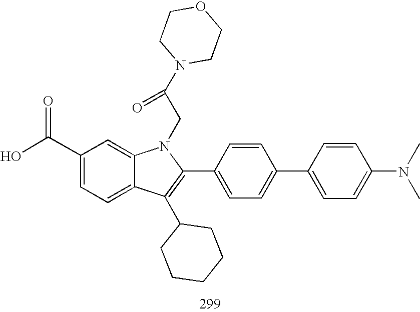

### Step 1. 3-Cyclohexyl-2-(4-hydroxy-phenyl)-1-(2-morpholin-4-yl-2-oxo-ethyl)-1H-indole-6-carboxylic acid methyl ester (139)

A mixture of 1.713 g (3.70 mmole) compound 121, 771 mg (5.55 mmole) 4-hydroxy-phenylboronic acid, 214 mg (0.185 mmole) Pd(Ph₃P)₄ 85 mL methanol and 8.5 mL sat. NaHCO₃ was heated overnight at 80 C.° under argon. It was evaporated to dryness and purified on a 300 mL silica pad using toluene-ethylacetate eluent system. Yield: 1.60 g (86%) compound 139. MS: 477.2 (M+H⁺); H¹-NMR (DMSO-d₆): δ (ppm) 99.77 (s, 1H), 7.93 (d, 1H, J=1.2 Hz), 7.90 (d, 1H, J=8.4), 7.62 (dd, 1H, J=8.1, 1.2 Hz), 7.08 (m, 2H), 6.86 (m,2H), 4.87 (s, 2H), 2.77 (s, 3H), 3.50-3.33 (m, 8H), 2.57 (m, 1H), 1.86-1.16 (m, 10H).

### Step 2. 3-Cyclohexyl-1-(2-morpholin-4-yl-2-oxo-ethyl)-2-(4-trifluoromethane-sulfonyloxy-phenyl)-1H-indole-6-carboxylic acid methyl ester (140)

To a cold (0 C.°) solution of 1.5 g (3.115 mmole) compound 139, 1.275 mL pyridine and 39 mg DMAP in DCM 1.59 mL (9.45 mmole) triflic anhydride was added dropwise in a period of about 1 minute. The reaction is instantaneous. The mixture was evaporated, the residue taken up in a mixture of ethylacetate and icy water, washed twice with cold water, dried with sodium sulfate. The drying agent was removed by filtration and the solution was evaporated to dryness to give compound 140 as a yellow solid foam which was pure enough to use without further purification. Yield: 1.76 g (92%). MS: 609.1 (M+H); H¹-NMR (DMSO-d₆): δ (ppm) 8.12 (d, 1H), 7.93 (dd, 1H), 7.76-7.72 (m, 3H), 7.58-7.55 (m, 2H), 5.03 (s, 2H), 3.93 (s, 3H), 3.54-3.44 (m, 8H), 2.60 (m, 1H), 2.0-1.22 (m, 10H); F¹⁹-NMR (DMSO-d₆): δ (ppm) −73.22.

### Step 3. 3-Cyclohexyl-2-(4′-dimethylamino-biphenyl-4-yl)-1-(2-morpholin-4-yl-2-oxo-ethyl)-1H-indole-6-carboxylic acid methyl ester (141)

A mixture of 104 mg (0.171 mmole) compound 140, 42.3 mg (0.256 mmole) 4-dimethylamino-phenylboronic acid, 10 mg (0.0085 mmole) Pd(Ph₃P)₄ 5 mL methanol and 1 mL sat. NaHCO₃ was heated overnight at 80 C.° under argon. It was evaporated to dryness and triturated with water. The solid compound 141 was filtered off and was used without drying in the following step.

### Step 4. 3-Cyclohexyl-2-(4′-dimethylamino-biphenyl-4-yl)-1-(2-morpholin-4-yl-2-oxo-ethyl)-1H-indole-6-carboxylic acid (299)

The wet compound 141 from the previous step was dissolved in a mixture of 5 mL THF, 1 mL methanol and 1 mL 2M NaOH. It was refluxed for 1 h then the solvent was removed by evaporation. The residue was purified using RP-HPLC to get 41 mg (40%) compound 299. MS: 566.3 (M+H⁺); H¹-NMR (DMSO-d₆): δ (ppm) 7.95 (d, 1H, J=1.5 Hz), 7.83-7.76 (m, 3H), 7.71-7.68 (m, 2H0, 7.65 (dd, 1H, J=8.7, 1.5 Hz), 7.33 (d, 2H), 7.2 (br, 2H), 4.93 (s, 2H), 3.50-3.33 (m, 8H), 3.01 (s, 6H), 2.63 (m, 1H), 2.0-1.10 (m, 10H).

### Example 100

### 3-Cyclohexyl-2-(4′-methyl-biphenyl-4-yl)-1-(2-morpholin-4-yl-2-oxo-ethyl)-1H-indole-6-carboxylic acid (300)

Following the full procedure and workup for compound 299, 140 (100 mg, 0.16 mmol) was reacted with 4-Methyl phenyl boronic acid (27 mg, 0.2 mmol) to produce compound 300 (7 mg, 8% yield). MS: 537.2 (M+H⁺); H¹-NMR (DMSO d₆): 7.96 (s, 1H), 7.82 (m, 3H), 7.65 (m, 3H), 7.36 (d, 2H, J=8.1 Hz), 7.31 (d, 2H, J=8.1 Hz), 4.95 (s, 2H), 3.47 (m, 8H), 2.63 (m, 1H), 2.37 (s, 3H), 1.85 (m, 7H), 1.28 (m, 4H).

### Example 101

### 3-Cyclohexyl-2-(4′-methoxy-biphenyl-4-yl)-1-(2-morpholin-4-yl-2-oxo-ethyl)-1H-indole-6-carboxylic acid (301)

Following the full procedure and workup for compound 299, 140 (100 mg, 0.16 mmol) was reacted with 4-Methoxy phenyl boronic acid (37 mg, 0.24 mmol) to produce compound 301 (20 mg, 23% yield). MS: 553.2 (M+H⁺); H¹-NMR (DMSO d₆): 7.94 (s, 1H), 7.78 (m, 3H), 7.69 (m, 3H), 7.66 (m, 3H), 7.33 (d, 2H, J=8.4 Hz), 7.04 (d, 2H, J=8.7 Hz), 4.92 (s, 2H), 3.80 (s, 3H), 3.45 (m, 8H), 2.63 (m, 1H), 1.83 (m, 7H), 1.25 (m, 3H).

### Example 102

### 3-Cyclohexyl-2-(2′-fluoro-biphenyl-4-yl)-1-(2-morpholin-4-yl-2-oxo-ethyl)-1H-indole-6-carboxylic acid (302)

Following the full procedure and workup for compound 299, 140 (100 mg, 0.16 mmol) was reacted with 2-Fluorophenyl boronic acid (34 mg, 0.24 mmol) to produce compound 302 (15 mg, 17% yield). MS: 541.2 (M+H⁺); H¹-NMR (DMSO d₆): 8.04 (s, 1H), 7.90 (d, 1H, J=8.7 Hz), 7.78 (d, 2H, J=8.4 Hz), 7.69 (m, 2H), 7.44 (m, 5H), 5.02 (s, 2H), 3.52 (m, 8H), 2.57 (m, 1H), 1.83 (m, 7H), 1.33 (m, 31′).

### Example 103

### 3-Cyclohexyl-2-[4-(2-fluoro-pyridin-3-yl)-phenyl]-1-(2-morpholin-4-yl-2-oxo-ethyl)-1H-indole-6-carboxylic acid (303)

Following the full procedure and workup for compound 299, 140 (100 mg, 0.16 mmol) was reacted with 2-Fluoro-pyridine-3-boronic acid (34 mg, 0.24 mmol) to produce compound 303 (22 mg, 26% yield). MS: 542.2 (M+H⁺); H¹-NMR (DMSO d₆): 8.23 (m, 2H), 7.97 (s, 1H), 7.80 (m, 3H), 7.63 (dd, 1H, J=8.4 Hz, 1.2 Hz), 7.50 (m, 1H), 7.42 (d, 2H, J=8.1 Hz), 4.95 (s, 2H), 3.4 (m, 8H), 2.62 (m, 1H), 1.76 (m, 7H), 1.23 (m, 3H).

### Example 104

### 3-Cyclohexyl-2-[4-(2-methoxy-pyridin-3-yl)-phenyl]-1-(2-morpholin-4-yl-2-oxo-ethyl)-1H-indole-6-carboxylic acid (304)

Following the full procedure and workup for compound 299, 140 (100 mg, 0.16 mmol) was reacted with 2-Methoxy-pyridine-3-boronic acid (37 mg, 0.24 mmol) to produce compound 304 (20 mg, 23% yield). MS: 554.2 (M+H⁺); H¹-NMR (DMSO d₆): 8.19 (dd, 1H, J=4.8 Hz, 1.5 Hz), 7.96 (s, 1H), 7.83 (m, 2H), 7.72 (d, 2H, J=8.4 Hz), 7.63 (dd, 1H, J=8.4 Hz, 1.5 Hz), 7.34 (d, 2H, J=8.1 Hz), 7.12 (dd, 1H, J=7.2 Hz, 5.1 Hz), 4.93 (s, 2H), 3.91 (s, 3H), 3.45 (m, 8H), 2.63 (m, 1H), 1.76 (m, 7H), 1.27 (m, 3H).

### Example 105

### 3-Cyclohexyl-2-[4-(2-methoxy-pyridin-3-yl)-phenyl]-1-(2-morpholin-4-yl-2-oxo-ethyl)-1H-indole-6-carboxylic acid (305)

Following the full procedure and workup for compound 299, 140 (100 mg, 0.16 mmol) was reacted with 4-Methoxy-pyridine-3-boronic acid (37 mg, 0.24 mmol) to produce compound 305 (20 mg, 23% yield). MS: 554.2 (M+H⁺); H¹-NMR (DMSO d₆): 8.58 (d, 1H, J=2.1 Hz), 8.10 (dd, 1H, J=8.4 Hz, 2.4 Hz), 7.95 (s, 1H), 7.83 (m, 3H), 7.63 (d, 1H, J=8.4 Hz), 7.37 (d, 2H, J=8.1 Hz), 6.94 (d, 1H, J=8.4 Hz), 4.94 (s, 2H), 3.91 (s, 3H), 3.47 (m, 8H), 2.61 (m, 1H), 1.75 (m, 7H), 1.25 (m, 3H).

### Example 106

### 2-(3′-Cyano-biphenyl-4-yl)-3-cyclohexyl-1-(2-morpholin-4-yl-2-oxo-ethyl)-1H-indole-6-carboxylic acid (306)

Following the full procedure and workup for compound 299, 140 (100 mg, 0.16 mmol) was reacted with 3-Cyanophenyl boronic acid (35 mg, 0.24 mmol) to produce compound 306 (5 mg, 6% yield). MS: 548.2 (M+H⁺); H¹-NMR (DMSO d₆): 8.26 (s, 1H), 8.12 (d, 1H, J=7.8 Hz), 7.93 (m, 3H), 7.85 (m, 2H), 7.68 (m, 2H), 7.41 (d, 2H, J=8.1 Hz), 4.93 (s, 2H), 3.42 (m, 8H), 2.56 (m, 1H), 1.76 (m, 7H), 1.23 (m, 3H).

### Example 107

### 2-(4′-Cyano-biphenyl-4-yl)-3-cyclohexyl-1-(2-morpholin-4-yl-2-oxo-ethyl)-1H-indole-6-carboxylic acid (307)

Following the full procedure and workup for compound 299, 140 (100 mg, 0.16 mmol) was reacted with 4-Cyanophenyl boronic acid (35 mg, 0.24 mmol) to produce compound 307 (18 mg, 20% yield). MS: 548.2 (M+H⁺); H¹-NMR (DMSO d₆): 7.95 (m, 7H), 7.83 (d, 1H, J=8.4 Hz), 7.64 (d, 1H, J=8.1 Hz), 7.43 (d, 2H, J=8.1 Hz), 4.95 (s, 2H), 3.47 (m, 8H), 2.63 (m, 1H), 1.77 (m, 7H), 1.26 (m, 3H).

### Example 108

### 1-Carboxymethyl-3-cyclohexyl-2-[4-(4-methoxy-pyridin-3-yl)-phenyl]-1H-indole-6-carboxylic acid (308)

Following the full procedure and workup for compound 299, 140 (100 mg, 0.16 mmol) was reacted with 4-Methoxy-pyridine-3-boronic acid (35 mg, 0.24 mmol), and allowed to soponify until you see hydrolysis of the morpholine group, to produce compound 308 (5 mg, 4% yield). MS: 485.2 (M+H⁺); H¹-NMR (DMSO d₆): 8.60 (d, 1H, J=2.7 Hz), 8.12 (dd, 1H, J=8.4 Hz, 2.4 Hz), 7.97 (d, 1H, J=1.5 Hz), 7.84 (m, 3H), 7.65 (dd, 1H, J=8.1 Hz, 1.2 Hz), 7.40 (d, 2H, J=8.4 Hz), 6.93 (d, 1H, J=8.7 Hz), 4.76 (s, 2H), 3.91 (s, 3H), 2.61 (m, 1H), 1.75 (m, 7H), 1.26 (m, 3H).

### Example 109

### 3-Cyclohexyl-2-(3′-methoxy-biphenyl-4-yl)-1-(2-morpholin-4-yl-2-oxo-ethyl)-1H-indole-6-carboxylic acid (309)

Following the full procedure and workup for compound 299, 140 (100 mg, 0.16 mmol) was reacted with 3-Methoxy phenylboronic acid (37 mg, 0.24 mmol) to produce compound 309 (48 mg, 55% yield). MS: 553.2 (M+H⁺); H¹-NMR (DMSO d₆): 7.96 (s, 1H), 7.83 (dd, 3H, J=8.1 Hz, 1.8 Hz), 7.63 (dd, 1H, J=8.7 Hz, 1.5 Hz), 7.34 (m, 5H), 6.97 (m, 1H), 4.94 (s, 2H), 3.84 (s, 3H), 3.45 (m, 8H), 2.64 (m, 1H), 1.75 (m, 7H), 1.25 (m, 3H).

### Example 110

### 3-Cyclohexyl-2-(3′-nitro-biphenyl-4-yl)-1-(2-morpholin-4-yl-2-oxo-ethyl)-1H-indole-6-carboxylic acid (310)

Following the full procedure and workup for compound 299, 140 (100 mg, 0.16 mmol) was reacted with 3-nitrophenyl boronic acid (41 mg, 0.24 mmol) to produce compound 310 (46 mg, 50% yield). MS: 568.2 (M+H⁺); H¹-NMR (DMSO d₆): 8.53 (m, 1H), 8.25 (dd, 2H, J=8.1 Hz, 2.1 Hz), 7.97 (m, 3H), 7.81 (m, 2H), 7.64 (dd, 1H, J=8.1 Hz, 1.2 Hz), 7.45 (d, 2H, J=8.1 Hz), 4.97 (s, 2H), 3.43 (m, 8H), 2.63 (m, 1H), 1.75 (m, 7H), 1.22 (m, 3H).

### Example 111

### 3-Cyclohexyl-2-(21′-methoxy-biphenyl-4-yl)-1′-(2-morpholin-4-yl-2-oxo-ethyl)-1H-indole-6-carboxylic acid (311)

Following the full procedure and workup for compound 299, 140 (100 mg, 0.16 mmol) was reacted with 2-Methoxyphenyl boronic acid (37 mg, 0.24 mmol) to produce compound 311 (46 mg, 50% yield). MS: 553.2 (M+H⁺); H¹-NMR (DMSO d₆): 7.97 (s, 1H), 7.83 (d, 1H, J=8.4 Hz), 7.63 (m, 3H), 7.35 (m, 4H), 7.11 (m, 2H), 4.94 (s, 2H), 3.81 (s, 3H), 3.41 (m, 8H), 2.62 (m, 1H), 1.77 (m, 7H), 1.26 (m, 3H).

### Example 112

### 3-Cyclohexyl-2-(3′-methyl-biphenyl-4-yl)-1-(2-morpholin-4-yl-2-oxo-ethyl)-1H-indole-6-carboxylic acid (312)

Following the full procedure and workup for compound 299, 140 (100 mg, 0.16 mmol) was reacted with m-tolyl boronic acid (33 mg, 0.24 mmol) to produce compound 312 (21 mg, 24% yield). MS: 537.3 (M+H⁺); H¹-NMR (DMSO d₆): 7.96 (s, 1H), 7.81 (m, 3H), 7.64 (m, 1H), 7.55 (m, 2H), 7.38 (m, 3H), 7.21 (d, 1H, J=7.5 Hz), 4.95 (s, 2H), 3.46 (m, 8H), 2.64 (m, 1H), 1.77 (m, 7H), 1.23 (m, 3H).

### Example 113

### 3-Cyclohexyl-2-(2′-methyl-biphenyl-4-yl)-1-(2-morpholin-4-yl-2-oxo-ethyl)-1H-indole-6-carboxylic acid (313)

Following the full procedure and workup for compound 299, 140 (100 mg, 0.16 mmol) was reacted with o-tolyl boronic acid (33 mg, 0.24 mmol) to produce compound 313 (19 mg, 22% yield). MS: 537.3 (M+H⁺); H¹-NMR (DMSO d₆): 7.99 (s, 1H), 7.83 (d, 1H, J=8.4 Hz), 7.64 (d, 1H, J=8.7 Hz), 7.49 (d, 2H, J=8.1 Hz), 7.31 (m, 6H), 4.96 (s, 2H), 3.41 (m, 8H), 2.65 (m, 1H), 2.30 (s, 3H), 1.78 (m, 7H), 1.23 (m, 3H).

### Example 114

### 3-Cyclohexyl-1-(2-morpholin-4-yl-2-oxo-ethyl)-2-(4′-vinyl-biphenyl-4-yl)-1H-indole-6-carboxylic acid (314)

Following the full procedure and workup for compound 299, 140 (100 mg, 0.16 mmol) was reacted with 4-Vinylphenyl boronic acid (36 mg, 0.24 mmol) to produce compound 314 (41 mg, 48% yield). MS: 549.3 (M+H⁺); H¹-NMR (DMSO d₆): 7.96 (s, 1H), 7.82 (m, 5H) 7.63 (m, 3H), 7.38 (d, 2H, J=8.1 Hz), 6.79 (m, 1H), 5.91 (m, 1H), 5.31 (m, 1H), 4.96 (s, 2H), 3.46 (m, 8H), 2.64 (m, 1H), 1.76 (m, 7H), 1.23 (m, 3H).

### Example 115

### 2-(3′-Amino-biphenyl-4-yl)-3-cyclohexyl-1-(2-morpholin-4-yl-2-oxo-ethyl)-1H-indole-6-carboxylic acid (315)

Following the full procedure and workup for compound 299, 140 (100 mg, 0.16 mmol) was reacted with 3-Aminophenyl boronic acid (33 mg, 0.24 mmol) to produce compound 315 (36 mg, 42% yield). MS: 538.3 (M+H⁺); H¹-NMR (DMSO d₆): 7.97 (m, 1H), 7.79 (m, 3H), 7.57 (m, 4H), 7.44 (m, 3H), 4.96 (s, 2H), 3.49 (m, 8H), 2.54 (m, 1H), 1.76 (m, 7H), 1.27 (m, 3H).

### Example 116

### 3-Cyclohexyl-2-[4-(5-methyl-thiophen-2-yl)-phenyl]-1-(2-morpholin-4-yl-2-oxo-ethyl)-1H-indole-6-carboxylic acid (316)

Following the full procedure and workup for compound 299, 140 (100 mg, 0.16 mmol) was reacted with 5-Methyl-thiophene-2-boronic acid (102 mg, 0.72 mmol) to produce compound 316 (29 mg, 34% yield). MS: 543.2 (M+H⁺); H¹-NMR (DMSO d₆): 7.95 (s, 1H), 7.81 (d, 1H, J=8.4 Hz), 7.71 (d, 2H, J=8.1 Hz), 7.64 (dd, 1H, J=8.1 Hz, 1.2 Hz), 7.41 (d, 1H, J=3.3 Hz), 7.30 (d, 2H, J=8.1 Hz), 6.85 (dd, 1H, J=3.6 Hz, 1.2 Hz), 4.93 (s, 2H), 3.49 (m, 8H), 2.62 (m, 1H), 2.41 (s, 3H), 1.74 (m, 7H), 1.26 (m, 3H).

### Example 117

### 3-Cyclohexyl-2-[4-(3,5-dim ethyl-isoxazol-4-yl)-phenyl]-1-(2-morpholin-4-yl-2-oxo-ethyl)-1H-indole-6-carboxylic acid (317)

Following the full procedure and workup for compound 299, 140 (100 mg, 0.16 mmol) was reacted with 3,5-Dimethyl-isoxazole-4-boronic acid (101 mg, 0.72 mmol) to produce compound 317 (52 mg, 60% yield). MS: 542.2 (M+H⁺); H¹-NMR (DMSO d₆): 7.94 (s, 1H), 7.83 (d, 1H, J=8.4 Hz), 7.64 (dd, 1H, J=8.4 Hz, 1.2 Hz), 7.56 (m, 2H), 7.38 (d, 2H, J=8.4 Hz), 4.95 (s, 2H), 3.43 (m, 8H), 2.63 (m, 1H), 2.48 (s, 3H), 2.31 (s, 3H), 1.76 (m, 7H), 1.28 (m, 3H).

### Example 118

### 2-[4-(5-Chloro-thiophen-2-yl)-phenyl]-3-cyclohexyl-1-(2-morpholin-4-yl-2-oxo-ethyl)-1H-indole-6-carboxylic acid (318)

Following the full procedure and workup for compound 299, 140 (100 mg, 0.16 mmol) was reacted with 5-Chloro-thiophene-2-boronic acid (101 mg, 0.72 mmol) to produce compound 318 (52 mg, 26% yield). MS: 563.2 (M+H⁺); H¹-NMR (DMSO d₆): 7.95 (s, 1H), 7.78 (m, 3H), 7.63 (dd, 1H, J=8.1 Hz, 0.9 Hz), 7.51 (d, 1H, J=3.9 Hz), 7.34 (d, 2H, J=8.4 Hz), 7.20 (d, 1H, J=4.2 Hz), 4.94 (s, 2H), 3.46 (m, 8H), 2.60 (m, 1H), 1.74 (m, 7H), 1.23 (m, 3H).

### Example 119

### 3-Cyclohexyl-2-[7-(2,4-dimethyl-thiazol-5-yl)-[1,8]naphthyridin-3-yl]-1-(2-orpholin-4-yl-2-oxo-ethyl)-1H-indole-6-carboxylic acid (319)

Compound 319 was synthesized in 6 steps as described for compound 125 (step 1), 126 (step 2), 127 (step 3), 128 (step 4), 129 (step 5), and 212 (step 6), replacing 110 with 2-Amino-5-iodo-pyridine-3-carbaldehyde in Step 1. MS: 610.24 (M+H⁺); H¹-NMR (DMSO-d₆): (ppm) 8.94 (s, 1H), 8.65 (d, 1H, J=8.7 Hz), 8.48 (s, 1H), 8.08-8.05 (m, 2H0, 7.88 (dd, 1H, J=8.4 Hz), 7.67 (dd, 1H, J=8.4 Hz), 5.10 (s, 2H), 3.43-3.37 (m, 8H), 2.78 (s, 3H), 2.70 (s, 3H), 2.59 (m, 1H), 1.90-1.1 (m, 10H).

### Example 120

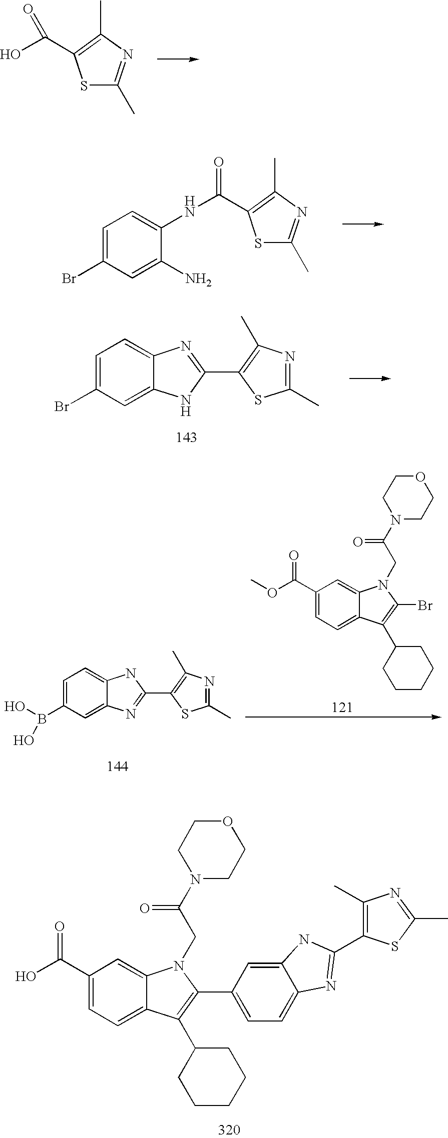

### Step 1. 6-Bromo-2-(2,4-dimethyl-thiazol-5-yl)-1H-benzoimidazole (143)

2,4-Dimethyl-thiazole-5-carboxylic acid (500 mg, 3.2 mmol), HATU (3.04 g, 8.0 mmol), N,N-diisopropylethylamine (2.79 mL, 16 mmol), and 40 mL DMF were combined with stirring under argon. After one hour, 4-bromo-benzene-1,2-diamine (773 mg, 4.1 mmol) was added and the reaction was stirred overnight. The reaction was diluted with ethyl acetate, washed with water and brine, dried (sodium sulfate), and concentrated. The crude product was dissolved in 50 mL acetic acid and heated at reflux for 4 hours. Upon cooling, the reaction was concentrated and the crude product was purified using RP-HPLC to give 719 mg (73%) of compound 143. MS: 307.9 (M+H⁺); H¹-NMR (DMSO-d₆): δ (ppm) 7.76 (s, 1H), 7.53 (d, 1H, J=8.4 Hz), 7.35 (dd, 1H, J=8.4,1.8 Hz), 2.68 (s, 6H).

### Step 2. 2-(2,4-Dimethyl-thiazol-5-yl)-3H-benzoimidazole-5-boronic acid (144)

A mixture of 143 (719 mg, 2.3 mmol), potassium acetate (1.35 g, 13.8 mmol), [P(Ph₃]₂Pd(II)Cl₂ (322 mg, 0.46 mmol) and bis(neopentylglycolato)diboron (3.12 g, 13.8 mmol) in 12 mL DMSO was heated at 80° C. under argon overnight. The reaction mixture was diluted with ethyl acetate, washed with water and brine, dried (sodium sulfate), and concentrated. The crude product was purified on a 400 mL silica gel pad using ethanol for elution to give 780 mg of an inseparable mixture of compound 144 and unreacted bis(neopentylglycolato)diboron. MS: 274.0 (M+H⁺); H¹-NMR (DMSO-d₆): δ (ppm) 7.87 (s, 1H), 7.52-7.51 (m, 2H), 2.69 (s, 3H), 2.66 (s, 3H).

### Step 3. 3-Cyclohexyl-2-[2-(2,4-dimethyl-thiazol-5-yl)-3H-benzoimidazol-5-yl]-1-(2-morpholin-4-yl-2-oxo-ethyl)-1H-indole-6-carboxylic acid (320)

Compounds 121 (318 mg, 0.69 mmol), 144 (300 mg, 1.1 mmol), tetrakis(triphenyl-phosphino)palladium (40 mg, 0.035 mmol), 1.75 mL saturated NaHCO₃ and 14 mL methanol were combined and heated under argon at 80° C. for four hours. An additional 1.2 equivalents of 144 was added. After 30 min, the solvents were evaporated and the solid was dissolved in 20 mL tetrahydrofuran, and 100 mg sodium hydroxide, 5 mL water and 3.5 mL methanol were added. The reaction mixture was stirred at 55° C. for three hours, neutralized with 1N HCl, and concentrated. The crude product was purified using RP-HPLC followed by conversion to the HCL salt as described for compound 200 to give 122 mg of compound 320 as a light yellow solid. MS: 598.2 (M+H⁺); H¹-NMR (DMSO-d₆): δ (ppm) 7.95 (s, 1H), 7.84-7.76 (m, 2H), 7.64 (d, 1H, J=9.6 Hz), 7.54 (s, 1H), 7.22 (d, 1H, J=8.1 Hz), 4.90 (d, 2H, J=6.3 Hz), 3.51-3.36 (m, 8H), 2.72 (s, 6H), 2.65 (m, 1H), 1.92-1.61 (m, 7H), 1.33-1.20 (m, 3H).

### Example 121

### 3-Cyclohexyl-1-(2-morpholin-4-yl-2-oxo-ethyl)-2-(6-m-tolyl-naphthalen-2-yl)-1H-indole-6-carboxylic acid (321)

Compound 321 was synthesized from compound 150 as described for compound 328 replacing phenylboronic acid with 3-methylphenylboronic acid. MS: 587.2 (M+H⁺); H¹-NMR (DMSO-d₆): δ (ppm) 8.30 (s, 1H), 8.11 (d, 1H, J=8.4 Hz), 8.05 (d, 1H, J=8.7 Hz), 7.99 (s, 1H), 7.92-7.83 (m, 3H), 7.67-7.61 (m, 3H), 7.44-7.38 (m, 2H), 7.22 (d, 1H, J=7.2 Hz), 4.97 (s, 2H), 3.47-3.33 (m, 8H), 2.65 (m, 1H), 2.42 (s, 3H), 1.92-1.61 (m, 7H), 1.27-1.15 (m, 3H).

### Example 122

### 3-Cyclohexyl-2-[6-(2-fluoro-phenyl)-naphthalen-2-yl]-1-(2-morpholin-4-yl-2-oxo-ethyl)-1H-indole-6-carboxylic acid (322)

Compound 322 was synthesized from compound 150 as described for compound 328 replacing phenylboronic acid with 3-fluorophenylboronic acid. MS: 591.2 (M+H⁺); H¹-NMR (DMSO-d₆): δ (ppm) 8.20 (s, 1H), 8.12 (d, 1H, J=8.4 Hz), 8.08 (d, 1H, J=8.4 Hz), 7.99 (s, 1H), 7.90 (s, 1H) 7.85 (d, 1H, J=8.7 Hz), 7.77 (d, 1H, J=8.4 Hz), 7.71-7.64 (m, 2H), 7.49-7.34 (m, 4H), 4.98 (s, 2H), 3.47-3.34 (m, 8H), 2.64 (m, 1H), 1.93-1.61 (m, 7H), 1.27-1.16 (m, 3H).

### Example 123

### 3-Cyclohexyl-2-[2-(2,4-dimethyl-thiazol-5-yl)-1,2,3,4-tetrahydro-quinolin-6-yl]-1-(2-morpholin-4-yl-2-oxo-ethyl)-1H-indole-6-carboxylic acid (323)

90 mg (0.143 mmole) of compound 210 was hydrogenated in 5 mL methanol at 50 psi in the presence of 62 mg PtO₂ for 6 days. The solvent was evaporated and the residue was purified by RP-HPLC to give 8.3 mg compound 323. MS: 613.2 (M+H⁺); H¹-NMR (DMSO-d₆): δ (ppm) 7.86 (d, 1H, J=1.2 Hz), 7.75 (d, 1H, J=8.4 Hz), 7.58 (dd, 1H, J=8.4, 1.5 Hz), 6.83-6.79 (m, 2H), 6.62 (d, 1H, J=8.1 Hz), 4.86 (s, 2H), 4.74 (m, 1H), 3.60-3.33 (m, 8H), 2.92 (m, 1H), 2.69 (m, 2H), 2.58 (s, 3H), 2.31 (s, 3H), 2.07-1.20 (m, 14H).

### Example 124

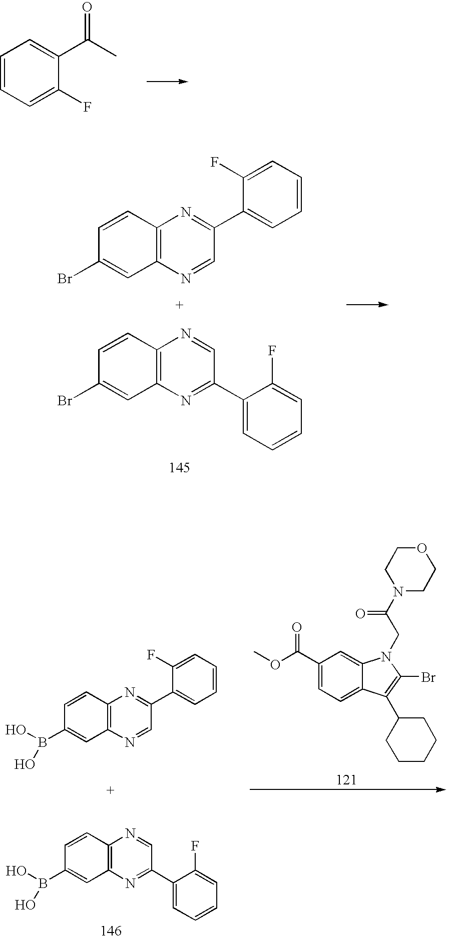

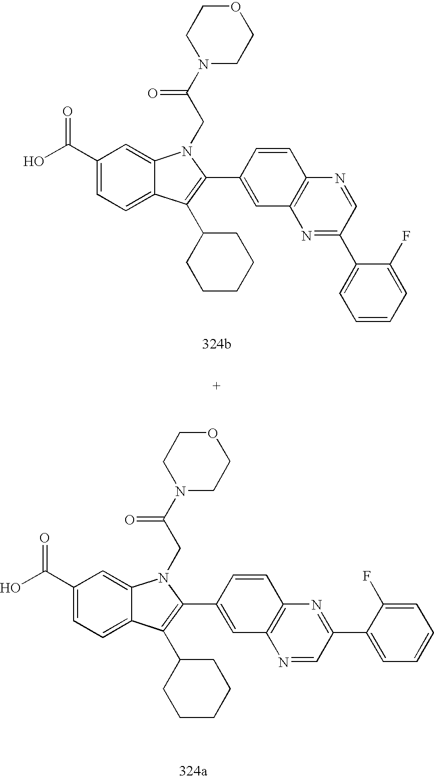

### Step 1. 7-Bromo-2-(2-fluoro-phenyl)-quinoxaline and 6-bromo-2-(2-fluoro-phenyl)-quinoxaline (145)

Selenium dioxide (4.57 mg, 41 mmol) was dissolved in 400 mL dioxane and 12.5 mL water was added. The reaction mixture was heated at 60° C. under argon until the solid dissolved. Fluoroacetophenone (5 mL, 41 mmol) was added and the reaction was heated at 103° C. overnight. The black precipitate was removed by filtration and the warm filtrate was added immediately to 4-bromo-1,2-diaminobenzene (7.7 mg, 41 mmol) in 10 mL ethanol. After stirring for 15 min, the reaction was concentrated and purified using RP-HPLC to give 11.5 mg (92%) of compound 145 as an inseparable mixture of isomers. MS: 302.9 (M+H⁺); H¹-NMR (DMSO-d₆): δ (ppm) 9.32-9.31 (m, 2H), 8.39-8.38 (m, 2H), 8.11-7.99 (m, 6H), 7.65-7.59 (m, 2H), 7.48-7.41 (m, 4H).

### Step 2. 2-(2-fluoro-phenyl)-quinoxaline-7-carboxylic acid and 2-(2-fluoro-phenyl)-quinoxaline-6-carboxylic acid (146)

Compound 145 (300 mg, 0.99 mmol) was dissolved in 10 mL anhydrous THF, triisopropyl borate (685 μL, 2.97 mmol) was added, and the reaction was cooled to −78° C. under argon. Butyl lithium (2.5M, 792 μL, 1.98 mmol) was added slowly. After 30 min, the reaction was treated with 1N HCl and allowed to warm to room temperature. The reaction was extracted with ethyl acetate, washed with brine, dried (sodium sulfate), and concentrated. The crude product was purified using RP-HPLC to give 80.9 mg (31%) of compound 146 as an inseparable mixture of isomers. MS: 269.0 (M+H⁺); H¹-NMR (DMSO-d₆): δ (ppm) 9.29-9.27 (m, 2H), 8.60-8.58 (m, 2H), 8.22-8.17 (m, 2H), 8.10-8.02 (m, 4H), 7.63-7.59 (m, 2H), 7.47-7.41 (m, 4H).

### Step 3. 3-Cyclohexyl-2-[2-(2-fluoro-phenyl)-quinoxalin-6-yl]-1-(2-morpholin-4-yl-2-oxo-ethyl)-1H-indole-6-carboxylic acid (324a); and 3-cyclohexyl-2-[3-(2-fluoro-phenyl)-quinoxalin-6-yl]-1-(2-morpholin-4-yl-2-oxo-ethyl)-1H-indole-6-carboxylic acid (324b)

Compounds 121 (80 mg, 0.17 mmol), 146 (75.5 mg, 0.28 mmol), tetrakis(triphenylphosphino)palladium (10 mg, 0.0087 mmol), 0.65 mL saturated NaHCO₃, 2.6 mL DMF, and 2.6 mL methanol were combined and heated under argon at 80° C. for three hours. The solvents were evaporated and the solid was dissolved in 2.5 mL tetrahydrofuran, and 20 mg sodium hydroxide, 2 mL water and 0.5 mL methanol were added. The reaction mixture was stirred at 55° C. overnight, neutralized with 1N HCl, and concentrated. The crude product was purified using RP-HPLC followed by conversion to the HCL salt as described for compound 200 to give 7 mg (7%) of 324a and 324b as an mixture of isomers. MS: 593.2 (M+H⁺); H¹-NMR (DMSO-d₆): δ (ppm) 9.37 (s, 1H), 9.36 (s, 1H), 8.30 (d, 1H, J=8.7 Hz), 8.28 (d, 1H, J=8.7 Hz), 8.08-8.01 (m, 6H), 7.89-7.77 (m, 4H), 7.69-7.62 (m, 4H), 7.49-7.41 (m, 4H), 5.04 (s, 4H), 3.47-3.33 (m, 16H), 2.67 (m, 2H), 1.95-1.62 (m, 14H), 1.27-1.17 (m, 6H).

### Example 125

### 3-Cyclohexyl-1-(2-morpholin-4-yl-2-oxo-ethyl)-2-(2-pyridin-4-yl-quinolin-6-yl)-1H-indole-6-carboxylic acid (325)

Compound 325 was synthesized from compound 123 as described for compound 207 replacing 5-acetyl salicylaldehyde with 4-acetylpyridine. MS: 575.2 (M+H⁺); H¹-NMR (DMSO-d₆): δ (ppm) 8.86 (br s, 2H), 8.68 (d, 1H, J=9.0 Hz), 8.42-8.39 (m, 3H), 8.26 (d, 1H, J=8.1 Hz), 8.02 (s, 2H), 7.87 (d, 1H, J=8.4 Hz), 7.74-7.65 (m, 2H), 5.02 (s, 2H), 3.43-3.33 (m, 8H), 2.65 (m, 1H), 1.91-1.62 (m, 7H), 1.30-1.16 (m, 3H).

### Example 126

### 3-Cyclohexyl-1-(2-morpholin-4-yl-2-oxo-ethyl)-2-(4-quinolin-2-yl-phenyl)-1H-indole-6-carboxylic acid (326)

Compound 326 was synthesized as described for compound 298 replacing 2-amino-5-bromo-benzaldehyde with 2-amino-benzaldehyde. MS: 574.26 (M+H⁺); H¹-NMR (DMSO-d₆): δ (ppm) 8.57 (d, 1H, J=8.7 Hz), 8.43-8.40 (m, 2H), 8.26 (d, 1H, J=8.1 Hz), 8.11 (d, 1H, J=8.4 Hz), 8.05 (d, 1H, J=7.8 Hz), 7.98 (s, 1H), 7.86-7.79 (m, 2H), 7.66-7.61 (m, 2H), 7.51-7.48 (m, 2H), 4.99 (s, 2H), 3.7-3.30 (m, 8H), 2.66 (m, 1H), 1.94-1.17 (m, 10H).

### Example 127

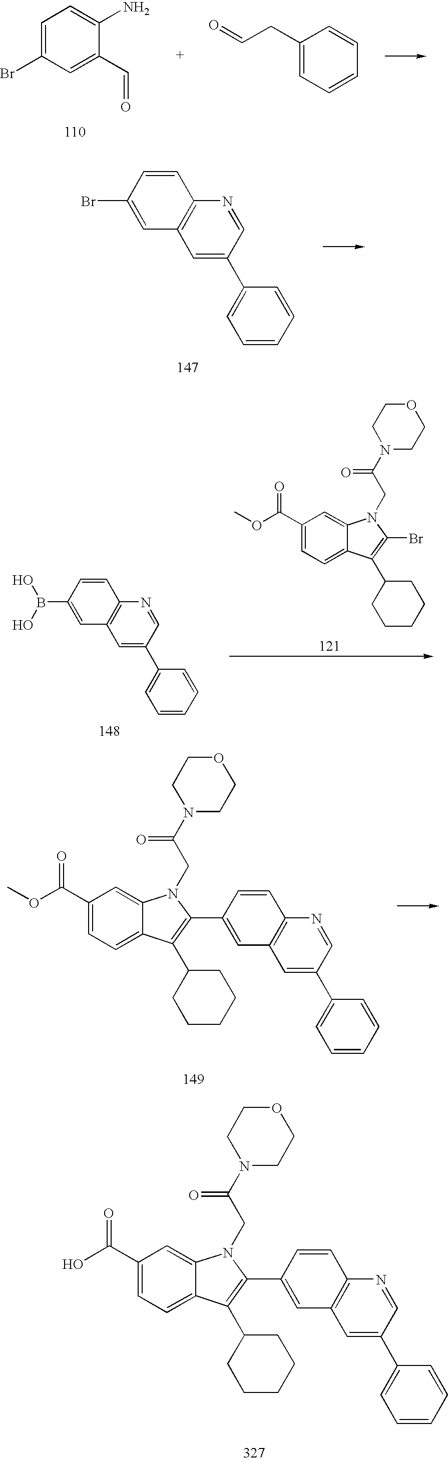

### Step 1. 6-Bromo-3-phenyl-quinoline (147)

Compound 110 (100 mg, 0.5 mmol), phenylacetaldehyde (61 μL, 0.55 mmol), 840 μL 10% KOH/ethanol solution (1.5 mmol KOH) and 5 mL ethanol were combined and heated at reflux for 1 h under argon. The reaction mixture was concentrated and purified using RP-HPLC to give 142 mg (82%) compound 147. MS: 283.9 (M+H⁺); H¹-NMR (DMSO-d₆): δ (ppm) 9.26 (d, 1H, J=2.1 Hz), 8.62 (d, 1H, J=2.1 Hz), 8.32 (d, 1H, J=2.4 Hz), 7.98 (d, 1H, J=9.0 Hz), 7.89-7.84 (m, 3H), 7.58-7.53 (m, 2H), 7.48-7.44 (m, 1H).

### Step 2. 3-Phenyl-quinoline-6-boronic acid (148)

A mixture of 147 (460 mg, 1.6 mmol), potassium acetate (480 mg, 4.9 mmol), [P(Ph₃)]₂Pd(II)Cl₂ (112 mg, 0.16 mmol) and bis(neopentylglycolato)diboron (1.1 g, 4.9 mmol) in 8 mL DMSO was heated at 50° C. under argon overnight. The reaction mixture was diluted with ethyl acetate, washed with water and brine, dried (sodium sulfate), and concentrated. The crude product was purified using RP-HPLC to give 412 mg (98%) of compound 148. MS: 249.0 (M+H⁺); H¹-NMR (DMSO-d₆): δ (ppm) 9.36-9.34 (m, 1H), 8.88-8.86 (m, 1H), 8.52 (d, 1H, J=13.5 Hz), 8.05-8.01 (m, 2H), 7.93-7.87 (m, 2H), 7.59-7.54 (m, 2H), 7.49-7.44 (1H).

### Step 3. 3-Cyclohexyl-1-(2-morpholin-4-yl-2-oxo-ethyl)-2-(3-phenyl-quinolin-6-yl)-1H-indole-6-carboxylic acid methyl ester (149)

Compounds 121 (421 mg, 0.91 mmol), 148 (362 mg, 1.5 mmol), tetrakis(triphenylphosphino)palladium (53 mg, 0.046 mmol), 2.25 mL saturated NaHCO₃ nd 18 mL methanol were combined and heated under argon at 80° C. for four hours. The solvents were evaporated and the crude product was purified on RP-HPLC to give 423 mg (79%) of compound 149. MS: 588.2 (M+H⁺); H¹-NMR (DMSO-d₆): δ (ppm) 9.37 (d, 1H, J=2.1 Hz), 8.84 (d, 1H, J=2.1 Hz), 8.19 (d, 1H, J=8.7 Hz), 8.05-8.03 (m, 2H), 7.94-7.88 (m, 3H), 7.68 (dd, 2H, J=9.3,1.8 Hz), 7.59-7.54 (m, 2H), 7.50-7.45 (m, 2H), 5.02 (s, 2H), 3.86 (s, 3H), 3.44-3.31 (m, 8H), 2.65 (m, 1H), 1.90-1.61 (m, 7H), 1.33-1.20 (m, 3H).

### Step 4. 3-Cyclohexyl-1-(2-morpholin-4-yl-2-oxo-ethyl)-2-(3-phenyl-quinolin-6-yl)-1H-indole-6-carboxylic acid (327)

Compound 149 was dissolved in 10 mL ethanol and 6 mL 1M NaOH was added. The reaction was heated at 95 C.° for 30 minutes under argon. The reaction mixture was neutralized using 7 mL 1N HCl and concentrated. The crude product was purified using RP-HPLC followed by conversion to the HCL salt as described for compound 200 to give 80 mg of compound 327 as an orange solid. MS: 574.2(M+H⁺); H¹-NMR (DMSO-d₆): δ (ppm) 9.44 (d, 1H, J=2.1 Hz), 8.97 (br s, 1H), 8.25 (d, 1H, J=8.4 Hz), 8.10 (s, 1H), 8.01 (s, 1H), 7.95 (s, 1H), 7.93 (s, 1H), 7.87 (d, 1H, J=8.4 Hz), 7.74 (dd, 1H, J=8.7,1.8 Hz), 7.67 (dd, 1H, J=9.0,1.2 Hz), 7.60-7.55 (m, 2H), 7.51-7.46 (m, 1H), 5.01 (s, 2H), 3.44-3.33 (m, 8H), 2.64 (m, 1H), 1.93-1.61 (m, 7H), 1.33-1.20 (m, 3H).

### Example 128

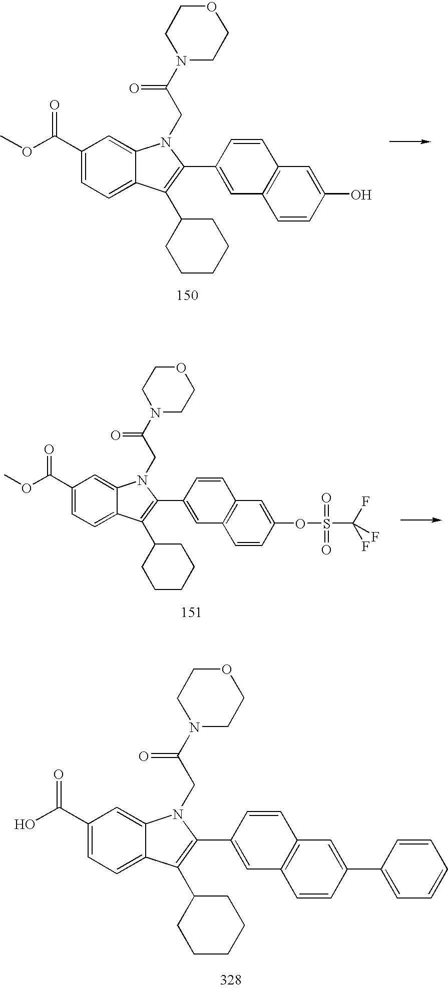

### Step 1. 3-Cyclohexyl-1-(2-morpholin-4-yl-2-oxo-ethyl)-2-(6-trifluoromethane-sulfonyloxy-naphthalen-2-yl)-1H-indole-6-carboxylic acid methyl ester (151)

Compound 150 (200 mg, 0.38 mmol, prepared from compound 121 and 6-hydroxynaphthalen-2-ylboronic acid) and pyridine (460 μL, 0.57 mmol) were dissolved in 4 mL CH₂Cl₂ under argon and cooled to 0° C. Trifluoroacetic anhydride (479 mL, 2.8 mmol) was added. After 5 min, the reaction was allowed to warm to room temperature and washed with saturated sodium bicarbonate and water, and concentrated. The crude product was purified using RP-HPLC to give 134 mg (54%) of compound 151. MS: 659.2 (M+H⁺); H¹-NMR (DMSO-d₆): δ (ppm) 8.25-8.19 (m, 3H), 8.02-8.00 (m, 2H), 7.88 (d, 1H, J=8.4 Hz), 7.71-7.65 (m, 2H), 7.54 (d, 1H, J=8.4 Hz), 4.99 (s, 2H), 3.86 (s, 3H), 3.44-3.31 (m, 8H), 2.63 (m, 1H), 1.87-1.61 (m, 7H), 1.30-1.15 (m, 3H).

Step 2. 3-Cyclohexyl-1-(2-morpholin-4-yl-2-oxo-ethyl)-2-(6-phenyl-naphthalen-2-yl)-1H-indole-6-carboxylic acid (328)

Compound 151 (290 mg, 0.44 mmol), phenylboronic acid (86 mg, 0.71 mmol), tetrakis(triphenylphosphino)palladium (25 mg, 0.022 mmol), 1 mL saturated NaHCO₃ and 9 mL methanol were combined and heated under argon at 80° C. for six hours. The solvents were evaporated and the solid was dissolved in 1 mL tetrahydrofuran, and 10 mg sodium hydroxide, 1 mL water and 0.5 mL methanol were added. The reaction mixture was stirred at 55° C. for six hours, neutralized with 1 mL 1N HCl, and concentrated. The crude product was purified using RP-HPLC followed by conversion to the HCL salt as described for compound 200 to give 70 mg of compound 328 as a light yellow solid. MS: 573.2 (M+H⁺); H¹-NMR (DMSO-d₆): δ (ppm) 8.31 (s, 1H), 8.11 (d, 1H, J=8.7 Hz), 8.07 (d, 1H, J=8.4 Hz), 7.99 (s, 1H), 7.93(dd, 1H, J=8.7,1.8 Hz), 7.87-7.83 (m, 4H), 7.65 (d, 1H, J=9.6 Hz), 7.55-7.50 (m, 2H), 7.45-7.41 (m, 2H), 4.98 (s, 2H), 3.47-3.33 (m, 8H), 2.65 (m, 1H), 1.92-1.61 (m, 7H), 1.27-1.15 (m, 3H).

### Examples 129 and 130

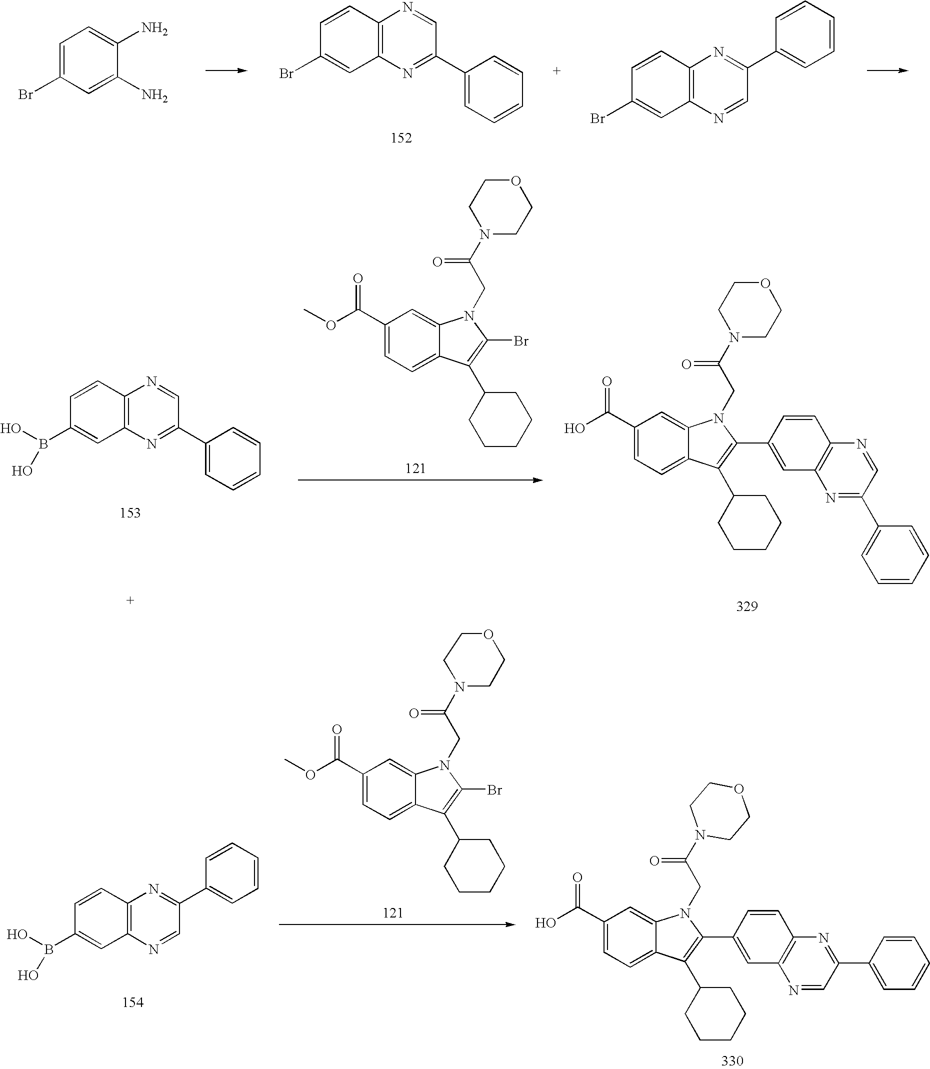

### Step 1. 7-Bromo-2-phenyl-quinoxaline and 6-bromo-2-phenyl-quinoxaline (152)

4-Bromo-1,2-diaminobenzene (500 mg, 2.7 mmol) and phenylglyoxal (357 mg, 2.7 mmol) were stirred in acetic acid. After 5 min, the reaction mixture was concentrated and lyophilized overnight to give 748 mg (97%) of compound 152 as an inseparable mixture of isomers. MS: 285.0 (M+H⁺); H¹-NMR (DMSO-d₆): δ (ppm) 9.61 (s, 2H), 8.36-8.31 (m, 6H), 8.07-7.94 (m, 4H), 7.60-7.56 (m, 6H).

### Step 2. 3-Phenyl-quinoxaline-6-carboxylic acid (153), and 2-phenyl-quinoxaline-6-carboxylic acid (154)

Compound 152 (300 mg, 1.1 mmol) was combined with potassium acetate (309 mg, 3.3 mmol), [P(Ph₃]₂Pd(II)Cl₂ (75 mg, 0.11 mmol) and bis(neopentylglycolato)diboron (714 mg, 3.3 mmol) in 6 mL DMSO and was heated at 80° C. under argon for one hour. The reaction mixture was diluted with ethyl acetate and washed with water and brine, dried (sodium sulfate), and concentrated. The crude products were purified and separated using RP-HPLC to give 88.9 mg (34%) of 153 and 72.2 mg (27%) of 154. Compound 153: MS: 251.0 (M+H⁺); H¹-NMR (DMSO-d₆): δ (ppm) 9.56 (s, 1H), 8.59 (s, 1H), 8.32 (dd, 2H, J=8.0,1.8 Hz), 8.14 (dd, 1H, J=8.4,1.5 Hz), 8.04 (d, 1H, J=8.4 Hz), 7.62-7.55 (m, 3H). Compound 154: MS: 251.0 (M+H⁺); H¹-NMR (DMSO-d₆): δ (ppm) 9.57 (s, 1H), 8.56 (s, 1H), 8.33 (dd, 2H, J=7.5,2.1 Hz), 8.18 (d, 1H, J=9.0 Hz), 8.06 (d, 1H, J=8.7 Hz), 7.63-7.52 (m, 3H).

### Step 3. 3-Cyclohexyl-1-(2-morpholin-4-yl-2-oxo-ethyl)-2-(3-phenyl-quinoxalin-6-yl)-1H-indole-6-carboxylic acid (329)

Compounds 121 (208 mg, 0.46 mmol), 153 (177.8 mg, 0.72 mmol), tetrakis(triphenylphosphino)palladium (26 mg, 0.024 mmol), 1.25 mL saturated NaHCO₃, 10 mL DMF, and 10 mL methanol were combined and heated under argon at 80° C. overnight. The solvents were evaporated and the solid was dissolved in 5 mL tetrahydrofuran, and 100 mg sodium hydroxide, 4 mL water and 1 mL methanol were added. The reaction mixture was stirred at 55° C. for four hours, neutralized with 2N HCl, and concentrated. The crude product was purified using RP-HPLC followed by conversion to the HCL salt as described for compound 200 to give 44 mg (17%) of compound 329 as a red-orange solid. MS: 575.2 (M+H⁺); H¹-NMR (DMSO-d₆): δ (ppm) 9.65 (s, 1H), 8.35 (dd, 2H, J=7.7,2.4 Hz), 8.25 (d, 1H, J=8.4 Hz), 8.02 (s, 2H), 7.88 (d, 1H, J=8.4 Hz), 7.74 (dd, 1H, J=8.4,1.8 Hz), 7.67 (dd, 1H, J=8.7,1.5 Hz), 7.63-7.57 (m, 2H), 5.03 (s, 2H), 3.49-3.30 (m, 8H), 2.68 (m, 1H), 1.95-1.62 (m, 7H), 1.27-1.17 (m, 3H).

### Step 3′. 3-Cyclohexyl-1-(2-morpholin-4-yl-2-oxo-ethyl)-2-(2-phenyl-quinoxalin-6-yl)-1H-indole-6-carboxylic acid (330)

Compound 330 was synthesized from 121 as described for compound 329 replacing 153 with 154. MS: 575.2 (M+H⁺); H¹-NMR (DMSO-d₆): δ (ppm) 9.65 (s, 1H), 8.39-8.35 (m, 2H), 8.27 (d, 1H, J=8.7 Hz), 8.02 (s, 2H), 7.88 (d, 1H, J=8.4 Hz), 7.77 (dd, 1H, J=8.4,1.8 Hz), 7.68-7.58 (m, 4H), 5.04 (s, 2H), 3.49-3.30 (m, 8H), 2.67 (m, 1H), 1.95-1.62 (m, 7H), 1.34-1.17 (m, 3H).

### Example 131

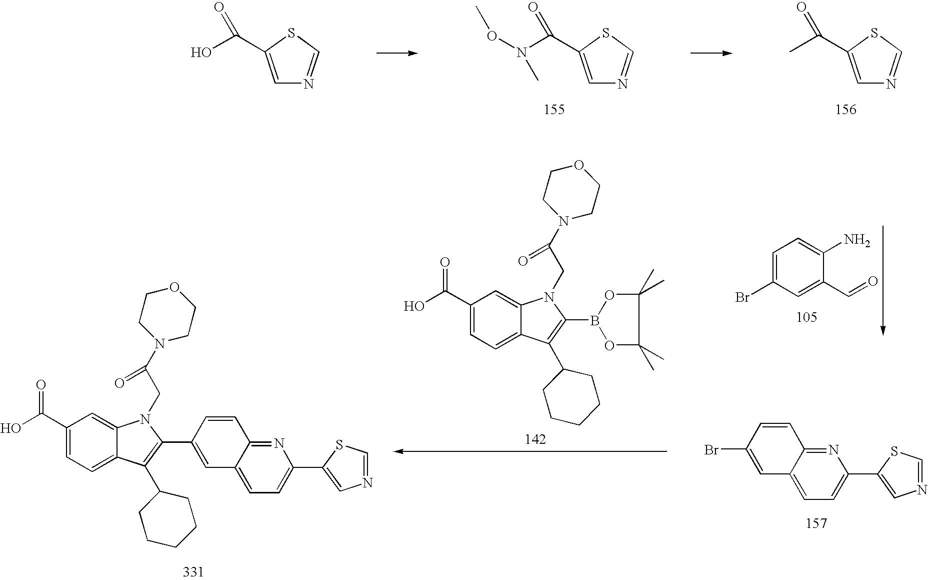

### Step 1. Thiazole-5-carboxylic acid methoxy-methyl-amide (155)

Thiazole-5-carboxylic acid (2 g, 15.48 mmol), HBTU (14 g, 38.8 mmol), and DIEA (16 mL, 92.88 mmol) were dissolved in DMF (50 mL) and stirred at rt until all starting material had been consumed. O,N-Dimethyl-hydroxylamine (6 g, 61.92 mmol) was then added to the reaction mixture and stirred for 16 hours. The reaction was then evaporated to dryness, and purified on silica gel to produce compound 155 (1.7 g, 65% yield). H¹-NMR (DMSO d₆): 9.30 (m, 1H), 8.50 (m, 1H), 3.76 (m, 3H), 3.30 (m, 3H).

### Step 2. 1-Thiazol-5-yl-ethanone (156)

Thiazole-5-carboxylic acid methoxy-methyl-amide (compound 155) (300 mg, 1.74 mmol) was dissolved in anhydrous THF (15 mL), and the temperature was reduced to 0° C. Methyl Grignard (2.5M, 1.16 mL, 3.48 mmol) was then added to the reaction dropwise. The reaction was warmed up to room temperature and stirred for 30 minutes. The reaction was then quenched with EtOH, evaporated to an oil, and taken on to the next reaction. MS: 128.0 (M+H⁺).

### Step 3. 6-Bromo-2-thiazol-5-yl-quinoline (157)

1-Thiazol-5-yl-ethanone (compound 156) (220 mg, 1.74 mmol), compound 105 (316 mg, 1.58 mmol), and KOH (4.74 mmol) were dissolved in EtOH (10 mL) and heated at 85° C. for 16 h. The reaction was then rotovaped down and purified via HPLC to produce compound 157 (200 mg, 43% yield). MS: 290.9 (M+H⁺); H¹-NMR (DMSO d₆): 9.23 (s, 1H), 8.80 (s, 1H), 8.42 (d, 1H, J=8.7 Hz), 8.27 (m, 2H), 7.90 (m, 2H).

### Step 4. 3-Cyclohexyl-1-(2-morpholin-4-yl-2-oxo-ethyl)-2-(2-thiazol-5-yl-quinolin-6-yl)-1H-indole-6-carboxylic acid (331)

6-Bromo-2-thiazol-5-yl-quinoline 157 (60 mg, 0.2 mmol), compound 142 (100 mg, 0.2 mmol), and Palladium Tetrakis (12 mg, 0.01 mmol) were dissolved in a solution of MeOH (2 mL), DMF (2 mL), and saturated sodium bicarbonate (0.8 mL), stirred at 90° C. for 16 h. The reaction was then evaporated to dryness, purified via HPLC, and converted to the HCl salt to produce compound 331 (46 mg, 40% yield). MS: 581.2 (M+H⁺); H¹-NMR (DMSO d₆): 9.21 (s, 1H), 8.83 (s, 1H), 8.53 (d, 1H, J=9 Hz), 8.26 (d, 1H, J=9 Hz), 8.10 (s, 1H, J=9 Hz), 8.00 (s, 1H), 7.93 (s, 1H), 7.85 (d, 1H, J=8.4 Hz), 7.65 (m, 2H), 4.99 (s, 2H), 3.40 (m, 8H), 2.63 (m, 1H), 1.75 (m, 7H), 1.22 (m, 3H).

### Example 132

### 2-Biphenyl-4-yl-3-cyclohexyl-1-(2-morpholin-4-yl-2-oxo-ethyl)-1H-indole-6-carboxylic acid (332)

Following the full procedure and workup for compound 299, 140 (100 mg, 0.16 mmol) was reacted with phenylboronic acid (28 mg, 0.24 mmol) to produce compound 332 (16 mg, 19% yield). MS: 485.2 (M+H⁺); H¹-NMR (DMSO d₆): 7.96 (d, 1H, J=1.2 Hz), 7.80 (m, 5H), 7.64 (dd, 1H, J=8.7 Hz, 1.5 Hz), 7.50 (m, 2H), 7.39 (m, 3H), 4.95 (s, 2H), 3.45 (m, 8H), 2.62 (m, 1H), 1.76 (m, 7H), 1.23 (m, 3H).

### Example 133

### Step 1. 3-Cyclohexyl-2-[2-(2,4-dimethyl-thiazol-5-yl)-quinolin-6-yl]-1H-indole-6-carboxylic acid methyl ester (158)

Following the full procedure and workup for compound 122 using compound 112 (2.04 g, 6 mmol), compound 126 (1.72 g, 6 mmol), tetrakistriphenylphosphine palladium (350 mg), sodium bicarbonate (sat. aq., 16 mL), in methanol (100 mL). Yield 2.3 g, 77%. H¹-NMR (DMSO d₆): 11.70 (s, 1H), 8.55 (s, 1H, J=9.3), 8.11-7.83 (m, 6H), 7.61 (d,1H, J=8.4), 3.85 (s, 3H), 2.96 (m, 1H), 2.72 (s, 3H), 2.66 (m, 1H) 2.00-1.34 (m, 10H).

### Step 2. 3-Cyclohexyl-2-[2-(2,4-dimethyl-thiazol-5-yl)-quinolin-6-yl]-1H-indole-6-carboxylic acid (333)

Compound 158 (25.2 mg) was saponified as described for compound 207. The crude product was purified using RP-HPLC 333. Yield 7.2 mg (30%) MS: 482.2 (M+H⁺); H¹-NMR (DMSO-d₆): δ (ppm) 11.6 (s, 1H), 8.54 (d, 1H, J=8.4), 8.04 (d, 1H, J=8.4), 8.12-7.62 (m, 6H), 7.60 (d, 1H, J=8.4), 2.99 (m, 1H), 2.73 (s, 3H), 2.69 (s, 3H), 1.95-1.10 (m, 10H).

### Example 134

### 3-Cyclohexyl-2-[2-(2,4-dimethyl-thiazol-5-yl)-quinolin-6-yl]-7-methyl-1H-indole-6 carboxylic acid (334)

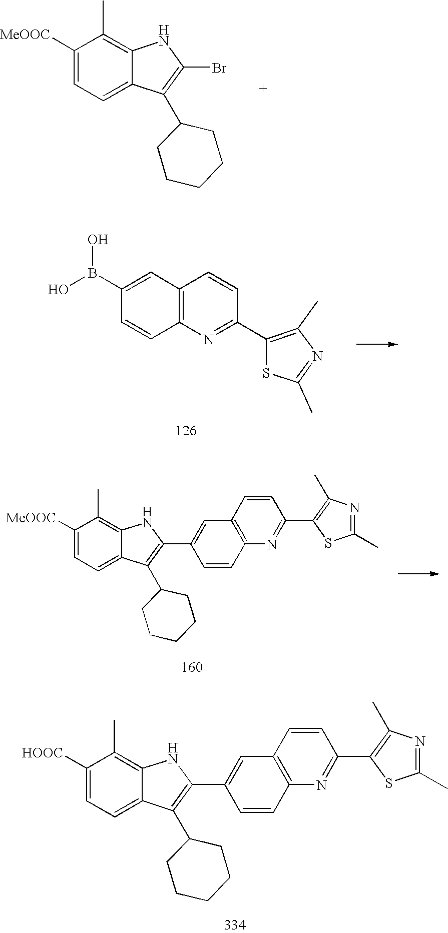

### Step 1. 3-Cyclohexyl-2-[2-(2,4-dimethyl-thiazol-5-yl)-quinolin-6-yl]-7-methyl-1H-indole-6-carboxylic acid methyl ester (160)

A mixture of 2-bromo-3-cyclohexyl-7-methyl-1H-indole-6-carboxylic acid methyl ester (0.63 g, 1.8 mmol, prepared from 3-amino-2-methylbenzoic acid according to WO 2004/065367 A1), compound 126 (0.664 g, 2.34 mmol), and Pd(PPh₃)₄ (0.166 g, 0.144 mmol) in toluene (25 mL) and MeOH (6 mL) in the presence of 2 M NaHCO₃ (2.5 mL) was stirred under Ar at 80° C. for 16 h. After evaporation of solvent, the residue was purified by chromatography on silica gel eluting with CH₂Cl₂-MeOH (80:1) to give a yellow solid compound 160 (0.67 g, 73%). MS: 510.38 (M+H⁺). ¹H-NMR (CDCl₃): δ (ppm) 8.22 (d, 1H, J=8.4 Hz), 8.19 (s, 1H), 8.16 (d, 1H, J=9.0 Hz), 7.90 (br s, 1H), 7.86 (dd, 1H, J=2.1, 8.4 Hz), 7.73-7.70 (m, 2H), 3.92 (s, 3H), 2.99 (m, 1H), 2.81 (s, 3H), 2.79 (s, 3H), 2.74 (s, 3H), 2.07-2.05 (m, 2H), 1.91-1.78 (m, 5H), 1.38-1.35 (m, 3H).

### Step 2. 3-Cyclohexyl-2-[2-(2,4-dimethyl-thiazol-5-yl)-quinolin-6-yl]-7-methyl-1H-indole-6-carboxylic acid (334)

Compound 160 (42 mg, 0.0824 mmol) was dissolved in THF (3 mL) and MeOH (1.5 mL), and 4 N NaOH (0.8 mL) was added. The mixture was stirred at 55° C. for 16 h and cooled down to room temperature. The mixture was neutralized to pH 7 with 5 N HCl. After evaporation of solvent, the residue was purified by reverse phase HPLC to give compound 334 (22.1 mg, 54%). MS: 496.21 (M+H⁺). ¹H-NMR (DMSO-d₆): δ (ppm) 11.29 (s, 1H), 8.56 (d, 1H, J=8.7 Hz), 8.13 (d, 1H, J=1.5 Hz), 8.09 (d, 1H, J=8.7 Hz), 7.93-7.90 (m, 2H), 7.65 (d, 1H, J=8.7 Hz), 7.55 (d, 1H, J=8.4 Hz), 2.99 (m, 1H), 2.78 (s, 3H), 2.74 (s, 3H), 2.69 (s, 3H), 2.07-1.95 (m, 2H), 1.82-1.74 (m, 5H), 1.38-1.28 (m, 3H).

### Example 135

### 3-Cyclohexyl-2-[2-(2,4-dimethyl-thiazol-5-yl)-8-fluoro-quinolin-6-yl]-1-(2-morpholin-4-yl-2-oxo-ethyl)-1H-indole-6-carboxylic acid (335)

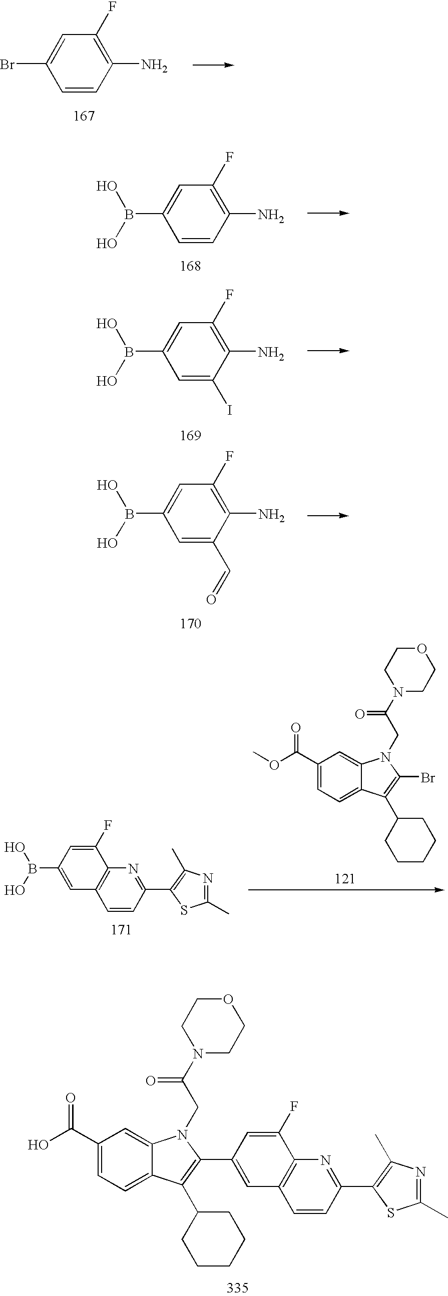

### Step 1. 4-Amino-3-fluoro-boronic acid (168)

A mixture of commercially available 4-bromo-2-fluoroaniline (167, 500 mg, 2.6 mmol), potassium acetate (764 mg, 7.8 mmol), [P(Ph₃)]₂Pd(II)Cl₂ (18 mg, 0.026 mmol) and bis(neopentylglycolato)diboron (1.76 g, 7.8 mmol) in 13 mL DMSO was heated at 60° C. under argon overnight. The reaction mixture was diluted with ethyl acetate, washed with water and brine, dried (sodium sulfate), and concentrated. The crude product was purified using RP-HPLC to give compound 168.

### Step 2. 4-Amino-3-fluoro-5-iodo-boronic acid (169)

Compound 168 is treated with N-iodosuccinimide in acetic acid. The reaction mixture is diluted with ethyl acetate, washed with water and brine, dried (sodium sulfate), and concentrated to give compound 169.

### Step 3. 4-Amino-3-fluoro-5-boronic acid (170)

Compound 169 is dissolved in THF while CO is bubbled through the reaction vessel. Tetrakis(triphenylphosphino)palladium is added and the reaction heated to 50 C. Tributyltin hydride is added. The reaction mixture is diluted with ethyl acetate, washed with water and brine, dried (sodium sulfate), concentrated, and purified to give compound 170.

### Step 4. 2-(2,4-Dimethyl-thiazol-5-yl)-8-fluoro-quinoline-6-boronic acid (171)

A mixture of compound 170, 5-acetyl-2,4-dimethylthiazole, and 10% KOH/ethanol in ethanol is refluxed overnight. The reaction is concentrated, triturated with water, and purified to give compound 171.

### Step 5. 3-Cyclohexyl-2-[2-(2,4-dimethyl-thiazol-5-yl)-8-fluoro-quinolin-6-yl]-1-(2-morpholin-4-yl-2-oxo-ethyl)-1H-indole-6-carboxylic acid (335)

Compounds 171, 121, tetrakis(triphenylphosphino)palladium, saturated NaHCO₃ and methanol are combined and heated under argon at 80° C. The solvents are evaporated and the solid is dissolved in tetrahydrofuran, and sodium hydroxide, water and methanol are added. The reaction mixture is stirred at 55° C., neutralized with 1N HCl, concentrated, and purified to give compound 335.

### Example 136

### 3-Cyclohexyl-2-[2-(2,4-dimethyl-thiazol-5-yl)-7-fluoro-quinolin-6-yl]-1-(2-morpholin-4-yl-2-oxo-ethyl)-1H-indole-6-carboxylic acid (336)

Compound 336 is synthesized in five steps as described for compound 335 replacing 4-bromo-2-fluoroaniline (167) with commercially available 4-bromo-3-fluoroaniline.

### BIOLOGICAL EXAMPLES

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application. Ser. No. 60/004,383, filed on September 1995, described an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1(Supp 4) 18-24.

Screens that measure reductions in kinase activity from HCV drugs were disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al., U.S. Pat. No. 5,739,002 to De Francesco et al., and U.S. Pat. No. 5,597,691 to Houghton et al.

### Example 2

### Replicon Assay

A cell line, ET (Huh-lucubineo-ET) was used for screening of compounds for inhibiting HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding test compound. The compounds were added to the cells to achieve a final concentration of 0.1 nM to 50 μm and a final DMSO concentration of 0.5%. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data was plotted relative to no compound control. Under the same condition, cytotoxicity of the compounds was determined using cell proliferation reagent, WST-1 (Roche, Germany). The compounds showing antiviral activities, but no significant cytotoxicities were chosen to determine IC₅₀ and TC₅₀. For these determinations, a 10 point, 2-fold serial dilution for each compound was used, which spans a concentration range of 1000 fold. IC₅₀ and TC₅₀ values were calculated by fitting % inhibition at each concentration to the following equation:
% inhibition=100%/[(IC50/[I])^{b}+1]
where b is Hill's coefficient.

### Example 3

### Cloning and Expression of Recombinant HCV-NS5b

The coding sequence of NS5b protein was cloned by PCR from pFKI₃₈₉luc/NS3-3′/ET as described by Lohmann, V., et al. (1999) Science 285, 110-113 using the following primers:
aggacatggatccgcggggtcgggcacgagacag (SEQ. ID. NO. 1)
aaggctggcatgcactcaatgtcctacacatggac (SEQ. ID. NO. 2)

The cloned fragment was missing the C terminus 21 amino acid residues. The cloned fragment is inserted into an IPTG-inducible expression plasmid that provides an epitope tag (His)6 at the carboxy terminus of the protein.

The recombinant enzyme was expressed in XL-1 cells and after induction of expression, the protein was purified using affinity chromatography on a nickel-NTA column. Storage condition is 10 mM Tris-HCl pH 7.5, 50 mM NaCl, 0.1 mM EDTA, 1 mM DTT, 20% glycerol at −20° C.

### Example 4

### HCV-NS5b Enzyme Assay

The polymerase activity was assayed by measuring incorporation of radiolabeled UTP into a RNA product using a biotinylated, heteropolymeric template, which includes a portion of the HCV genome. Typically, the assay mixture (34 μL) contains 10 mM Tris-HCl (pH 7.5), 5 mM MgCl₂, 0.2 mM EDTA, 10 mM KCl, 1 unit/μL RNAsin. 1 mM DTT, 10 μM each of NTP, including [³H]-UTP, and 10 ng/μL biotinylated heteropolymeric template. 20× test compound in 2 μl's was then added as a 100% DMSO solution to achieve a final DMSO concentration of 5%. For IC50 determination a 10-point dose response was used. The compounds were serial diluted 2-fold thus covering a range of 1000 fold. Typically for IC50's, compounds were tested starting at 50 uM or 2 μm depending on the potency. Reactions were started with addition of 10×NS5B in 4 μl's and allowed to incubate at 37° C. for 2 hours. Reactions were quenched with 8 μL of 100 mM EDTA and reaction mixtures (30 μL) were transferred to streptavidin-coated scintillation proximity microtiter plates (FlashPlates) and incubated at 4° C. overnight. Incorporation of radioactivity was determined by scintillation counting (cpm). The % Inhibition at a particular concentration was determined using the following equation,
% Inhibition=100−[100*(cpm with inhibitor−bg)/(cpm with no inhibitor−bg)]
where bg was the background with no enzyme.

The following table lists the % inhibition value at 1 μM.

TABLE VII

| Compound No. | % inhibition @ 1 μM |
|---|---|
| 200 | 96.2 |
| 201 | 77.7 |
| 202 | 98.3 |
| 203 | 98.7 |
| 204 | 95.9 |
| 205 | 97.0 |
| 206 | 99.0 |
| 207 | 95.7 |
| 208 | 84.5 |
| 209 | 98.5 |
| 210 | 97.3 |
| 211 | 91.0 |
| 212 | 96.8 |
| 213 | 92.1 |
| 214 | 92.1 |
| 215 | 94.3 |
| 216 | 98.8 |
| 217 | 98.0 |
| 218 | 96.2 |
| 219 | 96.0 |
| 220 | 96.4 |
| 221 | 99.6 |
| 222 | 92.3 |
| 223 | 95.1 |
| 224 | 98.2 |
| 225 | 98.0 |
| 226 | 94.5 |
| 227 | 98.4 |
| 228 | 96.2 |
| 229 | 96.5 |
| 230 | 92.2 |
| 231 | 98.7 |
| 232 | 97.5 |
| 233 | 95.7 |
| 234 | 97.4 |
| 235 | 95.5 |
| 236 | 92.5 |
| 237 | 94.7 |
| 238 | 85.6 |
| 239 | 96.9 |
| 240 | 97.7 |
| 241 | 94.1 |
| 242 | 63.6 |
| 243 | 37.8 |
| 244 | 97.2 |
| 245 | 91.3 |
| 246 | 96.9 |
| 247 | 98.4 |
| 248 | 95.2 |
| 249 | 99.5 |
| 250 | 99.9 |
| 251 | 98.5 |
| 252 | 95.0 |
| 253 | 97.5 |
| 254 | 96.9 |
| 255 | 97.4 |
| 256 | 98.1 |
| 257 | 98.6 |
| 258 | 97.4 |
| 259 | 95.8 |
| 260 | 96.0 |
| 261 | 97.1 |
| 262 | 97.3 |
| 263 | 94.4 |
| 264 | 93.0 |
| 265 | 96.3 |
| 266 | 97.1 |
| 267 | 94.6 |
| 268 | 96.1 |
| 269 | 93.6 |
| 270 | 95.0 |
| 271 | 100.2 |
| 272 | 100.6 |
| 273 | 99.4 |
| 274 | 96.0 |
| 275 | 98.9 |
| 276 | 97.6 |
| 277 | 96.3 |
| 278 | 91.8 |
| 279 | 100.1 |
| 280 | 97.7 |
| 281 | 77.6 |
| 282 | 87.8 |
| 284 | 70.1 |
| 286 | 85.7 |
| 287 | 95.2 |
| 289 | 95.7 |
| 290 | 79.1 |
| 291 | 96.4 |
| 292 | 98.7 |
| 293 | 69.2 |
| 298 | 101.0 |
| 299 | 94.9 |
| 300 | 98.2 |
| 301 | 97.0 |
| 302 | 99.3 |
| 303 | 98.0 |
| 304 | 98.5 |
| 305 | 98.5 |
| 306 | 97.8 |
| 307 | 97.4 |
| 308 | 97.5 |
| 309 | 98.8 |
| 310 | 99.6 |
| 311 | 101.8 |
| 312 | 99.5 |
| 313 | 99.6 |
| 314 | 98.0 |
| 315 | 95.8 |
| 316 | 101.3 |
| 317 | 96.7 |
| 318 | 98.1 |
| 319 | 74.3 |
| 320 | 84.1 |
| 321 | 98.2 |
| 322 | 95.1 |
| 323 | 97.6 |
| 324a/b | 99.9 |
| 325 | 93.6 |
| 326 | 99.8 |
| 327 | 96.3 |
| 328 | 100.8 |
| 329 | 99.8 |
| 330 | 96.8 |
| 331 | 97.0 |
| 332 | 100.2 |
| 333 | 95.1 |
| 334 | 86.9 |

### FORMULATION EXAMPLES The following are representative pharmaceutical formulations containing a compound of formula I.

### Formulation Example 1

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4

### Injectable Formulation

The following ingredients are mixed to form an injectable formulation.

| Ingredient | Amount |
|---|---|
| compound of this invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| Compound of the invention | 500 mg |
| Witepsol ® H-15 | balance |

What is claimed is:

1. A compound of formula Ia: 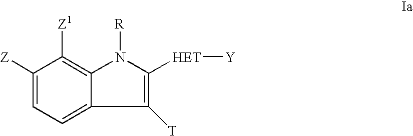
wherein:
Y is selected from the group consisting of substituted aryl and substituted heteroaryl;
HET is selected from the group consisting of a 6-membered arylene ring, a 6-membered heteroarylene ring containing 1, 2, or 3 heteroatoms selected from N, O, or S, and a bicyclic ring having the formula 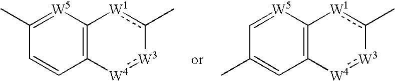
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;
R is selected from the group consisting of hydrogen, alkyl, and substituted alkyl;
T is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl;
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted with one to three groups selected from halo, hydroxy, carboxy, carboxy ester, alkyl, alkoxy, amino, and substituted amino; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e);
Z¹ is selected from the group consisting of hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino; or a
pharmaceutically acceptable salt, partial salt, or tautomer thereof.

2. A compound of claim 1 wherein Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized.

3. A compound of claim 2 wherein Y is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphen-2-yl, 4-methoxybiphen-2-yl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

4. A compound of claim 2 wherein said substituted phenyl is substituted with one to three substitutents selected from the group consisting of halo, heteroaryl, hydroxy, nitro, cyano, alkyl, substituted alkyl, alkenyl, alkoxy, substituted alkoxy, acyl, acylamino, aminoacyl, amino, substituted amino, carboxy, and carboxy ester.

5. A compound of claim 2 wherein Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl.

6. A compound of claim 5 wherein Y is substituted with one to three subsitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester.

7. A compound of claim 6 wherein Y is 2,4-dimethylthiazol-5-yl.

8. A compound of claim 1 wherein T is cycloalkyl.

9. A compound of claim 8 wherein T is cyclohexyl.

10. A compound of claim 1 wherein Z is carboxy, carboxy ester, carboxylic acid isostere, —C(O)NR⁸R⁹, or —C(O)NHS(O)₂R⁴, wherein R⁸ and R⁹ are as defined in claim 1 and R⁴ is alkyl or aryl.

11. A compound of claim 10 wherein Z is carboxy, methyl carboxylate, ethyl carboxylate, 6-(β-D-glucuronic acid)ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl.

12. A compound of claim 11 wherein Z is carboxy.

13. A compound of claim 1 wherein Z¹ is selected from the group consisting of hydrogen, halo, alkyl, and haloalkyl.

14. A compound of claim 1 wherein R is substituted alkyl, wherein said substituted alkyl is selected from the group consisting of aminoalkyl, substituted aminoalkyl, arylalkyl, substituted arylalkyl, heteroarylalkyl, substituted heteroarylalkyl, heterocyclylalkyl, substituted heterocyclylalkyl, —CH₂COOH, and —CH₂CONR²R¹³, wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

15. A compound of claim 1 wherein R is —CH₂CONR¹²R¹³ and at least one of R¹² or R¹³ is alkyl, substituted alkyl, or heteroaryl.

16. A compound of claim 15 wherein at least one of R¹² or R¹³ is methyl, carboxymethyl, 2-hydroxyethyl, 2-morpholin-4-ylethyl, or tetrazoyl-5-yl.

17. A compound of claim 14 wherein R is —CH₂CONR¹²R¹³ and R¹² and R¹³ and the nitrogen atom to which they are attached form a substituted or unsubstituted heterocyclic ring.

18. A compound of claim 17 wherein R¹² and R¹³ and the nitrogen atom to which they are attached form a substituted or unsubstituted morpholino, substituted or unsubstituted piperidinyl, or a substituted or unsubstituted pyrrolidinyl ring.

19. A compound of claim 18 wherein said substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

20. The compound of claim 14 wherein R is selected from N,N-dimethylamino-carbonylmethyl, [N-(4-hydroxy-1,1-dioxidotetrahydro-3-thienyl)amino]-carbonylmethyl, (cyclopropylmethylamino)-carbonylmethyl, (prop-2-yn-1-ylamino)-carbonylmethyl, (2-(morpholino)eth-1-ylamino)-carbonylmethyl, (phenylsulfonylamino)-carbonylmethyl, [N-benzylamino]-carbonylmethyl, (N-(4-methylsulfonyl-benzyl)amino)-carbonylmethyl, (tryptophanyl)-carbonylmethyl, (tyrosine)-carbonylmethyl, (N-(1-carboxyprop-1-ylamino)-carbonylmethyl, (N-(2-carboxyeth-1-yl)-amino)-carbonylmethyl, (N-(4-carboxybenzyl)-amino)-carbonylmethyl, N-[3-(N′-(4-(acrylic acid)-phenyl)carboxamido)pyrrolidin-3-yl]amino-carbonylmethyl, N-[4-(N′-(4-(acrylic acid)-phenyl)carboxamido)piperidin-4-yl]amino-carbonylmethyl, [2-(N,N-dimethylamino)eth-1-ylamino]-carbonylmethyl, [(1-(5-methyl-4H-1,2,4-triazol-3-yl)ethyl)amino]-carbonylmethyl, (1-methyl-1-[N-(1-methyl-2-carboxy-1H-indol-5-yl)aminocarbonyl]eth-1-ylamino-carbonylmethyl, [N-(1-methylpyrrolidin-3-yl-ethyl)-amino]-carbonylmethyl, (1-methyl-1-[N-(4-(acrylic acid)phenyl)aminocarbonyl]eth-1-ylamino-carbonylmethyl, (1-methyl-1-[N-(4-(2-carboxy-furan-5-yl)phenyl)aminocarbonyl]eth-1-ylamino-carbonylmethyl, (1-methyl-1-[N-(4-(4-carboxy-thiazol-2-yl)phenyl)aminocarbonyl]eth-1-ylamino-carbonylmethyl, (2-(4-methylpiperazin-1-yl)eth-1-ylamino)-carbonylmethyl, [(1-methylpyrrolidin-3-yl)methylamino]-carbonylmethyl, [N-(1-methylpiperidin-3-yl-methyl)-amino]-carbonylmethyl, (1-piperidin-1-ylcyclopentyl)methylamino]-carbonylmethyl, (1-(acetyl)-pyrrolidin-2-ylmethyl)amino)-carbonylmethyl, [(2-(N,N-dimethylamino)-carbonyl)methylamino]-carbonylmethyl, [N-(1,1-dioxidotetrahydro-3-thienyl)methylamino]-carbonylmethyl, (N-methyl-N-cyclohexyl-amino)-carbonylmethyl, (N-methyl-N-carboxymethyl-amino)-carbonylmethyl, [N-methyl-N-benzyl-amino]-carbonylmethyl, (N-methyl-N—(N′,N′-dimethylaminoacetyl)-amino)-carbonylmethyl, [N-methyl-N-phenyl-amino]-carbonylmethyl, (N-methyl-N-isopropyl-amino)-carbonylmethyl, (N-methyl-N—(N′-methylpiperidin-4-yl)amino)-carbonylmethyl, [N-methyl-N-(1-methylpiperidin-4-yl)amino]-carbonylmethyl, [N-methyl-N-(1-methylpiperidin-4-yl-methyl)-amino]-carbonylmethyl, [N-methyl-N-(1-methylpiperidin-3-yl-methyl)-amino]-carbonylmethyl, [N-methyl-N-(1-methylpyrazin-2-yl-methyl)-amino]-carbonylmethyl, [N-methyl-N-(5-methyl-1H-imidazol-2-ylmethyl)-amino]-carbonylmethyl, (N-methyl-N-[2-(hydroxy)eth-1-yl]amino)-carbonylmethyl, (N-methyl-N-[2-(N′,N′-dimethylamino)eth-1-yl]amino)-carbonylmethy, N-methyl-N-[2-(N′,N′-diethylamino)eth-1-yl]amino)-carbonylmethyl, (N-methyl-N-[2-(pyridin-2-yl)eth-1-yl]amino)-carbonylmethyl, (N-methyl-N-[2-(pyridin-4-yl)eth-1-yl]amino)-carbonylmethyl, [N-methyl-N-(1-(1,3-thiazol-2-yl)ethyl)-amino]-carbonylmethyl, (N-methyl-N-[3-(N′,N′-dimethylamino)prop-1-yl]amino)-carbonylmethyl, (N-methyl-N-(1-carboxy-2-methylprop-1-yl)-amino)-carbonylmethyl, (N-ethyl-N-propyl-amino)-carbonylmethyl, (N-ethyl-N-[2-(methoxy)eth-1-yl]amino)-carbonylmethyl, (N-ethyl-N-[2-(N′,N′-diethylamino)eth-1-yl]amino)-carbonylmethyl, [7-methyl-2,7-diazaspiro[4.4]non-2-yl]-carbonylmethyl, (5-methyl-2,5-diazabicyclo[2.2.1]heptyl-2-yl)-carbonylmethyl, (4-methyl-1,4-diazepan-1-yl)-carbonylmethyl, (piperidinyl)-carbonylmethyl, (4-carboxy-piperidinyl)-carbonylmethyl, (3-carboxypiperidinyl)-carbonylmethyl, (4-hydroxypiperidinyl)-carbonylmethyl, (4-(2-hydroxyeth-1-yl)piperidin-1-yl)-carbonylmethyl, [4-(N,N-dimethylamino)-piperidin-1-yl]-carbonylmethyl, (3-(N,N-dimethylamino)-methylpiperidin-1-yl)-carbonylmethyl, (2-(2-(N,N-dimethylamino)-eth-1-yl)piperidin-1-yl)-carbonylmethyl, [4-(4-methyl-4H-1,2,4-triazol-3-yl)piperidin-1-yl]-carbonylmethyl, (4-pyrrolidinyl-piperidinyl)-carbonylmethyl, (3-pyrrolidinyl-piperidinyl)-carbonylmethyl, [4-(N,N-diethylamino)-piperidin-1-yl]-carbonylmethyl, (4-(azetidin-1-yl)-piperidin-1-yl)-carbonylmethyl, (4-(piperidin-1-yl)-piperidin-1-yl)-carbonylmethyl, (hexahydropyrrolo[1,2-a]pyrazin-2(1H)-yl)-carbonylmethyl, [(2-(N,N-dimethylamino)-methyl)morpholino]-carbonylmethyl, (3,5-dimethylmorpholino)-carbonylmethyl, (thiomorpholino)-carbonylmethyl, morpholino-carbonylmethyl, (pyrrolidinyl)-carbonylmethyl, (2-carboxy-pyrrolidin-1-yl)-carbonylmethyl, (2-(carboxy)-4-hydroxy-pyrrolidin-1-yl)-carbonylmethyl, (2-carboxamide-pyrrolidin-1-yl)-carbonylmethyl, (2-(N,N-dimethylaminocarbonyl)-pyrrolidin-1-yl)-carbonylmethyl, (3-(N′,N′-dimethylamino)-pyrrolidin-1-yl)-carbonylmethyl, (3-(N′,N′-diethylamino)-pyrrolidin-1-yl)-carbonylmethyl, (3-(pyridin-3-yl)-pyrrolidin-1-yl)-carbonylmethyl, (2-pyidin-4-ylpyrrolidin-1-yl)-carbonylmethyl, piperazin-1-yl-carbonylmethyl, (4-methylpiperazinyl)-carbonylmethyl, (4-(carboxymethyl)-piperazin-1-yl)-carbonylmethyl, (4-(2-hydroxyeth-1-yl)piperazin-1-yl)-carbonylmethyl, (4-(isopropyl)piperazin-1-yl)-carbonylmethyl, (4-(2-methoxyeth-1-yl)piperazin-1-yl)-carbonylmethyl, (4-(ethyl)piperazin-1-yl)-carbonylmethyl, (4-(N′,N′-dimethylaminoacetyl)-piperazin-1-yl)-carbonylmethyl, and (4-(6-methoxypyridin-2-yl)piperazin-1-yl)-carbonylmethyl.

21. A compound of claim 1 wherein t is 0.

22. A compound of claim 1 wherein t is 1 and X is amino, nitro, methyl or halo.

23. A compound of claims 1 wherein HET is 1,4-phenylene optionally substituted with (X)ₜ where X and t are as defined in claim 1.

24. A compound of claims 1 wherein HET is 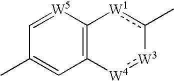
optionally substituted with (X)ₜ where X, t, W¹, W³, W⁴, and W⁵ are as defined in claim 1.

25. A compound of claim 24 wherein W¹ is nitrogen.

26. A compound of claim 25 where HET is selected from the group consisting of 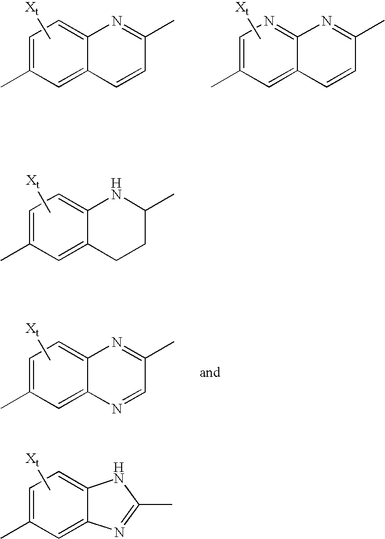
where X and t are as defined in claim 1.

27. A compound having formula Ib: 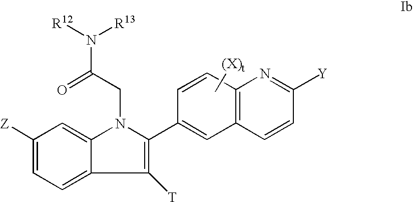
wherein:
Y is selected from the group consisting of substituted aryl and substituted heteroaryl;
X is independently selected from the group consisting of amino, nitro, alkyl, haloalkyl, and halo;
t is an integer equal to 0, 1 or 2;
T is selected from the group consisting of cyclohexyl and cyclopentyl;
R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms;
Z is selected from the group consisting of carboxy, carboxy ester, and a carboxylic acid isostere; or a
pharmaceutically acceptable salt, partial salt, or tautomer thereof.

28. A compound of claim 27 wherein Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized.

29. A compound of claim 28 wherein Y is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphen-2-yl, 4-methoxybiphen-2-yl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

30. A compound of claim 28 wherein said substituted phenyl is substituted with one to three substitutents selected from the group consisting of halo, heteroaryl, hydroxy, nitro, cyano, alkyl, substituted alkyl, alkenyl, alkoxy, substituted alkoxy, acyl, acylamino, aminoacyl, amino, substituted amino, carboxy, and carboxy ester.

31. A compound of claim 28 wherein Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl.

32. A compound of claim 31 wherein Y is substituted with one to three subsitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester.

33. A compound of claim 32 wherein Y is 2,4-dimethylthiazol-5-yl.

34. A compound of claim 27 wherein T is cyclohexyl.

35. A compound of claim 34 wherein Z is methyl carboxylate, ethyl carboxylate, 6-(β-D-glucuronic acid)ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl.

36. A compound of claim 35 wherein Z is carboxy.

37. A compound of claim 27 wherein at least one of R¹² or R¹³ is alkyl, substituted alkyl, or heteroaryl.

38. A compound of claim 37 wherein at least one of R¹² or R¹³ is methyl, carboxymethyl, 2-hydroxyethyl, 2-morpholin-4-ylethyl, or tetrazoyl-5-yl.

39. A compound of claim 27 wherein R¹² and R¹³ and the nitrogen atom to which they are attached form a substituted or unsubstituted heterocyclic ring.

40. A compound of claim 39 wherein R¹² and R¹³ and the nitrogen atom to which they are attached form a substituted or unsubstituted morpholino, substituted or unsubstituted piperidinyl, or a substituted or unsubstituted pyrrolidinyl ring.

41. A compound of claim 40 wherein said substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

42. A compound of claim 27 wherein R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-benzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido)pyrrolidin-3-yl]amino, N-[4-(N′-(4-(acrylic acid)-phenyl)carboxamido)piperidin-4-yl]amino, 2-(N,N-dimethylamino)eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, (1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N—(N′,N′-dimethylaminoacetyl)-amino, N-methyl-N-phenyl-amino, N-methyl-N-isopropyl-amino, N-methyl-N—(N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino)eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino)-piperidin-1-yl, 3-(N,N-dimethylamino)-methylpiperidin-1-yl, 2-(2-(N,N-dimethylamino)-eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl)-pyrrolidin-1-yl, 3-(N′,N′-dimethylamino)-pyrrolidin-1-yl, 3-(N′,N′-diethylamino)-pyrrolidin-1-yl, 3-(pyridin-3-yl)-pyrrolidin-1-yl, 2-pyidin-4-ylpyrrolidin-1-y, piperazin-1-yl, 4-methylpiperazinyl, 4-(carboxymethyl)-piperazin-1-yl, 4-(2-hydroxyeth-1-yl)piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, and 4-(6-methoxypyridin-2-yl)piperazin-1-yl.

43. A compound of claim 27 wherein t is 0.

44. A compound of claim 27 wherein t is 1 and X is amino, nitro, methyl, or halo.

45. A compound of formula III: 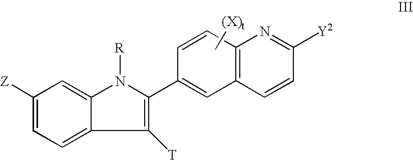
wherein:
Y² is selected from optionally substituted aryl and optionally substituted heteroaryl;
Z is selected from:
(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;
(b) carboxy and carboxy ester;
(c) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(d) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted halo, hydroxy, carboxy, alkyl, alkoxy, amino, substituted amino; or alternatively, R²¹ and R²² or R²² and R²³ together with the atoms bound thereto join together to form an optionally substituted heterocyclic group;
(e) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R² is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(f) —C(X²)—N(R³)CR²⁵R²⁶R²⁷, wherein X² and R³ are defined above, and R²⁵, R²⁶ and R²⁷ are substituted aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(g) carboxylic acid isostere;
R is hydrogen, alkyl, substituted alkyl, cycloalkyl, substituted cycloalkyl, heterocyclic, substituted heterocyclic, aryl, substituted aryl, heteroaryl, substituted heteroaryl;
T is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, and —NR¹⁴R¹⁵;
where each R¹⁴ and R¹⁵ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or alternatively, R¹⁴ and R¹⁵ may optionally be joined together with the nitrogen atom bound thereto to form a heterocyclic, substituted heterocyclic, heteroaryl or substituted heteroaryl;
X is chosen from alkyl, substituted alkyl, alkoxy, substituted alkoxy, halo, hydroxy, and nitro;
t is an integer equal to 0, 1 or 2; and
pharmaceutically acceptable salts, partial salts, or tautomers thereof.

46. A compound selected from the group consisting of:
2-[2-(4′-chloro-4-methoxy-biphen-2-yl)-quinolin-6-yl]-3-cyclohexyl-1H-indole-6-carboxylic acid (Compound 200);
1-[N-morpholinocarbonylmethyl]-2-[2-(4′-chloro-4-methoxy-biphen-2-yl)-quinolin-6-yl]-3-cyclohexyl-1H-indole-6-carboxylic acid (Compound 203);
1-[carboxymethyl]-2-[2-(4′-chloro-4-methoxy-biphen-2-yl)-quinolin-6-yl]-3-cyclohexyl-1H-indole-6-carboxylic acid (Compound 202);
1-[N-piperazinylcarbonylmethyl]-2-[2-(4′-chloro-4-methoxy-biphen-2-yl)-quinolin-6-yl]-3-cyclohexyl-1H-indole-6-carboxylic acid (Compound 204);
1-[(4-pyrrolidin-1-yl-piperidin-1-yl)carbonylmethyl]-2-[2-(4′-chloro-4-methoxy-biphen-2-yl)-quinolin-6-yl]-3-cyclohexyl-1H-indole-6-carboxylic acid (Compound 205);
1-[N,N-dimethylaminocarbonyl-methyl]-2-[2-(4′-chloro-4-methoxy-biphen-2-yl)-quinolin-6-yl]-3-cyclohexyl-1H-indole-6carboxylic acid (Compound 206);
1-[carboxymethyl]-2-[2-(4′-chloro-4-methoxybiphen-2-yl)-quinolin-6-yl]-3-cyclohexyl-1H-indole-6-carboxylic acid methyl ester (Compound 201);
1-[N-morpholinocarbonylmethyl]-2-[2-(4-(1H-imidazolyl)phenyl)-quinolin-6-yl]-3-cyclohexyl-1H-indole-6-carboxylic acid (Compound 209);
1-[N-morpholinocarbonylmethyl]-2-[2-(2,4-dimethylthiazol-5-yl)-quinolin-6-yl]-3-cyclohexyl-1H-indole-6-carboxylic acid (Compound 210);
1-[carboxymethyl]-2-[2-(2,4-dimethylthiazol-5-yl)-quinolin-6-yl]-3-cyclohexyl-1H-indole-6-carboxylic acid (Compound 211);
1-[N-morpholinocarbonylmethyl]-2-[2-(3-amido-4-hydroxy-phenyl)-quinolin-6-yl]-3-cyclohexyl-1H-indole-6-carboxylic acid (Compound 207);
1-carboxylmethyl-2-[2-(3-Carbamoyl-4-hydroxy-phenyl)-quinolin-6-yl]-3-cyclohexyl-1H-indole-6-carboxylic acid (Compound 208);
1-[N-morpholinocarbonylmethyl]-2-[2-(pyrrol-3-yl)-quinolin-6-yl]-3-cyclohexyl-1H-indole-6-carboxylic acid;
1-[N-morpholinocarbonylmethyl]-2-[2-(3-bromo-4-aminophenyl)-quinolin-6-yl]-3-cyclohexyl-1H-indole-6-carboxylic acid;
1-[N-morpholinocarbonylmethyl]-2-[2-(2-hydroxy-6-methoxy-phenyl)-quinolin-6-yl]-3-cyclohexyl-1H-indole-6-carboxylic acid;
1-[N-morpholinocarbonylmethyl]-2-[2-(4-acetylaminophenyl)-quinolin-6-yl]-3-cyclohexyl-1H-indole-6-carboxylic acid;
1-[N-morpholinocarbonylmethyl]-2-[2-(2,4-dihydroxyphenyl)-quinolin-6-yl-3-cyclohexyl-1H-indole-6-carboxylic acid;
1-[N-morpholinocarbonylmethyl]-2-[2-(2,4-dimethoxy-6-hydroxyphenyl)-quinolin-6-yl]-3-cyclohexyl-1H-indole-6-carboxylic acid;
1-N-morpholinocarbonylmethyl]-2-[2-(7-hydroxybenzofuran-2-yl)-quinolin-6-yl]-3-cyclohexyl-1H-indole-6-carboxylic acid;
1-[N-morpholinocarbonylmethyl]-2-[2-(2,4-dimethylthiazol-5-yl)-quinolin-6-yl]-3-cyclohexyl-1H-indole-6-1H-tetrazol-5-yl;
1-[N-morpholinocarbonylmethyl]-2-[2-(2,4-dimethylthiazol-5-yl)-quinolin-6-yl]-3-cyclohexyl-1H-indole-6-(5-oxo-4,5-dihydro-1,2,4-oxadiazol-3-yl);
1-[N-morpholinocarbonylmethyl]-2-[2-(2,4-dimethylthiazol-5-yl)-quinolin-6-yl]-3-cyclohexyl-1H-indole-6-(methylsulfonylaminocarbonyl);
1-[N-morpholinocarbonylmethyl]-2-[2-(2,4-dimethylthiazol-5-yl)-quinolin-6-yl]-3-cyclohexyl-1H-indole-6-(trifluoromethyl-sulfonylaminocarbonyl);
1-[N-morpholinocarbonylmethyl]-2-[2-(2,4-dimethylthiazol-5-yl)-quinolin-6-yl]-3-cyclohexyl-1H-indole-6-(phenyl-sulfonylaminocarbonyl);
1-[N-morpholinocarbonylmethyl]-2-[2-(2,4-dimethylthiazol-5-yl)-quinolin-6-yl]-3-cyclohexyl-7-methyl-1H-indole-6-carboxylic acid;
1-[N-morpholinocarbonylmethyl]-2-[2-(2,4-dimethylthiazol-5-yl)-quinolin-6-yl]-3-cyclohexyl-7-fluoro-1H-indole-6-carboxylic acid;
1-[N-morpholinocarbonyl]-2-[2-(2,4-dimethylthiazol-5-yl)-quinolin-6-yl]-3-cyclohexyl-1H-indole-6-carboxylic acid;
1-[N-morpholinocarbonylmethyl]-2-[2-(2,4-dimethylthiazol-5-yl)-7-fluoro-quinolin-6-yl]-3-cyclohexyl-1H-indole-6-carboxylic acid;
1-[N-morpholinocarbonylmethyl]-2-[2-(2,4-dimethylthiazol-5-yl)-7-methyl-quinolin-6-yl]-3-cyclohexyl-1H-indole-6-carboxylic acid;
1-[N-morpholinocarbonylmethyl]-2-[2-(2,4-dimethylthiazol-5-yl)-5-fluoro-quinolin-6-yl]-3-cyclohexyl-1H-indole-6-carboxylic acid;
1-[N-morpholinocarbonylmethyl]-2-[2-(2,4-dimethylthiazol-5-yl)-7-fluoro-quinolin-6-yl]-3-cyclohexyl-7-methyl-1H-indole-6-carboxylic acid;
1-[N-morpholinocarbonylmethyl]-2-[2-(pyrid-2-yl)-quinolin-6-yl]-3-cyclohexyl-1H-indole-6-carboxylic acid (Compound 217);
1-[N-morpholinocarbonylmethyl]-2-[2-(pyrazin-2-yl)-quinolin-6-yl]-3-cyclohexyl-1H-indole-6-carboxylic acid (Compound 218);
1-[N-morpholinocarbonylmethyl]-2-[2-(pyrrol-2-yl)-quinolin-6-yl]-3-cyclohexyl-1H-indole-6-carboxylic acid (Compound 219);
1-[N-morpholinocarbonylmethyl]-2-[2-phenylquinolin-6-yl]-3-cyclohexyl-1H-indole-6-carboxylic acid (Compound 220);
1-[N,N-dimethylaminocarbonyl-methyl]-2-[2-(2,4-dimethylthiazol-5-yl)-quinolin-6-yl]-3-cyclohexyl-1H-indole-6-carboxylic acid (Compound 212);
1-[(N-carboxymethycarbamoyl)-methyl]-[2-(2,4-dimethyl-thiazol-5-yl)-quinolin-6-yl]-3-cyclohexyl-1H-indole-6-carboxylic acid (Compound 213);
1-[2-oxo-2-(4-pyrrolidin-1-yl-piperidin-1-yl)-ethyl]-2-[2-(2,4-dimethylthiazol-5-yl)-quinolin-6-yl]-3-cyclohexyl-1H-indole-6-carboxylic acid (Compound 214);
1-[(2-hydroxyethylcarbamoyl)-methyl]-2-[2-(2,4-dimethyl-thiazol-5-yl)-quinolin-6-yl]-3-cyclohexyl-1H-indole-6-carboxylic acid (Compound 215);
1-(2-oxo-2-piperidin-1-yl-ethyl)-2-[2-(2,4-dimethylthiazol-5-yl)-3-cyclohexylquinolin-6-yl]-1H-indole-6-carboxylic acid (Compound 216);
1-[N-morpholinocarbonylmethyl]-2-(2-furan-2-yl-quinolin-6-yl)-3-cyclohexyl-1H-indole-6-carboxylic acid (Compound 222);
1-(N-morpholinocarbonylmethyl]-2-[2-(5-methyl-furan-2-yl)-quinolin-6-yl]-3-cyclohexyl-1H-indole-6-carboxylic acid (Compound 223);
1-[N-morpholinocarbonylmethyl]-2-[2-thien-2-ylquinolin-6-yl]-3-cyclohexyl-1H-indole-6-carboxylic acid (Compound 224);
1-[N-morpholinocarbonylmethyl]-2-[2-(5-chlorothien-2-yl)quinolin-6-yl]-3-cyclohexyl-1H-indole-6-carboxylic acid (Compound 225);
1-[N-morpholinocarbonylmethyl]-2-[2-pyrid-3-ylquinolin-6-yl]-3-cyclohexyl-1H-indole-6-carboxylic acid (Compound 226);
1-[N-morpholinocarbonylmethyl]-2-[2-thiazol-2-ylquinolin-6-yl]-3-cyclohexyl-1H-indole-6-carboxylic acid (Compound 227);
1-[N-morpholinocarbonylmethyl]-2-[2-thien-3-ylquinolin-6-yl]-3-cyclohexyl-1H-indole-6-carboxylic acid (Compound 228);
3-Cyclohexyl-2-[2-(3-methoxy-phenyl)-quinolin-6-yl]-1-(2-morpholin-4-yl-2-oxo-ethyl)-1H-indole-6-carboxylic acid;
3-Cyclohexyl-2-[2-(3-methyl-thiophen-2-yl)-quinolin-6-yl]-1-(2-morpholin-4-yl-2-oxo-ethyl)-1H-indole-6-carboxylic acid;
3-Cyclohexyl-2-[2-(2,5-dimethyl-furan-3-yl)-quinolin-6-yl]-1-(2-morpholin-4-yl-2-oxo-ethyl)-1H-indole-6-carboxylic acid;
3-Cyclohexyl-1-(2-morpholin-4-yl-2-oxo-ethyl)-2-(2-m-tolyl-quinolin-6-yl)-1H-indole-6-carboxylic acid;
3-Cyclohexyl-1-(2-morpholin-4-yl-2-oxo-ethyl)-2-(2-o-tolyl-quinolin-6-yl)-1H-indole-6-carboxylic acid;
3-Cyclohexyl-2-[2-(2-methoxy-phenyl)-quinolin-6-yl]-1-(2-morpholin-4-yl-2-oxo-ethyl)-1H-indole-6-carboxylic acid;
3-Cyclohexyl-2-[2-(4-methyl-thiophen-2-yl)-quinolin-6-yl]-1-(2-morpholin-4-yl-2-oxo-ethyl)-1H-indole-6-carboxylic acid;
3-Cyclohexyl-2-[2-(5-methyl-thiophen-2-yl)-quinolin-6-yl]-1-(2-morpholin-4-yl-2-oxo-ethyl)-1H-indole-6-carboxylic acid;
3-Cyclohexyl-2-[2-(2,4-dimethyl-thiazol-5-yl)-quinolin-6-yl]-1-[2-(4-hydroxy-piperidin-1-yl)-2-oxo-ethyl]-1H-indole-6-carboxylic acid;
3-Cyclohexyl-2-[2-(2,4-dimethyl-thiazol-5-yl)-quinolin-6-yl]-1-[(2-morpholin-4-yl-ethylcarbamoyl)-methyl]-1H-indole-6-carboxylic acid;
3-Cyclohexyl-2-[2-(2,4-dimethyl-thiazol-5-yl)-quinolin-6-yl]-7-methyl-1-(2-morpholin-4-yl-2-oxo-ethyl)-1H-indole-6-carboxylic acid;
3-Cyclohexyl-2-[2-(2,4-dimethyl-thiazol-5-yl)-quinolin-6-yl]-1-[(2H-tetrazol-5-ylcarbamoyl)-methyl]-1H-indole-6-carboxylic acid;
N-[3-Cyclohexyl-2-[2-(2,4-dimethyl-thiazol-5-yl)-quinolin-6-yl]-1-(2-morpholin-4-yl-2-oxo-ethyl)-1H-indole-6-carbonyl]-methanesulfonamide;
1-[2-(4-Carboxy-piperidin-1-yl)-2-oxo-ethyl]-3-cyclohexyl-2-[2-(2,4-dimethyl-thiazol-5-yl)-quinolin-6-yl]-1H-indole-6-carboxylic acid;
3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid (2-cyano-ethyl)-amide;
3-Cyclohexyl-2-[2-(2,4-dimethyl-thiazol-5-yl)-quinolin-6-yl]-1-(2-morpholin-4-yl-2-oxo-ethyl)-1H-indole-6-carbonitrile;
2-[3-Cyclohexyl-2-[2-(2,4-dimethyl-thiazol-5-yl)-quinolin-6-yl]-6-(1H-tetrazol-5-yl)-indol-1-yl]-1-morpholin-4-yl-ethanone;
3-Cyclohexyl-2-[2-(2,4-dimethyl-thiazol-5-yl)-quinolin-6-yl]-1-(2-morpholin-4-yl-2-oxo-ethyl)-1H-indole-6-carboxylic acid [2-(1H-tetrazol-5-yl)-ethyl]-amide;
3-Cyclohexyl-1-(2-morpholin-4-yl-2-oxo-ethyl)-2-[2-(3-trifluoromethoxy-phenyl)-quinolin-6-yl]-1H-indole-6-carboxylic acid;
3-Cyclohexyl-1-(2-morpholin-4-yl-2-oxo-ethyl)-2-[2-(3-trifluoromethyl-phenyl)-quinolin-6-yl]-1H-indole-6-carboxylic acid;
3-Cyclohexyl-2-[2-(4-methyl-2-trifluoromethyl-thiazol-5-yl)-quinolin-6-yl]-1-(2-morpholin-4-yl-2-oxo-ethyl)-1H-indole-6-carboxylic acid;
3-Cyclohexyl-2-[2-(4-methyl-pyridin-2-yl)-quinolin-6-yl]-1-(2-morpholin-4-yl-2-oxo-ethyl)-1H-indole-6-carboxylic acid;
3-Cyclohexyl-2-[2-(3,4-dimethyl-phenyl)-quinolin-6-yl]-1-(2-morpholin-4-yl-2-oxo-ethyl)-1H-indole-6-carboxylic acid;
3-Cyclohexyl-2-[2-(3,5-dimethoxy-phenyl)-quinolin-6-yl]-1-(2-morpholin-4-yl-2-oxo-ethyl)-1H-indole-6-carboxylic acid;
3-Cyclohexyl-1-(2-morpholin-4-yl-2-oxo-ethyl)-2-(2-p-tolyl-quinolin-6-yl)-1H-indole-6-carboxylic acid;
3-Cyclohexyl-2-[2-(3,4-dimethoxy-phenyl)-quinolin-6-yl]-1-(2-morpholin-4-yl-2-oxo-ethyl)-1H-indole-6-carboxylic acid;
3-Cyclohexyl-2-[2-(4-methoxy-phenyl)-quinolin-6-yl]-1-(2-morpholin-4-yl-2-oxo-ethyl)-1H-indole-6-carboxylic acid;
3-Cyclohexyl-2-[2-(2-fluoro-phenyl)-quinolin-6-yl]-1-(2-morpholin-4-yl-2-oxo-ethyl)-1H-indole-6-carboxylic acid;
3-Cyclohexyl-2-[2-(3-nitro-phenyl)-quinolin-6-yl]-1-(2-morpholin-4-yl-2-oxo-ethyl)-1H-indole-6-carboxylic acid;
3-Cyclohexyl-2-[2-(2-fluoro-4-methoxy-phenyl)-quinolin-6-yl]-1-(2-morpholin-4-yl-2-oxo-ethyl)-1H-indole-6-carboxylic acid;
3-Cyclohexyl-2-[2-(2,5-dimethyl-thiophen-3-yl)-quinolin-6-yl]-1-(2-morpholin-4-yl-2-oxo-ethyl)-1H-indole-6-carboxylic acid;
3-Cyclohexyl-2-[2-(2,6-difluoro-phenyl)-quinolin-6-yl]-1-(2-morpholin-4-yl-2-oxo-ethyl)-1H-indole-6-carboxylic acid;
3-Cyclohexyl-2-[2-(2,4-dimethyl-oxazol-5-yl)-quinolin-6-yl]-1-(2-morpholin-4-yl-2-oxo-ethyl)-1H-indole-6-carboxylic acid;
3-Cyclohexyl-2-[2-(3-fluoro-phenyl)-quinolin-6-yl]-1-(2-morpholin-4-yl-2-oxo-ethyl)-1H-indole-6-carboxylic acid;
2-[2-(3-Bromo-phenyl)-quinolin-6-yl]-3-cyclohexyl-1-(2-morpholin-4-yl-2-oxo-ethyl)-1H-indole-6-carboxylic acid;
3-Cyclohexyl-1-(2-morpholin-4-yl-2-oxo-ethyl)-2-[2-(4-trifluoromethyl-phenyl)-quinolin-6-yl]-1H-indole-6-carboxylic acid;
2-[2-(3-Amino-phenyl)-quinolin-6-yl]-3-cyclohexyl-1-(2-morpholin-4-yl-2-oxo-ethyl)-1H-indole-6-carboxylic acid;
3-Cyclohexyl-2-[2-(4-fluoro-phenyl)-quinolin-6-yl]-1-(2-morpholin-4-yl-2-oxo-ethyl)-1H-indole-6-carboxylic acid;
3-Cyclohexyl-2-[2-(3,4-difluoro-phenyl)-quinolin-6-yl]-1-(2-morpholin-4-yl-2-oxo-ethyl)-1H-indole-6-carboxylic acid;
3-Cyclohexyl-1-(2-morpholin-4-yl-2-oxo-ethyl)-2-[2-(2-trifluoromethyl-phenyl)-quinolin-6-yl]-1H-indole-6-carboxylic acid;
3-Cyclohexyl-2-[2-(3-methyl-pyrazin-2-yl)-quinolin-6-yl]-1-(2-morpholin-4-yl-2-oxo-ethyl)-1H-indole-6-carboxylic acid;
3-Cyclohexyl-2-[2-(2-ethoxy-4-methyl-pyrimidin-5-yl)-quinolin-6-yl]-1-(2-morpholin-4-yl-2-oxo-ethyl)-1H-indole-6-carboxylic acid;
3-Cyclohexyl-2-[2-(2-fluoro-5-methoxy-phenyl)-quinolin-6-yl]-1-(2-morpholin-4-yl-2-oxo-ethyl)-1H-indole-6-carboxylic acid;
3-Cyclohexyl-2-[2-(1-methyl-1H-pyrrol-2-yl)-quinolin-6-yl]-1-(2-morpholin-4-yl-2-oxo-ethyl)-1H-indole-6-carboxylic acid;
3-Cyclohexyl-1-(2-morpholin-4-yl-2-oxo-ethyl)-2-[2-(2,3,4-trimethoxy-phenyl)-quinolin-6-yl]-1H-indole-6-carboxylic acid;
3-Cyclohexyl-2-[2-(3-fluoro-4-methoxy-phenyl)-quinolin-6-yl]-1-(2-morpholin-4-yl-2-oxo-ethyl)-1H-indole-6-carboxylic acid;
3-Cyclohexyl-1-[2-(4-dimethylamino-piperidin-1-yl)-2-oxo-ethyl]-2-[2-(2,4-dimethyl-thiazol-5-yl)-quinolin-6-yl]-1H-indole-6-carboxylic acid;
3-Cyclohexyl-1-[2-(4-diethylamino-piperidin-1-yl)-2-oxo-ethyl]-2-[2-(2,4-dimethyl-thiazol-5-yl)-quinolin-6-yl]-1H-indole-6-carboxylic acid;
2-[2-(2-Chloro-phenyl)-quinolin-6-yl]-3-cyclohexyl-1-(2-morpholin-4-yl-2-oxo-ethyl)-1H-indole-6-carboxylic acid;
3-Cyclohexyl-2-[2-(2,4-dimethyl-thiazol-5-yl)-quinolin-6-yl]-1-[2-(2-methyl-pyrrolidin-1-yl)-2-oxo-ethyl]-1H-indole-6-carboxylic acid;
3-Cyclohexyl-2-[2-(2,4-dimethyl-thiazol-5-yl)-quinolin-6-yl]-1-[2-(4-morpholin-4-yl-piperidin-1-yl)-2-oxo-ethyl]-1H-indole-6-carboxylic acid;
3-Cyclohexyl-1-[2-(3,5-dimethyl-morpholin-4-yl)-2-oxo-ethyl]-2-[2-(2,4-dimethyl-thiazol-5-yl)-quinolin-6-yl]-1H-indole-6-carboxylic acid;
3-Cyclohexyl-2-[2-(2,4-dimethyl-thiazol-5-yl)-quinolin-6-yl]-1-[2-(4-methyl-piperidin-1-yl)-2-oxo-ethyl]-1H-indole-6-carboxylic acid;
3-Cyclohexyl-2-[2-(2,4-dimethyl-3-oxy-thiazol-5-yl)-quinolin-6-yl]-1-(2-morpholin-4-yl-2-oxo-ethyl)-1H-indole-6-carboxylic acid;
2-[8-Bromo-2-(2,4-dimethyl-thiazol-5-yl)-quinolin-6-yl]-3-cyclohexyl-1-(2-morpholin-4-yl-2-oxo-ethyl)-1H-indole-6-carboxylic acid;
2-[8-amino-2-(2,4-dimethyl-thiazol-5-yl)-quinolin-6-yl]-3-cyclohexyl-1-(2-morpholin-4-yl-2-oxo-ethyl)-1H-indole-6-carboxylic acid
3-Cyclohexyl-2-[2-(2,4-dimethyl-thiazol-5-yl)-quinolin-6-yl]-1-(2-morpholin-4-yl-2-oxo-ethyl)-1H-indole-6-(β-D-glucuronic acid)ester;
2-[8-nitro-2-(2,4-dimethyl-thiazol-5-yl)-quinolin-6-yl]-3-cyclohexyl-1-(2-morpholin-4-yl-2-oxo-ethyl)-1H-indole-6-carboxylic acid
3-Cyclohexyl-1-(3-dimethylamino-propyl)-2-[2-(2,4-dimethyl-thiazol-5-yl)-quinolin-6-yl]-1H-indole-6-carboxylic acid;
1-Benzyl-3-cyclohexyl-2-[2-(2,4-dimethyl-thiazol-5-yl)-quinolin-6-yl]-1H-indole-6-carboxylic acid;
3-Cyclohexyl-2-[2-(2,4-dimethyl-thiazol-5-yl)-quinolin-6-yl]-1-pyridin-4-ylmethyl-1H-indole-6-carboxylic acid;
3-Cyclohexyl-2-[2-(2,4-dimethyl-thiazol-5-yl)-quinolin-6-yl]-1-(2-morpholin-4-yl-ethyl)-1H-indole-6-carboxylic acid;
3-Cyclohexyl-2-[2-(2,4-dimethyl-thiazol-5-yl)-quinolin-6-yl]-1-(5-methyl-isoxazol-3-ylmethyl)-1H-indole-6-carboxylic acid;
1-(4-Carboxy-benzyl)-3-cyclohexyl-2-[2-(2,4-dimethyl-thiazol-5-yl)-quinolin-6-yl]-1H-indole-6-carboxylic acid;
3-Cyclohexyl-2-[2-(2,4-dimethyl-thiazol-5-yl)-quinolin-6-yl]-1-(3-methoxy-benzyl)-1H-indole-6-carboxylic acid;
2-[4-(6-Bromo-quinolin-2-yl)-phenyl]-3-cyclohexyl-1-(2-morpholin-4-yl-2-oxo-ethyl)-1H-indole-6-carboxylic acid;
3-Cyclohexyl-2-(4′-dimethylamino-biphenyl-4-yl)-1-(2-morpholin-4-yl-2-oxo-ethyl)-1H-indole-6-carboxylic acid;
3-Cyclohexyl-2-(4′-methyl-biphenyl-4-yl)-1-(2-morpholin-4-yl-2-oxo-ethyl)-1H-indole-6-carboxylic acid;
3-Cyclohexyl-2-(4′-methoxy-biphenyl-4-yl)-1-(2-morpholin-4-yl-2-oxo-ethyl)-1H-indole-6-carboxylic acid;
3-Cyclohexyl-2-(2′-fluoro-biphenyl-4-yl)-1-(2-morpholin-4-yl-2-oxo-ethyl)-1H-indole-6-carboxylic acid;
3-Cyclohexyl-2-[4-(2-fluoro-pyridin-3-yl)-phenyl]-1-(2-morpholin-4-yl-2-oxo-ethyl)-1H-indole-6-carboxylic acid;
3-Cyclohexyl-2-[4-(2-methoxy-pyridin-3-yl)-phenyl]-1-(2-morpholin-4-yl-2-oxo-ethyl)-1H-indole-6-carboxylic acid;
3-Cyclohexyl-2-[4-(4-methoxy-pyridin-3-yl)-phenyl]-1-(2-morpholin-4-yl-2-oxo-ethyl)-1H-indole-6-carboxylic acid;
2-(3′-Cyano-biphenyl-4-yl)-3-cyclohexyl-1-(2-morpholin-4-yl-2-oxo-ethyl)-1H-indole-6-carboxylic acid;
2-(4′-Cyano-biphenyl-4-yl)-3-cyclohexyl-1-(2-morpholin-4-yl-2-oxo-ethyl)-1H-indole-6-carboxylic acid;
1-Carboxymethyl-3-cyclohexyl-2-[4-(4-methoxy-pyridin-3-yl)-phenyl]-1H-indole-6-carboxylic acid;
3-Cyclohexyl-2-(3′-methoxy-biphenyl-4-yl)-1-(2-morpholin-4-yl-2-oxo-ethyl)-1H-indole-6-carboxylic acid;
3-Cyclohexyl-2-(3′-nitro-biphenyl-4-yl)-1-(2-morpholin-4-yl-2-oxo-ethyl)-1H-indole-6-carboxylic acid;
3-Cyclohexyl-2-(2′-methoxy-biphenyl-4-yl)-1-(2-morpholin-4-yl-2-oxo-ethyl)-1H-indole-6-carboxylic acid;
3-Cyclohexyl-2-(3′-methyl-biphenyl-4-yl)-1-(2-morpholin-4-yl-2-oxo-ethyl)-1H-indole-6-carboxylic acid;
3-Cyclohexyl-2-(2′-methyl-biphenyl-4-yl)-1-(2-morpholin-4-yl-2-oxo-ethyl)-1H-indole-6-carboxylic acid;
3-Cyclohexyl-1-(2-morpholin-4-yl-2-oxo-ethyl)-2-(4′-vinyl-biphenyl-4-yl)-1H-indole-6-carboxylic acid;
2-(3′-Amino-biphenyl-4-yl)-3-cyclohexyl-1-(2-morpholin-4-yl-2-oxo-ethyl)-1H-indole-6-carboxylic acid;
3-Cyclohexyl-2-[4-(5-methyl-thiophen-2-yl)-phenyl]-1-(2-morpholin-4-yl-2-oxo-ethyl)-1H-indole-6-carboxylic acid;
3-Cyclohexyl-2-[4-(3,5-dimethyl-isoxazol-4-yl)-phenyl]-1-(2-morpholin-4-yl-2-oxo-ethyl)-1H-indole-6-carboxylic acid;
2-[4-(5-Chloro-thiophen-2-yl)-phenyl]-3-cyclohexyl-1-(2-morpholin-4-yl-2-oxo-ethyl)-1H-indole-6-carboxylic acid;
3-Cyclohexyl-2-[7-(2,4-dimethyl-thiazol-5-yl)-[1,8]naphthyridin-3-yl]-1-(2-morpholin-4-yl-2-oxo-ethyl)-1H-indole-6-carboxylic acid;
3-Cyclohexyl-2-[2-(2,4-dimethyl-thiazol-5-yl)-3H-benzoimidazol-5-yl]-1-(2-morpholin-4-yl-2-oxo-ethyl)-1H-indole-6-carboxylic acid;
3-Cyclohexyl-1-(2-morpholin-4-yl-2-oxo-ethyl)-2-(6-m-tolyl-naphthalen-2-yl)-1H-indole-6-carboxylic acid;
3-Cyclohexyl-2-[6-(2-fluoro-phenyl)-naphthalen-2-yl]-1-(2-morpholin-4-yl-2-oxo-ethyl)-1H-indole-6-carboxylic acid;
3-Cyclohexyl-2-[2-(2,4-dimethyl-thiazol-5-yl)-1,2,3,4-tetrahydro-quinolin-6-yl]-1-(2-morpholin-4-yl-2-oxo-ethyl)-1H-indole-6-carboxylic acid;
3-Cyclohexyl-2-[2-(2-fluoro-phenyl)-quinoxalin-6-yl]-1-(2-morpholin-4-yl-2-oxo-ethyl)-1H-indole-6-carboxylic acid;
3-cyclohexyl-2-[3-(2-fluoro-phenyl)-quinoxalin-6-yl]-1-(2-morpholin-4-yl-2-oxo-ethyl)-1H-indole-6-carboxylic acid;
3-Cyclohexyl-1-(2-morpholin-4-yl-2-oxo-ethyl)-2-(2-pyridin-4-yl-quinolin-6-yl)-1H-indole-6-carboxylic acid;
3-Cyclohexyl-1-(2-morpholin-4-yl-2-oxo-ethyl)-2-(4-quinolin-2-yl-phenyl)-1H-indole-6-carboxylic acid;
3-Cyclohexyl-1-(2-morpholin-4-yl-2-oxo-ethyl)-2-(3-phenyl-quinolin-6-yl)-1H-indole-6-carboxylic acid;
3-Cyclohexyl-1-(2-morpholin-4-yl-2-oxo-ethyl)-2-(6-phenyl-naphthalen-2-yl)-1H-indole-6-carboxylic acid;
3-Cyclohexyl-1-(2-morpholin-4-yl-2-oxo-ethyl)-2-(3-phenyl-quinoxalin-6-yl)-1H-indole-6-carboxylic acid;
3-Cyclohexyl-1-(2-morpholin-4-yl-2-oxo-ethyl)-2-(2-phenyl-quinoxalin-6-yl)-1H-indole-6-carboxylic acid;
3-Cyclohexyl-1-(2-morpholin-4-yl-2-oxo-ethyl)-2-(2-thiazol-5-yl-quinolin-6-yl)-1H-indole-6-carboxylic acid;
2-Biphenyl-4-yl-3-cyclohexyl-1-(2-morpholin-4-yl-2-oxo-ethyl)-1H-indole-6-carboxylic acid;
3-Cyclohexyl-2-[2-(2,4-dimethyl-thiazol-5-yl)-quinolin-6-yl]-1H-indole-6-carboxylic acid;
3-Cyclohexyl-2-[2-(2,4-dimethyl-thiazol-5-yl)-quinolin-6-yl]-7-methyl-1H-indole-6-carboxylic acid;
2-[8-fluoro-2-(2,4-dimethyl-thiazol-5-yl)-quinolin-6-yl]-3-cyclohexyl-1-(2-morpholin-4-yl-2-oxo-ethyl)-1H-indole-6-carboxylic acid;
2-[7-fluoro-2-(2,4-dimethyl-thiazol-5-yl)-quinolin-6-yl]-3-cyclohexyl-1-(2-morpholin-4-yl-2-oxo-ethyl)-1H-indole-6-carboxylic acid; and
a tautomer, pharmaceutically acceptable salt, or partial salt thereof.

47. A pharmaceutical composition comprising a pharmaceutically acceptable carrier and a therapeutically effective amount of a compound of any one of claims 1, 27, 45, or 46 or a mixture of two or more of such compounds.

48. A method for treating or preventing a viral infection in a mammal mediated at least in part by a virus in the Flaviviridae family of viruses, comprising administering to said mammal a composition of claim 47.

49. The method of claim 48 in combination with a therapeutically effective amount of one or more agents active against hepatitis C virus.

50. The method of claim 49 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

51. The method of claim 49 wherein said active agent against HCV is Ribavirin, levovirin, viramidine, thymosin alpha-1, an inhibitor of NS3 serine protease, and inhibitor of inosine monophosphate dehydrogenase, interferon-alpha, or pegylated interferon-alpha, alone or in combination with Ribavirin or viramidine.

52. The method of claim 51 wherein said agent active against HCV is interferon-alpha or pegylated interferon-alpha alone or in combination with Ribavirin or viramidine.

53. The method of claim 48 wherein said mammal is a human.

54. A method for preparing a compound, pharmaceutically acceptable salt, partial salt, or tautomer of formula Ia 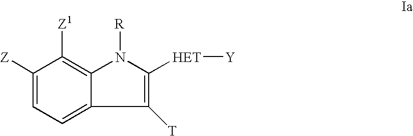
wherein the method comprises
(a) reacting a compound having formula IV 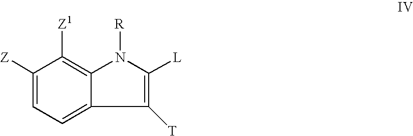
with L′-HET-Y in the presence of a transition metal catalyst, wherein P is a H or a nitrogen protecting group and one of L and L′ is halo and the other of L and L′ is a B(R³⁰)₂ or Sn(R³¹)₃ where R³⁰ is independently hydroxy, alkoxy, halo, or a suitable boron ligand and R³¹ is independently alkyl or aryl; and optionally
(b) reacting a compound having formula V 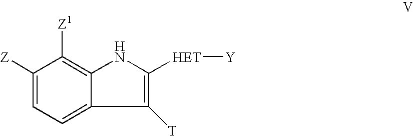
with L″-R, wherein
L″ is halo or —OSO₂R³ 2;
R³² is alkyl, substituted alkyl, aryl, or substituted aryl;
Y is selected from the group consisting of substituted aryl and substituted heteroaryl;
HET is selected from the group consisting of a 6-membered arylene ring, a 6-membered heteroarylene ring containing 1, 2, or 3 heteroatoms selected from N, O, or S, and a bicyclic ring having the formula 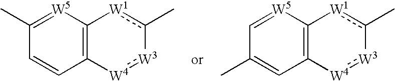
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;
R is selected from the group consisting of hydrogen, alkyl, and substituted alkyl;
T is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl;
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic substituted heterocyclic, and NR²²R²³ wherein each R²¹, R²² and R²³ is hydrogen, alkyl, substituted alkyl, cycloalkyl, or substituted cycloalkyl, and wherein each R²¹ and R²² is optionally substituted with one to three groups selected from halo, hydroxy, carboxy, carboxy ester, alkyl, alkoxy, amino, and substituted amino; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere, wherein said isostere is not as defined in (a)-(e);
Z¹ is selected from the group consisting of hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino.

55. An intermediate compound of claim 54 having the formula VI or VII 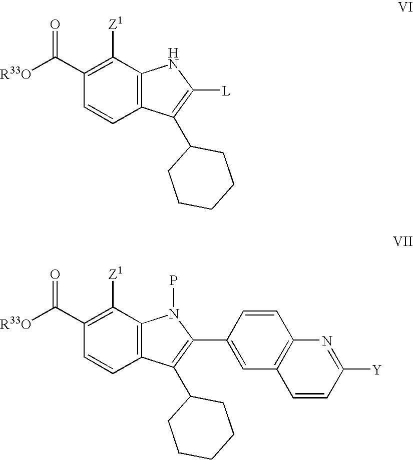
wherein R³³ is alkyl or arylalkyl;
Z¹ is selected from the group consisting of hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;
L is halo;
P is H or a nitrogen protecting group; and
Y is substituted aryl or substituted heteroaryl.
